(19) United States
(12) Patent Application Publication
(10) Pub. No.: US 2010/0298372 A1
(43) Pub. Date: Nov. 25, 2010

(54) GAMMA SECRETASE MODULATORS

(75) Inventors: Xianhai Huang, Warren, NJ (US); Robert G. Aslanian, Rockaway, NJ (US); Anandan Palani, Bridgewater, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/747,001

(22) PCT Filed: Dec. 9, 2008
(86) PCT No.: PCT/US08/86030

§ 371 Date: Jul. 27, 2010

Related U.S. Application Data
(60) Provisional application No. 61/012,863, filed on Dec. 11, 2007.

(51) Int. Cl. A61K 31/4375; C07D 455/02; A61P 25/28

(52) U.S. Cl. 514/306; 546/138

(57) ABSTRACT

This invention provides novel compounds that are modulators of gamma secretase. The compounds have the formula (I). Also disclosed are methods of modulating gamma secretase activity and methods of treating Alzheimer's disease using the compounds of formula (I).

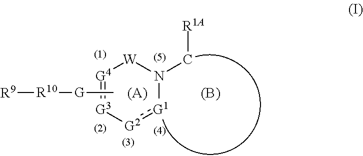

### REFERENCE TO RELATED APPLICATION

This application claims the benefit of U.S. Provisional Application Ser. No. 61/012,863 filed Dec. 11, 2007.

### FIELD OF THE INVENTION

The present invention relates to certain heterocyclic compounds useful as gamma secretase modulators (including inhibitors, antagonists and the like), pharmaceutical compositions containing the compounds, and methods of treatment using the compounds and compositions to treat various diseases including central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100(IB), p. 10417-22, suggest a molecular basis for reversible memory loss.

Nitsch R M, and 16 others, Antibodies against β-amyloid slow cognitive decline in Alzheimer's disease, Neuron, May 22, 2003, 38(4), p. 547-554) suggest that the main components of Aβ protein are Aβ40 consisting of 40 amino acids and Aβ42 having two additional amino acids at the C-terminal. The Aβ40 and Aβ42 tend to aggregate (for example, see Jarrell J T et al, The carboxy terminus of the β amyloid protein is critical for the seeding of amyloid formation: implications for the pathogenesis of Alzheimer's disease, Biochemistry, May 11, 1993, 32(IB), p. 4693-4697) and constitute the main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249.).

Furthermore, it is known that mutations of APP and presenelin genes, which is are observed in familial Alzheimer's disease, increase production of Aβ40 and Aβ42 (for example, see Gouras G K, et al, Intraneuronal Aβ42 accumulation in human brain, American Journal of Pathology, January 2000, 156(1), p. 15-20. Also, see Scheuner D, et al, Nature Medicine, August 1996, 2(8), p, 864-870; and Forman M S, et al, Differential effects of the Swedish mutant amyloid precursor protein on β-amyloid accumulation and secretion in neurons and nonneuronal cells, Journal of Biological Chemistry, Dec. 19, 1997, 272(51), p. 32247-32253.). Therefore, compounds which reduce production of Aβ40 and Aβ42 are expected to be agents for controlling progress of Alzheimer's disease or for preventing the disease.

These Aβs is are produced when APP is cleaved by beta secretase and subsequently cleaved by gamma secretase. In consideration of this, creation of inhibitors of γ-secretase and β-secretase has been attempted for the purpose of reducing production of Aβs. Many of these known secretase inhibitors are peptides or peptidomimetics such as L-685,458. L-685,458, an aspartyl protease transition state mimic, is a potent inhibitor of γ-secretase activity, Biochemistry, Aug. 1, 2000, 39(30), p, 8698-8704).

Also of interest in connection with the present invention are: US 2007/0117798 (Eisai, published May 24, 2007); US 2007/0117839 (Eisai, published May 24, 2007); US 2006/0004013 (Eisai, published Jan. 5, 2006); WO 20051110422 (Boehringer Ingelheim, published Nov. 24, 2005); WO 2006/045554 (Cellzone A G, published May 4, 2006); WO 2004/110350 (Neurogenetics, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with Aβ. It is, therefore, an object of this invention to provide compounds useful in the treatment or prevention or amelioration of such diseases and disorders.

### SUMMARY OF THE INVENTION

In its many embodiments, the present invention provides a novel class of compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

This invention provides novel compounds, that are gamma secretase modulators, of the formula:

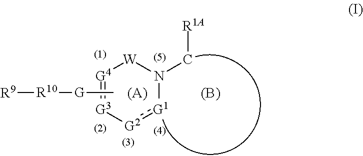

or a pharmaceutically acceptable salt, ester, or solvate thereof, wherein all substituents are defined below.

This invention also provides compounds of formula (I).

This invention also provides compounds of formula (I) in pure and isolated form.

This invention also provides compounds of formula (I) selected from the group consisting of: compounds of formulas IA to IH, 2 to 9, 12 to 18, 20, 21, 40 to 43, 55, 2A to 9A, 12A to 18A, 20A, 21A, 40A to 43A, 55A, 2B to 9B, 12B to 18B, 20B, 21B, 40B to 43B, 55B, 2C to 9C, 12C to 18C, 20C, 21C, 40C to 43C, 55C, 6.2, 9.1, 10.1, 10.2, 10.3, 14.1, 16.1, 16.2, 18.1, 19.1, 20.2, 21.2, 23.2, 25.1, 26.1, 27.1, 28.1, 30.1, 36.1, 37.1, 38.1, 39.1, 41.1, 43.1, 45.1, 46.1, 47.1, 48.1, 49.1, 50.1, 51.1, 52.1, 59.1, 60.1, 61.1, 64.1, 65.1, 68.1, 70.1, E1, E2, and E3.

This invention also provides compounds of formula (I) selected from the group consisting of: compounds of formulas IA to IH.

This invention also provides compounds of formula (I) selected from the group consisting of: compounds of formulas 2 to 9, 12 to 18, 20, 21, 40 to 43, and 55.

This invention also provides compounds of formula (I) selected from the group consisting of: compounds of formulas 2A to 9A, 12A to 18A, 20A, 21A, 40A to 43A, and 55A.

This invention also provides compounds of formula (I) selected from the group consisting of: compounds of formulas 2B to 9B, 12B to 18B, 20B, 21B, 40B to 43B, and 55B.

This invention also provides compounds of formula (I) selected from the group consisting of: compounds of formulas 2C to 9C, 12C to 18C, 20C, 21C, 40C to 43C, and 55C.

This invention also provides compounds of formula (I) selected from the group consisting of: compounds of formulas 6.2, 9.1, 10.1, 10.2, 10.3, 14.1, 16.1, 16.2, 18.1, 19.1, 20.2, 21.2, 23.2, 25.1, 26.1, 27.1, 28.1, 30.1, 36.1, 37.1, 38.1, 39.1, 41.1, 43.1, 45.1, 46.1, 47.1, 48.1, 49.1, 50.1, 51.1, 52.1, 59.1, 60.1, 61.1, 64.1, 65.1, 68.1, and 70.1.

This invention also provides compounds of formula (I) selected from the group consisting of: compounds of formulas E1, E2, and E3.

This invention also provides pharmaceutical compositions comprising an effective amount of one or more (e.g., one) compounds of formula (I), or a pharmaceutically acceptable salt, ester or solvate thereof, and a pharmaceutically acceptable carrier.

This invention also provides pharmaceutical compositions comprising an effective amount of one or more (e.g., one) compounds of formula (I), or a pharmaceutically acceptable salt, ester or solvate thereof, and an effective amount of one or more (e.g., one) other pharmaceutically active ingredients (e.g., drugs), and a pharmaceutically acceptable carrier.

The compounds of formula (I) can be useful as gamma secretase modulators and can be useful in the treatment and prevention of diseases such as, for example, central nervous system disorders such as Alzheimers disease and Downs Syndrome.

Thus, this invention also provides methods for: (1) method for modulating (including inhibiting, antagonizing and the like) gamma-secretase; (2) treating one or more neurodegenerative diseases; (3) inhibiting the deposition of amyloid protein (e.g., amyloid beta protein) in, on or around neurological tissue (e.g., the brain); (4) Alzheimer's disease; and (5) treating Downs syndrome; wherein each method comprises administering an effective amount of one or more (e.g., one) compounds of formula (I) to a patient in need of such treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula (I) and the administration of an effective amount of one or more (e.g., one) other pharmaceutical active ingredients (e.g., drugs).

This invention also provides methods for: (1) treating mild cognitive impairment; (2) treating glaucoma; (3) treating cerebral amyloid angiopathy; (4) treating stroke; (5) treating dementia; (6) treating microgliosis; (7) treating brain inflammation; and (8) treating olfactory function loss; wherein each method comprises administering an effective amount of one or more (e.g., one) compounds of formula (I) to a patient in need of such treatment.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described below), the combined quantities of the compound of formula (I) and the other pharmaceutically active ingredient being effective to treat the diseases or conditions mentioned in any of the above methods.

This invention also provides any of the above mentioned methods, pharmaceutical compositions or kit wherein the compound of formula (I) is selected from the group consisting of: compounds of formulas IA to IH, 6.2, 10.2, 10.3, 20.2, 21.2, 23.2, 2 to 9, 12 to 18, 20, 21, 40 to 43, 55, 2A to 9A, 12A to 18A, 20A, 21A, 40A to 43A, 55A, 2B to 9B, 12B to 18B, 20B, 21B, 40B to 43B, 55B, 2C to 9C, 12C to 18C, 20C, 21C, 40C to 43C, 55C, E1, E2, and E3.

This invention also provides any of the above mentioned methods, pharmaceutical compositions or kit wherein the compound of formula (I) is selected from the group consisting of: compounds IA to IH.

This invention also provides any of the above mentioned methods, pharmaceutical compositions or kit wherein the compound of formula (I) is selected from the group consisting of: compounds 6.2, 10.2, 10.3, 20, 2, 21.2, and 23.2.

This invention also provides any of the above mentioned methods, pharmaceutical compositions or kit wherein the compound of formula (I) is selected from the group consisting of: compounds 2 to 9, 12 to 18, 20, 21, 40 to 43, and 55.

This invention also provides any of the above mentioned methods, pharmaceutical compositions or kit wherein the compound of formula (I) is selected from the group consisting of: compounds 2A to 9A, 12A to 18A, 20A, 21A, 40A to 43A, and 55A.

This invention also provides any of the above mentioned methods, pharmaceutical compositions or kit wherein the compound of formula (I) is selected from the group consisting of: compounds 2B to 9B, 12B to 18B, 20B, 21B, 40B to 43B, and 55B.

This invention also provides any of the above mentioned methods, pharmaceutical compositions or kit wherein the compound of formula (I) is selected from the group consisting of: compounds 2C to 9C, 12C to 18C, 20C, 21C, 40C to 43C, and 55C.

This invention also provides any of the above mentioned methods, pharmaceutical compositions or kit wherein the compound of formula (I) is selected from the group consisting of: compounds E1, E2, and E3.

### DETAILED DESCRIPTION OF THE INVENTION

This invention provides compounds, useful as gamma secretase modulators, of formula (I):

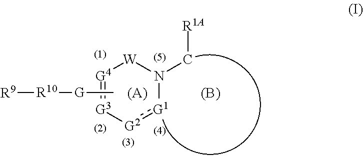

or a pharmaceutically acceptable salt, ester, or solvate thereof, wherein:

R^{1A}, G¹, G², G³, G⁴, (B), G, R⁹, R¹⁰, and W are independently selected;

letters (A) and (B) in formula (I) are reference letters to identify the rings present in formula (I);

the numbers (1), (2), (3), (4), and (5) are reference numbers to identify positions of the Ring (A); G⁴ is at position (1), G³ is at position (2), G² is at position (3), G¹ is at position (4) and the N is at position (5);

the moiety -G-R¹⁰-R⁹ is bound through G to G⁴ (i.e., position (1)) or G³ (i.e., position (2)), and when G is bound to G⁴ then G⁴ is a —C—, and when G is bound to G³ then G³ is a —C—;

the dotted line between G¹ and G² represents an optional bond;

Ring (B) is the ring formed from the N at position (5) and G¹, and G¹ is carbon or N (i.e., —N(R²)_{d}— wherein d is 0), and when G¹ is N the optional bond between G¹ and G² is absent;

said Ring (B) is a 4 to 8 (and in one example 5 to 6) membered heterocycloalkyl, heteroaryl, or heterocycloalkenyl ring;

said heterocycloalkyl, heterocycloalkenyl, or a heteroaryl ring Ring (B), in addition to the nitrogen common to Ring (A) and Ring (B), optionally comprises, at least one (e.g., 1 to 3, or 1 to 2, or 1) other heteroatom selected from the group consisting of: —NR²—, —O—, —S—, —S(O)—, and —S(O)₂—;

said Ring (B) is optionally substituted with 1 to 6 independently selected R²¹ substituents;

d is 0 or 1 (and those skilled in the art will appreciate that when d is 0 in the —N(R²)_{d}— moiety there is no substituent on the N, thus, the moiety —N(R²)_{d}— is —N═ or —NH— when d is 0, i.e., when d is 0 in a moiety there is the appropriate number of H atoms on the N to fill the required valences);

m is 0 to 6;

n is 1 to 5;

p is 0 to 5;

q is 0, 1 or 2, and each q is independently selected (and those skilled in the art will appreciate that when q is 0 in the moiety —C(R²¹)_{q} this means that there is no R²¹ substituent on the carbon, and the —C(R²¹)_{q} moiety is —CH═ or —CH₂—, i.e., when q is 0 in a moiety there is the appropriate number of H atoms on the carbon to fill the required valences);

r is 1 to 3;

t is 1 or 2;

W is selected from the group consisting of: —C(O)—, —S(O)₂—, —S(O)—, and —C(═NR²)— (and in one example W is —C(O)—);

G is selected from the group consisting of: a direct bond (i.e., R¹⁰ is bound directly to either G³ or G⁴), —C(O)—, —(C═NR²)—, —(C═C(R⁶)₂)—, —CHR³— (e.g., —CHOH), C(R⁴)₂, —CF₂—, —N(R²)— (and in one example, —NH—), —O—, —S—, —S(O)ₜ, —CR⁴(OH)—, —CR⁴(OR⁴)—, —C═C—, alkynyl, —(CH₂)ᵣN(R²)—, —(CHR⁴)ᵣN(R²)—, C(R⁴)₂)ᵣN(R²)—, —N(R²)(CH₂)ᵣ—, N(R²)(CHR⁴)ᵣ—, —N(R²)(C(R⁴)₂)ᵣ—, —(CH₂)ᵣ—O—, —(C(R⁴)₂)ᵣ—O—, —O—(CH₂)ᵣ—, O—(CHR⁴)ᵣ—, —O—(C(R⁴)₂)ᵣ—, —(CH₂)ᵣ—O—C(O)—, —(CHR⁴)ᵣ—O—C(O)—, —(C(R⁴)₂)ᵣ—O—C(O)—, —C(O)—O—(CH₂)ᵣ—, —C(O)—O—(CHR⁴)ᵣ—, —C(O)—O—(C(R⁴)₂)ᵣ—, —C(O)NR⁵—, —O—C(O)—, —C(O)—O, —C(O)—NR⁵—, —NR⁵C(O)—, —(CH₂)ᵣNR⁵—Cc(O)—, —(CHR⁴)ᵣNR⁵—C(O)—, —(C(R⁴)₂)ᵣNR⁵—C(O)—, —C(O)NR⁵(CH₂)ᵣ—C(O)NR⁵ (CHR⁴)ᵣ—, —C(O)NR⁵ (C(R⁴)₂)ᵣ—, —NR⁵S(O)ₜ—(CH₂)ᵣNR⁵S(O)ₜ—, —(CHR⁴)ᵣNR⁵S(O)ₜ—, —(C(R⁴)₂)ᵣNR⁵S(O)ₜ—, —S(O)ₜNR⁵—, —S(O)ₜNR⁵(CH₂)ᵣ—, —S(O)ₜNR⁵(CHR⁴)ᵣ—, —S(O)ₜNR⁵(C(R⁴)₂)ᵣ—, —NR⁵—C(O)—O—, —NR⁵—C(O)—NR^{S}—, —NR^{S}—S(O)ₜ—NR⁵—, —NR⁵—C(═NR²)—NR⁵—, —NR⁵—C(═NR²)—O—, —O—C(═NR²)—NR⁵—, —C(R⁴)═N—O—, —O—N═C(R⁴)—, —O—C(R⁴)═N—, —N═C(R⁴)—O—, —(CH₂)₂₋₃—(i.e., 2 to 3 —CH₂— groups), —(C(R⁴)₂)₂₋₃—(i.e., there are 2 to 3 —(C(R⁴)₂ groups), and —(CHR⁴)₂₋₃—(i.e., there are 2 to 3 —(CHR⁴)— groups), cycloalkyl (e.g., C₃ to C₁₀ cycloalkyl), heterocycloalkyl (comprising 1 to 4 heteroatoms independently selected from the group consisting of: —O—, —NR²—, —S—, —S(O)—, and —S(O)₂);

G¹ is selected from the group consisting of:
(1) —C(R²¹)_{q}— wherein q is 0 when the optional bond is present (i.e., G¹ is C),
(2) —C(R²¹)_{d}— wherein q is 1 when the optional bond is absent,
(3) —CH— when the optional bond is absent, and
(4) —N(R²)_{d}— wherein d is 0, and the optional bond is absent;

G² is selected from the group consisting of: a direct bond (i.e., G³ is bonded directly to G¹, and Ring A is a five membered ring), —C(R²¹)_{q}, —N(R)_{d}—, —C(O)—, S(O), S(O)₂, —C(N(R²)₂)—, and —C(═NR²)—; and with the provisos that:
(1) when the optional bond between G¹ and G² is not present (i.e., there is a single bond between G¹ and G²) then G² is not —C(N(R²)₂)—, and
(2) when the optional bond between G¹ and G² is present (i.e., there is a double bond between G¹ and G²), then:
   (a) q for the —C(R²¹)_{d} group is 0 or 1 (and when q is 0 then there is a H on the carbon), and
   (b) d for the —N(R²)_{d}— group is 0 (and there is no H on the N due to the double bond between G¹ and G²), and
   (c) G² is not a direct bond, —C(O)—, —C(═NR²)—)-, —S(O)₂, or S(O)—;

G³ is selected from the group consisting of: (a) —C(R²¹)_{q} wherein q is 0 (i.e., the —C(R²¹)_{q} is C and there are no valences to fill with a H atom), (b) —CH— (i.e., q is 0 and there is a valence to fill with a H), (c) —C(R²¹)_{q} wherein q is 1, and (d) —N(R²)_{d} wherein d is 0 (and there is no H on the N due to the double bond between G³ and G⁴); and with the proviso that: when moiety G is bound to G³, then G³ is carbon (i.e., the group G³ is the group —C(R²¹)_{q} wherein q is 0 and there is no valence to fill with a H atom);

G⁴ is selected from the group consisting of: (a) —C(R²¹)_{q} wherein q is 0 (i.e., the —C(R²¹)_{q} is C and there are no valences to fill with a H atom), (b) —CH— (i.e., q is 0 and there is a valence to fill with a H), (c) —C(R²¹)_{q} wherein q is 1, and (d) —N(R²)_{d} wherein d is 0 (and there is no H on the N due to the double bond between G³ and G⁴); and with the proviso that: when moiety G is bound to G⁴, then G⁴ is carbon (i.e., the group G⁴ is the group —C(R²¹)_{q} wherein q is 0 there is no valence to fill with a H atom); and

provided that 0 to 2 of the G¹, G², G³, and G⁴ moieties are —N(R²)_{d}— and each R² is independently selected and each d is independently selected, and provided that Ring (A) does not have three consecutive ring nitrogen atoms;

R^{1A} is selected from the group consisting of: alkyl-, alkenyl-, alkynyl-, aryl-, arylalkyl-, alkylaryl-, cycloalkyl, cycloalkenyl, cycloalkylalkyl-, fused benzocycloalkyl- (i.e., benzofusedcycloalkyl-), fused benzoheterocycloalkyl- (i.e., benzofusedheterocycloalkyl-), fused heteroarylcycloalkyl- (i.e., heteroarylfusedcycloalkyl-), fused heteroarylheterocycloalkyl- (i.e., heteroarylfusedheterocycloakyl-), fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-), fused heterocycloalkylheteroaryl- (i.e., heterocycloalkylfusedheteroarl-), fused benzocycloalkylalkyl- (i.e., benzofusedcycloalkylalkyl-), fused benzoheterocycloalkylalkyl- (i.e., benzofusedheterocycloalkylalkyl-), fused heteroarylcycloalkylalkyl- (i.e., heteroarylfusedcycloalkylalkyl-), fused heteroarylheterocycloalkylalkyl- (i.e., heteroarylfusedheterocycloalkylalkyl-), fused cycloalkylarylalkyl- (i.e., cycloalkyfusedlarylalkyl-), fused heterocycloalkylarylalkyl- (i.e., heterocycloalkylfusedarylalkyl-), fused cycloalkylheteroaryalkyl- (i.e., cycloalkylfusedheteroarylalkyl-), fused heterocycloalkylheteroarylalkyl- (i.e., heterocycloalkylfusedheteroarylalkyl-), heteroaryl-, heteroarylalkyl-, heterocyclyl-, heterocyclenyl-, and heterocyclyalkyl-; wherein each of said alkyl-, alkenyl-, alkynyl-, aryl-, arylalkyl-, alkylaryl-, cycloalkyl, cycloalkenyl, cycloalkylalkyl-, fused benzocycloalkyl, fused benzoheterocycloalkyl, fused heteroarylcycloalkyl, fused heteroarylheterocycloalkyl, fused cycloalkylaryl, fused heterocycloalkylaryl, fused cycloalkylheteroaryl, fused heterocycloalkylheteroaryl, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, fused heterocycloalkylheteroarylalkyl-, heteroaryl, heteroarylalkyl-, heterocyclyl, heterocyclenyl and heterocyclyalkyl-R^{1A} groups is optionally substituted with 1-5 independently selected R²¹ groups;

Each R² is independently selected from the group consisting of: H, —OH, —O-alkyl (i.e., alkoxy), —O-(halo substituted alkyl) (such as, for example, —O-fluoroalkyl), —NH(R⁴), —N(R⁴)₂ (wherein each R⁴ is independently selected), —NH₂, —S(O)R⁴, —S(O)(OR⁴), —S(O)₂R⁴, —S(O)₂(OR⁴), —S(O)NHR⁴, —S(O)N(R⁴)₂, —S(O)NH₂, —S(O)₂NHR⁴, —S(O)₂N(R⁴)₂, —S(O)₂NH₂, —CN, —C(O)₂R⁴, —C(O)NHR⁴, —C(O)N(R⁴)₂, —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;

R³ is selected from the group consisting of: H, —OH, halo, —O-alkyl (i.e., alkoxy), —O-(halo substituted alky) (such as, for example, —O-fluoroalkyl), —NH(R⁴), —N(R⁴)₂ (wherein each R⁴ is independently selected), —NH₂, —S(R⁴), —S(O)R⁴, —S(O)(OR⁴), —S(O)₂R⁴, —S(O)₂(OR⁴), —S(O)NHR⁴, —S(O)N(R⁴)₂, —S(O)NH₂, —S(O)₂NHR⁴, —S(O)₂N(R⁴)₂, —S(O)₂NH₂, —CN, —C(O)₂R⁴, —C(O)NHR⁴, —C(O)N(R⁴)₂, —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;

each R⁴ is independently selected from the group consisting of: unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl, heteroarylalkyl-, alkenyl, alkynyl and cycloalkyl groups are substituted with 1 to 5 independently selected R²¹ groups;

each R⁵ is independently selected from the group consisting of: H, unsubstituted alkyl, substituted alkyl, unsubstituted alkenyl, substituted alkenyl, unsubstituted alkynyl, substituted alkynyl, unsubstituted cycloalkyl, substituted cycloalkyl, unsubstituted aryl, substituted aryl, unsubstituted heteroaryl and substituted heteroaryl; wherein said substituted groups are substituted with one or more (e.g., 1 to 5) substituents independently selected from: R²;

Each R⁶ is independently selected from the group consisting of: H, halo, —CF₃, —O-alkyl (i.e., alkoxy), —O-(halo substituted alky) (such as, for example, —O-fluoroalkyl), —S(O)R⁴, —S(O)(OR⁴), —S(O)NHR⁴, —S(O)N(R⁴)₂ (wherein each R⁴ is independently selected), —S(O)NH₂, —S(O)₂NHR⁴, —S(O)₂N(R⁴)₂ (wherein each R⁴ is independently selected), —S(O)₂NH₂, —C(═O—NOR²⁴)R²⁵, and —S(O)₂R²⁴; —CN, —C(O)₂R⁴, —C(O)NHR⁴, —C(O)N(R⁴)₂ (wherein each R⁴ is independently selected), —C(O)NH₂, —C(O)R⁴, unsubstituted aryl, substituted aryl, unsubstituted heteroaryl, substituted heteroaryl, unsubstituted alkyl, substituted alkyl, unsubstituted aryalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, aryalkyl-, heteroarylalkyl-, alkenyl, alkynyl and cycloalkyl groups are substituted with 1 to 5 independently selected R²¹ groups;

R⁹ is selected from the group consisting of: arylalkoxy-, heteroarylalkoxy-, arylalkylamino-, heteroarylalkylamino-, aryl, aryalkyl-, heteroaryl, heteroarylalkyl-, heterocyclyl, heterocyclenyl, and heterocyclyalkyl-, wherein each of said R⁹ arylalkoxy-, heteroarylalkoxy-, arylalkylamino-, heteroarylalkylamino-, aryl, arylalkyl-, heteroaryl, heteroarylalkyl-, heterocyclyl, heterocyclenyl, and heterocyclyalkyl- is optionally substituted with 1-5 independently selected R²¹ groups;

R¹⁰ is selected from the group consisting of: aryl- (e.g., phenyl), heteroaryl- (e.g., pyridyl), cycloalkyl-, cycloalkenyl, cycloalkylalkyl-, heterocyclyl-, heterocyclenyl-, heterocyclylalkyl-, heterocyclylalkenyl-, fused benzocycloalkyl- (i.e., benzofusedcycloalkyl-), fused benzoheterocycloalkyl- (i.e., benzofusedheterocycloalkyl-), fused heteroarylcycloalkyl- (i.e., heteroarylfusedcycloalkyl-), fused heteroarylheterocycloalkyl- (i.e., heteroarylfusedheterocycloalkyl-), fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-), fused heterocycloalkylheteroaryl- (i.e., heterocycloalkylfusedheteroaryl-),

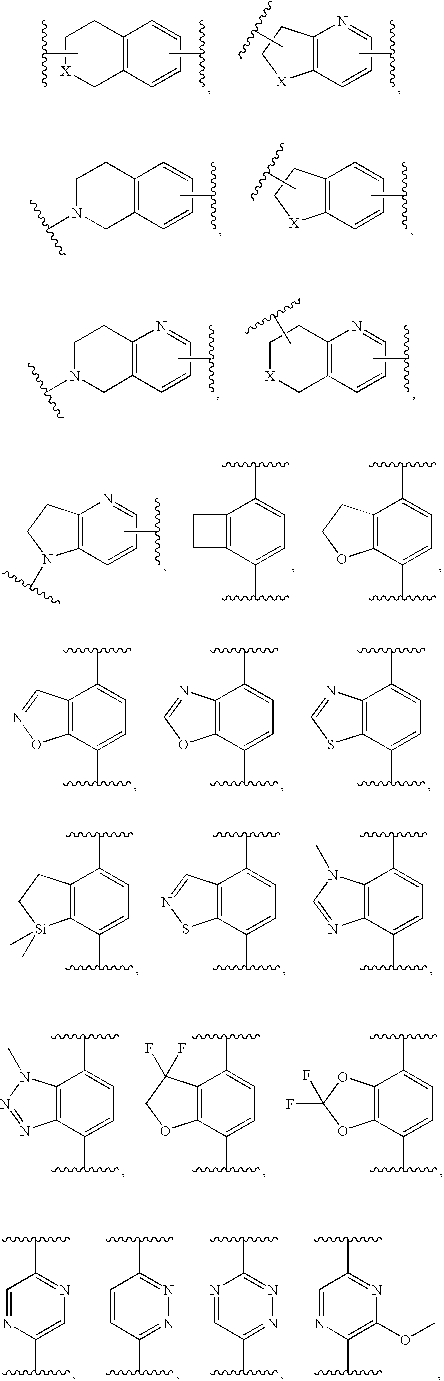

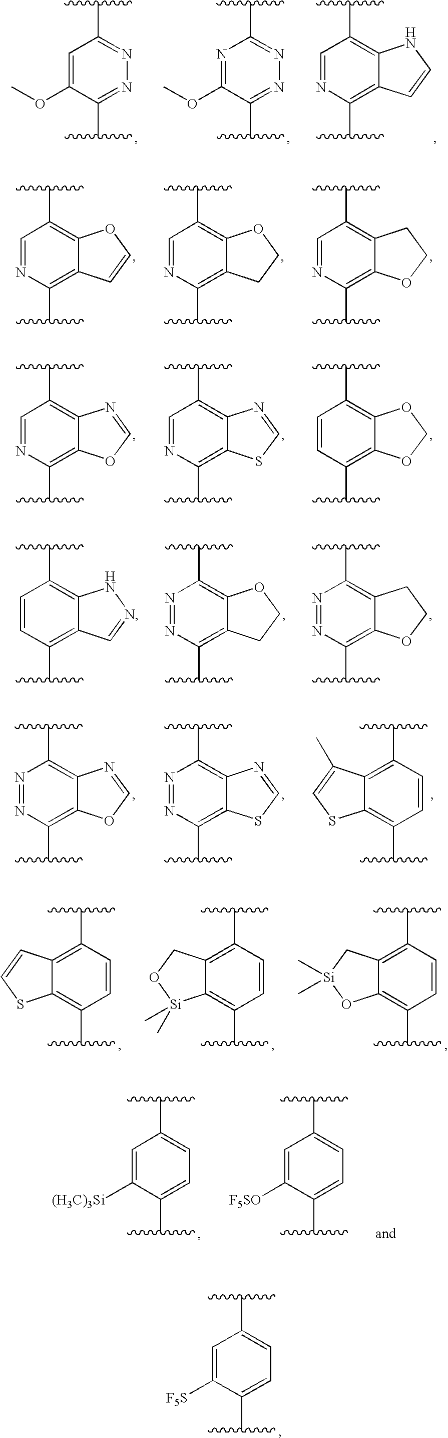

wherein X is selected from the group consisting of: O, —N(R¹⁴)— and —S—; and wherein each of said R¹⁰ moieties is optionally substituted with 1-5 independently selected R²¹ groups; or

R⁹ and R¹⁰ are linked together to form a fused tricyclic ring system wherein R⁹ and R¹⁰ are as defined above and the ring linking R⁹ and R¹⁰ is an alkyl ring, or a heteroalkyl ring, or an aryl ring, or a heteroaryl ring, or an alkenyl ring, or a heteroalkenyl ring (for example, the tricyclic ring system is formed by linking the atoms adjacent to the atoms by which R⁹ and R¹⁰ are bound together);

R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclylalkenyl-, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶);

R^{15A} and R^{16A} are independently selected from the group consisting of alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙheteroarylalkyl; or

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl;

each R¹⁸ is independently selected from the group consisting of alkyl, alkenyl, alkynyl, aryl, arylalkyl, arylalkenyl, arylalkynyl, —NO₂, halo, heteroaryl, HO-alkoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or

two R¹⁸ moieties on adjacent carbons can be linked together to form a

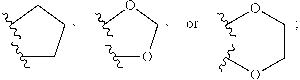

R¹⁹ is selected from the group consisting of: alkyl, cycloalkyl, aryl, arylalkyl and heteroarylalkyl;

R²⁰ is selected from the group consisting of: alkyl, cycloalkyl, aryl, halo substituted aryl, arylalkyl, heteroaryl and heteroarylalkyl;

each R²¹ is independently selected from the group consisting of: alkyl, alkenyl, alkynyl, cycloalkyl, cycloalkylalkyl, cycloalkenyl, heterocycloalkyl, ═O, ═N—R², heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —P(O)(CH₃)₂, —SO(═NR¹⁵)R¹⁶—, —SF₅, —OSF₅, —Si(R^{15A})₃ wherein each R^{15A} is independently selected —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R^{16A}, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R^{15A}═NOR¹⁵, —N₃, —NO₂, —S(O)₂R^{15A}, —O—N═C(R⁴)₂ (wherein each R⁴ is independently selected), and —O—N═C(R⁴)₂ wherein R⁴ is taken together with the carbon atom to which they are bound to form a 5 to 10 membered ring, said ring optionally containing 1 to 3 heteroatoms selected from the group consisting of —O—, —S—, —S(O)—, —S(O)₂—, and —NR²—; wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups;

each R²² group is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R^{16A}, —N(R¹⁵)S(O)₂R^{16A}, —CH₂—N(R¹⁵)S(O)₂N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R^{15A} and —S(O)₂R^{15A}; and

provided that:
when W is —(C═O)—, and G is bound to G⁴, and when G¹, G², G³, and G⁴ are the same or different —C(R²¹)_{q}— moiety, and G is —CHR³—, then R³ is not H, halo, unsubstituted aryl, substituted aryl, unsubstituted heteroaryl, substituted heteroaryl, unsubstituted arylalkyl, substituted arylalkyl, unsubstituted heteroarylalkyl, substituted heteroarylalkyl, unsubstituted alkyl, substituted alkyl, or —O-alkyl; and
when R²¹ is bound to a carbon that has three other filled valences (such as, for example, in the moiety

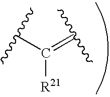

then R²¹ is not ═O, ═NR², or ═NOR¹⁵; and
when G is bound to G⁴, and G¹ is a C(R²¹)_{q} group, and the carbon of said G¹ group is bound to a ═N— in Ring B (i.e., there is a double bond between the G¹ carbon and a N in Ring B), and the optional bond between G¹ and G² is not present (i.e., there is a single bond between G¹ and G²), and G² is N(R²)_{d}, and G³ is —C(R²¹)_{q}—, then G is not CHR³; and
when G is bound to G⁴, and G¹ is a C(R²¹)_{q} group, and the carbon of said G¹ group is bound to an —NR²— in Ring B (i.e., there is a single bond between the G¹ carbon a N in Ring B), and the optional bond between G¹ and G² is present (i.e., there is a double bond between G¹ and G²), and G² is N(R²)_{d}, and G³ is —C(R²¹)_{q}—, then G is not CHR³; and
when G is bound to G⁴, and G¹ is a C(R²¹)_{q} group, and the carbon of said G¹ group is bound to a ═N— in Ring B (i.e., there is a double bond between the G¹ carbon and a N in Ring B), and G² is a direct bond (i.e., G¹ is bound directly to G³), and G³ is N, then G is not CHR³.

The compounds of this invention are useful for treating central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

Thus, for example, the compounds of this invention can be used to treat the following diseases or conditions: Alzheimers disease, mild cognitive impairment (MCI), Downs Syndrome, Glaucoma (Guo et. al., Proc. Natl. Acad. Sci. USA 104, 13444-13449 (2007)), Cerebral amyloid angiopathy, stroke or dementia (Frangione et al., Amyloid: J. Protein folding Disord. 8, suppl. 1, 36-42 (2001), Microgliosis and brain inflammation (M P Lamber, Proc. Natl. Acad. Sci. USA 95, 6448-53 (1998)), and Olfactory function loss (Getchell, et. al. Neurobiology of Aging, 663-673, 24, 2003).

In one embodiment of this invention R^{1A} is selected from the group consisting of: alkyl-, alkenyl-, alkynyl-, aryl-, arylalkyl-, alkylaryl-, cycloalkyl-, cycloalkenyl, cycloalkylalkyl-, fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), heteroaryl-, heteroarylalkyl-, heterocyclyl-, heterocyclenyl, -and heterocyclyalkyl-; wherein each of said alkyl-, alkenyl- and alkynyl-, aryl-, arylalkyl-, alkylaryl-, cycloalkyl-, cycloalkenyl-, cycloalkylalkyl-, fused benzocycloalkyl, fused benzoheterocycloalkyl, fused heteroarylcycloalkyl, fused heteroarylheterocycloalkyl, heteroaryl-, heteroarylalkyl-, heterocyclyl-, heterocyclenyl and heterocyclyalkyl- R^{1A} groups is optionally substituted with 1-5 independently selected R²¹ groups; or

Examples of moieties formed when R¹⁰ and R⁹ are linked together to form a fused tricyclic ring system include, but are not limited to:

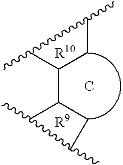

wherein R¹⁰ and R⁹ are as defined for formula (I), and Ring C is the ring linking R¹⁰ and R⁹, that is Ring C is an alkyl ring, or a heteroalkyl ring, or an aryl ring, or a heteroaryl ring, or an alkenyl ring, or a heteroalkenyl ring.

Examples of moieties formed when R¹⁰ and R⁹ are linked together to form a fused tricyclic ring system include, but are not limited to:

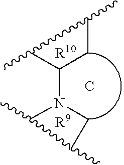

wherein R¹⁰ and R⁹ are as defined for formula (I), and Ring C is the ring linking R¹⁰ and R⁹, that is Ring C is a heteroalkyl ring, or a heteroaryl ring, or a heteroalkenyl ring.

In one example, the fused tricyclic ring system formed when R¹⁰ and R⁹ are linked together is

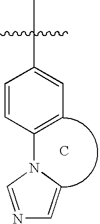

wherein Ring C is a heteroalkyl ring, or a heteroaryl ring, or a heteroalkenyl ring, thus, for example, the tricyclic ring system is formed by linking the atoms adjacent to the atoms by which R¹⁰ and R⁹ are bound together), and wherein said fused tricyclic ring system is optionally substituted with 1 to 5 independently selected R²¹ groups.

Other examples of moieties formed when R¹⁰ and R⁹ are linked together to form a fused tricyclic ring system include, but are not limited to:

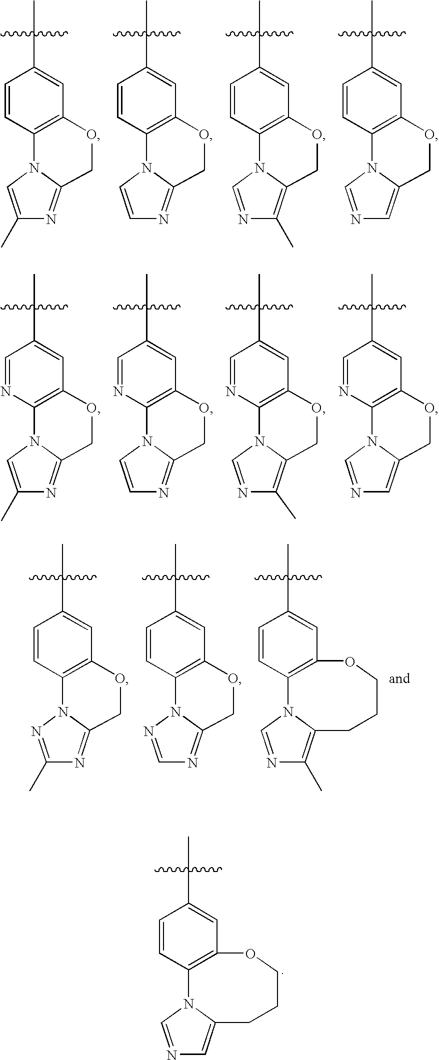

Another embodiment of this is directed to compounds of formula (I) wherein at least one (e.g., 1 to 3, or 1-2, or 1) group selected from the group consisting of: —SF₅, —OSF₅, and —Si(R^{15A})₃ is present, and wherein each R^{15A} is independently selected, and wherein when there is more than one group, each group is independently selected.

Another embodiment of this is directed to compounds of formula (I) wherein at least one (e.g., 1 to 3, or 1-2, or 1) group selected from the group consisting of: —SF₅ and —OSF₅ is present, and wherein when there is more than one group, each group is independently selected.

In one embodiment of this invention one group selected from the group consisting of: —SF₅, —OSF₅, and —Si(R^{15A})₃ (wherein each R^{15A} is independently selected) is present in the compounds of formula (I).

In another embodiment of this invention two groups selected from the group consisting of: —SF₅, —OSF₅, and —Si(R^{15A})₃ (wherein each R^{15A} is independently selected) are present in the compounds of formula (I).

In another embodiment of this invention three groups selected from the group consisting of: —SF₅, —OSF₅, and —Si(R^{15A})₃ (wherein each R^{15A} is independently selected) are present in the compounds of formula (I).

In another embodiment of this invention two groups selected from the group consisting of: —SF₅, —OSF₅, and —Si(R^{15A})₃ (wherein each R^{15A} is independently selected) are present in the compounds of formula (I), wherein at least one group is other than —Si(R^{15A})₃.

In another embodiment of this invention three groups selected from the group consisting of: —SF₅, —OSF₅, and —Si(R^{15A})₃ (wherein each R^{15A} is independently selected) are present in the compounds of formula (I), wherein at least one group is other than —Si(R^{15A})₃.

In another embodiment of this invention one group selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) is present in the compounds of formula (I).

In another embodiment of this invention two groups selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) are present in the compounds of formula (I).

In another embodiment of this invention three groups selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) are present in the compounds of formula (I).

In another embodiment of this invention two groups selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) are present in the compounds of formula (I), wherein at least one group is other than —Si(R^{15A})₃.

In another embodiment of this invention three groups selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) are present in the compounds of formula (I), wherein at least one group is other than —Si(R^{15A})₃.

In another embodiment of this invention one group selected from the group consisting of: —SF₅, —OSF₅, and —Si(R^{15A})₃ (wherein each R^{15A} is independently selected from the group consisting of methyl, ethyl and phenyl) is present in the compounds of formula (I).

In another embodiment of this invention two groups selected from the group consisting of: —SF₅, —OSF₅, and —Si(R^{15A})₃ (wherein each R^{15A} is independently selected from the group consisting of methyl, ethyl and phenyl) are present in the compounds of formula (I).

In another embodiment of this invention three groups selected from the group consisting of: —SF₅, —OSF₅, and —Si(R^{15A})₃ (wherein each R^{15A} is independently selected from the group consisting of methyl, ethyl and phenyl) are present in the compounds of formula (I).

In another embodiment of this invention two groups selected from the group consisting of: —SF₅, —OSF₅, and —Si(R^{15A})₃ (wherein each R^{15A} is independently selected from the group consisting of methyl, ethyl and phenyl) are present in the compounds of formula (I), wherein at least one group is other than —Si(R^{15A})₃.

In another embodiment of this invention three groups selected from the group consisting of: —SF₅, —OSF₅, and —Si(R^{15A})₃ (wherein each R^{15A} is independently selected from the group consisting of methyl, ethyl and phenyl) are present in the compounds of formula (I), wherein at least one group is other than —Si(R^{15A})₃.

In another embodiment of this invention one group selected from the group consisting of: —SF₅, —OSF₅, and —Si(R^{15A})₃ (wherein each R^{15A} is independently selected from the group consisting of methyl and ethyl) is present in the compounds of formula (I).

In another embodiment of this invention two groups selected from the group consisting of: —SF₅, —OSF₅, and —Si(R^{15A})₃ (wherein each R^{15A} is independently selected from the group consisting of methyl and ethyl) are present in the compounds of formula (I).

In another embodiment of this invention three groups selected from the group consisting of: —SF₅, —OSF₅, and —Si(R^{15A})₃ (wherein each R^{15A} is independently selected from the group consisting of methyl and ethyl) are present in the compounds of formula (I).

In another embodiment of this invention two groups selected from the group consisting of: —SF₅, —OSF₅, and —Si(R^{15A})₃ (wherein each R^{15A} is independently selected from the group consisting of methyl and ethyl) are present in the compounds of formula (I), wherein at least one group is other than —Si(R^{15A})₃.

In another embodiment of this invention three groups selected from the group consisting of: —SF₅, —OSF₅, and —Si(R^{15A})₃ (wherein each R^{15A} is independently selected from the group consisting of methyl and ethyl) are present in the compounds of formula (I), wherein at least one group is other than —Si(R^{15A})₃.

In another embodiment of this invention one group selected from the group consisting of: —SF₅, —OSF₅, and —Si(R^{15A})₃ is present in the compounds of formula (I), and said —Si(R^{15A})₃ group is selected from the group consisting of: —Si(CH₃)₃, —Si(CH₃)₂phenyl, and —Si(CH₂CH₃)₂CH₃.

In another embodiment of this invention two groups selected from the group consisting of: —SF₅, —OSF₅, and —Si(R^{15A})₃ are present in the compounds of formula (I), and said —Si(R^{15A})₃ group is selected from the group consisting of: —Si(CH₃)₃, —Si(CH₃)₂phenyl, and —Si(CH₂CH₃)₂CH₃.

In another embodiment of this invention three groups selected from the group consisting of: —SF₅, —OSF₅, and —Si(R^{15A})₃ are present in the compounds of formula (I), and said —Si(R^{15A})₃ group is selected from the group consisting of: —Si(CH₃)₃, Si(CH₃)₂phenyl, and —Si(CH₂CH₃)₂CH₃.

In another embodiment of this invention two groups selected from the group consisting of: —SF₅, —OSF₅, and —Si(R^{15A})₃ are present in the compounds of formula (I), wherein at least one group is other than —Si(R^{15A})₃, and said —Si(R^{15A})₃ group is selected from the group consisting of: —Si(CH₃)₃, —Si(CH₃)₂phenyl, and —Si(CH₂CH₃)₂CH₃.

In another embodiment of this invention three groups selected from the group consisting of: —SF₅, —OSF₅, and —Si(R^{15A})₃ are present in the compounds of formula (I), wherein at least one group is other than —Si(R^{15A})₃, and said —Si(R^{15A})₃ group is selected from the group consisting of: —Si(CH₃)₃, —Si(CH₃)₂phenyl, and —Si(CH₂CH₃)₂CH₃.

In another embodiment of this invention one group selected from the group consisting of: —SF₅, —OSF₅, and —Si(R^{15A})₃ is present in the compounds of formula (I), and said —Si(R^{15A})₃ group is selected from the group consisting of: —Si(CH₃)₃ and —Si(CH₂CH₃)₂CH₃.

In another embodiment of this invention two groups selected from the group consisting of: —SF₅, —OSF₅, and —Si(R^{15A})₃ are present in the compounds of formula (I), and said —Si(R^{15A})₃ group is selected from the group consisting of: —Si(CH₃)₃ and —Si(CH₂CH₃)₂CH₃.

In another embodiment of this invention three groups selected from the group consisting of: —SF₅, —OSF₅, and —Si(R^{15A})₃ are present in the compounds of formula (I), and said —Si(R^{15A})₃ group is selected from the group consisting of: —Si(CH₃)₃ and —Si(CH₂CH₃)₂CH₃.

In another embodiment of this invention two groups selected from the group consisting of: —SF₅, —OSF₅, and —Si(R^{15A})₃ are present in the compounds of formula (I), wherein at least one group is other than —Si(R^{15A})₃, and said —Si(R^{15A})₃ group is selected from the group consisting of: —Si(CH₃)₃ and —Si(CH₂CH₃)₂CH₃.

In another embodiment of this invention three groups selected from the group consisting of: —SF₅, —OSF₅, and —Si(R^{15A})₃ are present in the compounds of formula (I), wherein at least one group is other than —Si(R^{15A})₃, and said —Si(R^{15A})₃ group is selected from the group consisting of: —Si(CH₃)₃, —Si(CH₃)₂phenyl, and —Si(CH₂CH₃)₂CH₃.

In another embodiment of this invention one group selected from the group consisting of: —SF₅, —OSF₅, and —Si(CH₃)₃ is present.

In another embodiment of this invention two groups selected from the group consisting of: —SF₅, —OSF₅, and —Si(CH₃)₃ are present in the compounds of formula (I).

In another embodiment of this invention three groups selected from the group consisting of: —SF₅, —OSF₅, and —Si(CH₃)₃ are present in the compounds of formula (I).

In another embodiment of this invention two groups selected from the group consisting of: —SF₅, —OSF₅, and —Si(CH₃)₃ are present in the compounds of formula (I), wherein at least one group is other than —Si(CH₃)₃.

In another embodiment of this invention three groups selected from the group consisting of: —SF₅, —OSF₅, and —Si(R²⁴)₃ are present in the compounds of formula (I), wherein at least one group is other than —Si(CH₃)₃.

In another embodiment of this invention one group selected from the group consisting of: —SF₅ and —OSF₅ is present in the compounds of formula (I).

In another embodiment of this invention two groups selected from the group consisting of: —SF₅ and —OSF₅ are present in the compounds of formula (I).

In another embodiment of this invention three groups selected from the group consisting of: —SF₅ and —OSF₅ are present in the compounds of formula (I)

In another embodiment of this invention one —SF₅ group is present in the compounds of formula (I).

In another embodiment of this invention two —SF₅ groups are present in the compounds of formula (I).

In another embodiment of this invention three —SF₅ groups are present in the compounds of formula (I).

In another embodiment of this invention one —OSF₅ group is present in the compounds of formula (I).

In another embodiment of this invention two —OSF₅ groups are present in the compounds of formula (I).

In another embodiment of this invention three —OSF₅ groups are present in the compounds of formula (I).

In another embodiment of this invention one —Si(R^{15a})₃ (wherein each R^{15A} is independently selected) group is present in the compounds of formula (I).

In another embodiment of this invention two —Si(R^{15A})₃ (wherein each R^{15A} is independently selected) groups are present in the compounds of formula (I).

In another embodiment of this invention three —Si(R^{15A})₃ (wherein each R^{15A} is independently selected) groups are present in the compounds of formula (I).

In another embodiment of this invention one —Si(R^{15A})₃ (wherein each R^{15A} is independently selected from the group consisting of alkyl (e.g., methyl and ethyl) and aryl (e.g., phenyl)) is present in the compounds of formula (I).

In another embodiment of this invention two —Si(R^{15A})₃ (wherein each R^{15A} is independently selected from the group consisting of alkyl (e.g., methyl and ethyl) and aryl (e.g., phenyl)) is present in the compounds of formula (I).

In another embodiment of this invention three —Si(R^{15A})₃ (wherein each R^{15A} is independently selected from the group consisting of alkyl (e.g., methyl and ethyl) and aryl (e.g., phenyl)) is present in the compounds of formula (I).

In another embodiment of this invention one —Si(R^{15A})₃ (wherein each R^{15A} is independently selected from the group consisting of methyl, ethyl and phenyl) is present in the compounds of formula (I).

In another embodiment of this invention two —Si(R^{15A})₃ (wherein each R^{15A} is independently selected from the group consisting of methyl, ethyl and phenyl) is present in the compounds of formula (I).

In another embodiment of this invention three —Si(R^{15A})₃ (wherein each R^{15A} is independently selected from the group consisting of methyl, ethyl and phenyl) is present in the compounds of formula (I).

In another embodiment of this invention one —Si(R^{15A})₃ (wherein each R^{15A} is independently selected from the group consisting of methyl and ethyl) is present in the compounds of formula (I).

In another embodiment of this invention two —Si(R^{15A})₃ (wherein each R^{15A} is independently selected from the group consisting of methyl and ethyl) is present in the compounds of formula (I).

In another embodiment of this invention three —Si(R^{15A})₃ (wherein each R^{15A} is independently selected from the group consisting of methyl and ethyl) is present in the compounds of formula (I).

In another embodiment of this invention one —Si(R^{15A})₃ group is present in the compounds of formula (I), and said —Si(R^{15A})₃ group is selected from the group consisting of: —Si(CH₃)₃, —Si(CH₃)₂phenyl, and —Si(CH₂CH₃)₂CH₃.

In another embodiment of this invention two —Si(R^{15A})₃ groups are present in the compounds of formula (I), and said —Si(R^{15A})₃ groups are independently selected from the group consisting of: —Si(CH₃)₃, —Si(CH₃)₂phenyl, and —Si(CH₂CH₃)₂CH₃.

In another embodiment of this invention three —Si(R^{15A})₃ groups are present in the compounds of formula (I), and said —Si(R^{15A})₃ groups are independently selected from the group consisting of: —Si(CH₃)₃, —Si(CH₃)₂phenyl, and —Si(CH₂CH₃)₂CH₃.

In another embodiment of this invention one —Si(R^{15A})₃ group is present in the compounds of formula (I), and said —Si(R^{15A})₃ group is selected from the group consisting of: —Si(CH₃)₃ and —Si(CH₂CH₃)₂CH₃.

In another embodiment of this invention two —Si(R^{15A})₃ groups are present in the compounds of formula (I), and said —Si(R^{15A})₃ groups are independently selected from the group consisting of: —Si(CH₃)₃ and —Si(CH₂CH₃)₂CH₃.

In another embodiment of this invention three —Si(R^{15A})₃ groups are present in the compounds of formula (I), and said —Si(R^{15A})₃ groups are independently selected from the group consisting of: —Si(CH₃)₃ and —Si(CH₂CH₃)₂CH₃.

In another embodiment of this invention one —Si(R^{15A})₃ group is present in the compounds of formula (I), and said —Si(R^{15A})₃ group is —Si(CH₃)₃.

In another embodiment of this invention two —Si(R^{15A})₃ groups are present in the compounds of formula (I), and said —Si(R^{15A})₃ groups are —Si(CH₃)₃.

In another embodiment of this invention three —Si(R^{15A})₃ groups are present in the compounds of formula (I), and said —Si(R^{15A})₃ groups are —Si(CH₃)₃.

In another embodiment of this invention one group selected from the group consisting of: —SF₅, —OSF₅, —Si(CH₃)₃, —Si(CH₃)₂phenyl, and —Si(CH₂CH₃)₂CH₃) is present in the compounds of formula (I).

In another embodiment of this invention one group selected from the group consisting of: —SF₅, —OSF₅, —Si(CH₃)₃, and —Si(CH₂CH₃)₂CH₃) is present in the compounds of formula (I).

In another embodiment of this invention one group selected from the group consisting of: —SF₅, —OSF₅, and —Si(CH₃)₃, is present in the compounds of formula (I).

In another embodiment of this invention one —SF₅ group is present in the compounds of formula (I), and one or two additional groups selected from the group consisting of: —SF₅, —OSF₅, and —Si(R^{15A})₃ (wherein each R^{15A} is independently selected) are also present in the compounds of formula (I).

In another embodiment of this invention one —SF₅ group is present in the compounds of formula (I), and one or two additional groups selected from the group consisting of: —OSF₅, and —Si(R^{15A})₃ (wherein each R^{15A} is independently selected) are also present in the compounds of formula (I).

In another embodiment of this invention one —OSF₅ group is present in the compounds of formula (I), and one or two additional groups selected from the group consisting of: —SF₅, —OSF₅, and —Si(R^{15A})₃ (wherein each R^{15A} is independently selected) are also present in the compounds of formula (I).

In another embodiment of this invention one —OSF₅ group is present in the compounds of formula (I), and one or two additional groups selected from the group consisting of: —SF₅ and —Si(R^{15A})₃ (wherein each R^{15A} is independently selected) are also present in the compounds of formula (I).

In another embodiment of this invention one —SF₅ group is present in the compounds of formula (I), and one or two additional groups selected from the group consisting of: —SF₅ and —OSF₅ are also present in the compounds of formula (I).

In another embodiment of this invention one —OSF₅ group is present in the compounds of formula (I), and one or two additional groups selected from the group consisting of: —SF₅ and —OSF₅ are also present in the compounds of formula (I).

In another embodiment of this invention one —Si(R^{15A})₃ (wherein each R^{15A} is independently selected) group is present in the compounds of formula (I), and one or two groups selected from the group consisting of: —SF₅, —OSF₅, and —Si(R^{15A})₃ (wherein each R^{15A} is independently selected) are also present in the compounds of formula (I).

In another embodiment of this invention one —Si(R^{15A})₃ (wherein each R^{15A} is independently selected) group is present in the compounds of formula (I), and one or two groups selected from the group consisting of: —SF₅ and —OSF₅ are also present in the compounds of formula (I).

In another embodiment of this invention at least one group selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) is present in the compounds of formula (I).

In another embodiment of this invention at least one group selected from the group consisting of: —SF₅, —OSF₅, and —Si(R^{15A})₃ (wherein each R^{15A} is independently selected from the group consisting of alkyl (e.g., methyl and ethyl) and phenyl) is present in the compounds of formula (I).

In another embodiment of this invention at least one group selected from the group consisting of: —SF₅, —OSF₅, and —Si(R^{15A})₃ (wherein each R^{15A} is independently selected from the group consisting of methyl, ethyl and phenyl) is present in the compounds of formula (I).

In another embodiment of this invention at least one group selected from the group consisting of: —SF₅, —OSF₅, —Si(CH₃)₃, —Si(CH₃)₂phenyl, and —Si(CH₂CH₃)₂CH₃) is present in the compounds of formula (I).

In another embodiment of this invention at least one group selected from the group consisting of: —SF₅, —OSF₅, and —Si(CH₃)₃ is present in the compounds of formula (I).

In another embodiment of this invention one group selected from the group consisting of: —SF₅, —OSF₅, and —Si(R^{15A})₃ (wherein each R^{15A} is independently selected) is present in the compounds of formula (I).

In another embodiment of this invention one group selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) is present in the compounds of formula (I).

In another embodiment of this invention one group selected from the group consisting of: —SF₅, —OSF₅, and —Si(R^{15A})₃ (wherein each R^{15A} is independently selected from the group consisting of alkyl (e.g., methyl and ethyl) and phenyl) is present in the compounds of formula (I).

In another embodiment of this invention one group selected from the group consisting of: —SF₅, —OSF₅, and —Si(R^{15A})₃ (wherein each R^{15A} is independently selected from the group consisting of methyl, ethyl and phenyl) is present in the compounds of formula (I).

In another embodiment of this invention one group selected from the group consisting of: —SF₅, —OSF₅, —Si(CH₃)₃, —Si(CH₃)₂phenyl, and —Si(CH₂CH₃)₂CH₃) is present in the compounds of formula (I).

In another embodiment of this invention one group selected from the group consisting of: —SF₅, —OSF₅, —Si(CH₃)₃, and —Si(CH₂CH₃)₂CH₃) is present in the compounds of formula Q.

In another embodiment of this invention one group selected from the group consisting of: —SF₅, —OSF₅, and —Si(CH₃)₃, is present in the compounds of formula (I).

In another embodiment of this invention two groups selected from the group consisting of: —SF₅, —OSF₅, and —Si(R^{15A})₃ (wherein each R^{15A} is independently selected) are present in the compounds of formula (I).

In another embodiment of this invention two groups independently selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) are present in the compounds of formula (I).

In another embodiment of this invention two groups independently selected from the group consisting of: —SF₅, —OSF₅, and —Si(R^{15A})₃ (wherein each R^{15A} is independently selected from the group consisting of alkyl (e.g., methyl and ethyl) and phenyl) are present in the compounds of formula (I).

In another embodiment of this invention two groups selected from the group consisting of: —SF₅, —OSF₅, and —Si(R^{15A})₃ (wherein each R^{15A} is independently selected from the group consisting of methyl, ethyl and phenyl) are present in the compounds of formula (I).

In another embodiment of this invention two groups independently selected from the group consisting of: —SF₅, —OSF₅, —Si(CH₃)₃, —Si(CH₃)₂phenyl, and —Si(CH₂CH₃)₂CH₃) is present in the compounds of formula (I).

In another embodiment of this invention two groups independently selected from the group consisting of: —SF₅, —OSF₅, —Si(CH₃)₃, and —Si(CH₂CH₃)₂CH₃) are present in the compounds of formula (I).

In another embodiment of this invention two groups independently selected from the group consisting of: —SF₅, —OSF₅, and —Si(CH₃)₃ are present in the compounds of formula (I).

In another embodiment of this invention three groups selected from the group consisting of: —SF₅, —OSF₅, and —Si(R^{15A})₃ (wherein each R^{15A} is independently selected) are present in the compounds of formula (I).

In another embodiment of this invention three groups independently selected from the group consisting of: —SF₅, —OSF₅, and —Si(R^{15A})₃ (wherein each R^{15A} is independently selected from the group consisting of alkyl (e.g., methyl and ethyl) and aryl (e.g., phenyl)) are present in the compounds of formula (I).

In another embodiment of this invention three groups independently selected from the group consisting of: —SF₅, —OSF₅, and —Si(R^{15A})₃ (wherein each R^{15A} is independently selected from the group consisting of alkyl (e.g., methyl and ethyl) and phenyl) are present in the compounds of formula (I).

In another embodiment of this invention three groups selected from the group consisting of: —SF₅, —OSF₅, and —Si(R^{15A})₃ (wherein each R^{15A} is independently selected from the group consisting of methyl, ethyl and phenyl) are present in the compounds of formula (I).

In another embodiment of this invention three groups independently selected from the group consisting of: —SF₅, —OSF₅, —Si(CH₃)₃, —Si(CH₃)₂phenyl, and —Si(CH₂CH₃)₂CH₃) is present in the compounds of formula (I).

In another embodiment of this invention three groups independently selected from the group consisting of: —SF₅, —OSF₅, —Si(CH₃)₃, and —Si(CH₂CH₃)₂CH₃) are present in the compounds of formula (I).

In another embodiment of this invention three groups independently selected from the group consisting of: —SF₅, —OSF₅, and —Si(CH₃)₃ are present in the compounds of formula (I).

In another embodiment of this invention at least one group selected from the group consisting of: —SF₅, —OSF₅, and —Si(R^{15A})₃ (wherein each R^{15A} is the same or different alkyl group) is present in the compounds of formula (I).

In another embodiment of this invention at least one group selected from the group consisting of: —SF₅, —OSF₅, and —Si(R^{15A})₃ (wherein each R^{15A} is independently selected from the group consisting of methyl and ethyl) is present in the compounds of formula (I).

In another embodiment of this invention one —SF₅ group is present in the compounds of formula (I), and one or two groups selected from the group consisting of: —SF₅ and —OSF₅ are also present in the compounds of formula (I).

In another embodiment of this invention one —OSF₅ group is present in the compounds of formula (I), and one or two groups selected from the group consisting of: —SF₅ and —OSF₅ are also present in the compounds of formula (I).

Those skilled in the art will appreciate that the G moiety —(C═NR²)— represents

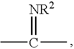

and the G moiety —(C═C(R⁶)₂)- represents

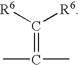

In one embodiment of this invention, the cycloalkyl G moiety is unsubstituted

In another embodiment of this invention, the cycloalkyl G moiety is substituted with 1 to 6 independently selected R²¹ groups.

In another embodiment of this invention, the cycloalkyl G moiety is a C₃ to C₁₀ cycloalkyl substituted with 1 to 6 independently selected R²¹ groups. In one example G is a cyclobutanone ring.

In one embodiment of this invention, the cycloalkyl G moiety is a C₃ to C₁₀ cycloalkyl. In one example, said cycloalkyl is selected from the group consisting of: cyclopropyl, cyclobutyl, cyclopentyl and cyclohexyl. In another example of said cycloalkyl G moiety the cycloalkyl ring carbon by which said cycloalkyl moiety is bound to position (1) or (2) is different from the cycloalkyl ring carbon by which said cycloalkyl moiety is bound to moiety R¹⁰. In another example of said cycloalkyl G moiety the cycloalkyl ring is bound to position (1) or (2) and the R¹⁰ moiety by the same cycloalkyl ring carbon.

In another embodiment of this invention, the heterocycloalkyl G moiety is unsubstituted.

In another embodiment of this invention, the heterocycloalkyl G moiety is unsubstituted and said heterocycloalkyl G moiety comprises 1 to 4 heteroatoms independently selected from the group consisting of: —O—, —NR²—, —S—, —S(O)—, and —S(O)₂.

In another embodiment of this invention, the heterocycloalkyl G moiety is substituted with 1 to 6 independently selected R²¹ groups, and said heterocycloalkyl G moiety comprises 1 to 4 ring heteroatoms independently selected from the group consisting of: —O—, —NR²—, —S—, —S(O)—, and —S(O)₂.

In one embodiment of this invention, the heterocycloalkyl G moiety comprises 1 to 4 heteroatoms. In one example, said heterocycloalkyl G moiety comprises 1 to 4 heteroatoms. In another example, said heterocycloalkyl G moiety comprises 1 to 3 heteroatoms. In another example, said heterocycloalkyl G moiety comprises 1 to 2 heteroatoms. In another example, said heterocycloalkyl G moiety comprises 1 heteroatom. The heteroatoms in said heterocycloalkyl G moiety are independently selected from the group consisting of —O—, —NR²—, —S—, —S(O)—, and —S(O)₂. In one example, said heterocycloalkyl G moiety is bound to the R¹⁰ moiety and position (1) or (2) by the same heterocycloalkyl ring atom. In another example, said heterocycloalkyl moiety is bound to the R¹⁰ moiety and position (1) or (2) by different heterocycloalkyl ring atoms, and wherein the heterocycloalkyl ring atoms that bind the heterocycloalkyl moiety to R¹⁰ and position (1) or (2) are selected from the group consisting of carbon and nitrogen.

An example of said alkynyl G moiety is:

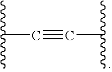

Those skilled in the art will appreciate that when W is —S(O)—, the —S(O)— moiety can be:

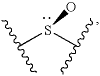

or the —S(O)— moiety can be;

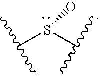

The compounds of formula (I) do not have three consecutive nitrogen atoms in the ring. Thus, in addition to the nitrogen at position (5) in formula (I), there are 0 to 2 additional nitrogens in the ring (i.e., 0 to 2-N(R²)_{d}— groups in the ring) provided that the nitrogens are not in consecutive ring positions. Thus, (a) when G¹ is —N(R²)_{d}— then G² is not —N(R²)_{d}—, and (b) when G³ is —N(R²)_{d}— and G² is —N(R²)_{d}— then G¹ is not —N(R²)_{d}—, and (c) when G³ is —N(R²)_{d}— and G¹ is —N(R²)_{d}— then G² is not —N(R²)_{d}—).

In formula (I), 0 to 2 of the G¹, G², G³, and G⁴ moieties are —N(R²)_{d}— wherein each d and each R² is independently selected. Thus Ring (A) in formula (I) comprises a total of 1 to 3 nitrogen atoms (the N at position (5) and 0 to 2-N(R²)_{d}-moieties) in the ring such that the Ring (A) does not comprise three consecutive ring nitrogens, and each d and each R² is independently selected.

In one embodiment of this invention the moiety -G-R¹⁰-R⁹ is bound through G to position (1).

In another embodiment of this invention the moiety -G-R¹⁰-R⁹ is bound through G to position (2).

In another embodiment of this invention G is selected from the group consisting of: G is selected from the group consisting of: a direct bond (i.e., R¹⁰ is bound directly to either G³ or G⁴), cycloalkyl (e.g., C₃ to C₁₀, and also for example, cyclopropyl, cyclobutyl, cyclopentyl and cyclohexyl, and wherein in one example the cycloalkyl ring carbon by which said cycloalkyl moiety is bound to positions (1) or (2) is different from the cycloalkyl ring carbon by which said cycloalkyl moiety is bound to moiety R¹⁰, and wherein in another example said cycloalkyl ring is bound to positions (1) or (2) and the R¹⁰ moiety by the same cycloalkyl ring carbon), heterocycloalkyl (wherein said heterocycloalkyl comprises 1 to 4 heteroatoms, and in one example, 1 to 4 heteroatoms, and in another example 1 to 3 heteroatoms, and in another example 1 to 2 heteroatoms, and in another example 1 heteroatom, and wherein said heteroatoms are selected from the group consisting of —O—, —NR²—, —S—, —S(O)—, and —S(O)₂, and wherein in one example said heterocycloalkyl moiety is bound to the R¹⁰ moiety and positions (1) or (2) by the same heterocycloalkyl ring atom, and in another example said heterocycloalkyl moiety is bound to the R¹⁰ moiety and positions (1) or (2) by different heterocycloalkyl ring atoms, and wherein the heterocycloalkyl ring atoms that bind the heterocycloalkyl moiety to R¹⁰ and positions (1) or (2) are selected from the group consisting of carbon and nitrogen), —C═C—, —CF₂-alkynyl (e.g., —C≡C—), —NH—, —N(R²)— (and in one example, —NH—), —O—, —CR⁴(OH)—, —CR⁴(OR⁴)—, —(CH₂)ᵣN(R²)—, —N(R²)(CH₂)ᵣ—, —(CH₂)₂₋₃—, —(C(R⁴)₂)ᵣ— (wherein each R⁴ is independently selected), —(CHR⁴)₂₋₃—(wherein each R⁴ is independently selected), —S—, —S(O)—, and —S(O)₂.

In one embodiment of this invention the moiety -G-R¹⁰-R⁹ is bound through G to position (1).

In another embodiment of this invention the moiety -G-R¹⁰-R⁹ is bound through G to position (2).

In another embodiment of this invention t is 1.

In another embodiment of this invention t is 2.

In another embodiment of this invention r is 1.

In another embodiment of this invention r is 2.

In another embodiment of this invention r is 3.

In another embodiment of this invention G is selected from the group consisting of: a direct bond, and —N(R²)— (e.g., —NH—).

In another embodiment of this invention G is a direct bond.

In another embodiment of this invention G is —N(R²)— (e.g., —NH—).

In another embodiment of this invention G is a cycloalkyl.

In another embodiment of this invention G is a heterocycloalkyl.

In another embodiment of this invention G is —C═C—.

In another embodiment of this invention G is —CF₂—.

In another embodiment of this invention G is alkynyl.

In another embodiment of this invention G is —O—.

In another embodiment of this invention G is —CR⁴(OH)—.

In another embodiment of this invention G is —CR⁴(OR⁴)—.

In another embodiment of this invention G is —(CH₂)ᵣN(R²)—.

In another embodiment of this invention G is —N(R²)(CH₂)ᵣ—.

In another embodiment of this invention G is —(CH₂)₂₋₅—.

In another embodiment of this invention G is —(C(R⁴)₂)ᵣ— (wherein each R⁴ is independently selected).

In another embodiment of this invention G is —(CHR⁴)₂₋₅— (wherein each R⁴ is independently selected).

In another embodiment of this invention G is —S—.

In another embodiment of this invention G is —S(O)—.

In another embodiment of this invention G is —S(O)₂.

In another embodiment of this invention G is —C(O)—.

In another embodiment of this invention G is —(C═NR²)—.

In another embodiment of this invention G is —(C═C(R⁶)₂)—.

In another embodiment of this invention G is —(CHR³)—.

In another embodiment of this invention G¹ is —C(R²¹)_{q}—.

In another embodiment of this invention G¹ is —N(R²)_{d}—.

In another embodiment of this invention G² is a direct bond.

In another embodiment of this invention G² is —C(R²¹)_{q}—.

In another embodiment of this invention G² is —N(R²)_{d}—.

In another embodiment of this invention G² is —C(O)—.

In another embodiment of this invention G² is —C(═NR²)—.

In another embodiment of this invention G² is —S(O)₂.

In another embodiment of this invention G² is —S(O)—.

In another embodiment of this invention G² is —C(N(R²)₂)—.

In another embodiment of this invention G³ is —C(R²¹)_{q}—.

In another embodiment of this invention G³ is —N(R²)_{d}—.

In another embodiment of this invention W is —C(O)—.

In another embodiment of this invention W is —S(O)—.

In another embodiment of this invention W is —S(O)₂—.

In another embodiment of this invention W is —C(═NR²)—.

In another embodiment of this invention G is —C(O)—, and W is —C(O)—.

In another embodiment of this invention G is —(C═NR²)—, and W is —C(O)—.

In another embodiment of this invention G is —(C═C(R⁶)₂)- wherein each R⁶ is independently selected, and W is —C(O)—.

In another embodiment of this invention G is —(CHR³)—, and W is —C(O)—.

In another embodiment of this invention G is —(CHR³)—. and R³ is H (i.e., G is —CH₂—), and W is —C(O)—.

In another embodiment of this invention G is —(CHR³)—. and R³ is —OH (i.e., G is —(CHOH)—), and W is —C(O)—.

In another embodiment of this invention. G is —(CHR³)—. and R³ is —O-alkyl (i.e., alkoxy, such as, for example, —OCH₃), and W is —C(O)—.

In another embodiment of this invention G¹ is —C(R²¹)_{q}—, and W is —C(O)—.

In another embodiment of this invention G¹ is —N(R²)_{d}—, and W is —C(O)—.

In another embodiment of this invention G² is —C(R²¹)_{q}—, and W is —C(O)—.

In another embodiment of this invention G² is —N(R²)_{d}—, and W is —C(O)—.

In another embodiment of this invention G² is —C(O)—, and W is —C(O)—.

In another embodiment of this invention G² is —S(O)—, and W is —C(O)—.

In another embodiment of this invention G² is —S(O)₂—, and W is —C(O)—.

In another embodiment of this invention G² is —C(═NR²)—, and W is —C(O)—.

In another embodiment of this invention G² is —C(N(R²)₂)- wherein each R² is independently selected, and W is —C(O)—.

In another embodiment of this invention G³ is —C(R²¹)_{d}—, and W is —C(O)—.

In another embodiment of this invention G³ is —N(R²)_{d}—, and W is —C(O)—.

In another embodiment of this invention R²¹ is selected from the group consisting of: alkyl, —OR¹⁵, —C(O)OR¹⁵, —C(O)NR¹⁵R¹⁶, and alkyl substituted with 1 to 5 independently selected R²² groups (e.g., halo, such as, for example, F, Cl, and Br).

In another embodiment of this invention R²¹ is selected from the group consisting of: alkyl, —OR¹⁵, —C(O)OR¹⁵, —C(O)NR¹⁵R¹⁶, and alkyl substituted with 1 to 5 independently selected R²² groups (e.g., halo, such as, for example, F, Cl, and Br, and wherein in one example the alkyl substituted R²¹ group is —CFA wherein R¹⁵ and R¹⁶ are independently selected from the group consisting of: H, alkyl, (R¹⁸)ₙ-arylalkyl- (wherein, for example, n is 1, and R¹⁸ is —OR²⁰, and R²⁰ is alkyl (e.g., methyl), cycloalkyl (e.g., cyclobutyl), and (R¹⁸)ₙ-alkyl (e.g., n is 1, R¹⁸ is —OR²⁰, and R²⁰ is alkyl (e.g., methyl).

In another embodiment of this invention R²¹ is selected from the group consisting of: (a) alkyl, —OR¹⁵ (wherein R¹⁵ is alkyl, e.g., methyl and ethyl), (b) —C(O)OR¹⁵ (wherein R¹⁵ is alkyl, e.g., methyl), (c) —C(O)NR¹⁵R¹⁶ (wherein R¹⁵ and R¹⁶ are independently selected from the group consisting of: H, alkyl, (R¹⁸)ₙ-arylalkyl- (wherein, for example, n is 1, and R¹⁸ is —OR²⁰, and R²⁰ is alkyl (e.g., methyl), cycloalkyl (e.g., cyclobutyl), and (R¹⁸)ₙ-alkyl (e.g., n is 1, R¹⁸ is —OR²⁰, and R²⁰ is alkyl (e.g., methyl), and in one example, only one of R¹⁵ and R¹⁶ is H), and (d) alkyl substituted with 1 to 5 independently selected R²² groups (e.g., halo, such as, for example, F, Cl, and Br, and wherein in one example the alkyl substituted R²¹ group is —CF₃).

In one embodiment of this invention R¹⁰ is selected from the group consisting of: aryl- (e.g., phenyl), heteroaryl- (e.g., pyridyl) cycloalkyl-, cycloalkenyl, cycloalkylalkyl-, heterocyclyl-, heterocyclenyl-, heterocyclylalkyl-, heterocyclylalkenyl-, fused benzocycloalkyl- (i.e., benzofusedcycloalkyl-), fused benzoheterocycloalkyl- (i.e., benzofusedheterocycloalkyl-), fused heteroarylcycloalkyl- (i.e., heteroarylfusedcycloalkyl-), fused heteroarylheterocycloalkyl- (i.e., heteroarylfusedheterocycloalkyl-), fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-), and fused heterocycloalkylheteroaryl- (i.e., heterocycloalkylfusedheteroaryl-), and wherein each of said R¹⁰ moieties is optionally substituted with 1-5 independently selected R²¹ groups.

In another embodiment of this invention R¹⁰ is selected from the group consisting of:

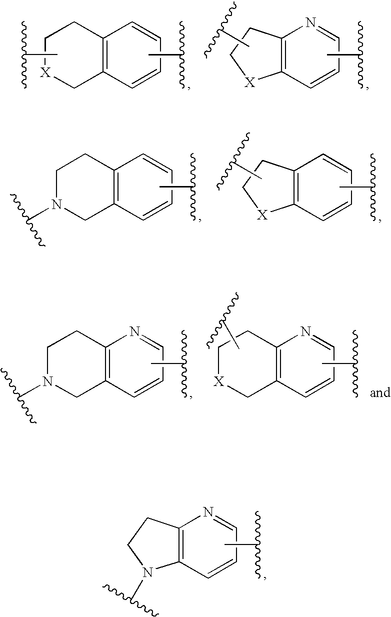

wherein X is selected from the group consisting of: O, —N(R¹⁴)— and —S—; and wherein each of said R¹⁰ moieties is optionally substituted with 1-5 independently selected R²¹ groups.

In another embodiment of this invention R¹⁰ is selected from the group consisting of:

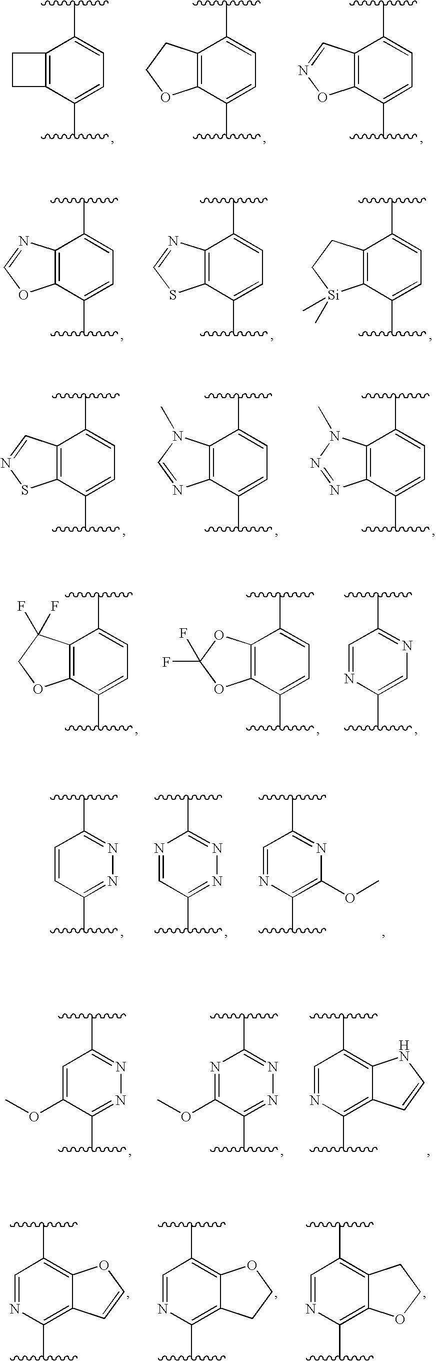

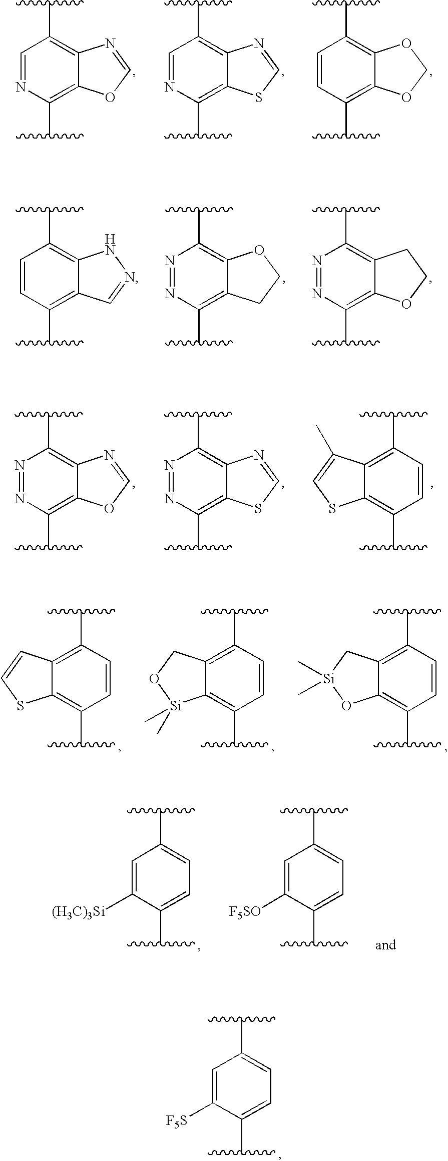

wherein each of said R¹⁰ moieties is optionally substituted with 1-5 independently selected R²¹ groups.

In another embodiment of this invention R¹⁰ in formula (I) is selected from the group consisting of:

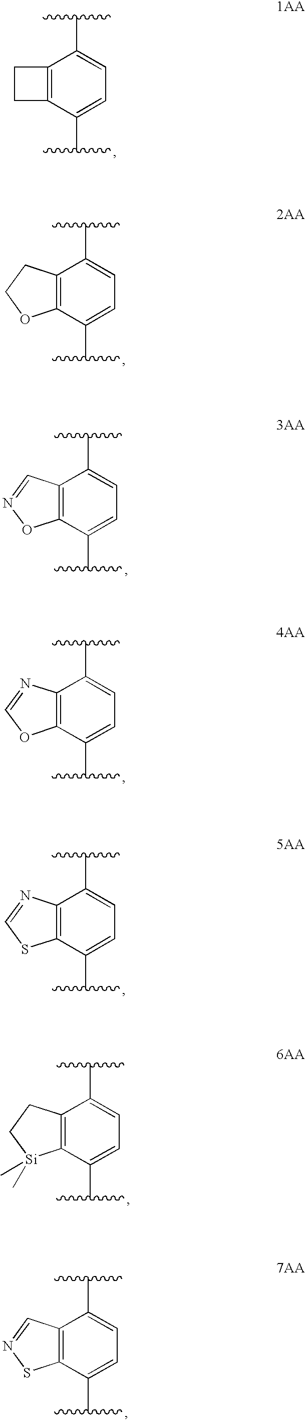

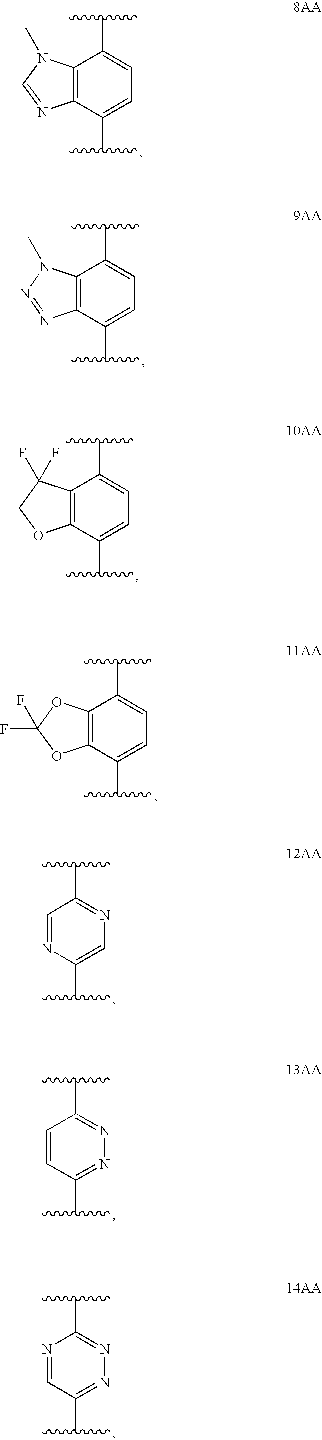

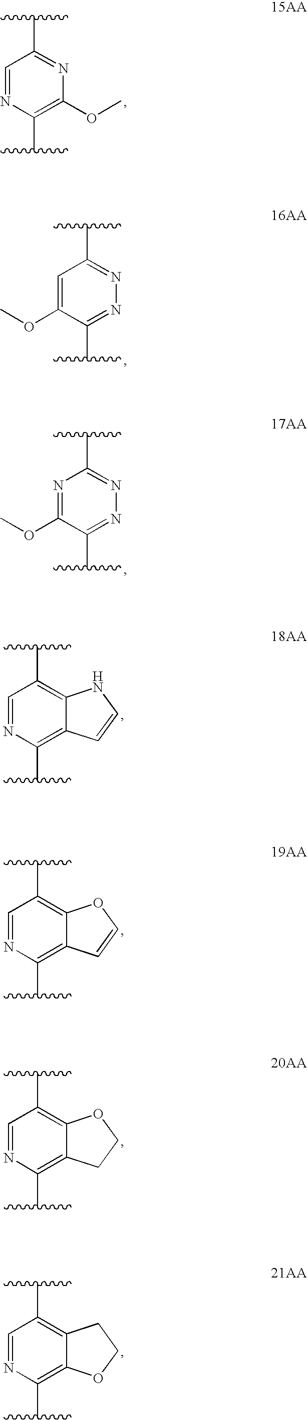

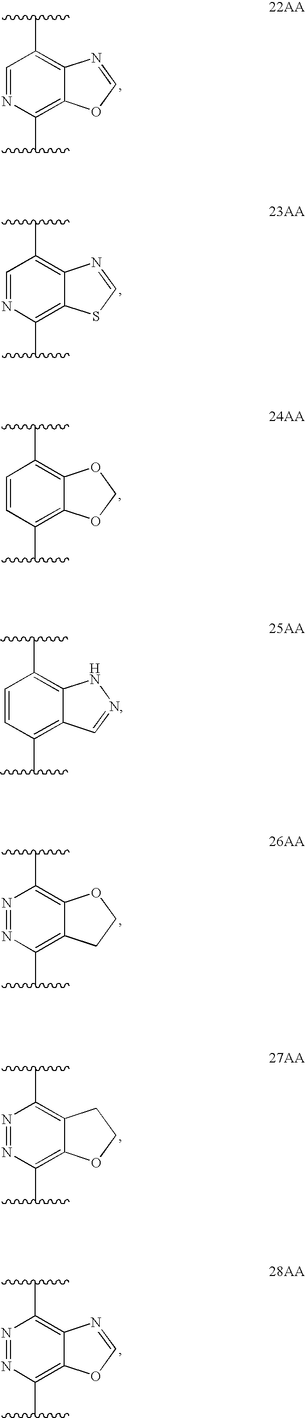

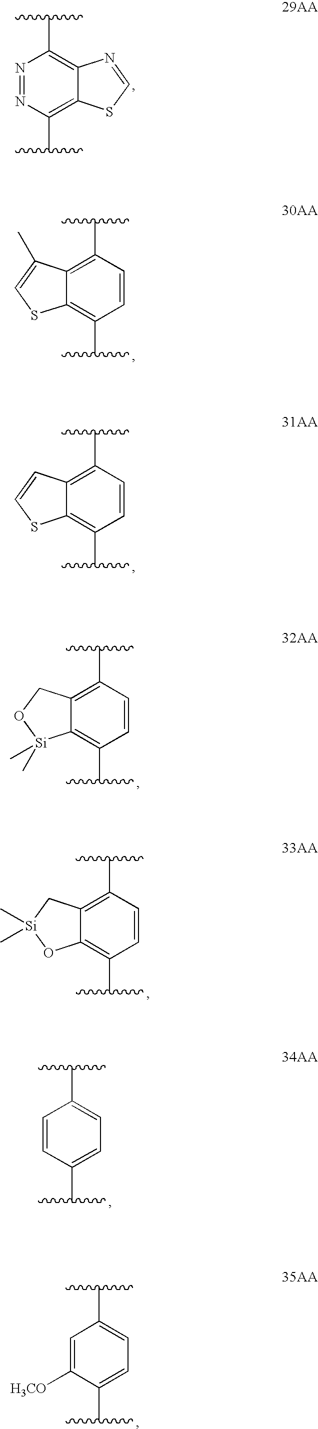

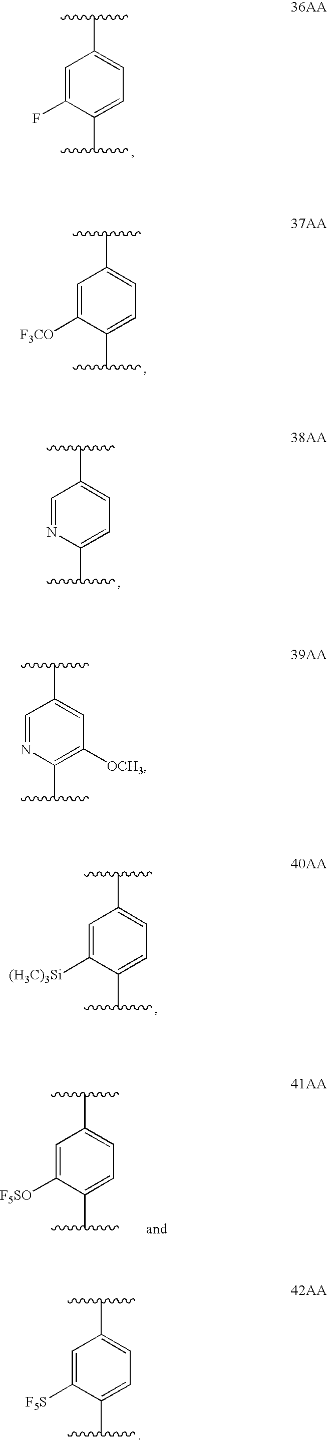

In another embodiment of this invention R¹⁰ is group 1AA. In another embodiment of this invention R¹⁰ is group 2AA. In another embodiment of this invention R¹⁰ is group 3AA. In another embodiment of this invention R¹⁰ is group 4AA. In another embodiment of this invention R¹⁰ is group 5AA. In another embodiment of this invention R¹⁰ is group 6AA. In another embodiment of this invention R¹⁰ is group 7AA. In another embodiment of this invention R¹⁰ is group 8AA. In another embodiment of this invention R¹⁰ is group 9AA. In another embodiment of this invention R¹⁰ is group 10AA. In another embodiment of this invention R¹⁰ is group 11AA. In another embodiment of this invention R¹⁰ is group 12AA. In another embodiment of this invention R¹⁰ is group 13AA. In another embodiment of this invention R¹⁰ is group 14AA. In another embodiment of this invention R¹⁰ is group 15AA. In another embodiment of this invention R¹⁰ is group 16AA. In another embodiment of this invention R¹⁰ is group 17AA. In another embodiment of this invention R¹⁰ is group 18AA. In another embodiment of this invention R¹⁰ is group 19AA. In another embodiment of this invention R¹⁰ is group 20AA. In another embodiment of this invention R¹⁰ is group 21AA. In another embodiment of this invention R¹⁰ is group 22AA, in another embodiment of this invention R¹⁰ is group 23AA. In another embodiment of this invention R¹⁰ is group 24AA. In another embodiment of this invention R¹⁰ is group 25AA. In another embodiment of this invention R¹⁰ is group 26AA. In another embodiment of this invention R¹⁰ is group 27AA. In another embodiment of this invention R¹⁰ is group 28AA. In another embodiment of this invention R¹⁰ is group 29AA. In another embodiment of this invention R¹⁰ is group 30AA. In another embodiment of this invention R¹⁰ is group 31AA. In another embodiment of this invention R¹⁰ is group 32AA. In another embodiment of this invention R¹⁰ is group 33AA. In another embodiment of this invention R¹⁰ is group 34AA. In another embodiment of this invention R¹⁰ is group 35AA. In another embodiment of this invention R¹⁰ is group 36AA. In another embodiment of this invention R¹⁰ is group 37AA. In another embodiment of this invention R¹⁰ is group 38AA. In another embodiment of this invention R¹⁰ is group 39A. In another embodiment of this invention R¹⁰ is group 40AA. In another embodiment of this invention R¹⁰ is group 41AA. In another embodiment of this invention R¹⁰ is group 42AA.

In another embodiment of this invention R¹⁰ is aryl.

In another embodiment of this invention R¹⁰ aryl is aryl and said aryl is phenyl.

In another embodiment of this invention R¹⁰ is aryl substituted with one or more R²¹ groups.

In another embodiment of this invention R¹⁰ is aryl substituted with one or more R²¹ groups, and said aryl is phenyl, i.e., said R¹⁰ group is phenyl substituted with one or more R²¹ groups.

In another embodiment of this invention R¹⁰ is phenyl substituted with one or more R²¹ groups, and each R²¹ group is the same or different —OR¹⁵ group.

In another embodiment of this invention R¹⁰ is phenyl substituted with one or more R²¹ groups, and each R²¹ group is the same or different —OR¹⁵ group, and said R¹⁵ is alkyl, and each alkyl is independently selected.

In another embodiment of this invention R¹⁰ is phenyl substituted with one R²¹ group, and said R²¹ group is —OR¹⁵, and said R¹⁵ is alkyl.

In another embodiment of this invention R¹⁰ is phenyl substituted with one R²¹ group, and said R²¹ group is —OR¹⁵, and said R¹⁵ is alkyl, and said alkyl is methyl.

In another embodiment of this invention R¹⁰ is phenyl substituted with one or more (e.g., one or two, or one) independently selected R²¹ halo groups.

In another embodiment of this invention R¹⁰ is phenyl substituted with one R²¹ group, and said R²¹ group is halo.

In another embodiment of this invention R¹⁰ is phenyl substituted with one R²¹ group, and said R²¹ group is F.

In another embodiment of this invention R¹⁰ is phenyl substituted with one R²¹ group and said R²¹ is an —OR¹⁵ group, and R¹⁵ is an (R¹⁸)ₙalkyl group, and R¹⁸ is halo, and n is 1 to 3, and each halo is independently selected.

In another embodiment of this invention R¹⁰ is phenyl substituted with one R²¹ group, and R²¹ is an —OR¹⁵ group, and R¹⁵ is an (R¹⁸)ₙalkyl group, and R¹⁸ is F, and n is 3.

In another embodiment of this invention R¹⁰ is phenyl substituted with one R²¹ group and said R²¹ is an —OR¹⁵ group, and R¹⁵ is an (R¹⁸)ₙalkyl group, and R¹⁸ is F, and n is 3, and the alkyl is methyl (i.e., the R²¹ substituent is —OCF₃).

In another embodiment of this invention R¹⁰ is heteroaryl.

In another embodiment of this invention R¹⁰ is heteroaryl substituted with one or more R²¹ groups.

In another embodiment of this invention R⁹ is selected from the group consisting of:

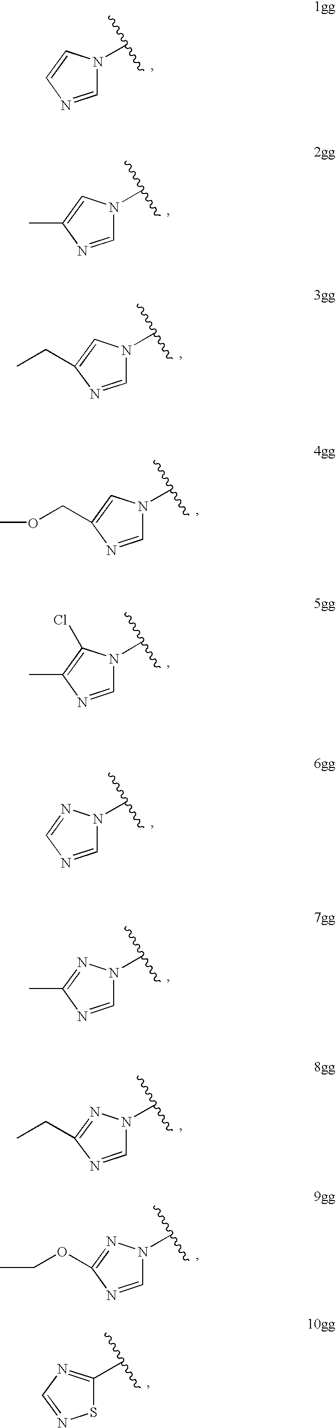

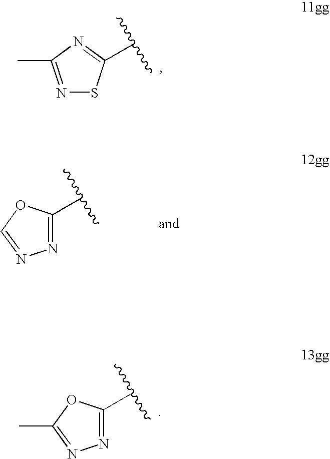

In another embodiment of this invention R¹⁰ is selected from the group consisting of 1AA to 42AA, and R⁹ is selected from the group consisting of 1gg to 13gg.

In another embodiment of this invention R¹⁰ is selected from the group consisting of 1AA to 42AA, and R⁹ is 2gg.

Examples of the R⁹-R¹⁰- moiety include, but are not limited to:

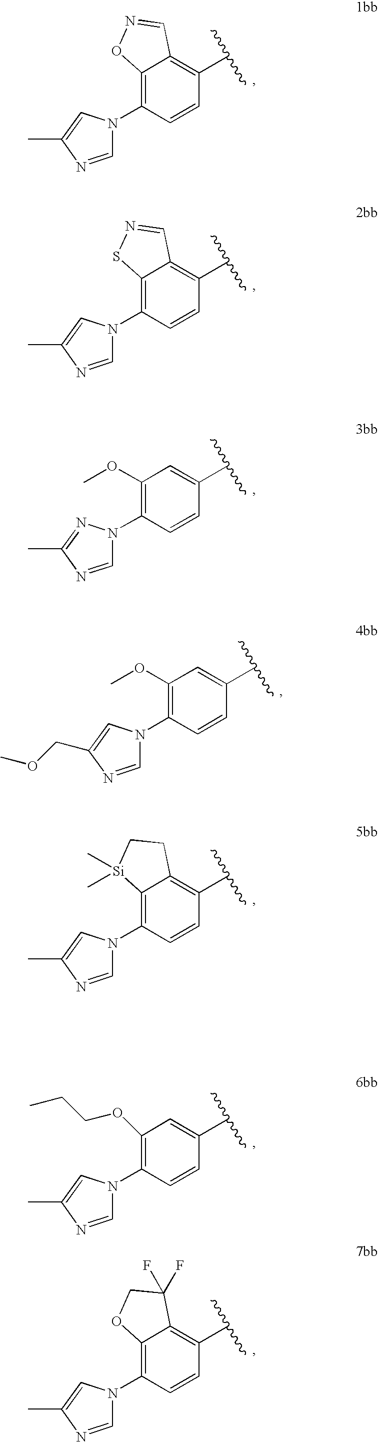

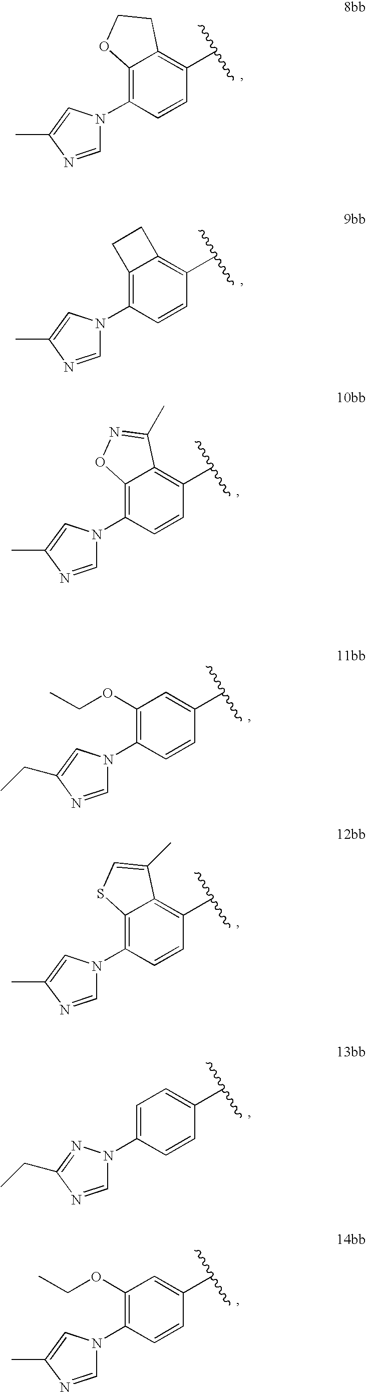

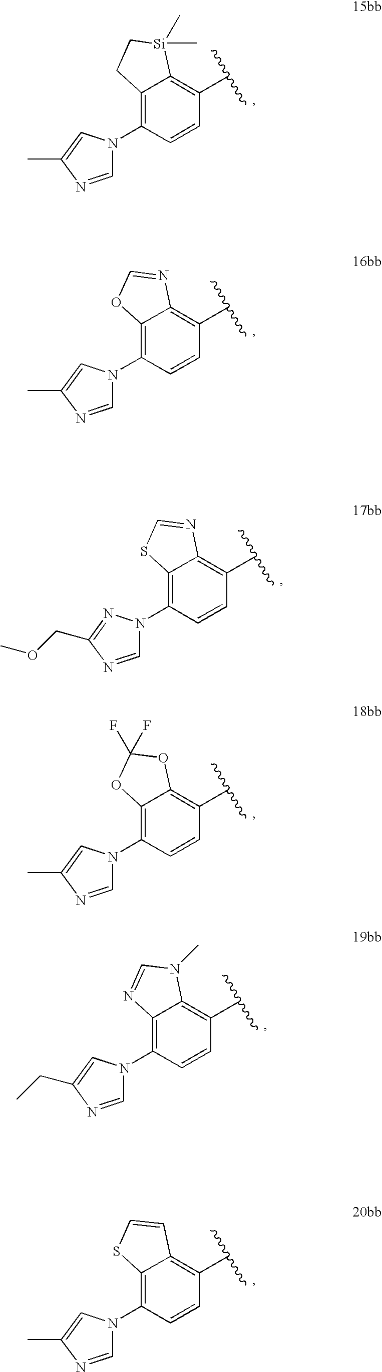

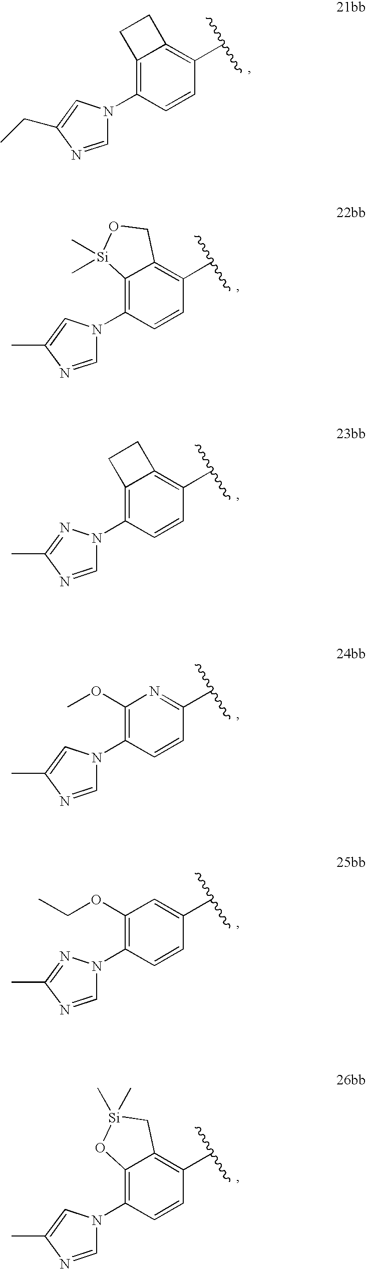

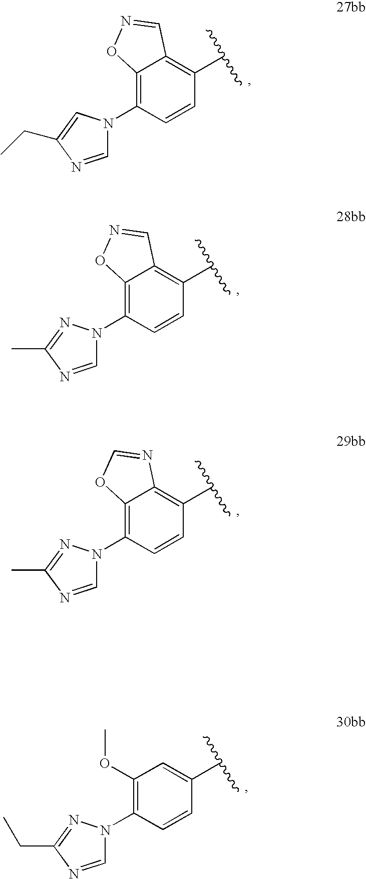

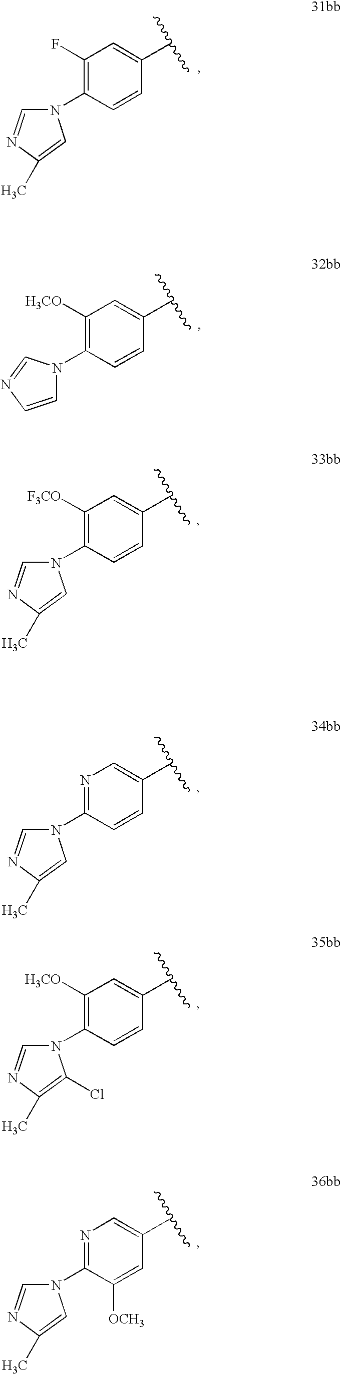

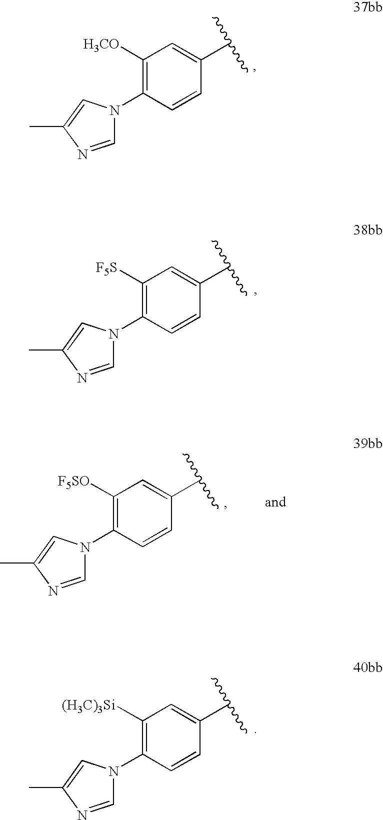

In another embodiment the R⁹-R¹⁰- moiety is 1 bb. In another embodiment the R⁹-R¹⁰- moiety is 2bb. In another embodiment the R⁹-R¹⁰- moiety is 3bb. In another embodiment the R⁹-R¹⁰- moiety is 4bb. In another embodiment the R⁹-R¹⁰- moiety is 5bb. In another embodiment the R⁹-R¹⁰- moiety is Ebb. In another embodiment the R⁹-R¹⁰- moiety is 7bb. In another embodiment the R⁹-R¹⁰- moiety is 8bb. In another embodiment the R⁹-R¹⁰- moiety is 9bb. In another embodiment the R⁹-R¹⁰- moiety is 10bb. In another embodiment the R⁹-R¹⁰- moiety is 11 bb. In another embodiment the R⁹-R¹⁰- moiety is 12bb. In another embodiment the R⁹-R¹⁰- moiety is 13bb. In another embodiment the R⁹-R¹⁰- moiety is 14bb. In another embodiment the R⁹-R¹⁰-moiety is 15bb. In another embodiment the R⁹-R¹⁰- moiety is 16bb. In another embodiment the R⁹-R¹⁰-moiety is 17bb. In another embodiment the R⁹-R¹⁰- moiety is 18bb. In another embodiment the R⁹-R¹⁰- moiety is 19bb. In another embodiment the R⁹-R¹⁰- moiety is 20bb. In another embodiment the R⁹-R¹⁰- moiety is 21bb. In another embodiment the R⁹-R¹⁰- moiety is 22bb. In another embodiment the R⁹-R¹⁰-moiety is 23bb. In another embodiment the R⁹-R¹⁰- moiety is 24bb. In another embodiment the R⁹-R¹⁰- moiety is 25bb. In another embodiment the R⁹-R¹⁰- moiety is 26bb. In another embodiment the R⁹-R¹⁰- moiety is 27bb. In another embodiment the R⁹-R¹⁰- moiety is 28bb. In another embodiment the R⁹-R¹⁰- moiety is 29bb. In another embodiment the R⁹-R¹⁰- moiety is 30bb. In another embodiment the R⁹-R¹⁰- moiety is 31 bb. In another embodiment the R⁹-R¹⁰- moiety is 32bb. In another embodiment the R⁹-R¹⁰- moiety is 33bb. In another embodiment the R⁹-R¹⁰- moiety is 34bb. In another embodiment the R⁹-R¹⁰- moiety is 35bb. In another embodiment the R⁹-R¹⁰- moiety is 36bb. In another embodiment the R⁹-R¹⁰- moiety is 37bb. In another embodiment the R⁹-R¹⁰- moiety is 38bb. In another embodiment the R⁹-R¹⁰-moiety is 39bb. In another embodiment the R⁹-R¹⁰- moiety is 40bb.

In another embodiment of this invention R⁹ is heteroaryl.

In another embodiment of this invention R⁹ is heteroaryl substituted with one or more R²¹ groups.

In another embodiment of this invention R⁹ is heteroaryl substituted with one or more R²¹ groups, and said R²¹ groups are the same or different alkyl.

In another embodiment of this invention R⁹ is heteroaryl substituted with one R²¹ group, and said R²¹ is alkyl.

In another embodiment of this invention R⁹ is heteroaryl substituted with one R²¹ group, and said R²¹ is alkyl, and said alkyl is methyl.

In another embodiment of this invention R⁹ is and said heteroaryl is imidazoyl.

In another embodiment of this invention R⁹ is imidazolyl substituted with one or more R²¹ groups.

In another embodiment of this invention R⁹ is imidazolyl substituted with one or more R²¹ groups, and said R²¹ groups are the same or different alkyl.

In another embodiment of this invention R⁹ is imidazolyl substituted with one R²¹ group, and said R²¹ is alkyl.

In another embodiment of this invention R⁹ is imidazolyl substituted with one R²¹ group, and said R²¹ is alkyl, and said alkyl is methyl.

In another embodiment of this invention R¹⁰ is selected from the group consisting of aryl and aryl substituted with one or more R²¹ groups, and said R⁹ group is selected from the group consisting of heteroaryl and heteroaryl substituted with one or more R²¹ groups, wherein each R²¹ is independently selected.

In another embodiment of this invention R¹⁰ is phenyl substituted with one or more R²¹ groups, and said R⁹ is imidazolyl substituted with one or more R²¹ groups, wherein each R²¹ is independently selected.

In another embodiment of this invention R¹⁰ is phenyl substituted with one R²¹ group, and said R⁹ is imidazolyl substituted with one R²¹ group, wherein each R²¹ is independently selected.

In another embodiment of this invention R¹⁰ is phenyl substituted with one or more independently selected —OR¹⁵ groups, and said R⁹ is imidazolyl substituted with one or more independently selected alkyl groups.

In another embodiment of this invention R¹⁰ is phenyl substituted with one or more independently selected —OR¹⁵ groups, and said R⁹ is imidazolyl substituted with one or more independently selected alkyl groups, and each R¹⁵ is the same or different alkyl group.

In another embodiment of this invention R¹⁰ is phenyl substituted with one —OR¹⁵ group, and said R⁹ is imidazolyl substituted with one alkyl group.

In another embodiment of this invention R¹⁰ is phenyl substituted with one —OR¹⁵ group, and said R⁹ is imidazolyl substituted with one alkyl group, and R¹⁵ is alkyl, and wherein the R¹⁵ alkyl group, and the alkyl group on said imidazolyl are independently selected.

In another embodiment of this invention R¹⁰ is phenyl substituted with one —OR¹⁵ group, and said R⁹ is imidazolyl substituted with one methyl group, and R¹⁵ is methyl, and wherein the R¹⁵ alkyl group, and the alkyl group on said imidazolyl are independently selected.

In another embodiment of this invention the R⁹-R¹⁰- moiety is:

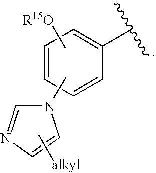

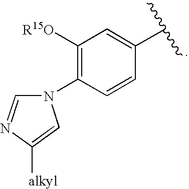

In another embodiment of this invention the R⁹-R¹⁰- moiety is:

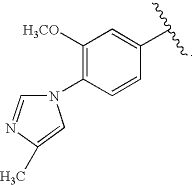

In another embodiment of this invention the R⁹-R¹⁰- moiety is:

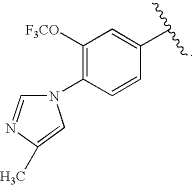

In another embodiment of this invention the R⁹-R¹⁰- moiety is:

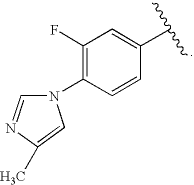

In another embodiment of this invention the R⁹-R¹⁰- moiety is:

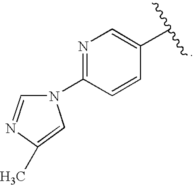

In another embodiment of this invention R^{1A} is an unsubstituted or substituted aryl (e.g., phenyl) group.

In another embodiment of this invention R^{1A} is an unsubstituted aryl (e.g., phenyl) or aryl (e.g., phenyl) substituted with one or more independently selected R²¹ groups.

In another embodiment of this invention R^{1A} is an aryl group.

In another embodiment of this invention R^{1A} is an aryl group, and said aryl group is substituted with one or more independently selected R²¹ groups.

In another embodiment of this invention R^{1A} is an aryl group, and said aryl group is substituted with 1 to 3 independently selected R²¹ groups.

In another embodiment of this invention R^{1A} is an aryl group, and said aryl group is substituted with one or more R²¹ groups, and each R²¹ group is the same or different halo.

In another embodiment of this invention R^{1A} is an aryl group, and said aryl group is substituted with 1 to 3 R²¹ groups, and each R²¹ group is the same or different halo.

In another embodiment of this invention R^{1A} is an aryl group, and said aryl group is substituted with three R²¹ halo groups, and each R²¹ group is the same or different halo.

In another embodiment of this invention R^{1A} is an aryl group, and said aryl group is substituted with two R²¹ halo groups, and each R²¹ group is the same or different halo.

In another embodiment of this invention R^{1A} is an aryl group, and said aryl group is substituted with one R²¹ halo group.

In another embodiment of this invention R^{1A} is an aryl group, and said aryl group is substituted with one R²¹ halo groups, and each R²¹ group is the same or different halo.

In another embodiment of this invention R^{1A} is an aryl group, and said aryl group is substituted with one F (i.e., said aryl is substituted with one R²¹ group, and said R²¹ group is halo, and said halo is F).

In another embodiment of this invention R^{1A} is an aryl group, and said aryl group is substituted with two F atoms (i.e., said aryl is substituted with two R²¹ groups, and said R²¹ groups are halo, and said halo is F).

In another embodiment of this invention R^{1A} is an aryl group, and said aryl group is substituted with three F atoms (i.e., said aryl is substituted with three R²¹ groups, and said R²¹ groups are halo, and said halo is F).

In another embodiment of this invention R^{1A} is phenyl.

In another embodiment of this invention R^{1A} is phenyl, and said phenyl is substituted with one or more independently selected R²¹ groups.

In another embodiment of this invention R^{1A} is phenyl, and said phenyl is substituted with 1 to 3 independently selected R²¹ groups.

In another embodiment of this invention R^{1A} is a phenyl, and said phenyl is substituted with one or more R²¹ groups, and each R²¹ group is the same or different halo.

In another embodiment of this invention R^{1A} is phenyl, and said phenyl is substituted with 1 to 3 R²¹ groups, and each R²¹ group is the same or different halo.

In another embodiment of this invention R^{1A} is phenyl, and said phenyl is substituted with three R²¹ halo groups, and each R²¹ group is the same or different halo.

In another embodiment of this invention R^{1A} is phenyl, and said phenyl is substituted with two R²¹ halo groups, and each R²¹ group is the same or different halo.

In another embodiment of this invention R^{1A} is phenyl, and said phenyl is substituted with one R²¹ halo group.

In another embodiment of this invention R^{1A} is phenyl, and said phenyl is substituted with one R²¹ halo group.

In another embodiment of this invention R^{1A} is phenyl, and said phenyl is substituted with one F (i.e., said aryl is substituted with one R²¹ group, and said R²¹ group is halo, and said halo is F).

In another embodiment of this invention R^{1A} is phenyl, and said phenyl is substituted with two F atoms (i.e., said aryl is substituted with two R²¹ groups, and said R²¹ groups are halo, and said halo is F),

In another embodiment of this invention R^{1A} is phenyl, and said phenyl is substituted with three F atoms (i.e., said aryl is substituted with three R²¹ groups, and said R²¹ groups are halo, and said halo is F).

In another embodiment of this invention R^{1A} is selected from the group consisting of:

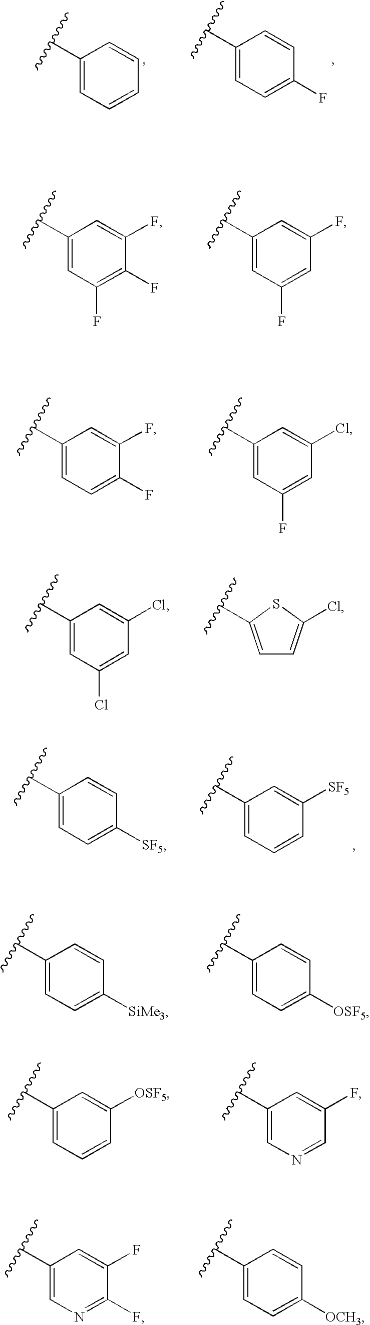

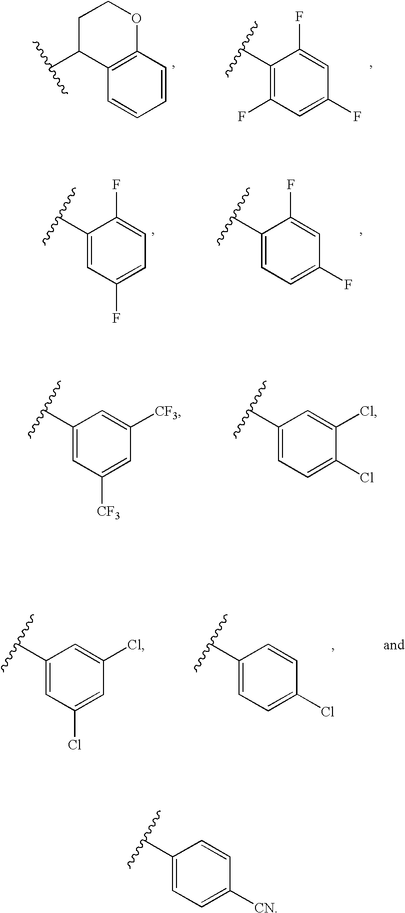

In another embodiment of this invention R^{1A} is selected from the group consisting of:

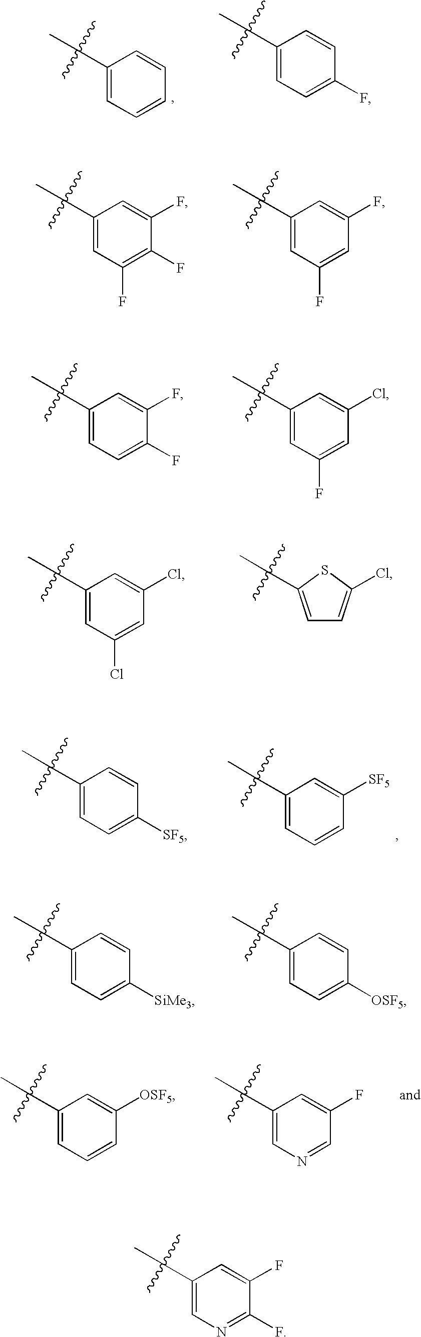

In another embodiment of this invention R^{1A} is selected from the group consisting of:

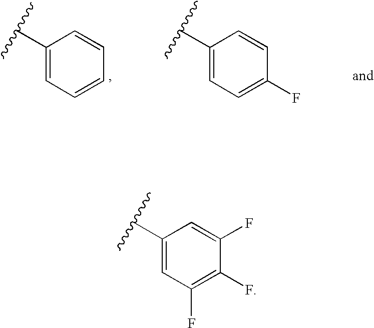

In another embodiment of this invention R^{1A} is selected from the group consisting of:

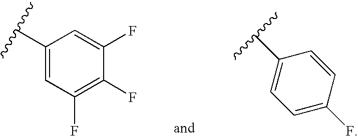

In another embodiment of this invention R^{1A} is:

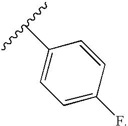

In another embodiment of this invention R^{1A} is:

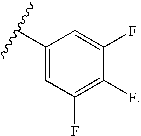

In another embodiment of this invention R^{1A} is:

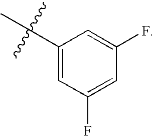

In another embodiment of this invention R^{1A} is:

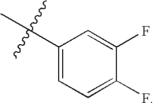

In another embodiment of this invention R^{1A} is:

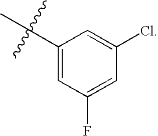

In another embodiment of this invention R^{1A} is:

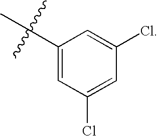

In another embodiment of this invention R^{1A} is:

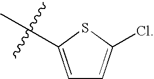

In another embodiment of this invention R^{1A} is:

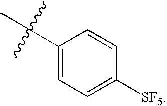

In another embodiment of this invention R^{1A} is:

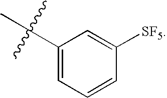

In another embodiment of this invention R^{1A} is:

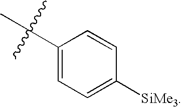

In another embodiment of this invention R^{1A} is:

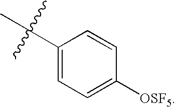

In another embodiment of this invention R^{1A} is:

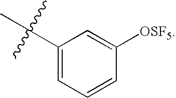

In another embodiment of this invention R^{1A} is:

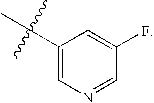

In another embodiment of this invention R^{1A} is:

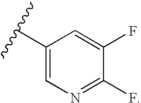

In another embodiment of this invention R^{1A} is:

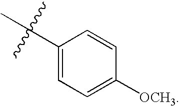

In another embodiment of this invention R^{1A} is:

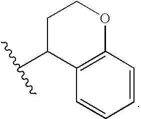

In another embodiment of this invention R^{1A} is:

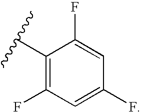

In another embodiment of this invention R^{1A} is:

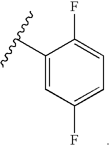

In another embodiment of this invention R^{1A} is:

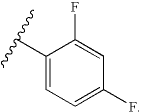

In another embodiment of this invention R^{1A} is:

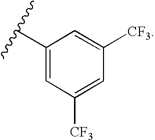

In another embodiment of this invention R^{1A} is:

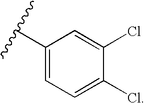

In another embodiment of this invention R^{1A} is:

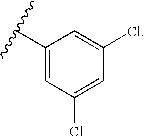

In another embodiment of this invention R^{1A} is:

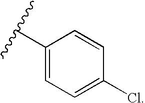

In another embodiment of this invention R^{1A} is:

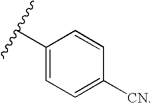

In another embodiment, R^{1A} is phenyl substituted with 1-3 halos independently selected from the group consisting of F and Cl. In one example said phenyl is substituted with one F and one Cl.

In another embodiment R^{1A} is aryl (e.g., phenyl) substituted with 1 to 3 independently selected R²¹ moieties wherein at feast one R²¹ moiety is selected from the group consisting of —SF₅, —OSF₅ and —Si(R^{15A})₃ (and in one example each R^{15A} is the same or different alkyl, and in another example the —Si(R²⁴)₃ group is —Si(CH₃)₃ or —Si(CH₂CH₃)₂CH₃, and in another example the —Si(R²⁴)₃ group is —Si(CH₃)₃).

In another embodiment R^{1A} is aryl (e.g., phenyl) substituted with 1 to 3 independently selected R²¹ moieties wherein at least one R²¹ moiety is selected from the group consisting of —SF₅ and —OSF₅.

In another embodiment R^{1A} is aryl (e.g., phenyl) substituted with 1 to 3 R²¹ moieties independently selected from the group consisting of: halo (e.g., F), —SF₅, —OSF₅ and —Si(R^{15A})₃ (and in one example each R^{15A} is the same or different alkyl, and in another example the —Si(R^{15A})₃ group is —Si(CH₃)₃ or —Si(CH₂CH₃)₂CH₃, and in another example the —Si(R^{15A})₃ group is —Si(CH₃)₃), and wherein at least one R²¹ moiety is selected from the group consisting of —SF₅, —OSF₅ and —Si(R^{15A})₃ (and in one example each R^{15A} is the same or different alkyl, and in another example the —Si(R^{15A})₃ group is —Si(CH₃)₃ or —Si(CH₂CH₃)₂CH₃, and in another example the —Si(R²⁴)₃ group is —Si(CH₃)₃).

In another embodiment R^{1A} is aryl (e.g., phenyl) substituted with 1 to 3 R²¹ moieties independently selected from the group consisting of: halo (e.g., F), —SF₅ and —OSF₅, and wherein at least one R²¹ moiety is selected from the group consisting of —SF₅ and —OSF₅.

In another embodiment R^{1A} is aryl (e.g., phenyl) substituted with 1 to 3 independently selected R²¹ moieties wherein at least one R²¹ moiety is selected from the group consisting of —SF₅, —OSF₅ and —Si(R^{15A})₃ (and in one example each R^{15A} is the same or different alkyl, and in another example the —Si(R^{15A})₃ group is —Si(CH₃)₃ or —Si(CH₂CH₃)₂CH₃, and in another example the —Si(R^{15A})₃ group is —Si(CH₃)₃).

In another embodiment, R^{1A} is phenyl substituted with 1-3 R²¹ groups independently selected from the group consisting of halos, —SF₅ and —OSF₅, wherein at least one R²¹ group is —SF₅ or —OSF₅.

In another embodiment, R^{1A} is phenyl substituted with 1-3 R²¹ groups independently selected from the group consisting of halos, —SF₅ and —OSF₅, wherein at least one R²¹ group is —SF₅ or —OSF₅.

In another embodiment, R^{1A} is phenyl substituted with 1-3 R²¹ groups independently selected from the group consisting of F, Cl, —SF₅ and —OSF₅.

In another embodiment, R^{1A} is phenyl substituted with 1-3 R²¹ groups independently selected from the group consisting of —SF₅ and —OSF₅.

In another embodiment, R^{1A} is phenyl substituted with 1-3 R²¹ groups independently selected from the group consisting of F, —SF₅ and —OSF₅, wherein at least one R²¹ group is —SF₅ or —OSF₅.

In another embodiment, R^{1A} is phenyl substituted with one —SF₅ group.

In another embodiment, R^{1A} is phenyl substituted with two —SF₅ groups.

In another embodiment, R^{1A} is phenyl substituted with three —SF₅ groups.

In another embodiment, R^{1A} is phenyl substituted with one —OSF₅ group.

In another embodiment, R^{1A} is phenyl substituted with two —OSF₅ groups.

In another embodiment, R^{1A} is phenyl substituted with three —OSF₅ groups.

In another embodiment, R^{1A} is phenyl substituted with 1 F.

In another embodiment, R^{1A} is phenyl substituted with 1 F, and also substituted with 1 to 2 groups independently selected from the group consisting of —SF₅ and —OSF₅.

In another embodiment R^{1A} is phenyl substituted with 2 F.

In another embodiment R^{1A} is phenyl substituted with 3F,

In another embodiment of this invention R¹⁰ is selected from the group consisting of aryl and aryl substituted with one or more R²¹ groups, and said R⁹ group is selected from the group consisting of heteroaryl and heteroaryl substituted with one or more R²¹ groups, and wherein each R²¹ is independently selected.

In another embodiment of this invention: (a) R^{1A} is an aryl group, or R^{1A} is an aryl group substituted with 1 to 3 independently selected R²¹ groups, and (b) R¹⁰ is selected from the group consisting of aryl and aryl substituted with one or more independently selected R²¹ groups, and (c) R⁹ is selected from the group consisting of heteroaryl and heteroaryl substituted with one or more independently selected R²¹ groups.

In another embodiment of this invention: (a) R^{1A} is phenyl, or R^{1A} is phenyl substituted with 1 to 3 independently selected R²¹ groups, and (b) R¹⁰ is selected from the group consisting of aryl and aryl substituted with one or more independently selected R²¹ groups, and (c) R⁹ is selected from the group consisting of heteroaryl and heteroaryl substituted with one or more independently selected R²¹ groups.

In another embodiment of this invention: (a) R^{1A} is phenyl, or R^{1A} is phenyl substituted with 1 to 3 independently selected R²¹ groups, and (b) R¹⁰ is selected from the group consisting of phenyl and phenyl substituted with one or more independently selected R²¹ groups, and (c) R⁹ is selected from the group consisting of imidazolyl and imidazolyl substituted with one or more independently selected R²¹ groups.

In another embodiment of this invention: (a) R^{1A} is phenyl, or R^{1A} is phenyl substituted with 1 to 3 independently selected R²¹ halo groups, and (b) R¹⁰ is selected from the group consisting of phenyl and phenyl substituted with one or more independently selected —OR¹⁵ groups, and (c) R⁹ is selected from the group consisting of imidazolyl and imidazolyl substituted with one or more independently selected alkyl groups groups.

In another embodiment of this invention: (a) R^{1A} is phenyl, or R^{1A} is phenyl substituted with 1 to 2 independently selected R²¹ halo groups, and (b) R¹⁰ is selected from the group consisting of phenyl and phenyl substituted with one or two independently selected —OR¹⁵ groups, wherein R¹⁵ is alkyl, and (c) R⁹ is selected from the group consisting of imidazolyl and imidazolyl substituted with one or two independently selected alkyl groups groups.

In another embodiment of this invention: (a) R^{1A} is phenyl, or R^{1A} is phenyl substituted with 1 R²¹ halo group, and (b) R¹⁰ is selected from the group consisting of phenyl and phenyl substituted with one or two independently selected —OR¹⁵ groups, wherein R¹⁵ is alkyl, and (c) R⁹ is selected from the group consisting of imidazolyl and imidazolyl substituted with one or two independently selected alkyl groups groups.

In another embodiment of this invention: (a) R^{1A} is phenyl, or R^{1A} is phenyl, substituted with 1 to 3 F (i.e., R^{1A} is phenyl substituted with 1 to 3 R²¹ groups, and said R²¹ groups are halo, and said halo is F), and (b) R¹⁰ is selected from the group consisting of phenyl and phenyl substituted with one or two independently selected —OR¹⁵ groups, wherein R¹⁵ is methyl, and (c) R⁹ is selected from the group consisting of imidazolyl and imidazolyl substituted with one or two independently selected methyl groups groups.

In another embodiment of this invention: (a) R^{1A} is phenyl, or R^{1A} is phenyl, substituted with 1 to 2 F (i.e., R^{1A} is phenyl substituted with 1 to 2 R²¹ groups, and said R²¹ groups are halo, and said halo is F), and (b) R¹⁰ is selected from the group consisting of phenyl and phenyl substituted with one or two independently selected —OR¹⁵ groups, wherein R¹⁵ is methyl, and (c) R⁹ is selected from the group consisting of imidazolyl and imidazolyl substituted with one or two independently selected methyl groups groups.

In another embodiment of this invention: (a) R^{1A} is phenyl, or R^{1A} is phenyl, substituted with 1 F (i.e., R^{1A} is phenyl substituted with 1 R²¹ group, and said R²¹ group is halo, and said halo is F), and (b) R¹⁰ is selected from the group consisting of phenyl and phenyl substituted with one or two independently selected —OR¹⁵ groups, wherein R¹⁵ is methyl, and (c) R⁹ is selected from the group consisting of imidazolyl and imidazolyl substituted with one or two independently selected methyl groups groups.

In another embodiment of this invention R^{1A} is selected from the group consisting of:

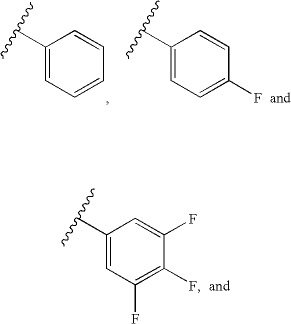

wherein the R⁹-R¹⁰- moiety is:

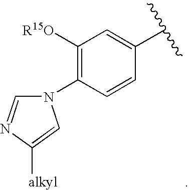

In another embodiment of this invention R^{1A} is selected from the group consisting of:

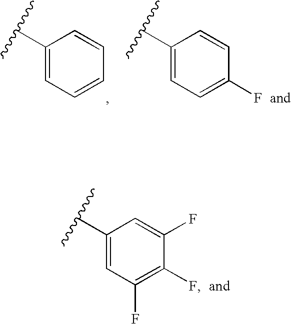

wherein the R⁹-R¹⁰- moiety is:

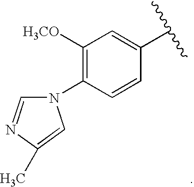

In another embodiment of this invention R^{1A} is selected from the group consisting of:

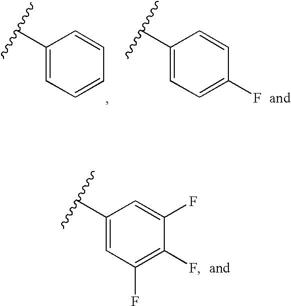

wherein the R⁹-R¹⁰- moiety is:

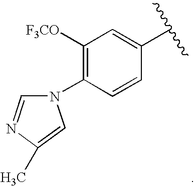

In another embodiment of this invention R^{1A} is selected from the group consisting of:

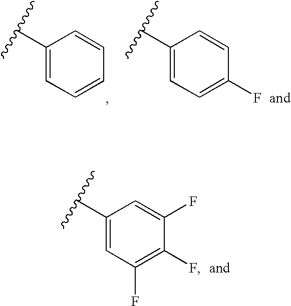

wherein the R⁹-R¹⁰- moiety is:

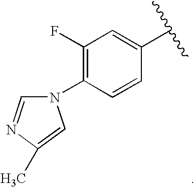

In another embodiment of this invention R^{1A} is selected from the group consisting of:

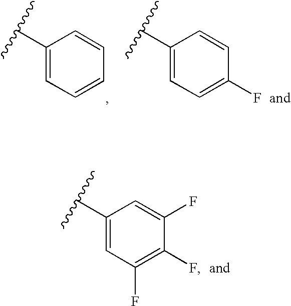

wherein the R⁹-R¹⁰- moiety is:

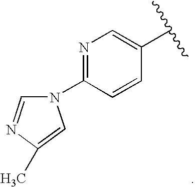

In another embodiment of this invention: (a) R^{1A} is phenyl, or R^{1A} is phenyl substituted with 1 to 3 independently selected R²¹ halo groups, and (b) R¹⁰ is selected from the group consisting of phenyl and phenyl substituted with one or two independently selected —OR¹⁵ groups, wherein R¹⁵ is alkyl, and (c) R⁹ is selected from the group consisting of imidazolyl and imidazolyl substituted with one or two independently selected alkyl groups groups.

In another embodiment of this invention: (a) R^{1A} is phenyl, or R^{1A} is phenyl substituted with 1 to 3 independently selected R²¹ halo groups, and (b) R¹⁰ is selected from the group consisting of phenyl and phenyl substituted with one or two independently selected —OR¹⁵ groups, wherein R¹⁵ is alkyl, and (c) R⁹ is selected from the group consisting of imidazolyl and imidazolyl substituted with one or two independently selected alkyl groups groups, and (d) G is —C(O)—.

In another embodiment of this invention: (a) R^{1A} is phenyl, or R^{1A} is phenyl substituted with 1 to 3 independently selected R²¹ halo groups, and (b) R¹⁰ is selected from the group consisting of phenyl and phenyl substituted with one or two independently selected —OR¹⁵ groups, wherein R¹⁵ is alkyl, and (c) R⁹ is selected from the group consisting of imidazolyl and imidazolyl substituted with one or two independently selected alkyl groups groups, and (d) G is —(C═NR²)—.

In another embodiment of this invention: (a) R^{1A} is phenyl, or R^{1A} is phenyl substituted with 1 to 3 independently selected R²¹ halo groups, and (b) R¹⁰ is selected from the group consisting of phenyl and phenyl substituted with one or two independently selected —OR¹⁵ groups, wherein R¹⁵ is alkyl, and (c) R⁹ is selected from the group consisting of imidazolyl and imidazolyl substituted with one or two independently selected alkyl groups groups, and (d) G is —(C═C(R⁶)₂)—.

In another embodiment of this invention: (a) R^{1A} is phenyl, or R^{1A} is phenyl substituted with 1 to 3 independently selected R²¹ halo groups, and (b) R¹⁰ is selected from the group consisting of phenyl and phenyl substituted with one or two independently selected —OR¹⁵ groups, wherein R¹⁵ is alkyl, and (c) R⁹ is selected from the group consisting of imidazolyl and imidazolyl substituted with one or two independently selected alkyl groups groups, and (d) G is —CHR³—.

In another embodiment of this invention: (a) R^{1A} is phenyl, or R^{1A} is phenyl substituted with 1 to 3 independently selected R²¹ halo groups, and (b) R¹⁰ is selected from the group consisting of phenyl and phenyl substituted with one or two independently selected —OR¹⁵ groups, wherein R¹³ is alkyl, and (c) R⁹ is selected from the group consisting of imidazolyl and imidazolyl substituted with one or two independently selected alkyl groups groups, and (d) G is —CH₂—.

In another embodiment of this invention: (a) R^{1A} is phenyl, or R^{1A} is phenyl substituted with 1 to 3 independently selected R²¹ halo groups, and (b) R¹⁰ is selected from the group consisting of phenyl and phenyl substituted with one or two independently selected —OR¹⁵ groups, wherein R¹⁵ is alkyl, and (c) R⁹ is selected from the group consisting of imidazolyl and imidazolyl substituted with one or two independently selected alkyl groups groups, and (d) G is —(CHOH)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —C(O)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —(C═NR²)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —(C═C(R⁶)₂)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —CHR³—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —CH₂—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —(CHOH)—.

In another embodiment of this invention R^{1A} is selected from the group consisting of:

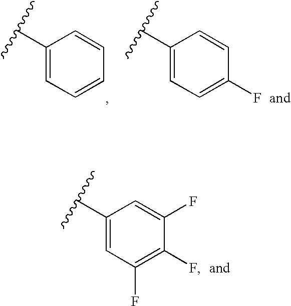

wherein the R⁹-R¹⁰- moiety is:

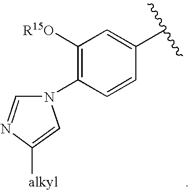

In another embodiment of this invention R^{1A} is selected from the group consisting of

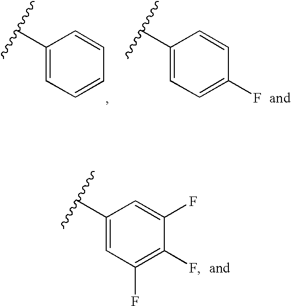

wherein the R⁹-R¹⁰- moiety is:

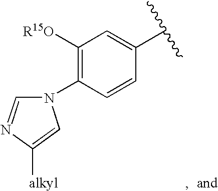

### G is —C(O)—.

In another embodiment of this invention R^{1A} is selected from the group consisting of:

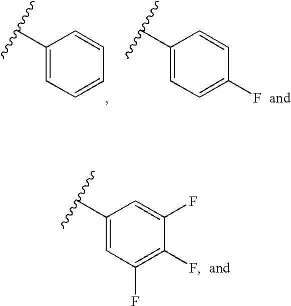

wherein the R⁹-R¹⁰- moiety is:

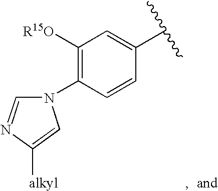

### G is —(C═NR²)—.

In another embodiment of this invention R^{1A} is selected from the group consisting of:

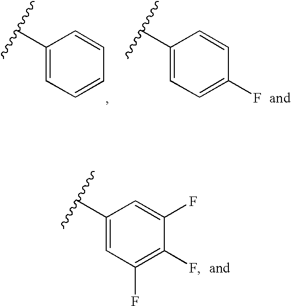

wherein the R⁹-R¹⁰- moiety is:

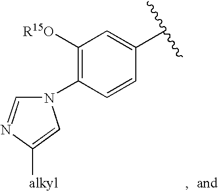

### G is —NHC(O)—

In another embodiment of this invention R^{1A} is selected from the group consisting of:

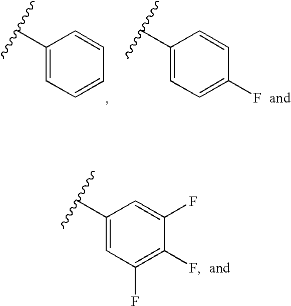

wherein the R⁹-R¹⁰- moiety is:

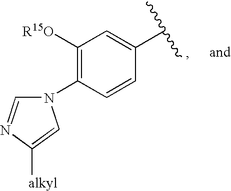

### G is —CHR³—.

In another embodiment of this invention R^{1A} is selected from the group consisting of:

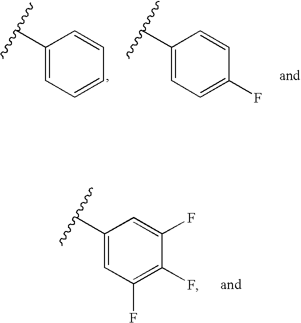

wherein the R⁹-R¹⁰- moiety is:

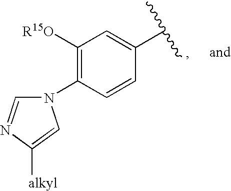

### G is —═—.

In another embodiment of this invention R^{1A} is selected from the group consisting of:

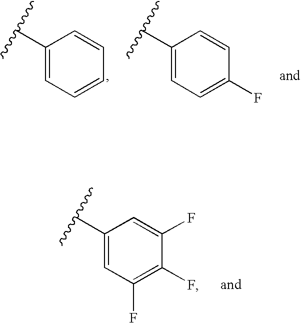

wherein the R⁹-R¹⁰- moiety is:

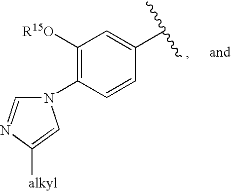

### G is —≡—.

In another embodiment of this invention R^{1A} is selected from the group consisting of:

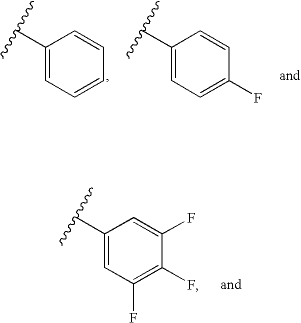

wherein the R⁹-R¹⁰- moiety is:

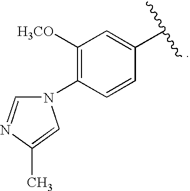

In another embodiment of this invention R^{1A} is selected from the group consisting of:

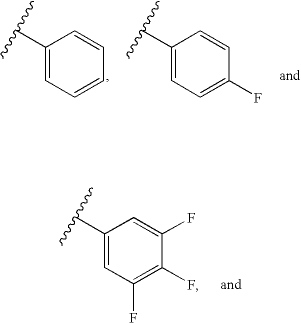

wherein the R⁹-R¹⁰- moiety is:

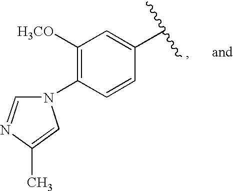

### G is —C(O)—.

In another embodiment of this invention R^{1A} is selected from the group consisting of:

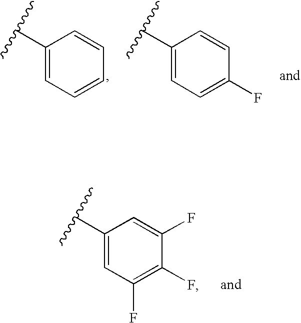

wherein the R⁹-R¹⁰- moiety is:

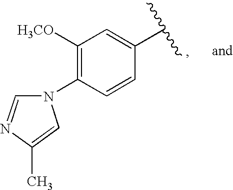

### G is —(C═NR²)—.

In another embodiment of this invention R^{1A} is selected from the group consisting of:

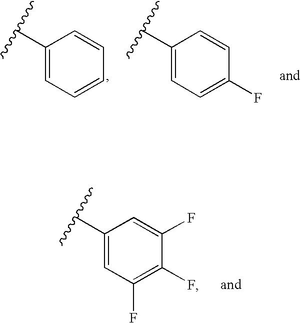

wherein the R⁹-R¹⁰- moiety is:

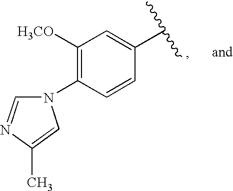

### G is —NHC(O)—.

In another embodiment of this invention R^{1A} is selected from the group consisting of:

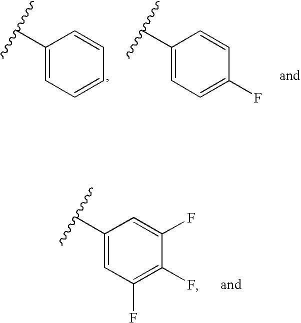

wherein the R⁹-R¹⁰- moiety is:

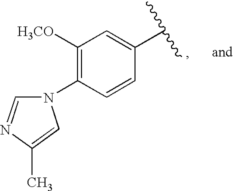

### G is —CHR³—.

In another embodiment of this invention R^{1A} is selected from the group consisting of:

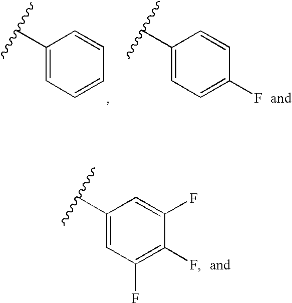

wherein the R⁹-R¹⁰- moiety is:

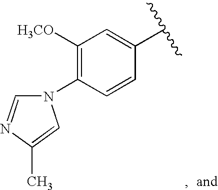

### G is —═—.

In another embodiment of this invention R^{1A} is selected from the group consisting of:

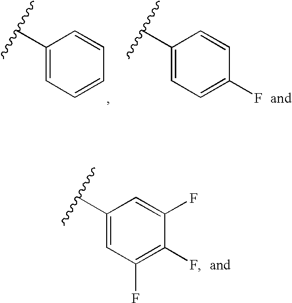

wherein the R⁹-R¹⁰- moiety is:

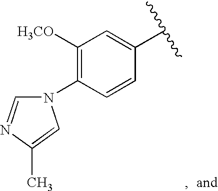

### G is —≡—.

In another embodiment of this invention R^{1A} is selected from the group consisting of:

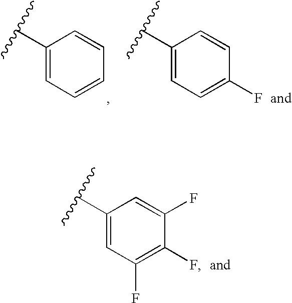

wherein the R⁹-R¹⁰- moiety is:

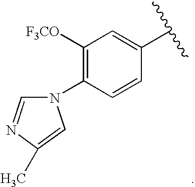

In another embodiment of this invention R^{1A} is selected from the group consisting of:

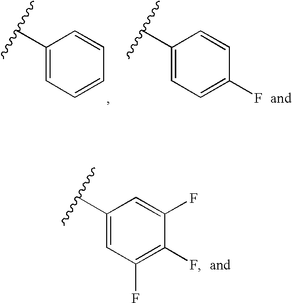

wherein the R⁹-R¹⁰- moiety is:

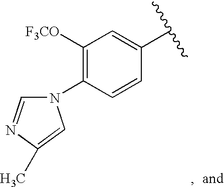

### G is —C(O)—.

In another embodiment of this invention R^{1A} is selected from the group consisting of:

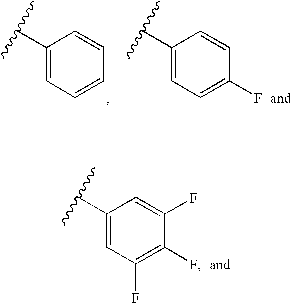

wherein the R⁹-R¹⁰- moiety is:

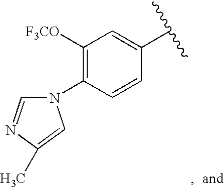

### G is —(C═NR²)—.

In another embodiment of this invention R^{1A} is selected from the group consisting of:

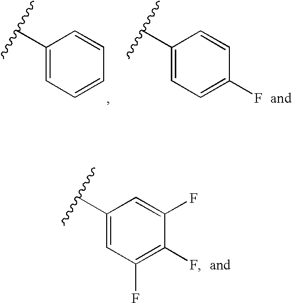

wherein the R⁹-R¹⁰- moiety is:

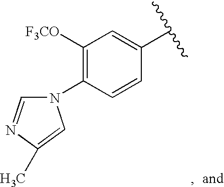

### G is —NHC(O)—.

In another embodiment of this invention R^{1A} is selected from the group consisting of:

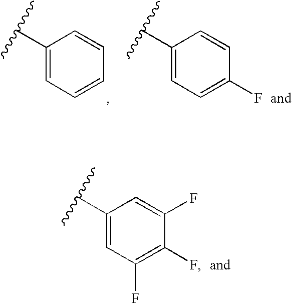

wherein the R⁹-R¹⁰- moiety is:

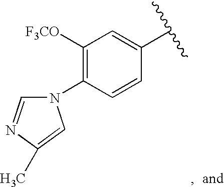

### G is —CHR³—.

In another embodiment of this invention R^{1A} is selected from the group consisting of:

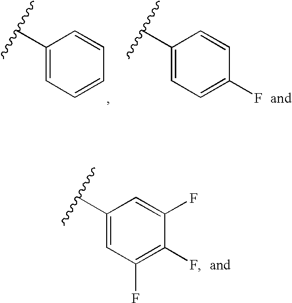

wherein the R⁹-R¹⁰- moiety is:

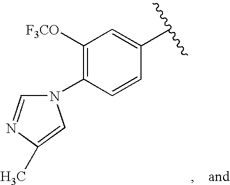

### G is —═—.

In another embodiment of this invention R^{1A} is selected from the group consisting of:

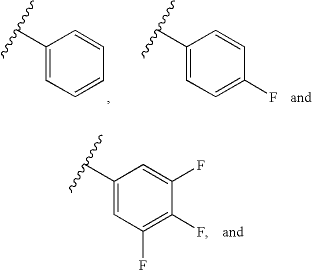

wherein the R⁹-R¹⁰- moiety is:

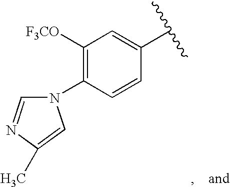

### G is —≡—.

In another embodiment of this invention R^{1A} is selected from the group consisting of:

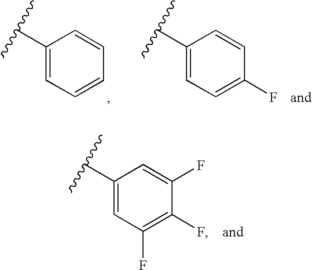

wherein the R⁹-R¹⁰- moiety is:

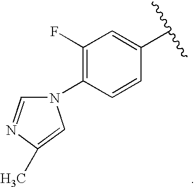

In another embodiment of this invention R^{1A} is selected from the group consisting of:

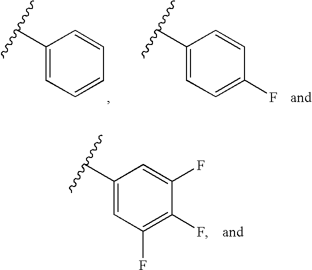

wherein the R⁹-R¹⁰- moiety is:

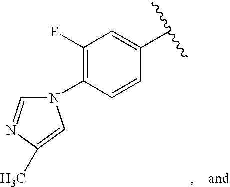

### G is —C(O)—.

In another embodiment of this invention R^{1A} is selected from the group consisting of:

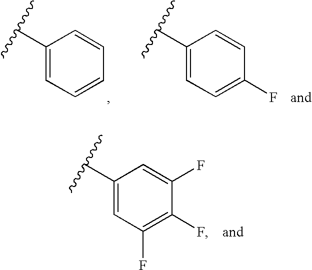

wherein the R⁹-R¹⁰- moiety is:

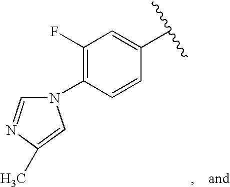

### G is —(C═NR²)—.

In another embodiment of this invention R^{1A} is selected from the group consisting of:

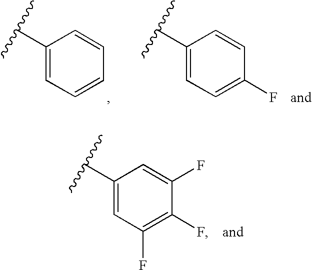

wherein the R⁹-R¹⁰- moiety is:

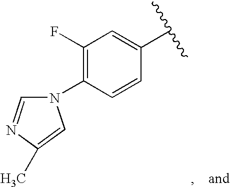

### G is —NHC(O)—.

In another embodiment of this invention R^{1A} is selected from the group consisting of:

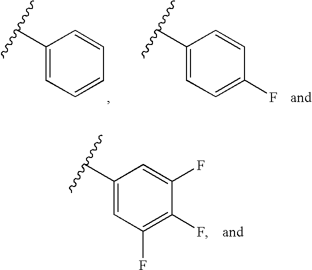

wherein the R⁹-R¹⁰- moiety is:

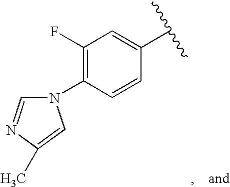

### G is —CHR³—.

In another embodiment of this invention R^{1A} is selected from the group consisting of:

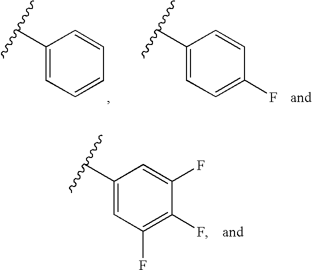

wherein the R⁹-R¹⁰- moiety is:

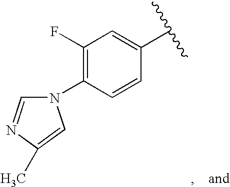

### G is —═—.

In another embodiment of this invention R^{1A} is selected from the group consisting of:

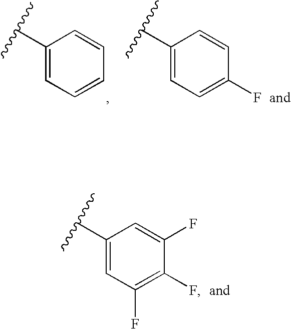

wherein the R⁹-R¹⁰- moiety is:

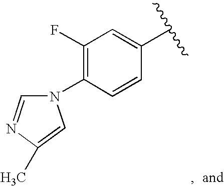

### G is —≡—.

In another embodiment of this invention R^{1A} is selected from the group consisting of:

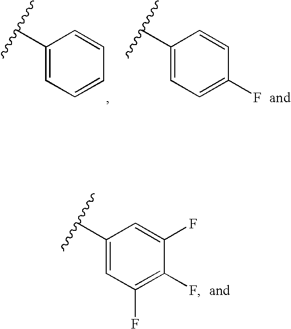

wherein the R⁹-R¹⁰- moiety is:

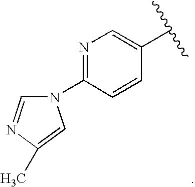

In another embodiment of this invention R^{1A} is selected from the group consisting of:

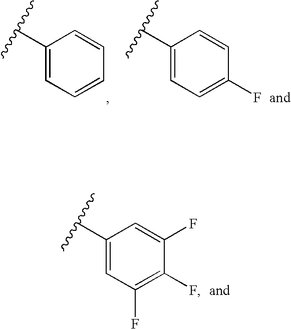

wherein the R⁹-R¹⁰- moiety is:

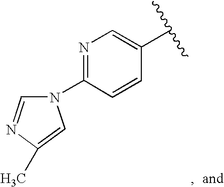

### G is —C(O)—.

In another embodiment of this invention R^{1A} is selected from the group consisting of:

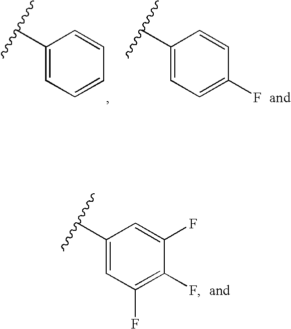

wherein the R⁹-R¹⁰- moiety is:

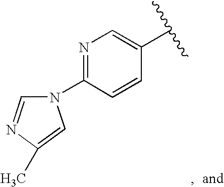

### G is —(C═NR²)—.

In another embodiment of this invention R^{1A} is selected from the group consisting of:

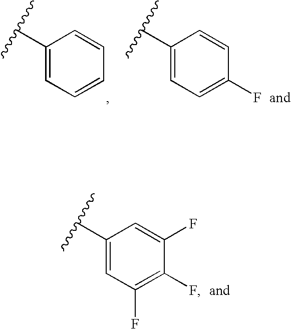

wherein the R⁹-R¹⁰- moiety is:

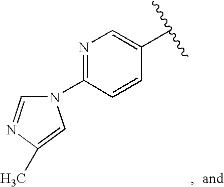

### G is —NHC(O)—.

In another embodiment of this invention R^{1A} is selected from the group consisting of:

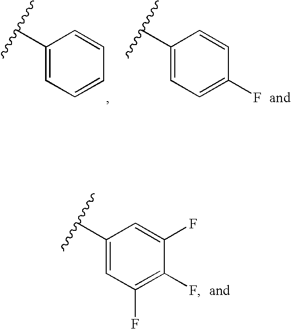

wherein the R⁹-R¹⁰- moiety is:

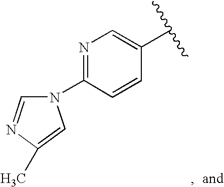

### G is —CHR³—.

In another embodiment of this invention R^{1A} is selected from the group consisting of:

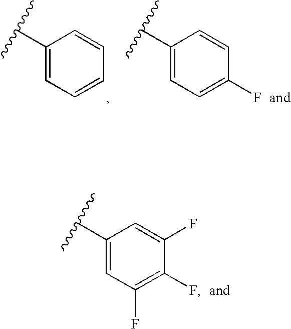

wherein the R⁹-R¹⁰- moiety is:

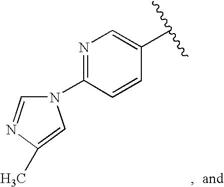

### G is —═—.

In another embodiment of this invention R^{1A} is selected from the group consisting of:

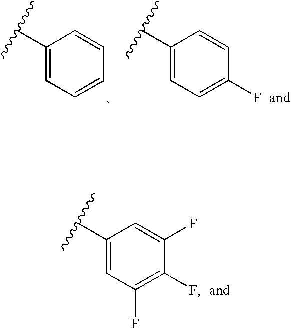

wherein the R⁹-R¹⁰- moiety is

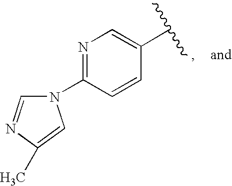

### G is —≡—.

In another embodiment of this invention: (a) R^{1A} is phenyl, or R^{1A} is phenyl substituted with 1 to 3 independently selected R²¹ halo groups, and (b) R¹⁰ is selected from the group consisting of phenyl and phenyl substituted with one or two independently selected —OR¹⁵ groups, wherein R¹⁵ is alkyl, and (c) R⁹ is selected from the group consisting of imidazolyl and imidazolyl substituted with one or two independently selected alkyl groups groups, and (d) G is selected from the group consisting of —NH—, —O—, —S—, —S(O)—, —S(O)₂— and a direct bond.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is selected from the group consisting of —NH—, —O—, —S—, —S(O)—, —S(O)₂— and a direct bond.

In another embodiment of this invention R^{1A} is selected from the group consisting of:

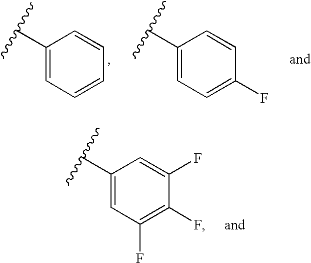

wherein the R⁹-R¹⁰- moiety is:

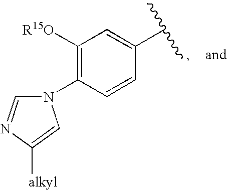

G is selected from the group consisting of —NH—, —O—, —S—, —S(O)—, —S(O)₂— and a direct bond.

In another embodiment of this invention R^{1A} is selected from the group consisting of:

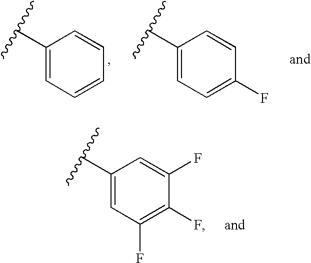

wherein the R⁹-R¹⁰-moiety is:

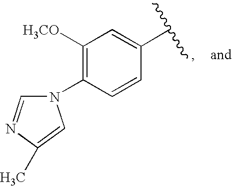

G is selected from the group consisting of —NH—, —O—, —S—, —S(O)—, —S(O)₂— and a direct bond.

In another embodiment of this invention R^{1A} is selected from the group consisting of:

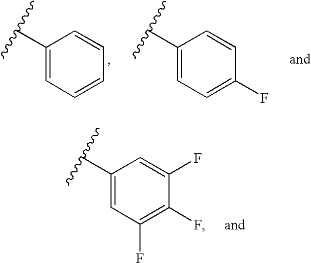

wherein the R⁹-R¹⁰- moiety is:

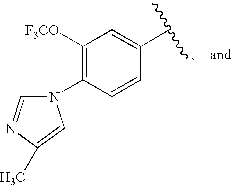

G is selected from the group consisting of —NH—, —O—, —S—, —S(O)—, —S(O)₂— and a direct bond.

In another embodiment of this invention R^{1A} is selected from the group consisting of:

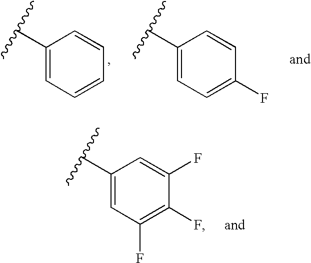

wherein the R⁹-R¹⁰- moiety is:

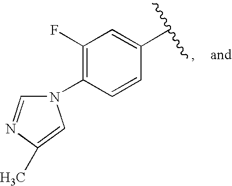

G is selected from the group consisting of —NH—, —O—, —S—, —S(O)—, —S(O)₂— and a direct bond.

In another embodiment of this invention R^{1A} is selected from the group consisting of:

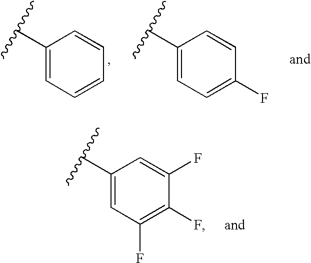

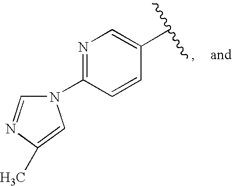

G is selected from the group consisting of —NH—, —O—, —S—, —S(O)—, —S(O)₂— and a direct bond.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is selected from the group consisting of —NH—, —O—, —S—, —S(O)—, —S(O)₂— and a direct bond, and (e) W is —C(O)—.

In another embodiment of this invention R^{1A} is selected from the group consisting of:

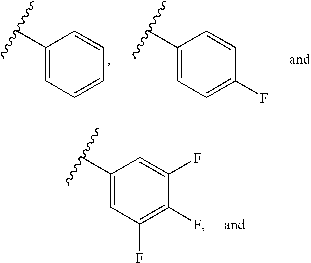

wherein the R⁹-R¹⁰- moiety is:

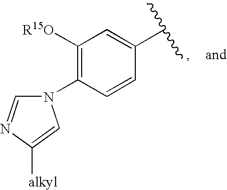

G is selected from the group consisting of —NH—, —O—, —S—, —S(O)—, —S(O)₂— and a direct bond, and W is —C(O)—.

In another embodiment of this invention R^{1A} is selected torn the group consisting of:

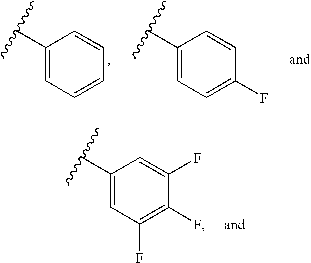

wherein the R⁹-R¹⁰- moiety is:

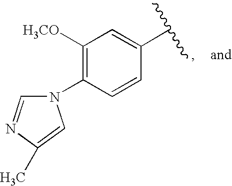

G is selected from the group consisting of —NH—, —O—, —S—, —S(O)—, —S(O)₂— and a direct bond, and W is —C(O)—.

In another embodiment of this invention R^{1A} is selected from the group consisting of:

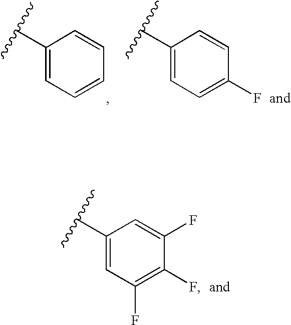

wherein the R⁹-R¹⁰- moiety is:

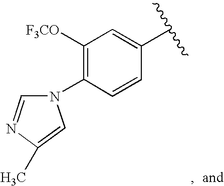

G is selected from the group consisting of —NH—, —O—, —S—, —S(O)—-, —S(O)₂— and a direct bond, and W is —C(O)—.

In another embodiment of this invention R^{1A} is selected from the group consisting of:

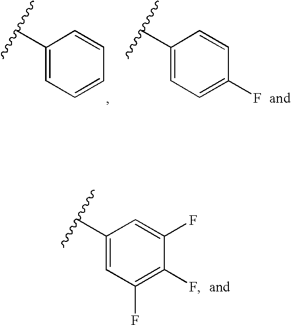

wherein the R⁹-R¹⁰- moiety is:

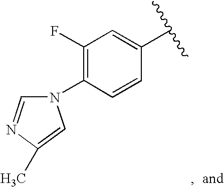

G is selected from the group consisting of —NH—, —O—, —S—, —S(O)—, —S(O)₂— and a direct bond, and W is —C(O)—.

In another embodiment of this invention R^{1A} is selected from the group consisting of:

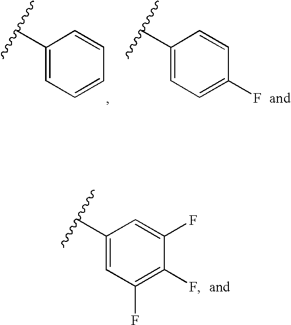

wherein the R⁹-R¹⁰- moiety is:

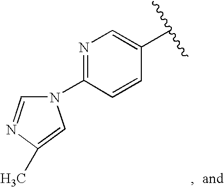

G is selected from the group consisting of —NH—, —O—, —S— —S(O)—, —S(O)₂— and a direct bond, and W is —C(O)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is selected from the group consisting of —NH—, —O—, —S—, —S(O)—, —S(O)₂— and a direct bond, and (e) W is —S(O)—.

In another embodiment of this invention R^{1A} is selected from the group consisting of:

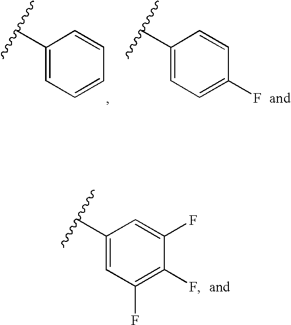

wherein the R⁹-R¹⁰- moiety is:

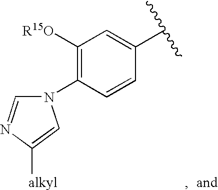

G is selected from the group consisting of —NH—, —O—, —S—, —S(O)—, —S(O)₂— and a direct bond, and W is —S(O)—.

In another embodiment of this invention R^{1A} is selected from the group consisting of:

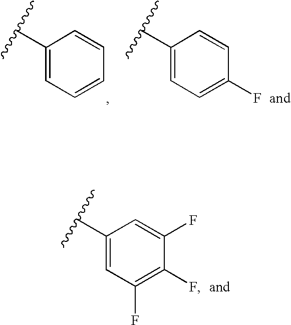

wherein the R⁹-R¹⁰- moiety is:

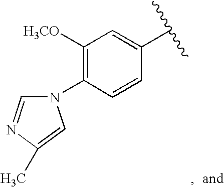

G is selected from the group consisting of —NH—, —O—, —S—, —S(O)—, —S(O)₂— and a direct bond, and W is —S(O)—.

In another embodiment of this invention R^{1A} is selected from the group consisting of:

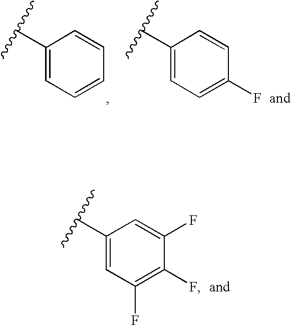

wherein the R⁹-R¹⁰- moiety is:

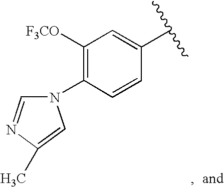

G is selected from the group consisting of —NH—, —O—, —S—, —S(O)—, —S(O)₂— and a direct bond, and W is —S(O)—.

In another embodiment of this invention R^{1A} is selected from the group consisting of:

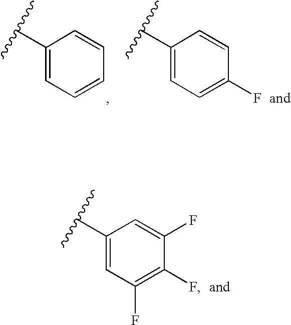

wherein the R⁹-R¹⁰- moiety is:

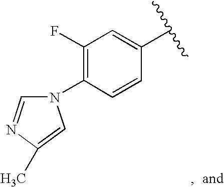

G is selected from the group consisting of —NH—, —O—, —S—, —S(O)—, —S(O)₂— and a direct bond, and W is —S(O)—.

In another embodiment of this invention R^{1A} is selected from the group consisting of:

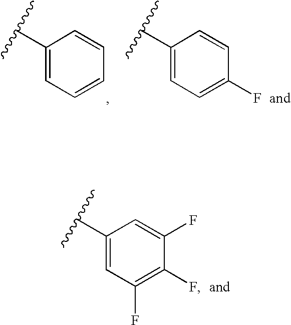

wherein the R⁹-R¹⁰- moiety is:

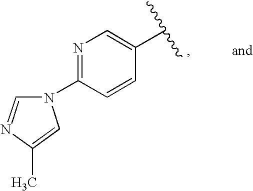

G is selected from the group consisting of —NH—, —O—, —S—, —S(O)—, —S(O)₂— and a direct bond, and W is —S(O)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is selected from the group consisting of —NH—, —O—, —S—, —S(O)—, —S(O)₂— and a direct bond, and (e) W is —S(O)₂—.

In another embodiment of this invention R^{1A} is selected from the group consisting of:

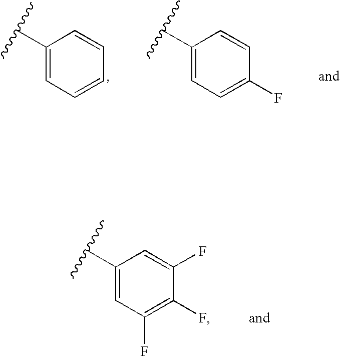

wherein the R⁹-R¹⁰- moiety is:

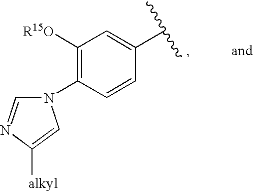

G is selected from the group consisting of —NH—, —O—, —S—, —S(O)—, —S(O)₂— and a direct bond, and W is —S(O)₂—.

In another embodiment of this invention R^{1A} is selected from the group consisting of:

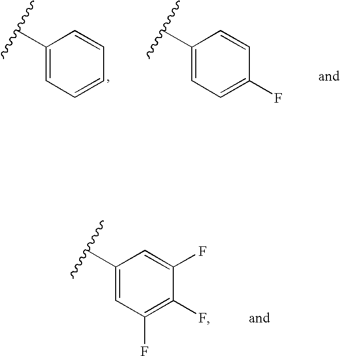

wherein the R⁹-R¹⁰- moiety is:

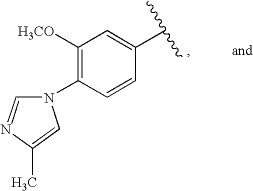

G is selected from the group consisting of —NH—, —O—, —S—, —S(O)—, —S(O)₂— and a direct bond, and W is —S(O)₂—.

In another embodiment of this invention R^{1A} is selected from the group consisting of:

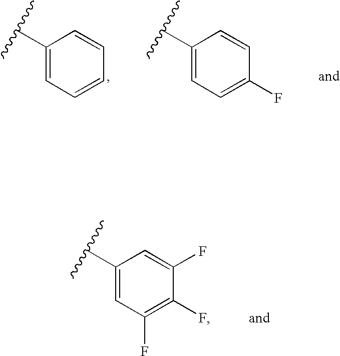

wherein the R⁹-R¹⁰- moiety is:

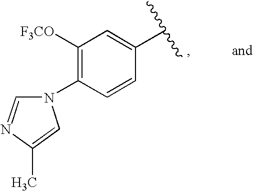

G is selected from the group consisting of —NH—, —O—, —S—, —S(O)—, —S(O)₂— and a direct bond, and W is —S(O)₂—,

In another embodiment of this invention R^{1A} is selected from the group consisting of:

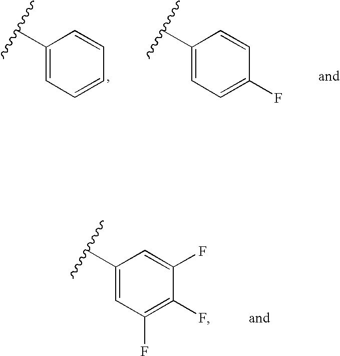

wherein the R⁹-R¹⁰- moiety is:

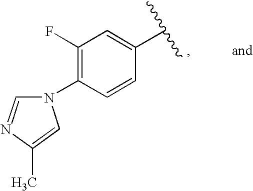

G is selected from the group consisting of —NH—, —O—, —S—, —S(O)—, —S(O)₂— and a direct bond, and W is —S(O)₂—.

In another embodiment of this invention R^{1A} is selected from the group consisting of:

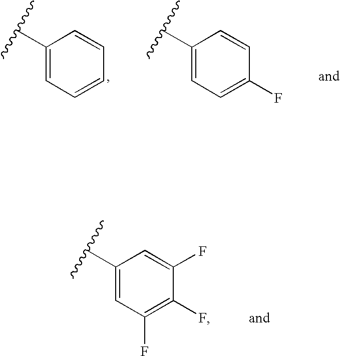

wherein the R⁹-R¹⁰- moiety is:

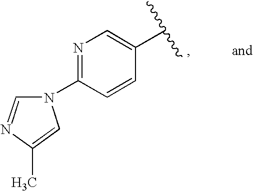

G is selected from the group consisting of —NH—, —O—, —S—, —S(O)—, —S(O)₂— and a direct bond, and W is —S(O)₂—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is selected from the group consisting of —NH—, —O—, —S—, —S(O)—, —S(O)₂— and a direct bond, and (e) W is —C(═NR²)—.

In another embodiment of this invention R^{1A} is selected from the group consisting of:

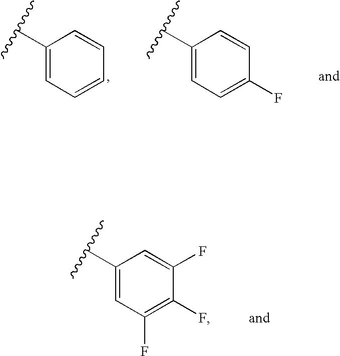

wherein the R⁹-R¹⁰- moiety is:

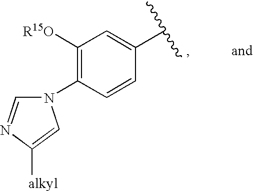

G is selected from the group consisting of —NH—, —O—, —S—, —S(O)—, —S(O)₂— and a direct bond, and W is —C(═NR²)—.

In another embodiment of this invention R^{1A} is selected from the group consisting of:

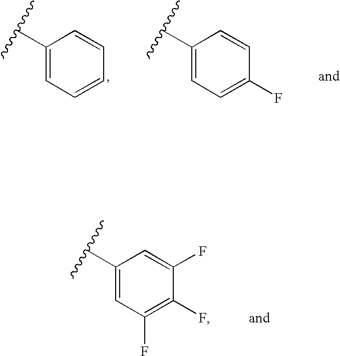

wherein the R⁹-R¹⁰- moiety is:

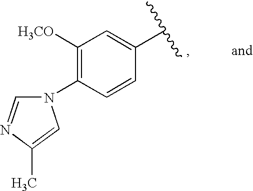

G is selected from the group consisting of —NH—, —O—, —S—, —S(O)—, —S(O)₂— and a direct bond, and W is —C(═NR²)—.

In another embodiment of this invention R^{1A} is selected from the group consisting of:

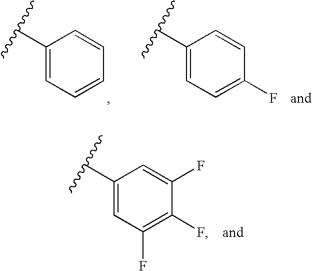

wherein the R⁹-R¹⁰- moiety is:

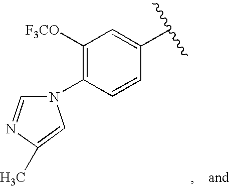

G is selected from the group consisting of —NH—, —O—, —S—, —S(O)—, —S(O)₂— and a direct bond, and W is —C(═NR²)—.

In another embodiment of this invention R^{1A} is selected from the group consisting of:

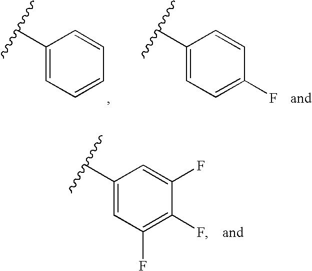

wherein the R⁹-R¹⁰- moiety is:

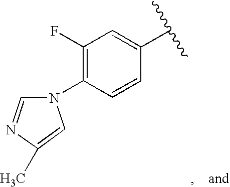

G is selected from the group consisting of —NH—, —O—, —SW, —S(O)—, —S(O)₂— and a direct bond, and W is —C(═NR²)—.

In another embodiment of this invention R^{1A} is selected from the group consisting of:

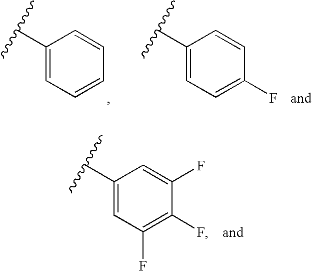

wherein the R⁹-R¹⁰- moiety is:

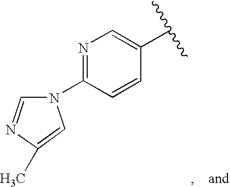

G is selected from the group consisting of —NH— —O—, —S—, —S(O)—, —S(O)₂— and a direct bond, and W is —C(═NR²)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —C(O)—, and (e) W is —C(O)—.

In another embodiment of this invention R^{1A} is selected from the group consisting of:

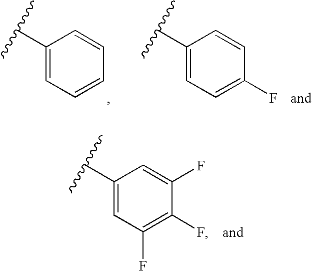

wherein the R⁹-R¹⁰- moiety is:

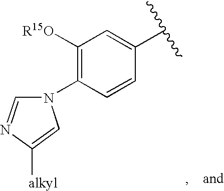

### G is —C(O)—, and W is —C(O)—.

In another embodiment of this invention R^{1A} is selected from the group consisting of:

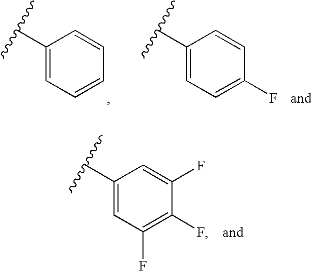

wherein the R⁹-R¹⁰- moiety is:

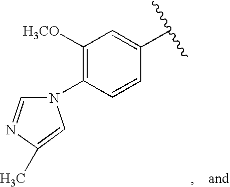

### G is —C(O)—, and W is —C(O)—.

In another embodiment of this invention R^{1A} is selected from the group consisting of:

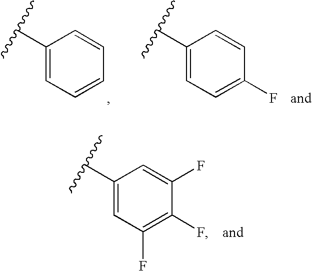

wherein the R⁹-R¹⁰- moiety is:

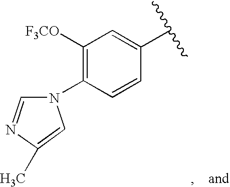

### G is —C(O)—, and W is —O(O)—.

In another embodiment of this invention R^{1A} is selected from the group consisting of:

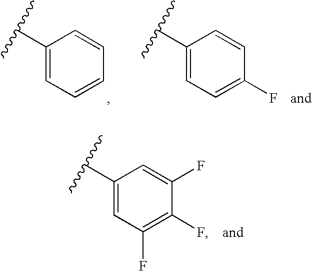

wherein the R⁹-R¹⁰- moiety is:

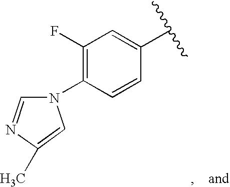

### G is —C(O)—, and W is —C(O)—.

In another embodiment of this invention R^{1A} is selected from the group consisting of:

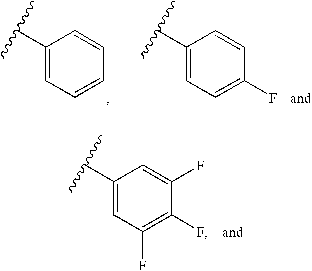

wherein the R⁹-R¹⁰- moiety is:

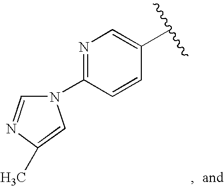

### G is —C(O)—, and W is —C(O)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —C(O)—, and (e) W is —S(O)—.

In another embodiment of this invention R^{1A} is selected from the group consisting of:

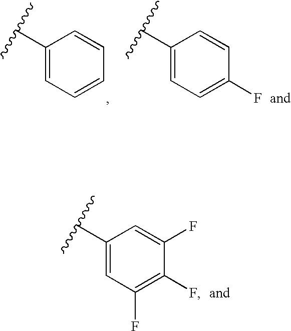

wherein the R⁹-R¹⁰- moiety is

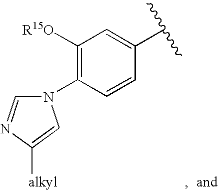

### G is —C(O)—, and W is —S(O)—.

In another embodiment of this invention R^{1A} is selected from the group consisting of:

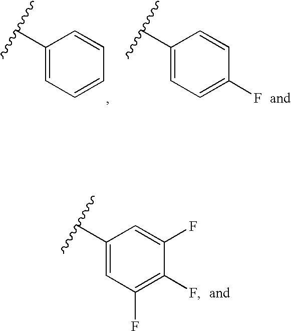

wherein the R⁹-R¹⁰- moiety is:

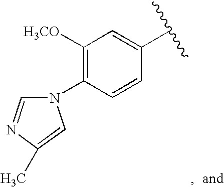

### G is —C(O)—, and W is —S(O)—.

In another embodiment of this invention R^{1A} is selected from the group consisting of:

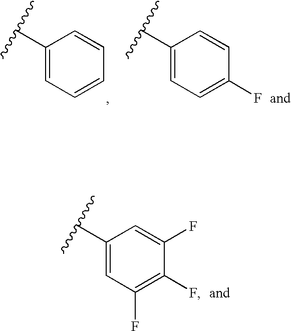

wherein the R⁹-R¹⁰- moiety is:

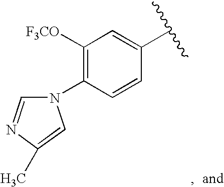

### G is —C(O)—, and W is —S(O)—.

In another embodiment of this invention R^{1A} is selected from the group consisting of:

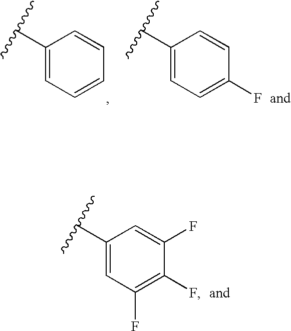

wherein the R⁹-R¹⁰- moiety is

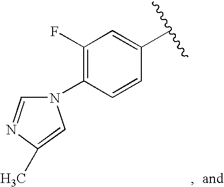

### G is —C(O)—, and W is —S(O)—.

In another embodiment of this invention R^{1A} is selected from the group consisting of:

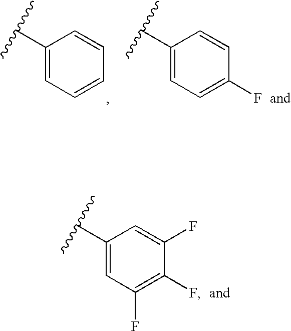

wherein the R⁹-R¹⁸— moiety is:

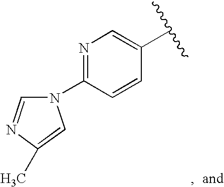

### G is —C(O)—, and W is —S(O)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —O(O)—, and (e) W is —S(O)₂—.

In another embodiment of this invention R^{1A} is selected from the group consisting of:

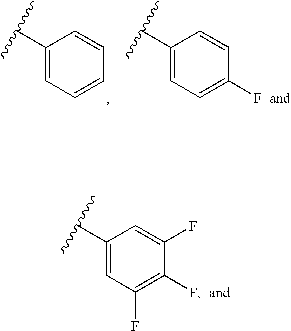

wherein the R⁹-R¹⁰- moiety is

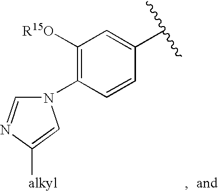

### G is —O(O)—, and W is —S(O)₂—.

In another embodiment of this invention R^{1A} is selected from the group consisting of:

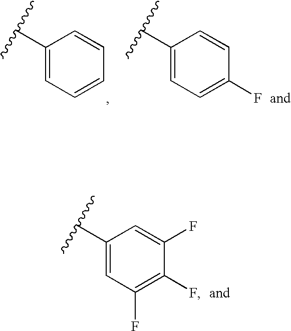

wherein the R⁹-R¹⁰- moiety is:

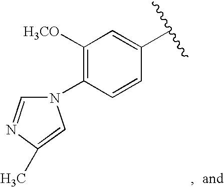

### G is —C(O)—, and W is —S(O)₂—.

In another embodiment of this invention R^{1A} is selected from the group consisting of:

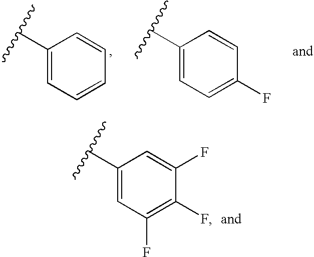

wherein the R⁹-R¹⁰- moiety is:

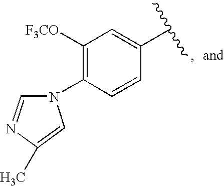

### G is —C(O)—, and W is —S(O)₂—.

In another embodiment of this invention R^{1A} is selected from the group consisting of:

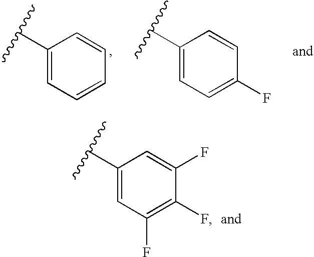

wherein the R⁹-R¹⁰- moiety is:

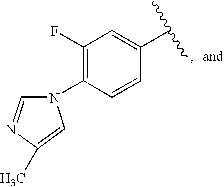

### G is —C(O)—, and W is —S(O)₂—.

In another embodiment of this invention R^{1A} is selected from the group consisting of:

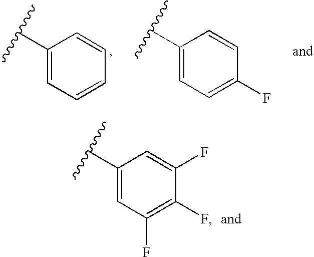

wherein the R⁹-R¹⁰- moiety is:

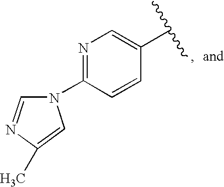

### G is —C(O)—, and W is —S(O)₂—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —C(O)—, and (e) W is —C(═NR²)—.

In another embodiment of this invention R^{1A} is selected from the group consisting of:

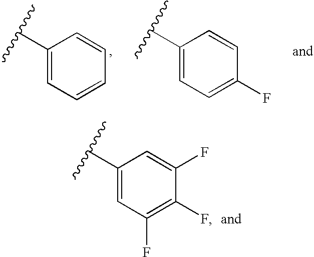

wherein the R⁹-R¹⁰- moiety is:

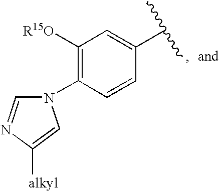

### G is —C(O)—, and W is —C(═NR²)—.

In another embodiment of this invention R^{1A} is selected from the group consisting of:

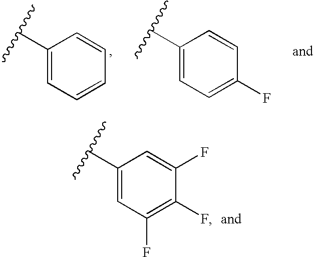

wherein the R⁹-R¹⁰- moiety is:

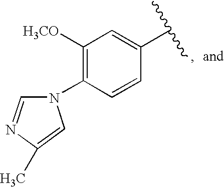

### G is —C(O)—, and W is —C(═NR²)—.

In another embodiment of this invention R^{1A} is selected from the group consisting of:

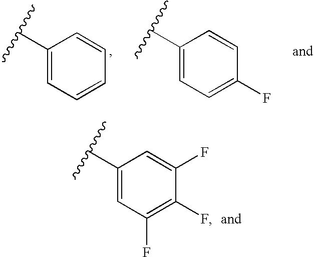

wherein the R⁹-R¹⁰- moiety is:

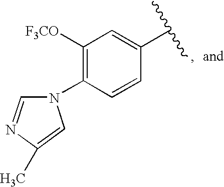

### G is —C(O)—, and W is —C(═NR²)—.

In another embodiment of this invention R^{1A} is selected from the group consisting of:

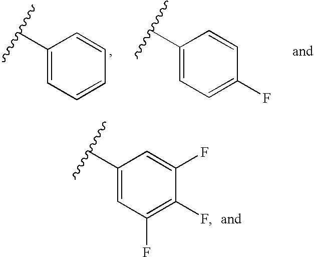

wherein the R⁹-R¹⁰- moiety is:

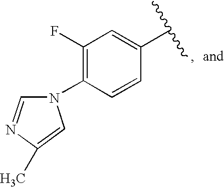

### G is —C(O)—, and W is —C(═NR²)—.

In another embodiment of this invention R^{1A} is selected from the group consisting of:

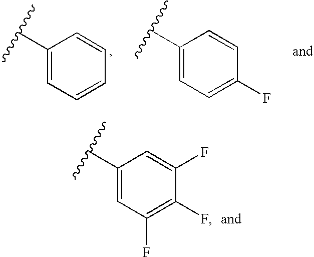

wherein the R⁹-R¹⁰- moiety is:

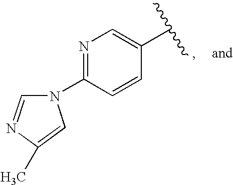

### G is —C(O)—, and W is —C(═NR²)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —(C═NR²)— and (e) W is —C(O)—.

In another embodiment of this invention R^{1A} is selected from the group consisting of:

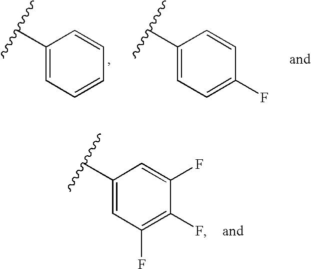

wherein the R⁹-R¹⁰- moiety is:

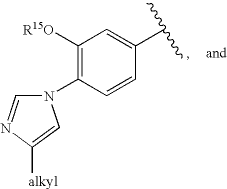

### G is —(C═NR²)—, and W is —C(O)—.

In another embodiment of this invention R^{1A} is selected from the group consisting of:

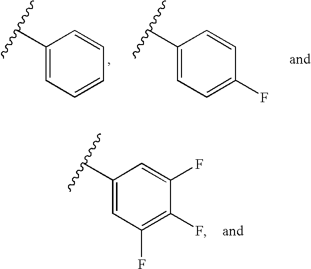

wherein the R⁹-R¹⁰- moiety is:

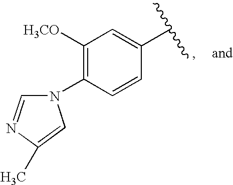

### G is —(C═NR²)—, and W is —C(O)—.

In another embodiment of this invention R^{1A} is selected from the group consisting of:

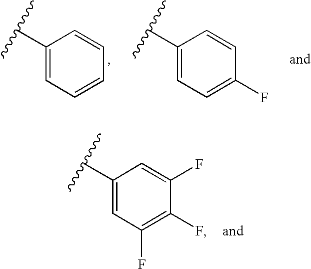

wherein the R⁹-R¹⁰- moiety is:

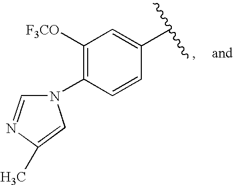

### G is —(C═NR²)—, and W is —C(O)—.

In another embodiment of this invention R^{1A} is selected from the group consisting of:

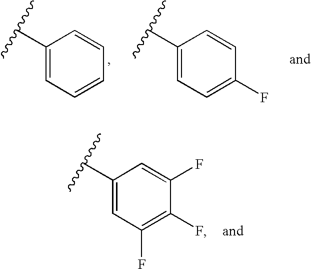

wherein the R⁹-R¹⁰- moiety is

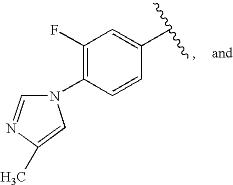

### G is —(C═NR²)—, and W is —C(O)—.

In another embodiment of this invention R^{1A} is selected from the group consisting of:

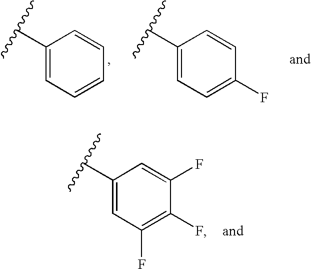

wherein the R⁹-R¹⁰- moiety is:

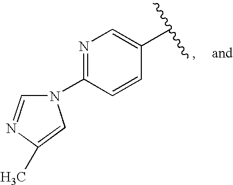

### G is —(C═NR²)—, and W is —C(O)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —(C═NR²)—, and (e) W is —S(O)—.

In another embodiment of this invention R^{1A} is selected from the group consisting of:

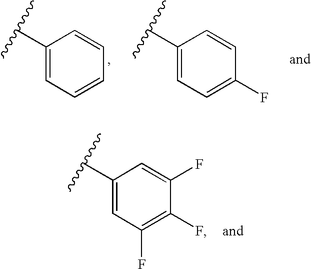

wherein the R⁹-R¹⁰- moiety is:

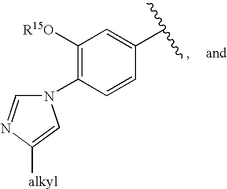

### G is —(C═NR²)—, and W is —S(O)—.

In another embodiment of this invention R^{1A} is selected from the group consisting of:

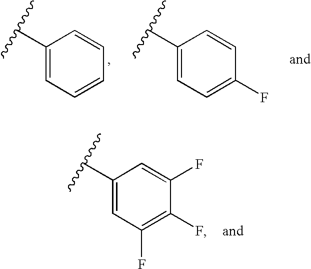

wherein the R⁹-R¹⁰- moiety is:

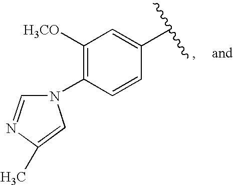

### G is —(C═NR²)—, and W is —S(O)—.

In another embodiment of this invention R^{1A} is selected from the group consisting of:

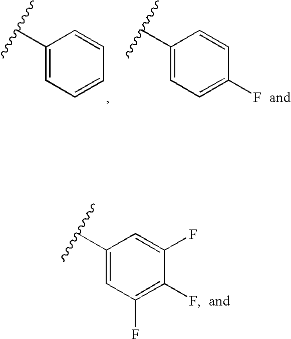

wherein the R⁹-R¹⁰- moiety is:

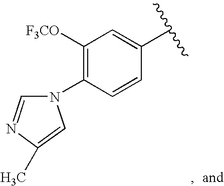

### G is —(C═NR²)—, and W is —S(O)—.

In another embodiment of this invention R^{1A} is selected from the group consisting of:

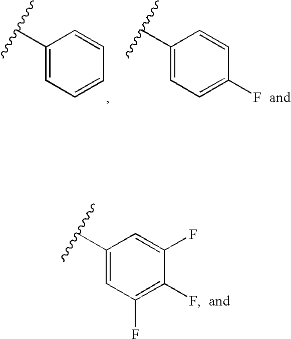

wherein the R⁹-R¹⁰- moiety is:

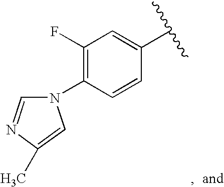

### G is —(C═NR²)—, and W is —S(O)—.

In another embodiment of this invention R^{1A} is selected from the group consisting of:

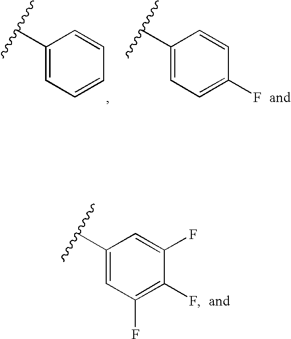

wherein the R⁹-R¹⁰- moiety is:

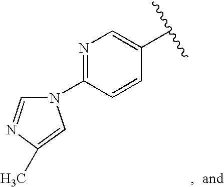

### G is —(C═NR²)—, and W is —S(O)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —(C═NR²)—, and (e) W is —S(O)₂—.

In another embodiment of this invention R^{1A} is selected from the group consisting of:

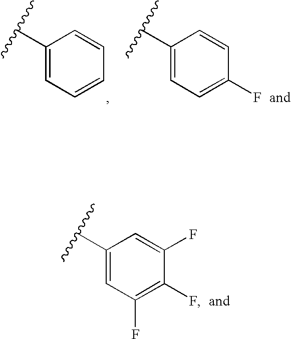

wherein the R⁹-R¹⁰- moiety is:

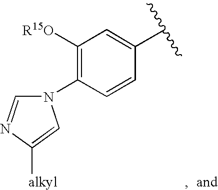

### G is —(C═NR²)—, and W is —S(O)₂—.

In another embodiment of this invention R^{1A} is selected from the group consisting of:

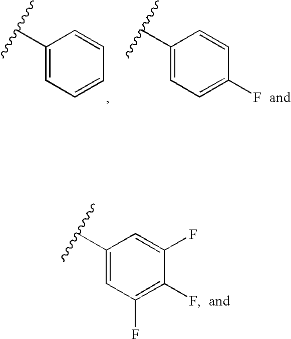

wherein the R⁹-R¹⁰- moiety is:

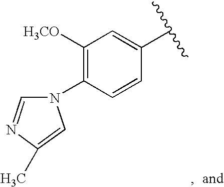

### G is —(C═NR²)—, and W is —S(O)₂—.

In another embodiment of this invention R^{1A} is selected from the group consisting of:

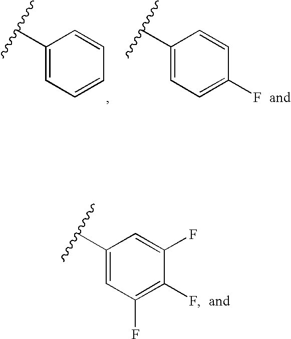

wherein the R⁹-R¹⁰-moiety is:

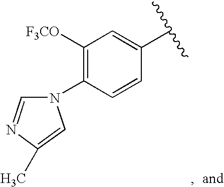

### G is —(C═NR²)—, and W is —S(O)₂—.

In another embodiment of this invention R^{1A} is selected from the group consisting of:

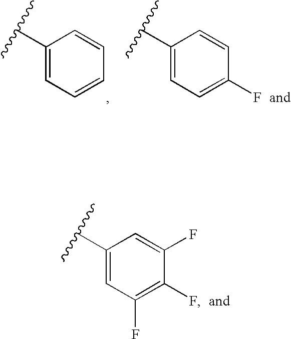

wherein the R⁹-R¹⁰- moiety is:

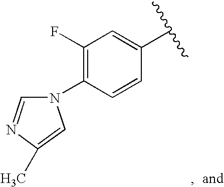

### G is —(C═NR²)—, and W is —S(O)₂—.

In another embodiment of this invention R^{1A} is selected from the group consisting of:

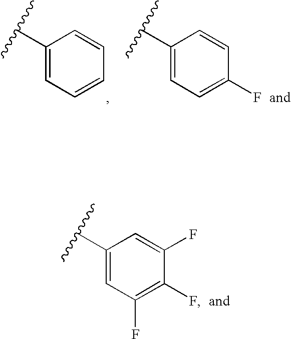

wherein the R⁹-R¹⁰- moiety is:

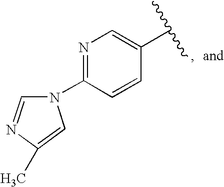

### G is —(C═NR²)—, and W is —S(O)₂—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —(C═NR²)—, and (e) W is —C(═NR²)—.

In another embodiment of this invention R^{1A} is selected from the group consisting of:

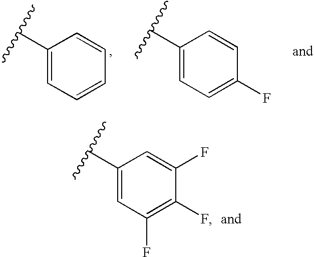

wherein the R⁹-R¹⁰- moiety is:

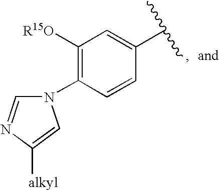

### G is —(C═NR²)—, and W is —C(═NR²)—.

In another embodiment of this invention R^{1A} is selected from the group consisting of:

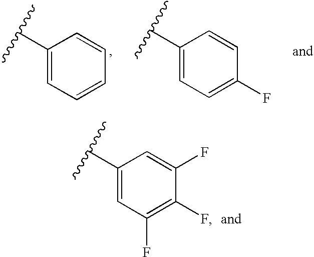

wherein the R⁹-R¹⁰- moiety is:

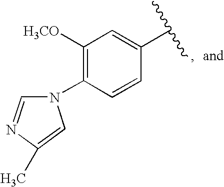

### G is —(C═NR²)—, and W is —C(═NR²)—.

In another embodiment of this invention R^{1A} is selected from the group consisting of:

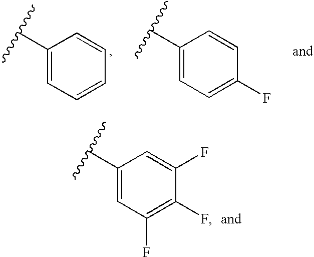

wherein the R⁹-R¹⁰ moiety is:

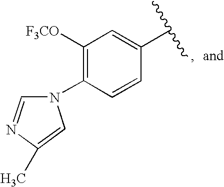

### G is —(C═NR²)—, and W is —C(═NR²)—.

In another embodiment of this invention R^{1A} is selected from the group consisting of:

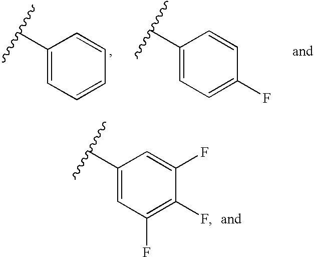

wherein the R⁹-R¹⁰- moiety is:

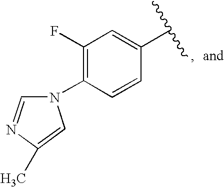

### G is —(C═NR²)—, and W is —C(═NR²)—.

In another embodiment of this invention R^{1A} is selected from the group consisting of:

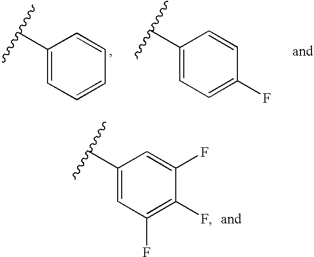

wherein the R⁹-R¹⁰- moiety is:

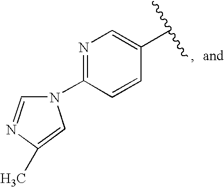

### G is —(C═NR²)—, and W is —C(═NR²)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —NHC(O)—, and (e) W is —C(O)—.

In another embodiment of this invention R^{1A} is selected from the group consisting of:

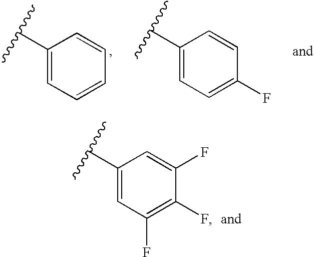

wherein the R⁹-R¹⁰- moiety is:

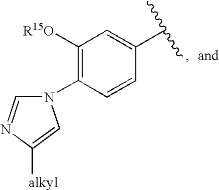

### G is —NHC(O)—, and W is —C(O)—.

In another embodiment of this invention R^{1A} is selected from the group consisting of:

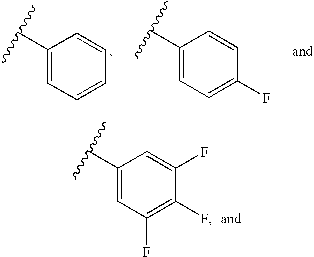

wherein the R⁹-R¹⁰- moiety is:

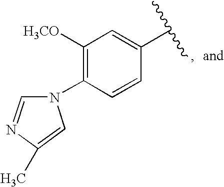

### G is —NHC(O)—, and W is —C(O)—.

In another embodiment of this invention R^{1A} is selected from the group consisting of:

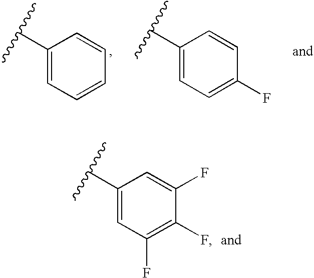

wherein the R⁹-R¹⁰- moiety is:

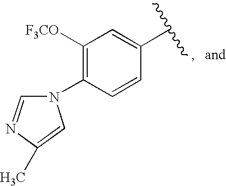

### G is —NHC(O)—, and W is —C(O)—.

In another embodiment of this invention R^{1A} is selected from the group consisting of:

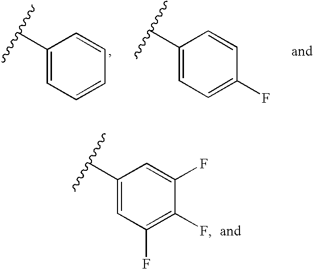

wherein the R⁹-R¹⁰- moiety is:

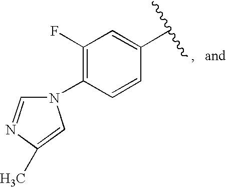

### G is —NHC(O)—, and W is —C(O)—.

In another embodiment of this invention R^{1A} is selected from the group consisting of:

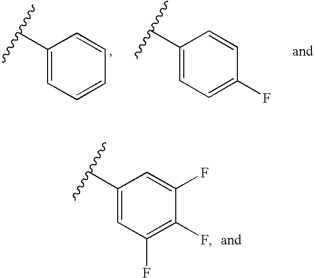

wherein the R⁹-R¹⁰- moiety is:

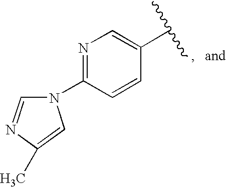

### G is —NHC(O)—, and W is —C(O)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —NHC(O)—, and (e) W is —S(O)—.

In another embodiment of this invention R^{1A} is selected from the group consisting of:

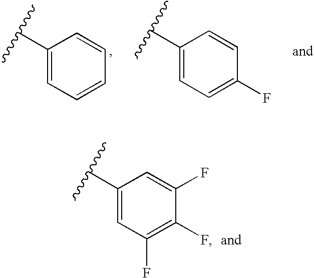

wherein the R⁹-R¹⁰- moiety is:

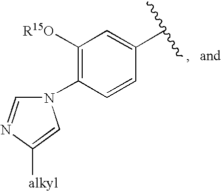

### G is —NHC(O)—, and W is —S(O)—.

In another embodiment of this invention R^{1A} is selected from the group consisting of:

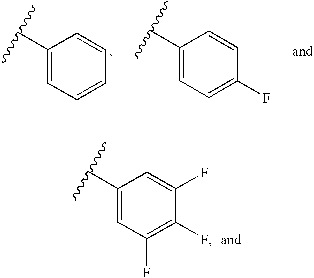

wherein the R⁹-R¹⁰- moiety is:

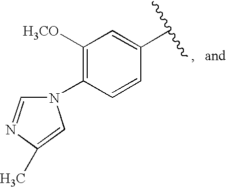

### G is —NHC(O)—, and W is —S(O)—.

In another embodiment of this invention R^{1A} is selected from the group consisting of:

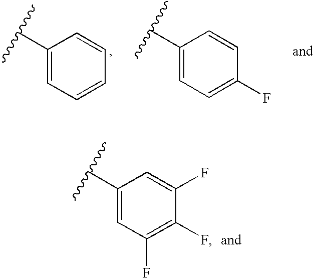

wherein the R⁹-R¹⁰- moiety is:

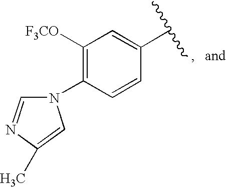

### G is —NHC(O)—, and W is —S(O)—.

In another embodiment of this invention R^{1A} is selected from the group consisting of:

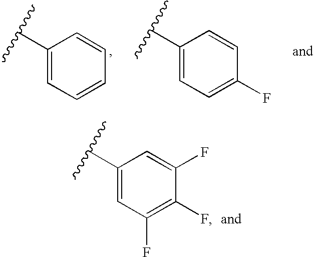

wherein the R⁹-R¹⁰- moiety is:

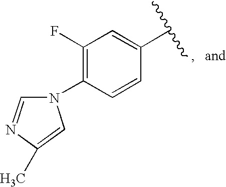

### G is —NHC(O)—, and W is —S(O)—.

In another embodiment of this invention R^{1A} is selected from the group consisting of:

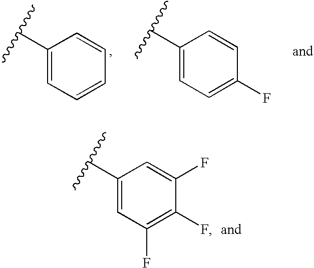

wherein the R⁹-R¹⁰- moiety is:

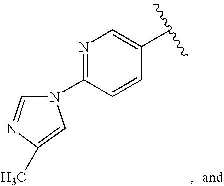

### G is —NHC(O)—, and W is —S(O)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —(C═C(R⁶)₂)—, and (e) W is —S(O)₂—.

In another embodiment of this invention R^{1A} is selected from the group consisting of:

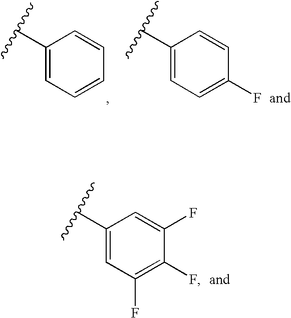

wherein the R⁹-R¹⁰- moiety is:

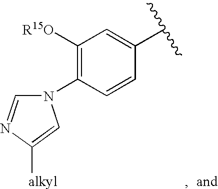

### G is —NHC(O)—, and W is —S(O)₂—.

In another embodiment of this invention R^{1A} is selected from the group consisting of:

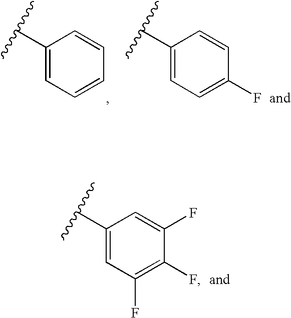

wherein the R⁹-R¹⁰- moiety is:

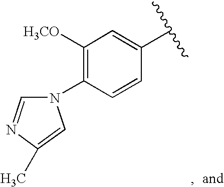

### G is —NHC(O)—, and W is —S(O)₂—.

In another embodiment of this invention R^{1A} is selected from the group consisting of:

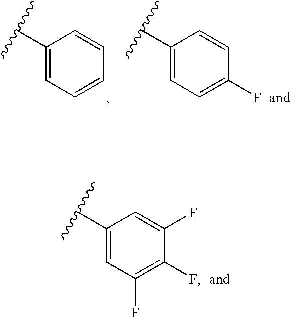

wherein the R⁹-R¹⁰- moiety is:

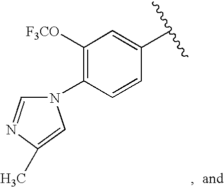

### G is —NHC(O)—, and W is —S(O)₂—.

In another embodiment of this invention R^{1A} is selected from the group consisting of:

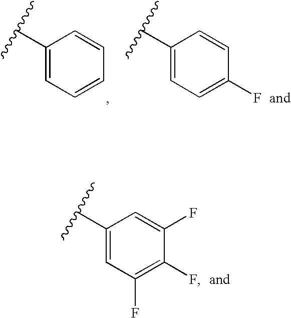

wherein the R⁹-R¹⁰- moiety is:

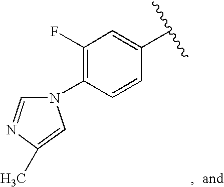

### G is —NHC(O)—, and W is —S(O)₂—.

In another embodiment of this invention R^{1A} is selected from the group consisting of:

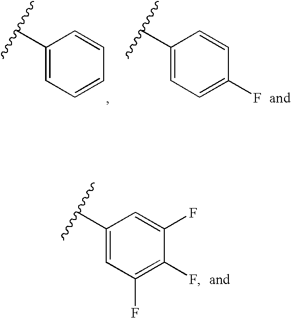

wherein the R⁹-R¹⁰- moiety is:

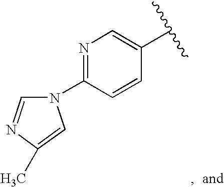

### G is —NHC(O)—, and W is —S(O)₂—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —NHC(O)—, and (e) W is —C(═NR²)—.

In another embodiment of this invention R^{1A} is selected from the group consisting of:

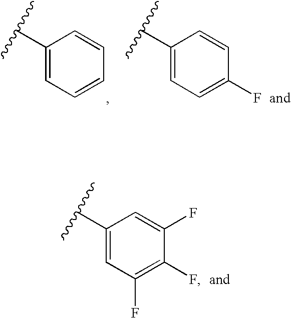

wherein the R⁹-R¹⁰- moiety is:

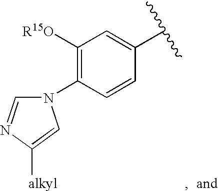

### G is —NHC(O)—, and W is —C(═NR²)—.

In another embodiment of this invention R^{1A} is selected from the group consisting of:

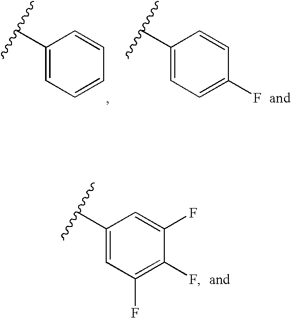

wherein the R⁹-R¹⁰- moiety is:

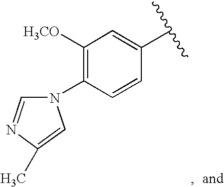

### G is —NHC(O)—, and W is —C(═NR²)—.

In another embodiment of this invention R^{1A} is selected from the group consisting of

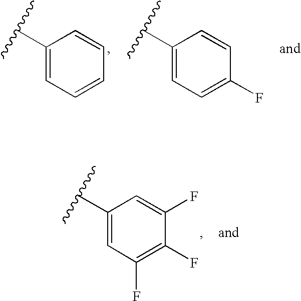

wherein the R⁹-R¹⁰- moiety is:

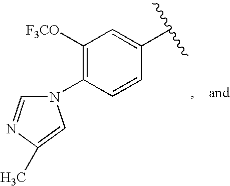

### G is —NHC(O)—, and W is —C(═NR²)—.

In another embodiment of this invention R^{1A} is selected from the group consisting of:

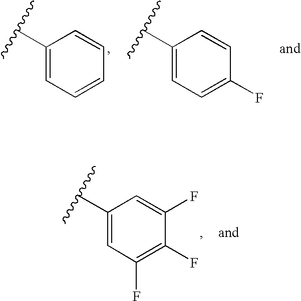

wherein the R⁹-R¹⁰- moiety is:

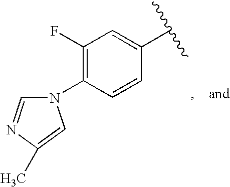

### G is —NHC(O)—, and W is —C(═NR²)—.

In another embodiment of this invention R^{1A} is selected from the group consisting of:

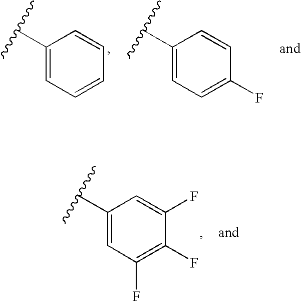

wherein the R⁹-R¹⁰- moiety is:

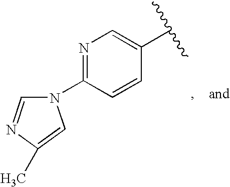

### G is —NHC(O)—, and W is —C(═NR²)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —CHR³— (e.g., —CHOH), and (e) W is —C(O)—.

In another embodiment of this invention R^{1A} is selected from the group consisting of:

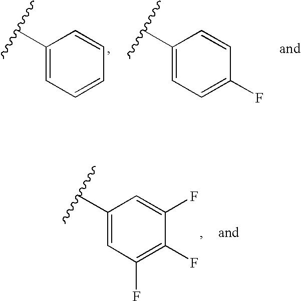

wherein the R⁹-R¹⁰- moiety is:

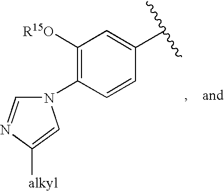

### G is —CHR³— (e.g., —CHOH), and W is —C(O)—.

In another embodiment of this invention R^{1A} is selected from the group consisting of:

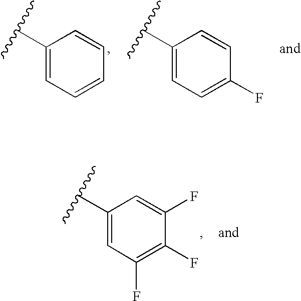

wherein the R⁹ moiety is:

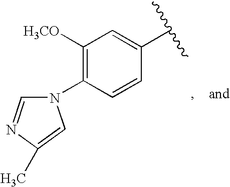

### G is —CHR³— (e.g., —CHOH), and W is —C(O)—.

In another embodiment of this invention R^{1A} is selected from the group consisting of:

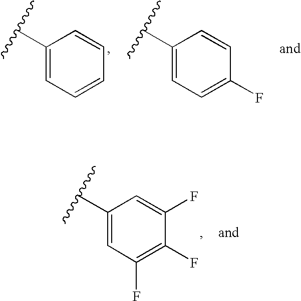

wherein the R⁹-R¹⁰- moiety is:

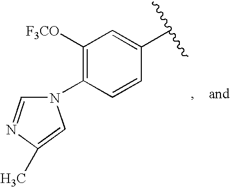

### G is —CHR³— (e.g., —CHOH), and W is —C(O)—.

In another embodiment of this invention R^{1A} is selected from the group consisting of:

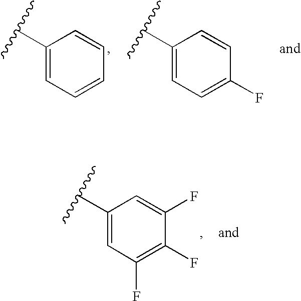

wherein the R⁹-R¹⁰- moiety is:

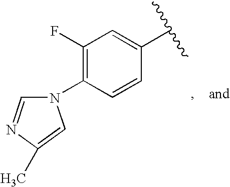

### G is —CHR³— (e.g., —CHOH), and W is —C(O)—.

In another embodiment of this invention R^{1A} is selected from the group consisting of:

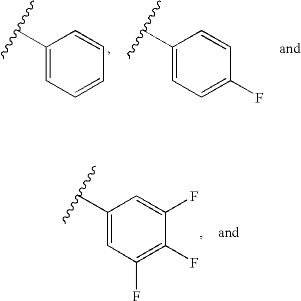

wherein the R⁹-R¹⁰- moiety is:

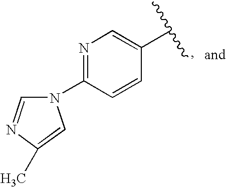

### G is —CHR³— (e.g., —CHOH), and W is —C(O)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —CHR³— (e.g., —CHOH), and (e) W is —S(O)—.

In another embodiment of this invention R^{1A} is selected from the group consisting of:

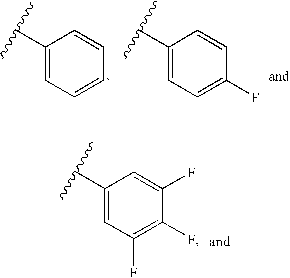

wherein the R⁹-R¹⁰-moiety is:

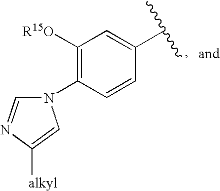

### G is —CHR³— (e.g., —CHOH), and W is —S(O)—.

In another embodiment of this invention R^{1A} is selected from the group consisting of:

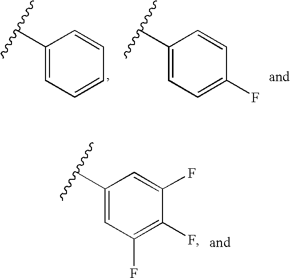

wherein the R⁹-R¹⁰- moiety is:

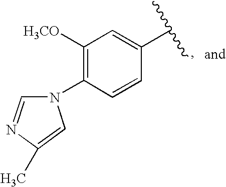

### G is —CHR³— (e.g., —CHOH), and W is —S(O)—.

In another embodiment of this invention R^{1A} is selected from the group consisting of:

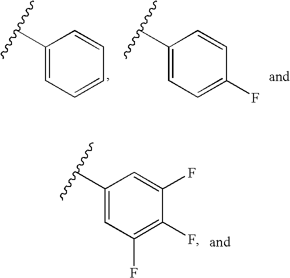

wherein the R⁹-R¹⁰- moiety is:

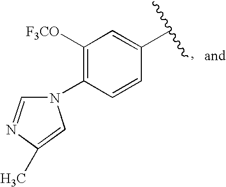

### G is —CHR³— (e.g., —CHOH), and W is —S(O)—.

In another embodiment of this invention R^{1A} is selected from the group consisting of:

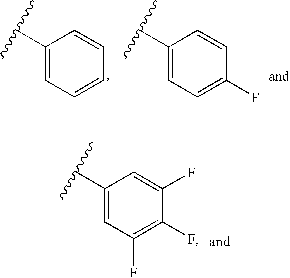

wherein the R⁹-R¹⁰- moiety is:

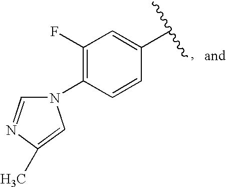

### G is —CHR³— (e.g., —CHOH), and W is —S(O)—.

In another embodiment of this invention R^{1A} is selected from the group consisting of:

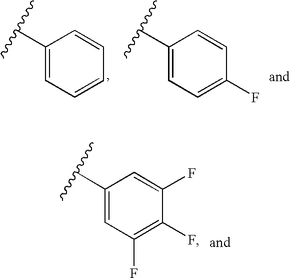

wherein the R⁹-R¹⁰- moiety is:

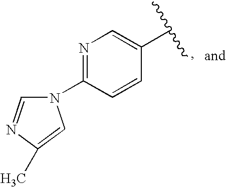

### G is —CHR³— (e.g., —CHOH), and W is —S(O)—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —CHR³— (e.g., —CHOH), and (e) W is —S(O)₂—.

In another embodiment of this invention R^{1A} is selected from the group consisting of:

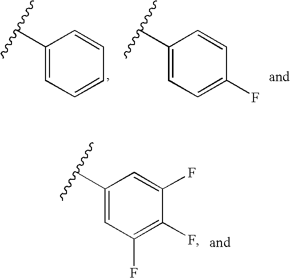

wherein the R⁹-R¹⁰- moiety is:

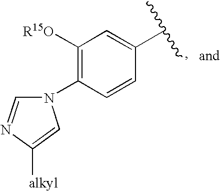

### G is —CHR³— (e.g., —CHOH), and W is —S(O)₂—.

In another embodiment of this invention R^{1A} is selected from the group consisting of:

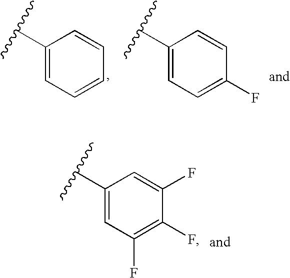

wherein the R⁹-R¹⁰- moiety is:

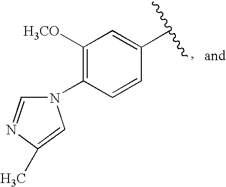

### G is —CHR³— (e.g., —CHOH), and W is —S(O)₂—.

In another embodiment of this invention R^{1A} is selected from the group consisting of:

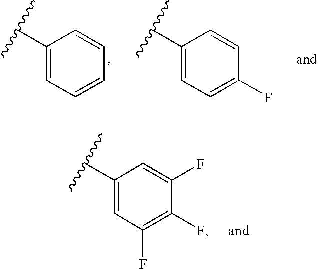

wherein the R⁹-R¹⁰- moiety is:

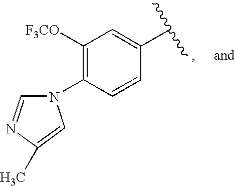

### G is —CHR³— (e.g., —CHOH), and W is —S(O)₂—.

In another embodiment of this invention R^{1A} is selected from the group consisting of:

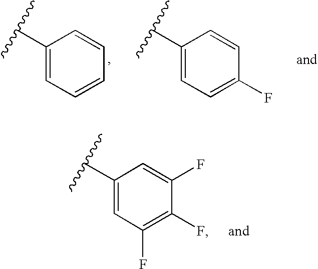

wherein the R⁹-R¹⁰- moiety is:

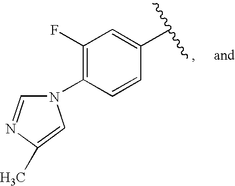

### G is —CHR³— (e.g., —CHOH), and W is —S(O)₂—.

In another embodiment of this invention R^{1A} is selected from the group consisting of:

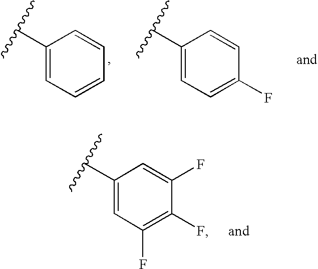

wherein the R⁹-R¹⁰- moiety is:

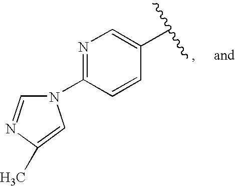

### G is —CHR³— (e.g., —CHOH), and W is —S(O)₂—.

In another embodiment of this invention: (a) R^{1A} is phenyl, or R^{1A} is phenyl substituted with 1 to 3 F atoms, and (b) R¹⁰ is phenyl substituted with one —OR¹⁵ group, wherein R¹⁵ is methyl, and (c) R⁹ is selected from the group consisting of imidazolyl and imidazolyl substituted with one methyl group, and (d) G is —CHR³— (e.g., —CHOH), and (e) W is —C(═NR²)—.

In another embodiment of this invention R^{1A} is selected from the group consisting of:

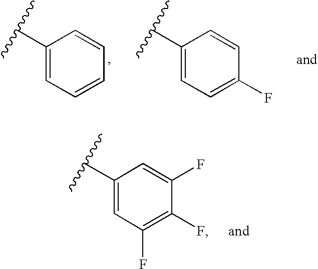

wherein the R⁹-R¹⁰- moiety is:

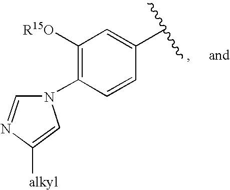

### G is —CHR³— (e.g., —CHOH), and W is —C(═NR²)—.

In another embodiment of this invention R^{1A} is selected from the group consisting of:

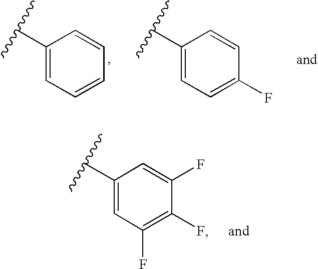

wherein the R⁹-R¹⁰- moiety is:

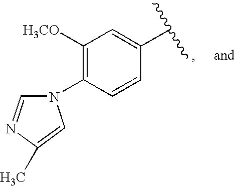

### G is —CHR³— (e.g., —CHOH), and W is —C(═NR²)—.

In another embodiment of this invention R^{1A} is selected from the group consisting of:

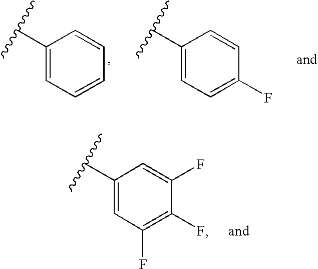

wherein the R⁹-R¹⁰- moiety is:

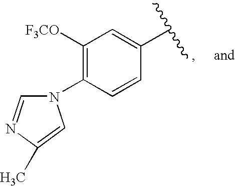

### G is —CHR³— (e.g., —CHOH), and W is —C(═NR²)—.

In another embodiment of this invention R^{1A} is selected from the group consisting of:

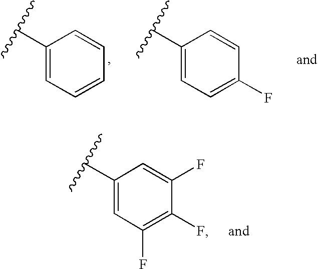

wherein the R⁹-R¹⁰- moiety is:

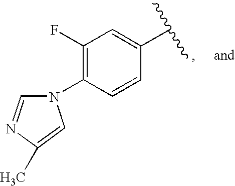

### G is —CHR³— (e.g., —CHOH), and W is —C(═NR²)—.

In another embodiment of this invention R^{1A} is selected from the group consisting of:

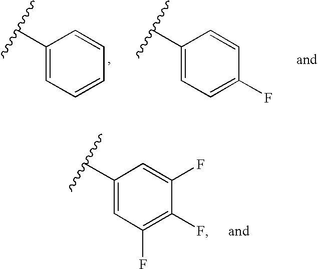

wherein the R⁹-R¹⁰- moiety is:

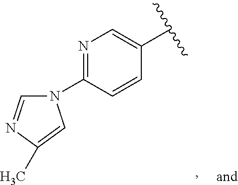

### G is —CHR³— (e.g., —CHOH), and W is —C(═NR²)—.

Other embodiments of this invention are directed to compounds of formula (I) wherein R^{1A} is selected from the group consisting of: benzofusedcycloalkyl (i.e., fused benzocycloalkyl), fused benzoheterocycloalkyl, fused heteroarylcycloalkyl, fused heteroarylheterocycloalkyl, and wherein said R^{1A} groups are optionally substituted with 1-5 independently selected R²¹ groups. In one example, the R²¹ groups are halo (e.g., F).

Examples of the fused ring R^{1A} groups include, but are not limited to:

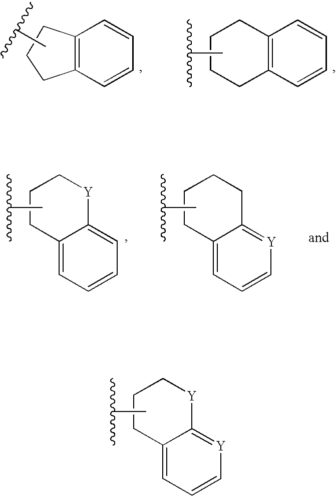

wherein each Y is independently selected from the group consisting of: —O—, —NR¹⁴— and —C(R²¹)_{q}—, wherein q is as defined above (i.e., 0, 1 or 2 and each R²¹ is independently selected), and wherein R¹⁴ and R²¹ are as defined for formula (I). Examples of these R^{1A} groups include, for example:

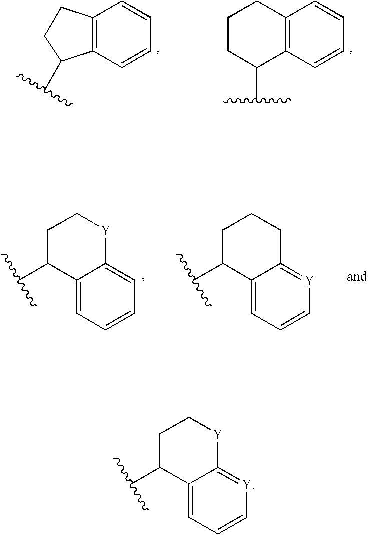

Compounds of formula (I) also include compounds wherein R^{1A} is an alkyl group (e.g., ethyl) substituted with one R²¹ group. Examples of said R^{1A} groups include alkyl (e.g., methyl or ethyl) substituted with the R²¹ moiety aryl (e.g., phenyl or naphthyl). Examples of said. R^{1A} groups also include alkyl (e.g., methyl or ethyl) substituted with the R²¹ moiety aryl (e.g., phenyl or naphthyl), which in turn is substituted with one or more (e.g., one or two) independently selected R²² groups (e.g., R²² is halo, such as, for example, F).

Examples of the substituted R^{1A} alkyl groups include, but are not limited to:

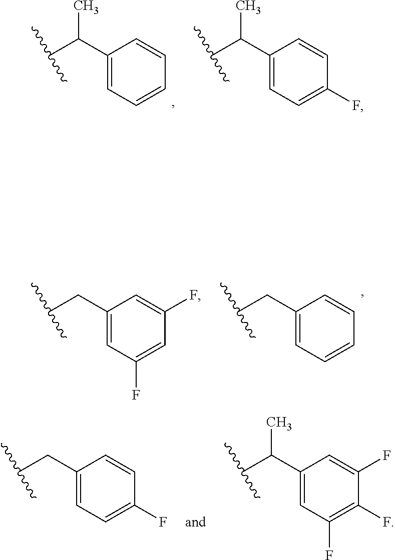

Other embodiments of this invention are directed to compounds of formula (I) wherein R^{1A} is a cycloalkyl group (e.g., cyclopropyl or cyclobutyl) substituted with one R²¹ group (e.g., aryl, such as, for example, phenyl), or a cycloalkyl group (e.g., cyclopentyl or cyclohexyl) substituted with one R²¹ group (e.g., aryl, such as, for example, phenyl) which in turn is substituted with one or more (e.g., one or two) independently selected R²² groups (e.g., halo, such as, for example, F). In one example the R²¹ group is bound to the same carbon of the R^{1A} group that binds the R^{1A} group to the rest of the molecule.

Examples of the cycloalkyl R^{1A} groups include, but are not limited to:

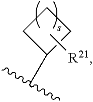

such as, for example,

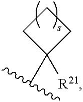

wherein s is 0 (i.e., the ring is cyclopropyl), or 1 (i.e., the ring is cyclobutyl). Examples of these R^{1A} groups include, but are not limited to:

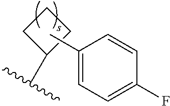

such as, for example,

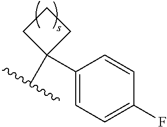

wherein s is 0 (i.e., the ring is cyclopropyl), or 1 (i.e., the ring is cyclobutyl).

Other embodiments of this invention are directed to compounds of formula (I) wherein R^{1A} is

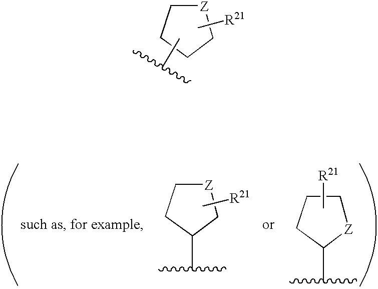

wherein Z is selected from the group consisting of: (1) —O—, (2) —NR¹⁴—, (3) —C(R²¹)_{q}— wherein q is 0, 1 or 2, and each R²¹ is independently selected, (4) —C(R²¹)_{q}—C(R²¹)_{q}— wherein each q is independently 0, 1 or 2 and each R²¹ is independently selected, (5) —(C(R²¹)_{q})_{q}—O—(C(R²¹)_{q})_{q}— wherein each q is independently 0, 1 or 2, and each R²¹ is independently selected, and (6) —(C(R²¹)_{q})_{q}—N(R¹⁴)—(C(R²¹)_{q})_{q}— wherein each q is independently 0, 1 or 2, and each R²¹ is independently selected. Examples of R²¹ include, but are not limited to, aryl (e.g., phenyl) and aryl (e.g., phenyl) substituted with one or more (e.g., one or two, or one) independently selected R²² groups (e.g., halo, such as, for example, F). Examples of this R^{1A} include, but are not limited to:

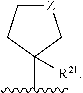

Thus, examples of this R^{1A} group include, but are not limited to:

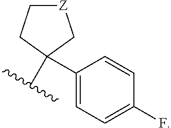

Examples of R^{1A} also include, but are not limited to:

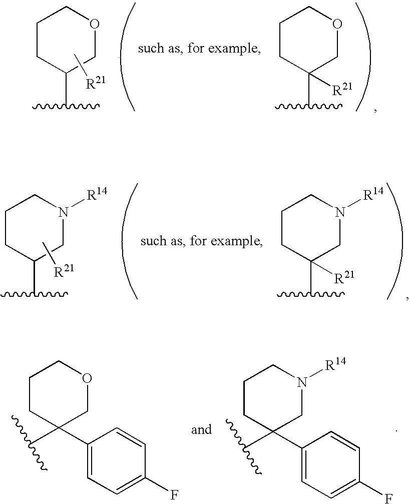

Examples of the R^{1A} group

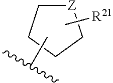

also include, but are not limited to:

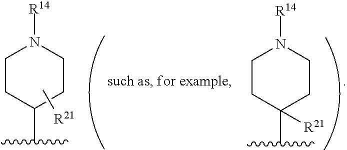

Examples of the R^{1A} group

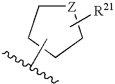

also include, but are not limited to:

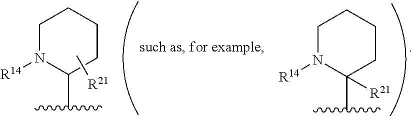

Examples of the R^{1A} group

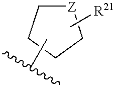

also include, but are not limited to:

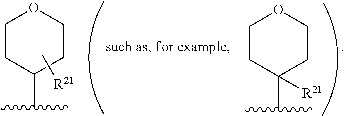

Examples of the R^{1A} group

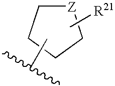

also include, but are not limited to:

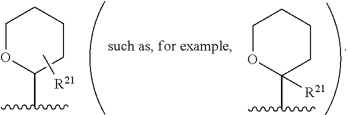

Other embodiments of this invention are directed to compounds of formula (I) wherein R¹⁰ is aryl (e.g., phenyl) or aryl (e.g., phenyl) substituted with one or more (e.g., one or two, or one) R²¹ groups (e.g., —OR¹⁵, wherein, for example, R¹⁵ is alkyl, such as, for example, methyl), and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

Thus, examples of the

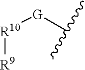

moiety of the compounds of this invention include, but are not limited to:

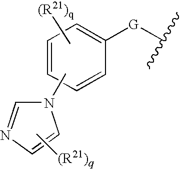

wherein q is 0, 1 or 2, such as, for example,

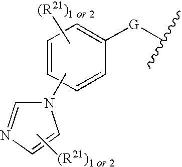

such as, for example,

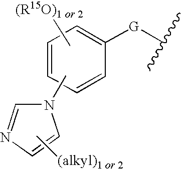

wherein R¹⁵ is alkyl (e.g., methyl), such as, for example,

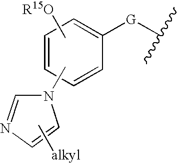

wherein R¹⁵ is alkyl (e.g., methyl), such as, for example,

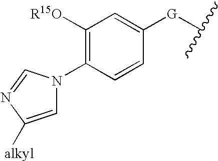

wherein R¹⁵ is alkyl (e.g., methyl), such as, for example,

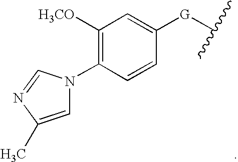

Other embodiments of this invention are directed to the compounds of formula (I) wherein R¹⁰ is heteroaryl or heteroaryl substituted with one or more R²¹ groups, and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

In another embodiment of the compounds of formula (I) R¹⁰ is aryl substituted with one R²¹ group, wherein said R²¹ group is —OR¹⁵. In one example, R¹⁵ is alkyl. In another example R¹⁵ is methyl.

In another embodiment of the compounds of formula (I) R¹⁰ is phenyl substituted with one R²¹ group, wherein said R²¹ group is —OR¹⁵. In one example, R¹⁵ is alkyl. In another example R¹⁵ is methyl.

In another embodiment of the compounds of formula (I) R¹⁰ is heteroaryl.

In another embodiment of the compounds of formula (I) R⁹ is heteroaryl.

In another embodiment of the compounds of formula (I) R⁹ is heteroaryl substituted with one or more (e.g., one) independently selected R²¹ groups.

In another embodiment of the compounds of formula (I) R⁹ is heteroaryl substituted with one or more (e.g., one) independently selected R²¹ groups, wherein each R²¹ group is the same or different alkyl group (e.g., methyl).

In another embodiment of the compounds of formula (I) R⁹ is heteroaryl substituted with one R²¹ group.

In another embodiment of the compounds of formula (I) R⁹ is heteroaryl substituted with one R²¹ group, wherein R²¹ is an alkyl group (e.g., methyl).

In another embodiment of the compounds of formula (I) R⁹ is imidazolyl.

In another embodiment of the compounds of formula (I) R⁹ is imidazolyl substituted with one or more (e.g., one) independently selected R²¹ groups.

In another embodiment of the compounds of formula (I) R⁹ is imidazolyl substituted with one or more (e.g., one) independently selected R²¹ groups, wherein each R²¹ group is the same or different alkyl group (e.g., methyl).

In another embodiment of the compounds of formula (I) R⁹ is imidazolyl substituted with one R²¹ group.

In another embodiment of the compounds of formula (I) R⁹ is imidazolyl substituted with one R²¹ group, wherein R²¹ is an alkyl group (e.g., methyl).

In another embodiment of the compounds of formula (I) R⁹ is heteroaryl, optionally substituted with one or more R²¹ groups, and R¹⁰ is aryl optionally substituted with one or more (e.g., one) R²¹ groups.

In another embodiment of the compounds of formula (I) R⁹ is heteroaryl, optionally substituted with one R²¹ group, and R¹⁰ is aryl optionally substituted with one R²¹ group.

In another embodiment of the compounds of formula (I) R⁹ is heteroaryl, optionally substituted with one or more R²¹ groups, and R¹⁰ is phenyl optionally substituted with one or more (e.g., one) R²¹ groups.

In another embodiment of the compounds of formula (I) R⁹ is heteroaryl, optionally substituted with one R²¹ group, and R¹⁰ is phenyl optionally substituted with one R²¹ group.

In another embodiment of the compounds of formula (I) R⁹ is imidazolyl, optionally substituted with one or more R²¹ groups, and R¹⁰ is aryl optionally substituted with one or more (e.g., one) R²¹ groups.

In another embodiment of the compounds of formula (I) R⁹ is imidazolyl, optionally substituted with one R²¹ group, and R¹⁰ is aryl optionally substituted with one R²¹ group.

In another embodiment of the compounds of formula (I) R⁹ is imidazolyl, optionally substituted with one or more R²¹ groups, and R¹⁰ is phenyl optionally substituted with one or more (e.g., one) R²¹ groups.

In another embodiment of the compounds of formula (I) R⁹ is imidazolyl, optionally substituted with one R²¹ group, and R¹⁰ is phenyl optionally substituted with one R²¹ group.

Other embodiments of the compounds of formula (I) are directed to any one of the above embodiments wherein R⁹ is:

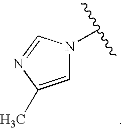

Other embodiments of the compounds of formula (I) are directed to any one of the above embodiments wherein R¹⁰ is:

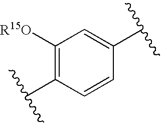

(wherein the —OR¹⁵ is ortho to the carbon to which R⁹ is bound to, i.e., the R⁹-R¹⁰- moiety is:

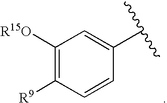

Other embodiments for the compounds of formula (I) are directed to any one of the above embodiments wherein R¹⁰ is:

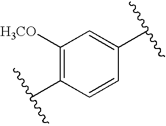

(wherein the —OCH₃ is ortho to the carbon to which R⁹ is bound to, i.e., the R⁹-R¹⁰- moiety is:

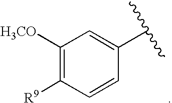

In another embodiment of the compounds of formula (I) R^{1A} is benzofusedcycloalkyl.

In another embodiment of the compounds of formula (I) R^{1A} is:

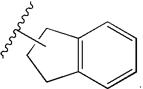

In another embodiment of the compounds of formula (I) R^{1A} is:

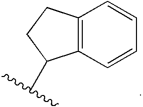

In another embodiment of the compounds of formula (I) R^{1A} is:

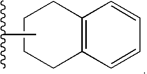

In another embodiment of the compounds of formula (I) R^{1A} is:

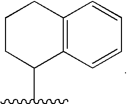

In another embodiment of the compounds of formula (I) R^{1A} is alkyl substituted with one R²¹ group.

In another embodiment of the compounds of formula (I) R^{1A} is alkyl substituted with one R²¹ group, and said alkyl is

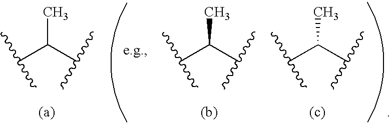

In another embodiment of the compounds of formula (I) R^{1A} is alkyl (e.g., (a), (b) or (c) described above) substituted with one R²¹ group wherein said R²¹ group is aryl.

In another embodiment of the compounds of formula (I) R^{1A} is alkyl (e.g., (a), (b) or (c) described above) substituted with one R²¹ group wherein said R²¹ group is phenyl.

In another embodiment of the compounds of formula (I) R^{1A} is alkyl (e.g., (a), (b) or (c) described above) substituted with one R²¹ group wherein said R²¹ group is naphthyl.

In another embodiment of the compounds of formula (I) R^{1A} is alkyl substituted with one R²¹ group, and said R²¹ group is substituted with two independently selected R²² groups.

In another embodiment of the compounds of formula (I) R^{1A} is alkyl substituted with one R²¹ group, and said R²¹ group is substituted with one R²² group.

In another embodiment of the compounds of formula (I) R^{1A} is alkyl substituted with one R²¹ group, wherein said alkyl group is (a) (e.g., (b) or (c)), as described above, and said R²¹ group is substituted with two independently selected R²² groups.

In another embodiment of the compounds of formula (I) R^{1A} is alkyl substituted with one R²¹ group, wherein said alkyl group is (a) (e.g., (b) or (c)), as described above, and said R²¹ group is substituted with one R²² group.

In another embodiment of the compounds of formula (I) R^{1A} is alkyl substituted with one R²¹ group, wherein said R²¹ group is aryl, and said R²¹ group is substituted with two independently selected R²² groups.

In another embodiment of the compounds of formula (I) R^{1A} is alkyl substituted with one R²¹ group, wherein said R²¹ group is aryl, and said R²¹ group is substituted with one R²² group.

In another embodiment of the compounds of formula (I) R^{1A} is alkyl substituted with one R²¹ group, wherein said R²¹ group is aryl, said alkyl group is (a) (e.g., (b) or (c)), as described above, and said R²¹ group is substituted with two independently selected R²² groups.

In another embodiment of the compounds of formula (I) R^{1A} is alkyl substituted with one R²¹ group, wherein said R²¹ group is aryl, wherein said alkyl group is (a) (e.g., (b) or (c)), as described above, and said R²¹ group is substituted with one R²² group.

In another embodiment of the compounds of formula (I) R^{1A} is alkyl substituted with one R²¹ group, wherein said R²¹ group is aryl, said R²¹ group is substituted with two independently selected R²² groups, and each R²² is halo.

In another embodiment of the compounds of formula (I) R^{1A} is alkyl substituted with one R²¹ group, wherein said R²¹ group is aryl, and said R²¹ group is substituted with one R²² group, and said R²² is halo.

In another embodiment of the compounds of formula (I) R^{1A} is alkyl substituted with one R²¹ group, wherein said R²¹ group is aryl, said alkyl group is (a) (e.g., (b) or (c)), as described above, and said R²¹ group is substituted with two independently selected R²² groups, and each R²² is halo.

In another embodiment of the compounds of formula (I) R^{1A} is alkyl substituted with one R²¹ group, wherein said R²¹ group is aryl, wherein said alkyl group is (a) (e.g., (b) or (c)), as described above, and said R²¹ group is substituted with one R²² group. and said R²² is halo.

In another embodiment of the compounds of formula (I) R^{1A} is alkyl substituted with one R²¹ group, wherein said R²¹ group is aryl, said R²¹ group is substituted with two independently selected R²² groups, and each R²² is F.

In another embodiment of the compounds of formula (I) R^{1A} is alkyl substituted with one R²¹ group, wherein said R²¹ group is aryl, and said R²¹ group is substituted with one R²² group, and said R²² is F.

In another embodiment of the compounds of formula (I) R^{1A} is alkyl substituted with one R²¹ group, wherein said R²¹ group is aryl, said alkyl group is (a) (e.g., (b) or (c)), as described above, and said R²¹ group is substituted with two independently selected R²² groups, and each R²² is F.

In another embodiment of the compounds of formula (I) R^{1A} is alkyl substituted with one R²¹ group, wherein said R²¹ group is aryl, wherein said alkyl group is (a) (e.g., (b) or (c)), as described above, and said R²¹ group is substituted with one R²² group. and said R²² is F.

In another embodiment of the compounds of formula (I) R^{1A} is.

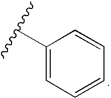

In another embodiment of the compounds of formula (I) R^{1A} is:

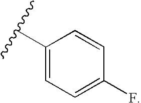

In another embodiment of the compounds of formula (I) R^{1A} is:

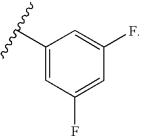

In another embodiment of this invention R^{1A} is:

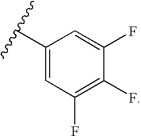

Examples of R²¹ groups include —OR¹⁵ wherein, for example, R¹⁵ is alkyl (such as methyl or ethyl), or R¹⁵ is cycloalkylalkyl (such as, for example, —CH₂-cyclopropyl), or R¹⁵ is -alkyl-(R¹⁸)ₙ (wherein, for example, said R¹⁸ is —OR²⁰, and said R²⁰ is alkyl, and wherein examples of said -alkyl-(R¹⁸)ₙ moiety is —(CH₂)₂OCH₃).

Examples of R²¹ also include —C(O)OR¹⁵ wherein, for example, R¹⁵ is alkyl, such as, for example, methyl).

Examples of R²¹ also include —C(O)NR¹⁵R¹⁶, wherein, for example, one of R¹⁵ or R¹⁶ is H, and the other is selected from the group consisting of: (R¹⁸)ₙ-arylalkyl, (R¹⁸)ₙ-alkyl-, and cycloalkyl. In one example of this —C(O)NR¹⁵R¹⁶ moiety the R¹⁸ is —OR²⁰, n is 1, R²⁰ is alkyl, said cycloalkyl is cyclobutyl, and said arylalkyl- is benzyl.

Examples of R²¹ also include halo (e.g., Br, Cl or F).

Examples of R²¹ also include arylalkyl, such as, for example, benzyl.

The dashed line between G¹ and the C that R^{1A} is bound to in the formulas below represents the presence of Ring B (i.e., Ring B is present in the formulas with the dashed line between G¹ and the C that R^{1A} is bound to). Thus, for example, Ring B is present in formulas IA to IH, 6.2, 10.2, 10.3, 20.2 21.2, and 23.2.

In another embodiment of this invention, the compound of formula (I) is a compound of formula (IA):

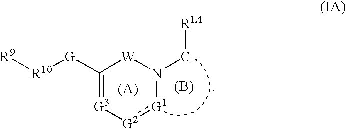

In another embodiment of this invention, the compound of formula (I) is a compound of formula (IB):

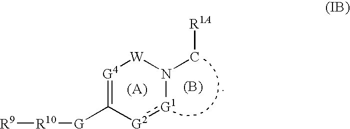

In another embodiment of this invention, the optional bond between G¹ and G² is present in formula (I).

In another embodiment of this invention, the optional bond between G¹ and G² is absent in formula (I).

In another embodiment of this invention, the optional bond between G¹ and G² is present in formula (IA).

In another embodiment of this invention, the optional bond between G¹ and G² is absent in formula (IA).

In another embodiment of this invention, the optional bond between G¹ and G² is present in formula (IB).

In another embodiment of this invention, the optional bond between G¹ and G² is absent in formula (IB).

In another embodiment of this invention, the compound of formula (I) is a compound of formula (IC):

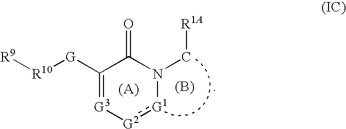

In one embodiment of this invention, the compound of formula (I) is a compound of formula (ID):

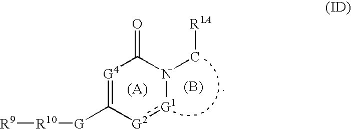

In one embodiment of this invention, the compound of formula (I) is a compound of formula (IE):

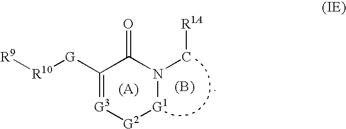

In one embodiment of this invention, the compound of formula (I) is a compound of formula (IF):

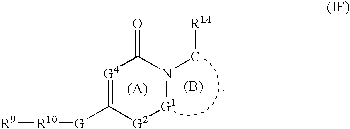

In one embodiment of this invention, the compound of formula (I) is a compound of formula (IG):

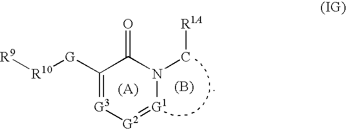

In one embodiment of this invention, the compound of formula (I) is a compound of formula (IH):

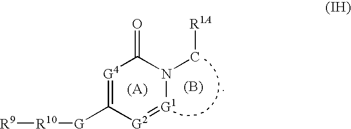

In one embodiment of this invention, the compound of formula (I) is a compound of formula (6.2):

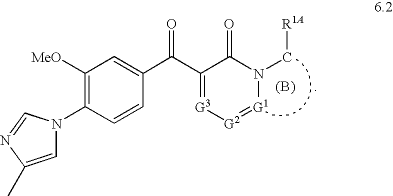

In one embodiment of this invention, the compound of formula (I) is a compound of formula (10.2):

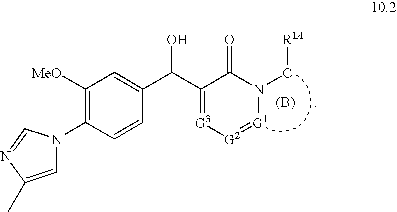

In one embodiment of this invention, the compound of formula (I) is a compound of formula (10.3):

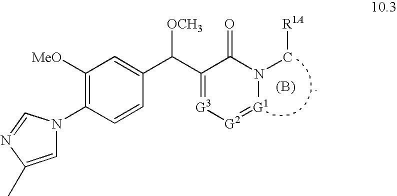

In one embodiment of this invention, the compound of formula (I) is a compound of formula (20.2):

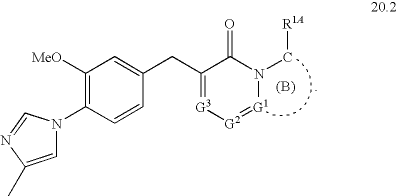

In one embodiment of this invention, the compound of formula (I) is a compound of formula (21.2):

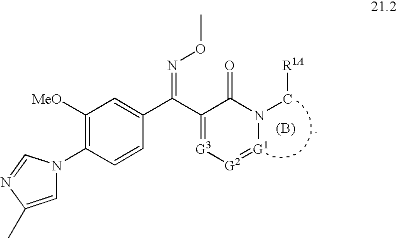

In one embodiment of this invention, the compound of formula (I) is a compound of formula (23.2):

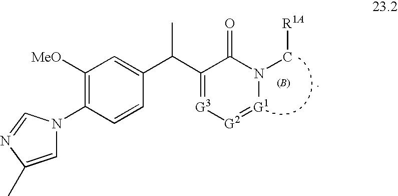

In another embodiment of this invention, Ring (B) is a 6 membered (including the atoms common to Rings (A) and (B)) heterocycloalkyl ring, optionally comprising one additional heteroatom (e.g., —NR²— or —O—).

In another embodiment of this invention, Ring (B) is a 5 membered (including the atoms common to Rings (A) and (B)) heterocycloalkyl ring, optionally comprising one additional heteroatom (e.g., —NR²— or —O—).

In another embodiment of this invention, Ring (B) is a 6 membered (including the atoms common to Rings (A) and (B)) heteroaryl ring, optionally comprising one or two additional heteroatoms (e.g., each independently selected from —NR²— or —O—).

In another embodiment of this invention, Ring (B) is a 5 membered (including the atoms common to Rings (A) and (B)) heterocycloalkyl ring, optionally comprising one or two additional heteroatom (e.g., each independently selected from —NR²— or —O—).

Another embodiment of this invention is directed to compounds of formula (I) having the formula:

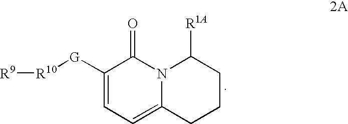

In one embodiment of this invention R^{1A} in compound 2A is aryl or substituted aryl. In another embodiment R^{1A} in compound 2A is phenyl or substituted phenyl. In another embodiment. R^{1A} in compound 2A is phenyl substituted with 1 to 3 independently selected R²¹ groups. In another embodiment of this invention R^{1A} in compound 2A is phenyl substituted with 1 to 3 (e.g., 1 to 3, or 1 to 2, or 1) independently selected halos. In another embodiment of this invention R^{1A} in compound 2A is phenyl substituted with 1 to 3 (e.g., 1 to 3, or 1 to 2, or 1) F atoms. In another embodiment of this invention R^{1A} in compound 2A is phenyl substituted with 1 F atom.

Another embodiment of this invention is directed to compounds of formula (I) having the formula:

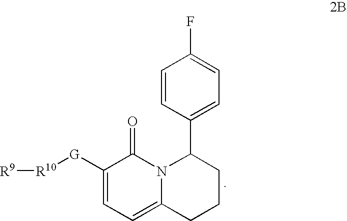

Another embodiment of this invention is directed to compounds of formula (I) having the formula:

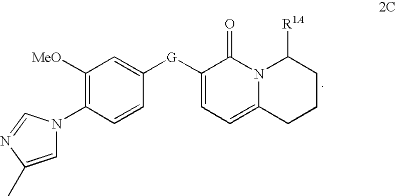

In one embodiment of this invention R^{1A} in compound 2C is aryl or substituted aryl. In another embodiment R^{1A} in compound 2C is phenyl or substituted phenyl. In another embodiment R^{1A} in compound 2C is phenyl substituted with 1 to 3 independently selected R²¹ groups. In another embodiment of this invention R^{1A} in compound 2C is phenyl substituted with 1 to 3 (e.g., 1 to 3, or 1 to 2, or 1) independently selected halos. In another embodiment of this invention R^{1A} in compound 2C is phenyl substituted with 1 to 3 (e.g., 1 to 3, or 1 to 2, or 1) F atoms. In another embodiment of this invention R^{1A} in compound 2C is phenyl substituted with 1 F atom.

Another embodiment of this invention is directed to compounds of formula (I) having the formula:

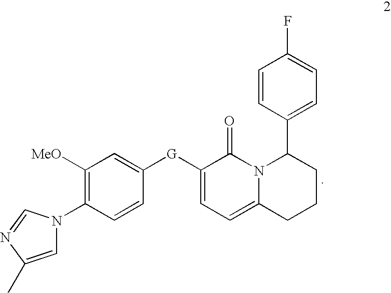

Another embodiment of this invention is directed to compounds of formula (I) having the formula:

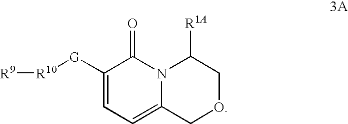

In one embodiment of this invention R^{1A} in compound 3A is aryl or substituted aryl. In another embodiment R^{1A} in compound 3A is phenyl or substituted phenyl. In another embodiment R^{1A} in compound 3A is phenyl substituted with 1 to 3 independently selected R²¹ groups. In another embodiment of this invention R^{1A} in compound 3A is phenyl substituted with 1 to 3 (e.g., 1 to 3, or 1 to 2, or 1) independently selected halos. In another embodiment of this invention R^{1A} in compound 3A is phenyl substituted with 1 to 3 (e.g., 1 to 3, or 1 to 2, or 1) F atoms. In another embodiment of this invention R^{1A} in compound 3A is phenyl substituted with 1 F atom.

Another embodiment of this invention is directed to compounds of formula (I) having the formula:

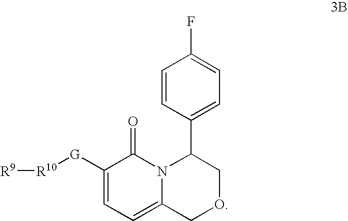

Another embodiment of this invention is directed to compounds of formula (I) having the formula:

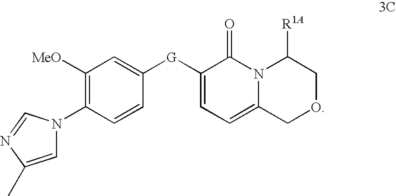

In one embodiment of this invention R^{1A} in compound 3C is aryl or substituted aryl. In another embodiment R^{1A} in compound 3C is phenyl or substituted phenyl. In another embodiment R^{1A} in compound 3C is phenyl substituted with 1 to 3 independently selected R²¹ groups. In another embodiment of this invention R^{1A} in compound 3C is phenyl substituted with 1 to 3 (e.g., 1 to 3, or 1 to 2, or 1) independently selected halos. In another embodiment of this invention R^{1A} in compound 3C is phenyl substituted with 1 to 3 (e.g., 1 to 3, or 1 to 2, or 1) F atoms. In another embodiment of this invention R^{1A} in compound 3C is phenyl substituted with 1 F atom.

Another embodiment of this invention is directed to compounds of formula (I) having the formula:

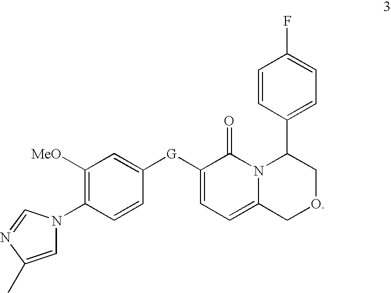

Another embodiment of this invention is directed to compounds of formula (I) having the formula:

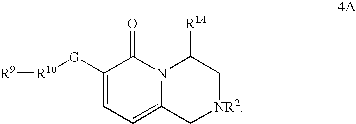

In one embodiment of this invention R^{1A} in compound 4A is aryl or substituted aryl. In another embodiment R^{1A} in compound 4A is phenyl or substituted phenyl. In another embodiment R^{1A} in compound 4A is phenyl substituted with 1 to 3 independently selected R²¹ groups. In another embodiment of this invention R^{1A} in compound 4A is phenyl substituted with 1 to 3 (e.g., 1 to 3, or 1 to 2, or 1) independently selected halos. In another embodiment of this invention R^{1A} in compound 4A is phenyl substituted with 1 to 3 (e.g., 1 to 3, or 1 to 2, or 1) F atoms. In another embodiment of this invention R^{1A} in compound 4A is phenyl substituted with 1 F atom.

Another embodiment of this invention is directed to compounds of formula (I) having the formula:

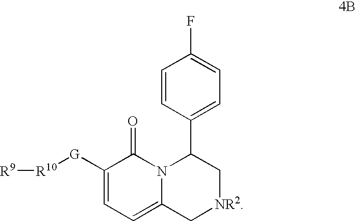

Another embodiment of this invention is directed to compounds of formula (I) having the formula:

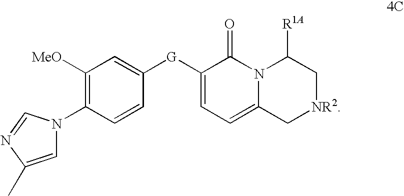

In one embodiment of this invention R^{1A} in compound 4C is aryl or substituted aryl. In another embodiment R^{1A} in compound 4C is phenyl or substituted phenyl. In another embodiment R^{1A} in compound 4C is phenyl substituted with 1 to 3 independently selected R²¹ groups. In another embodiment of this invention R^{1A} in compound 4C is phenyl substituted with 1 to 3 (e.g., 1 to 3, or 1 to 2, or 1) independently selected halos. In another embodiment of this invention R^{1A} in compound 4C is phenyl substituted with 1 to 3 (e.g., 1 to 3, or 1 to 2, or 1) F atoms. In another embodiment of this invention R^{1A} in compound 4C is phenyl substituted with 1 F atom.

Another embodiment of this invention is directed to compounds of formula (I) having the formula:

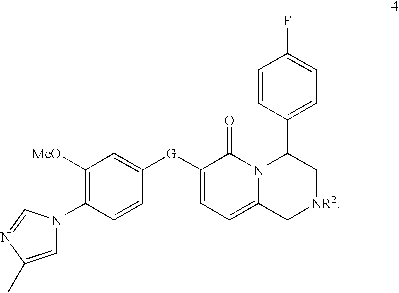

Another embodiment of this invention is directed to compounds of formula (I) having the formula:

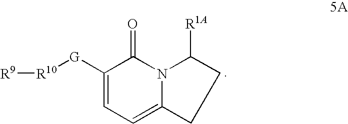

In one embodiment of this invention R^{1A} in compound 5A is aryl or substituted aryl. In another embodiment R^{1A} in compound 5A is phenyl or substituted phenyl. In another embodiment R^{1A} in compound 5A is phenyl substituted with 1 to 3 independently selected R²¹ groups. In another embodiment of this invention R^{1A} in compound 5A is phenyl substituted with 1 to 3 (e.g., 1 to 3, or 1 to 2, or 1) independently selected halos. In another embodiment of this invention R^{1A} in compound 5A is phenyl substituted with 1 to 3 (e.g., 1 to 3, or 1 to 2, or 1) F atoms. In another embodiment of this invention R^{1A} in compound 5A is phenyl substituted with 1 F atom.

Another embodiment of this invention is directed to compounds of formula (I) having the formula:

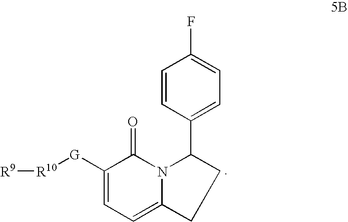

Another embodiment of this invention is directed to compounds of formula (I) having the formula:

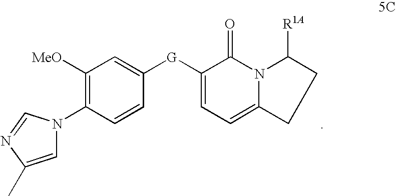

In one embodiment of this invention R^{1A} in compound 5C is aryl or substituted aryl. In another embodiment R^{1A} in compound 5C is phenyl or substituted phenyl. In another embodiment R^{1A} in compound 5C is phenyl substituted with 1 to 3 independently selected R²¹ groups. In another embodiment of this invention R^{1A} in compound 5C is phenyl substituted with 1 to 3 (e.g., 1 to 3, or 1 to 2, or 1) independently selected halos. In another embodiment of this invention R^{1A} in compound 5C is phenyl substituted with 1 to 3 (e.g., 1 to 3, or 1 to 2, or 1) F atoms. In another embodiment of this invention R^{1A} in compound 5C is phenyl substituted with 1 F atom.

Another embodiment of this invention is directed to compounds of formula (I) having the formula:

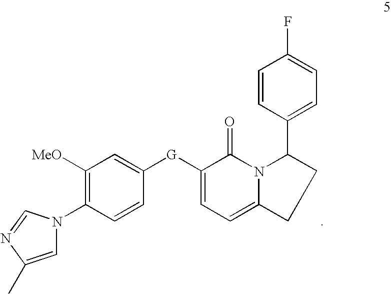

Another embodiment of this invention is directed to compounds of formula (I) having the formula:

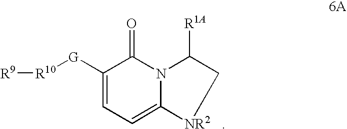

In one embodiment of this invention R^{1A} in compound 6A is aryl or substituted aryl. In another embodiment R^{1A} in compound 6A is phenyl or substituted phenyl. In another embodiment R^{1A} in compound 6A is phenyl substituted with 1 to 3 independently selected R²¹ groups. In another embodiment of this invention R^{1A} in compound 6A is phenyl substituted with 1 to 3 (e.g., 1 to 3, or 1 to 2, or 1) independently selected halos. In another embodiment of this invention R^{1A} in compound 6A is phenyl substituted with 1 to 3 (e.g., 1 to 3, or 1 to 2, or 1) F atoms. In another embodiment of this invention R^{1A} in compound 5A is phenyl substituted with 1 F atom.

Another embodiment of this invention is directed to compounds of formula (I) having the formula:

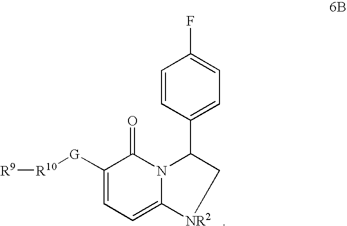

Another embodiment of this invention is directed to compounds of formula (I) having the formula:

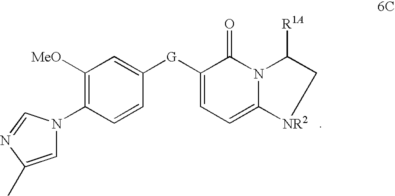

In one embodiment of this invention R^{1A} in compound 6C is aryl or substituted aryl. In another embodiment R^{1A} in compound 6C is phenyl or substituted phenyl. In another embodiment R^{1A} in compound 6C is phenyl substituted with 1 to 3 independently selected R²¹ groups. In another embodiment of this invention R^{1A} in compound 6C is phenyl substituted with 1 to 3 (e.g., 1 to 3, or 1 to 2, or 1) independently selected halos. In another embodiment of this invention R^{1A} in compound 6C is phenyl substituted with 1 to 3 (e.g., 1 to 3, or 1 to 2, or 1) F atoms. In another embodiment of this invention R^{1A} in compound BC is phenyl substituted with 1 F atom.

Another embodiment of this invention is directed to compounds of formula (I) having the formula:

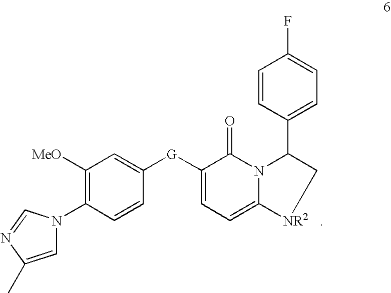

Another embodiment of this invention is directed to compounds of formula (I) having the formula:

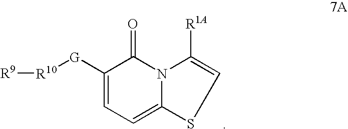

In one embodiment of this invention R^{1A} in compound 7A is aryl or substituted aryl. In another embodiment R^{1A} in compound 7A is phenyl or substituted phenyl. In another embodiment R^{1A} in compound 7A is phenyl substituted with 1 to 3 independently selected R²¹ groups. In another embodiment of this invention R^{1A} in compound 7A is phenyl substituted with 1 to 3 (e.g., 1 to 3, or 1 to 2, or 1) independently selected halos. In another embodiment of this invention R^{1A} in compound 7A is phenyl substituted with 1 to 3 (e.g., 1 to 3, or 1 to 2, or 1) F atoms. In another embodiment of this invention R^{1A} in compound 7A is phenyl substituted with 1 F atom.

Another embodiment of this invention is directed to compounds of formula (I) having the formula:

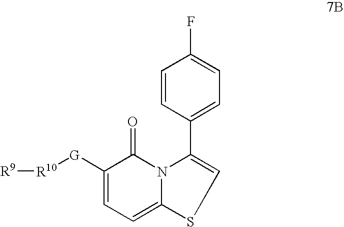

Another embodiment of this invention is directed to compounds of formula (I) having the formula:

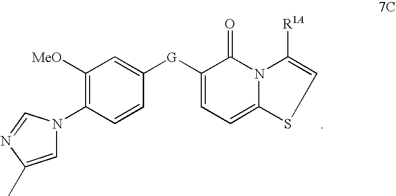

In one embodiment of this invention R^{1A} in compound 7C is aryl or substituted aryl. In another embodiment R^{1A} in compound 7C is phenyl or substituted phenyl. In another embodiment R^{1A} in compound 7C is phenyl substituted with 1 to 3 independently selected R²¹ groups. In another embodiment of this invention R^{1A} in compound 7C is phenyl substituted with 1 to 3 (e.g., 1 to 3, or 1 to 2, or 1) independently selected halos. In another embodiment of this invention R^{1A} in compound 7C is phenyl substituted with 1 to 3 (e.g., 1 to 3, or 1 to 2, or 1) F atoms. In another embodiment of this invention R^{1A} in compound 7C is phenyl substituted with 1 F atom.

Another embodiment of this invention is directed to compounds of formula (I) having the formula:

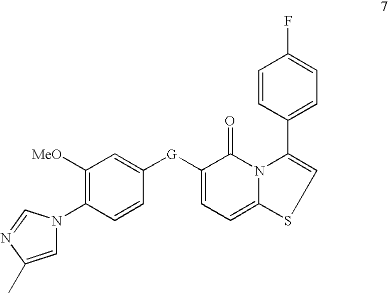

Another embodiment of this invention is directed to compounds of formula (I) having the formula:

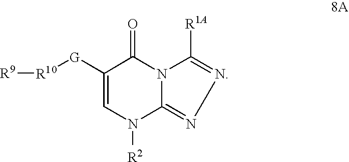

In one embodiment of this invention R^{1A} in compound 8A is aryl or substituted aryl. In another embodiment R^{1A} in compound 8A is phenyl or substituted phenyl. In another embodiment R^{1A} in compound 8A is phenyl substituted with 1 to 3 independently selected R²¹ groups. In another embodiment of this invention R^{1A} in compound 8A is phenyl substituted with 1 to 3 (e.g., 1 to 3, or 1 to 2, or 1) independently selected halos. In another embodiment of this invention R^{1A} in compound 8A is phenyl substituted with 1 to 3 (e.g., 1 to 3, or 1 to 2, or 1) F atoms. In another embodiment of this invention R^{1A} in compound 8A is phenyl substituted with 1 F atom.

Another embodiment of this invention is directed to compounds of formula (I) having the formula:

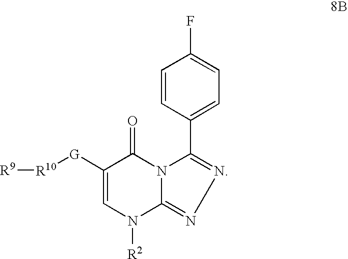

Another embodiment of this invention is directed to compounds of formula (I) having the formula:

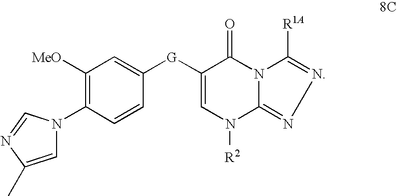

In one embodiment of this invention R^{1A} in compound 8C is aryl or substituted aryl. In another embodiment R^{1A} in compound 8C is phenyl or substituted phenyl. In another embodiment R^{1A} in compound 8C is phenyl substituted with 1 to 3 independently selected R²¹ groups. In another embodiment of this invention R^{1A} in compound 8C is phenyl substituted with 1 to 3 (e.g., 1 to 3, or 1 to 2, or 1) independently selected halos. In another embodiment of this invention R^{1A} in compound 8C is phenyl substituted with 1 to 3 (e.g., 1 to 3, or 1 to 2, or 1) F atoms. In another embodiment of this invention R^{1A} in compound 8C is phenyl substituted with 1 F atom.

Another embodiment of this invention is directed to compounds of formula (I) having the formula:

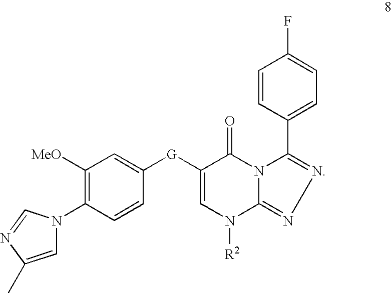

Another embodiment of this invention is directed to compounds of formula (I) having the formula:

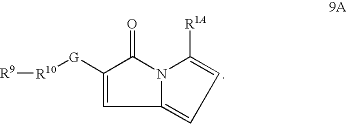

In one embodiment of this invention R^{1A} in compound 9A is aryl or substituted aryl. In another embodiment R^{1A} in compound 9A is phenyl or substituted phenyl. In another embodiment R^{1A} in compound 9A is phenyl substituted with 1 to 3 independently selected R²¹ groups. In another embodiment of this invention R^{1A} in compound 9A is phenyl substituted with 1 to 3 (e.g., 1 to 3, or 1 to 2, or 1) independently selected halos. In another embodiment of this invention R^{1A} in compound 9A is phenyl substituted with 1 to 3 (e.g., 1 to 3, or 1 to 2, or 1) F atoms. In another embodiment of this invention R^{1A} in compound 9A is phenyl substituted with 1 F atom.

Another embodiment of this invention is directed to compounds of formula (I) having the formula:

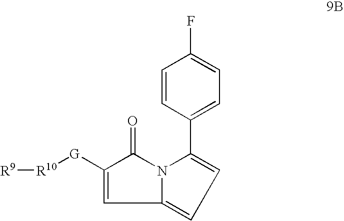

Another embodiment of this invention is directed to compounds of formula (I) having the formula:

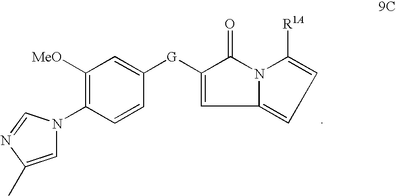

In one embodiment of this invention R^{1A} in compound 9C is aryl or substituted aryl. In another embodiment R^{1A} in compound 9C is phenyl or substituted phenyl. In another embodiment R^{1A} in compound 9C is phenyl substituted with 1 to 3 independently selected R²¹ groups. In another embodiment of this invention R^{1A} in compound 9C is phenyl substituted with 1 to 3 (e.g., 1 to 3, or 1 to 2, or 1) independently selected halos. In another embodiment of this invention R^{1A} in compound 9C is phenyl substituted with 1 to 3 (e.g., 1 to 3, or 1 to 2, or 1) F atoms. In another embodiment of this invention R^{1A} in compound 9C is phenyl substituted with 1 F atom.

Another embodiment of this invention is directed to compounds of formula (I) having the formula:

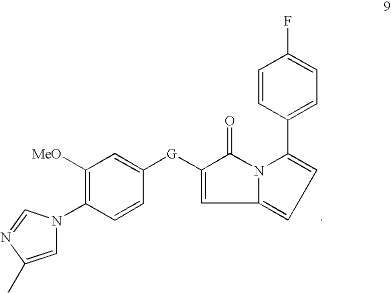

Another embodiment of this invention is directed to compounds of formula (I) having the formula:

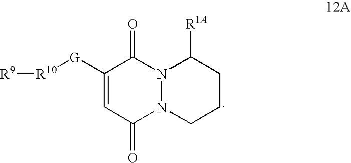

In one embodiment of this invention R^{1A} in compound 12A is aryl or substituted aryl. In another embodiment R^{1A} in compound 12A is phenyl or substituted phenyl. In another embodiment R^{1A} in compound 12A is phenyl substituted with 1 to 3 independently selected R²¹ groups. In another embodiment of this invention R^{1A} in compound 12A is phenyl substituted with 1 to 3 (e.g., 1 to 3, or 1 to 2, or 1) independently selected halos. In another embodiment of this invention R^{1A} in compound 12A is phenyl substituted with 1 to 3 (e.g., 1 to 3, or 1 to 2, or 1) F atoms. In another embodiment of this invention R^{1A} in compound 12A is phenyl substituted with 1 F atom.

Another embodiment of this invention is directed to compounds of formula (I) having the formula:

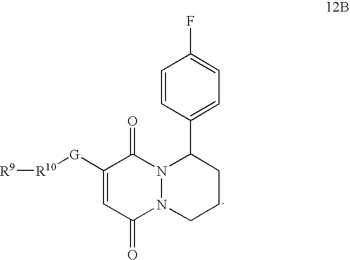

Another embodiment of this invention is directed to compounds of formula (I) having the formula:

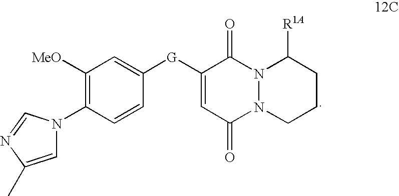

In one embodiment of this invention R^{1A} in compound 12C is aryl or substituted aryl. In another embodiment R^{1A} in compound 12C is phenyl or substituted phenyl. In another embodiment R^{1A} in compound 12C is phenyl substituted with 1 to 3 independently selected R²¹ groups. In another embodiment of this invention R^{1A} in compound 12C is phenyl substituted with 1 to 3 (e.g., 1 to 3, or 1 to 2, or 1) independently selected halos. In another embodiment of this invention R^{1A} in compound 12C is phenyl substituted with 1 to 3 (e.g., 1 to 3, or 1 to 2, or 1) F atoms. In another embodiment of this invention R^{1A} in compound 12C is phenyl substituted with 1 F atom.

Another embodiment of this invention is directed to compounds of formula (I) having the formula:

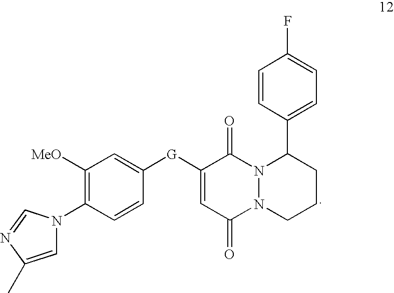

Another embodiment of this invention is directed to compounds of formula (I) having the formula:

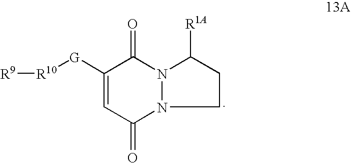

In one embodiment of this invention R^{1A} in compound 13A is aryl or substituted aryl. In another embodiment R^{1A} in compound 13A is phenyl or substituted phenyl. In another embodiment R^{1A} in compound 13A is phenyl substituted with 1 to 3 independently selected R²¹ groups. In another embodiment of this invention R^{1A} in compound 13A is phenyl substituted with 1 to 3 (e.g., 1 to 3, or 1 to 2, or 1) independently selected halos. In another embodiment of this invention R^{1A} in compound 13A is phenyl substituted with 1 to 3 (e.g., 1 to 3, or 1 to 2, or 1) F atoms. In another embodiment of this invention R^{1A} in compound 13A is phenyl substituted with 1 F atom.

Another embodiment of this invention is directed to compounds of formula (I) having the formula:

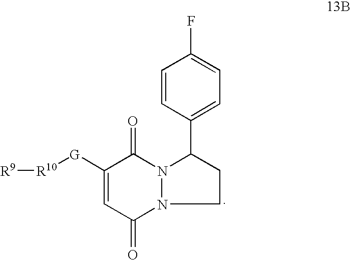

Another embodiment of this invention is directed to compounds of formula (I) having the formula:

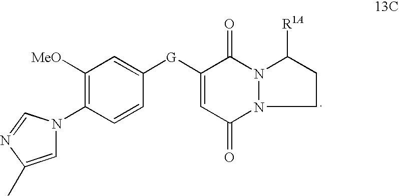

In one embodiment of this invention R^{1A} in compound 13C is aryl or substituted aryl. In another embodiment R^{1A} in compound 13C is phenyl or substituted phenyl. In another embodiment R^{1A} in compound 13C is phenyl substituted with 1 to 3 independently selected R²¹ groups. In another embodiment of this invention R^{1A} in compound 13C is phenyl substituted with 1 to 3 (e.g., 1 to 3, or 1 to 2, or 1) independently selected halos. In another embodiment of this invention R^{1A} in compound 13C is phenyl substituted with 1 to 3 (e.g., 1 to 3, or 1 to 2, or 1) F atoms.

Another embodiment of this invention is directed to compounds of formula (I) having the formula:

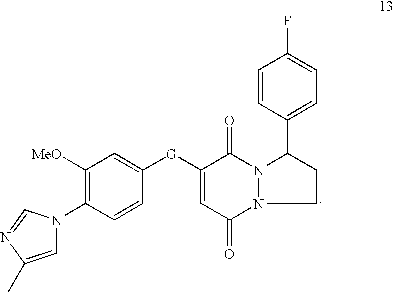

Another embodiment of this invention is directed to compounds of formula (I) having the formula:

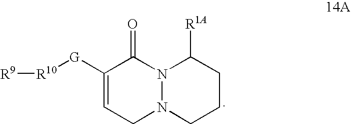

In one embodiment of this invention R^{1A} in compound 14A is aryl or substituted aryl. In another embodiment R^{1A} in compound 14A is phenyl or substituted phenyl. In another embodiment R^{1A} in compound 14A is phenyl substituted with 1 to 3 independently selected R²¹ groups. In another embodiment of this invention R^{1A} in compound 14A is phenyl substituted with 1 to 3 (e.g., 1 to 3, or 1 to 2, or 1) independently selected halos. In another embodiment of this invention R^{1A} in compound 14A is phenyl substituted with 1 to 3 (e.g., 1 to 3, or 1 to 2, or 1) F atoms. In another embodiment of this invention R^{1A} in compound 14A is phenyl substituted with 1 F atom.

Another embodiment of this invention is directed to compounds of formula (I) having the formula:

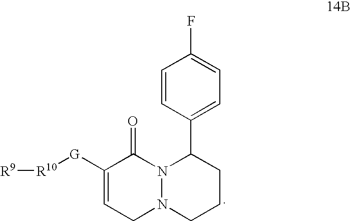

Another embodiment of this invention is directed to compounds of formula (I) having the formula:

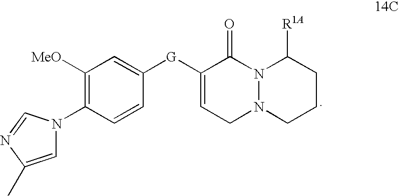

In one embodiment of this invention R^{1A} in compound 14C is aryl or substituted aryl. In another embodiment R^{1A} in compound 14C is phenyl or substituted phenyl. In another embodiment R^{1A} in compound 14C is phenyl substituted with 1 to 3 independently selected R²¹ groups. In another embodiment of this invention R^{1A} in compound 14C is phenyl substituted with 1 to 3 (e.g., 1 to 3, or 1 to 2, or 1) independently selected halos. In another embodiment of this invention R^{1A} in compound 14C is phenyl substituted with 1 to 3 (e.g., 1 to 3, or 1 to 2, or 1) F atoms. In another embodiment of this invention R^{1A} in compound 15C is phenyl substituted with 1 F atom.

Another embodiment of this invention is directed to compounds of formula (I) having the formula:

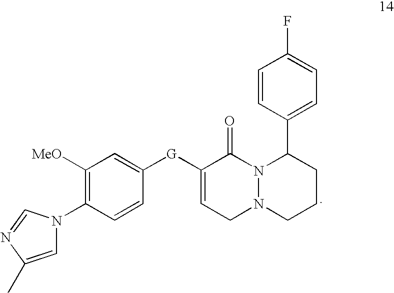

Another embodiment of this invention is directed to compounds of formula (I) having the formula:

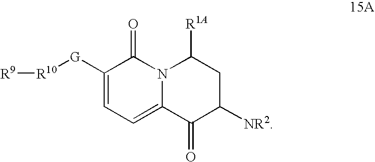

In one embodiment of this invention R^{1A} in compound 15A is aryl or substituted aryl. In another embodiment R^{1A} in compound 15A is phenyl or substituted phenyl. In another embodiment R^{1A} in compound 15A is phenyl substituted with 1 to 3 independently selected R²¹ groups. In another embodiment of this invention R^{1A} in compound 15A is phenyl substituted with 1 to 3 (e.g., 1 to 3, or 1 to 2, or 1) independently selected halos. In another embodiment of this invention R^{1A} in compound 15A is phenyl substituted with 1 to 3 (e.g., 1 to 3, or 1 to 2, or 1) F atoms. In another embodiment of this invention R^{1A} in compound 15A is phenyl substituted with 1 F atom

Another embodiment of this invention is directed to compounds of formula (I) having the formula:

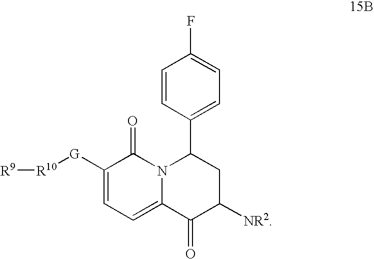

Another embodiment of this invention is directed to compounds of formula (I) having the formula:

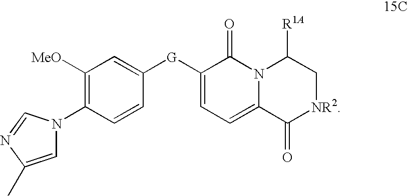

In one embodiment of this invention R^{1A} in compound 15C is aryl or substituted aryl. In another embodiment R^{1A} in compound 15C is phenyl or substituted phenyl. In another embodiment R^{1A} in compound 15C is phenyl substituted with 1 to 3 independently selected R²¹ groups. In another embodiment of this invention R^{1A} in compound 15C is phenyl substituted with 1 to 3 (e.g., 1 to 3, or 1 to 2, or 1) independently selected halos. In another embodiment of this invention R^{1A} in compound 15C is phenyl substituted with 1 to 3 (e.g., 1 to 3, or 1 to 2, or 1) F atoms. In another embodiment of this invention R^{1A} in compound 15C is phenyl substituted with 1 F atom.

Another embodiment of this invention is directed to compounds of formula having the formula:

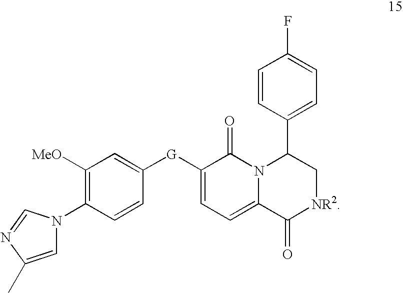

Another embodiment of this invention is directed to compounds of formula (I) having the formula:

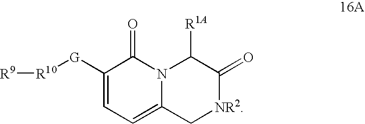

In one embodiment of this invention R^{1A} in compound 16A is aryl or substituted aryl. In another embodiment R^{1A} in compound 16A is phenyl or substituted phenyl. In another embodiment R^{1A} in compound 16A is phenyl substituted with 1 to 3 independently selected R²¹ groups. In another embodiment of this invention R^{1A} in compound 16A is phenyl substituted with 1 to 3 (e.g., 1 to 3, or 1 to 2, or 1) independently selected halos. In another embodiment of this invention R^{1A} in compound 16A is phenyl substituted with 1 to 3 (e.g., 1 to 3, or 1 to 2, or 1) F atoms. In another embodiment of this invention R^{1A} in compound 16A is phenyl substituted with 1 F atom.

Another embodiment of this invention is directed to compounds of formula (I) having the formula:

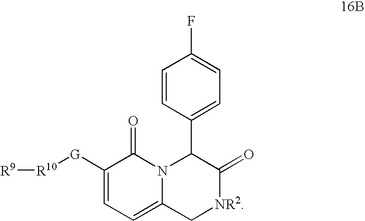

Another embodiment of this invention is directed to compounds of formula (I) having the formula:

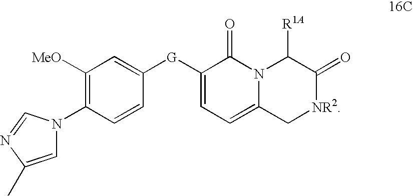

In one embodiment of this invention R^{1A} in compound 16C is aryl or substituted aryl. In another embodiment R^{1A} in compound 16C is phenyl or substituted phenyl. In another embodiment R^{1A} in compound 16C is phenyl substituted with 1 to 3 independently selected R²¹ groups. In another embodiment of this invention R^{1A} in compound 16C is phenyl substituted with 1 to 3 (e.g., 1 to 3, or 1 to 2, or 1) independently selected halos. In another embodiment of this invention R^{1A} in compound 16C is phenyl substituted with 1 to 3 (e.g., 1 to 3, or 1 to 2, or 1) F atoms. In another embodiment of this invention R^{1A} in compound 16C is phenyl substituted with 1 F atom.

Another embodiment of this invention is directed to compounds of formula (I) having the formula:

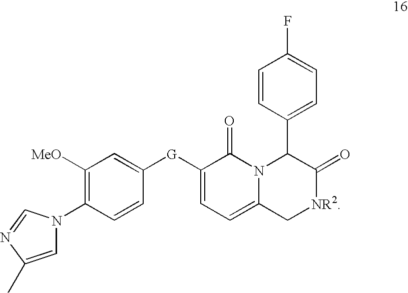

Another embodiment of this invention is directed to compounds of formula (I) having the formula:

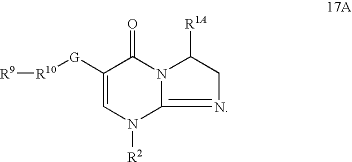

In one embodiment of this invention R^{1A} in compound 17A is aryl or substituted aryl. In another embodiment R^{1A} in compound 17A is phenyl or substituted phenyl. In another embodiment R^{1A} in compound 17A is phenyl substituted with 1 to 3 independently selected R²¹ groups. In another embodiment of this invention R^{1A} in compound 17A is phenyl substituted with 1 to 3 (e.g., 1 to 3, or 1 to 2, or 1) independently selected halos. In another embodiment of this invention R^{1A} in compound 17A is phenyl substituted with 1 to 3 (e.g., 1 to 3, or 1 to 2, or 1) F atoms. In another embodiment of this invention R^{1A} in compound 17A is phenyl substituted with 1 F atom.

Another embodiment of this invention is directed to compounds of formula (I) having the formula:

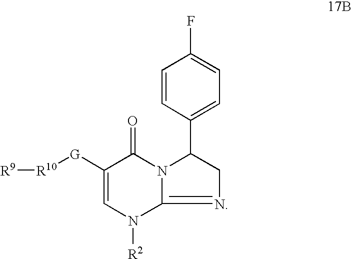

Another embodiment of this invention is directed to compounds of formula (I) having the formula:

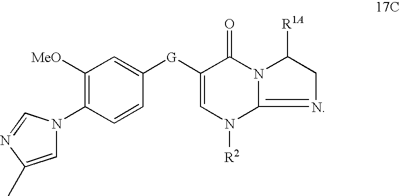

In one embodiment of this invention R^{1A} in compound 17C is aryl or substituted aryl. In another embodiment R^{1A} in compound 17C is phenyl or substituted phenyl. In another embodiment R^{1A} in compound 17C is phenyl substituted with 1 to 3 independently selected R²¹ groups. In another embodiment of this invention R^{1A} in compound 17C is phenyl substituted with 1 to 3 (e.g., 1 to 3, or 1 to 2, or 1) independently selected halos. In another embodiment of this invention R^{1A} in compound 17C is phenyl substituted with 1 to 3 (e.g., 1 to 3, or 1 to 2, or 1) F atoms. In another embodiment of this invention R^{1A} in compound 17C is phenyl substituted with 1 F atom.

Another embodiment of this invention is directed to compounds of formula (I) having the formula:

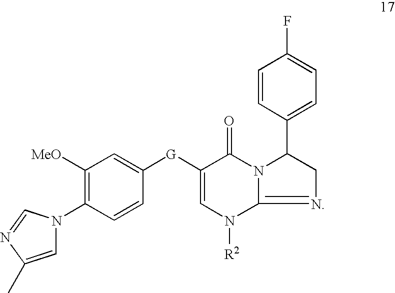

Another embodiment of this invention is directed to compounds of formula (I) having the formula:

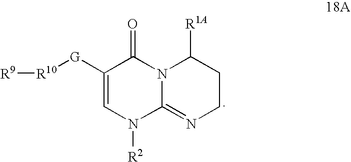

In one embodiment of this invention R^{1A} in compound 18A is aryl or substituted aryl. In another embodiment R^{1A} in compound 18A is phenyl or substituted phenyl. In another embodiment R^{1A} in compound 18A is phenyl substituted with 1 to 3 independently selected R²¹ groups. In another embodiment of this invention R^{1A} in compound 18A is phenyl substituted with 1 to 3 (e.g., 1 to 3, or 1 to 2, or 1) independently selected halos. In another embodiment of this invention R^{1A} in compound 18A is phenyl substituted with 1 to 3 (e.g., 1 to 3, or 1 to 2, or 1) F atoms. In another embodiment of this invention R^{1A} in compound 18A is phenyl substituted with 1 F atom.

Another embodiment of this invention is directed to compounds of formula (I) having the formula:

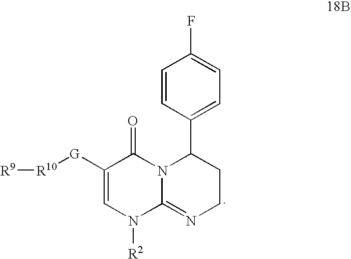

Another embodiment of this invention is directed to compounds of formula (I) having the formula:

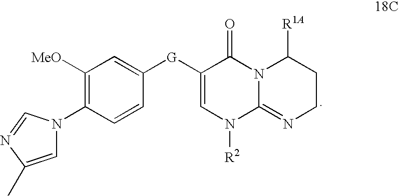

In one embodiment of this invention R^{1A} in compound 18C is aryl or substituted aryl. In another embodiment R^{1A} in compound 18C is phenyl or substituted phenyl. In another embodiment R^{1A} in compound 18C is phenyl substituted with 1 to 3 independently selected R²¹ groups. In another embodiment of this invention R^{1A} in compound 18C is phenyl substituted with 1 to 3 (e.g., 1 to 3, or 1 to 2, or 1) independently selected halos. In another embodiment of this invention R^{1A} in compound 18C is phenyl substituted with 1 to 3 (e.g., 1 to 3, or 1 to 2, or 1) F atoms. In another embodiment of this invention R^{1A} in compound 18C is phenyl substituted with 1 F atom.

Another embodiment of this invention is directed to compounds of formula (I) having the formula:

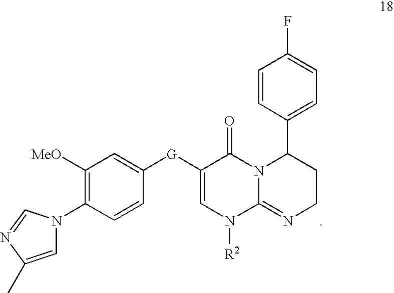

Another embodiment of this invention is directed to compounds of formula (I) having the formula:

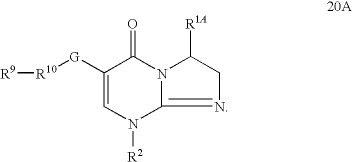

In one embodiment of this invention R^{1A} in compound 20A is aryl or substituted aryl. In another embodiment R^{1A} in compound 20A is phenyl or substituted phenyl. In another embodiment R^{1A} in compound 20A is phenyl substituted with 1 to 3 independently selected R²¹ groups. In another embodiment of this invention R^{1A} in compound 20A is phenyl substituted with 1 to 3 (e.g., 1 to 3, or 1 to 2, or 1) independently selected halos. In another embodiment of this invention R^{1A} in compound 20A is phenyl substituted with 1 to 3 (e.g., 1 to 3, or 1 to 2, or 1) F atoms. In another embodiment of this invention R^{1A} in compound 20A is phenyl substituted with 1 F atom.

Another embodiment of this invention is directed to compounds of formula (I) having the formula:

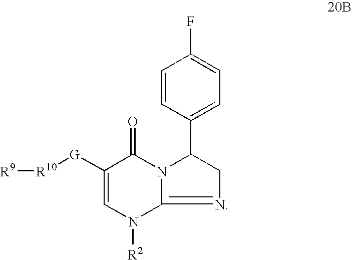

Another embodiment of this invention is directed to compounds of formula (I) having the formula:

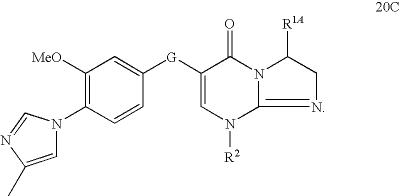

In one embodiment of this invention R^{1A} in compound 20C is aryl or substituted aryl. In another embodiment R^{1A} in compound 20C is phenyl or substituted phenyl. In another embodiment R^{1A} in compound 20C is phenyl substituted with 1 to 3 independently selected R²¹ groups. In another embodiment of this invention R^{1A} in compound 20C is phenyl substituted with 1 to 3 (e.g., 1 to 3, or 1 to 2, or 1) independently selected halos. In another embodiment of this invention R^{1A} in compound 20C is phenyl substituted with 1 to 3 (e.g., 1 to 3, or 1 to 2, or 1) F atoms. In another embodiment of this invention R^{1A} in compound 20C is phenyl substituted with 1 F atom.

Another embodiment of this invention is directed to compounds of formula (I) having the formula:

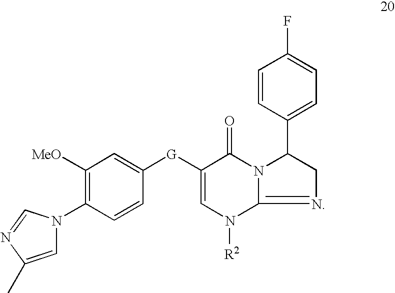

Another embodiment of this invention is directed to compounds of formula (I) having the formula:

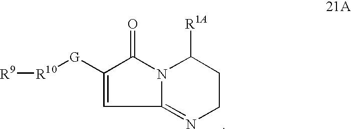

In one embodiment of this invention R^{1A} in compound 21A is aryl or substituted aryl. In another embodiment R^{1A} in compound 21A is phenyl or substituted phenyl. In another embodiment R^{1A} in compound 21A is phenyl substituted with 1 to 3 independently selected R²¹ groups. In another embodiment of this invention R^{1A} in compound 21A is phenyl substituted with 1 to 3 (e.g., 1 to 3, or 1 to 2, or 1) independently selected halos. In another embodiment of this invention R^{1A} in compound 21A is phenyl substituted with 1 to 3 (e.g., 1 to 3, or 1 to 2, or 1) F atoms. In another embodiment of this invention R^{1A} in compound 21A is phenyl substituted with 1 F atom.

Another embodiment of this invention is directed to compounds of formula (I) having the formula:

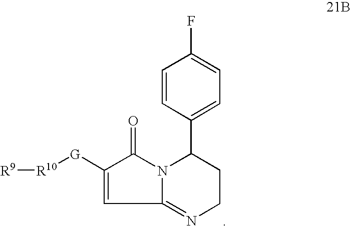

Another embodiment of this invention is directed to compounds of formula (I) having the formula:

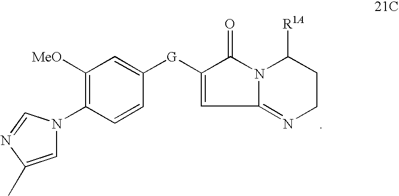

In one embodiment of this invention R^{1A} in compound 21 C is aryl or substituted aryl. In another embodiment R^{1A} in compound 21C is phenyl or substituted phenyl. In another embodiment R^{1A} in compound 21C is phenyl substituted with 1 to 3 independently selected R²¹ groups. In another embodiment of this invention R^{1A} in compound 21C is phenyl substituted with 1 to 3 (e.g., 1 to 3, or 1 to 2, or 1) independently selected halos. In another embodiment of this invention R^{1A} in compound 21C is phenyl substituted with 1 to 3 (e.g., 1 to 3, or 1 to 2, or 1) F atoms. In another embodiment of this invention R^{1A} in compound 21C is phenyl substituted with 1 F atom.

Another embodiment of this invention is directed to compounds of formula (I) having the formula:

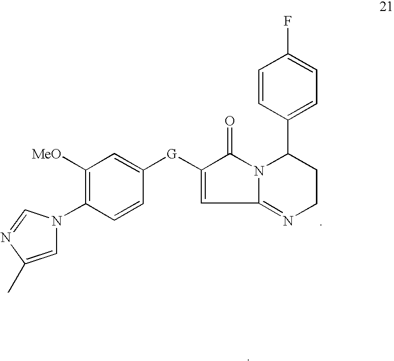

Another embodiment of this invention is directed to compounds of formula (I) having the formula:

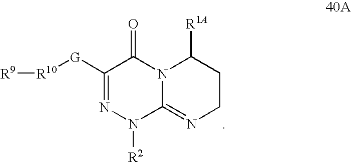

In one embodiment of this invention R^{1A} in compound 40A is aryl or substituted aryl. In another embodiment R^{1A} in compound 40A is phenyl or substituted phenyl. In another embodiment R^{1A} in compound 40A is phenyl substituted with 1 to 3 independently selected R²¹ groups. In another embodiment of this invention R^{1A} in compound 40A is phenyl substituted with 1 to 3 (e.g., 1 to 3, or 1 to 2, or 1) independently selected halos. In another embodiment of this invention R^{1A} in compound 40A is phenyl substituted with 1 to 3 (e.g., 1 to 3, or 1 to 2, or 1) F atoms. In another embodiment of this invention R^{1A} in compound 40A is phenyl substituted with 1 F atom

Another embodiment of this invention is directed to compounds of formula (I) having the formula:

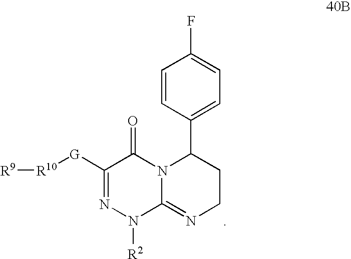

Another embodiment of this invention is directed to compounds of formula (I) having the formula:

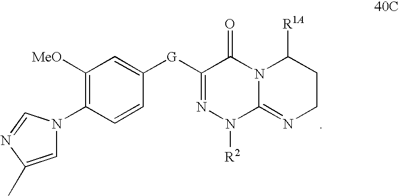

In one embodiment of this invention R^{1A} in compound 40C is aryl or substituted aryl. In another embodiment R^{1A} in compound 40C is phenyl or substituted phenyl. In another embodiment R^{1A} in compound 40C is phenyl substituted with 1 to 3 independently selected R²¹ groups. In another embodiment of this invention R^{1A} in compound 40C is phenyl substituted with 1 to 3 (e.g., 1 to 3, or 1 to 2, or 1) independently selected halos. In another embodiment of this invention R^{1A} in compound 40C is phenyl substituted with 1 to 3 (e.g., 1 to 3, or 1 to 2, or 1) F atoms. In another embodiment of this invention R^{1A} in compound 40C is phenyl substituted with 1 F atom.

Another embodiment of this invention is directed to compounds of formula (I) having the formula:

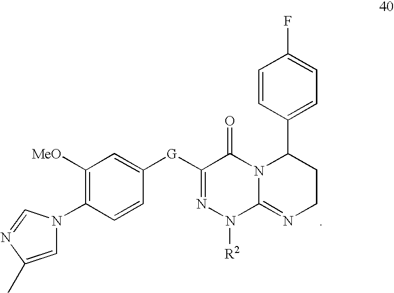

Another embodiment of this invention is directed to compounds of formula (I) having the formula:

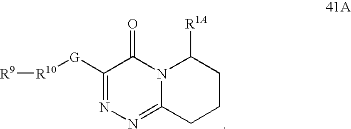

In one embodiment of this invention R^{1A} in compound 41A is aryl or substituted aryl. In another embodiment R^{1A} in compound 41A is phenyl or substituted phenyl. In another embodiment R^{1A} in compound 41A is phenyl substituted with 1 to 3 independently selected R²¹ groups. In another embodiment of this invention R^{1A} in compound 41A is phenyl substituted with 1 to 3 (e.g., 1 to 3, or 1 to 2, or 1) independently selected halos. In another embodiment of this invention R^{1A} in compound 41A is phenyl substituted with 1 to 3 (e.g., 1 to 3, or 1 to 2, or 1) F atoms. In another embodiment of this invention R^{1A} in compound 41A is phenyl substituted with 1 F atom

Another embodiment of this invention is directed to compounds of formula (I) having the formula:

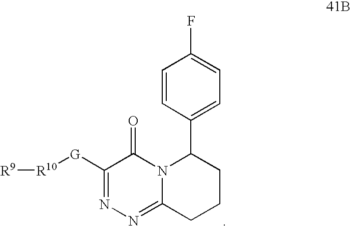

Another embodiment of this invention is directed to compounds of formula (I) having the formula:

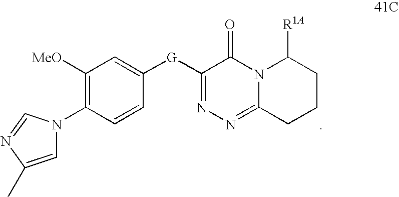

In one embodiment of this invention R^{1A} in compound 41C is aryl or substituted aryl. In another embodiment R^{1A} in compound 41 C is phenyl or substituted phenyl. In another embodiment R^{1A} in compound 41C is phenyl substituted with 1 to 3 independently selected R²¹ groups. In another embodiment of this invention R^{1A} in compound 410 is phenyl substituted with 1 to 3 (e.g., 1 to 3, or 1 to 2, or 1) independently selected halos. In another embodiment of this invention R^{1A} in compound 410 is phenyl substituted with 1 to 3 (e.g., 1 to 3, or 1 to 2, or 1) F atoms. In another embodiment of this invention R^{1A} in compound 41C is phenyl substituted with 1 F atom.

Another embodiment of this invention is directed to compounds of formula (I) having the formula:

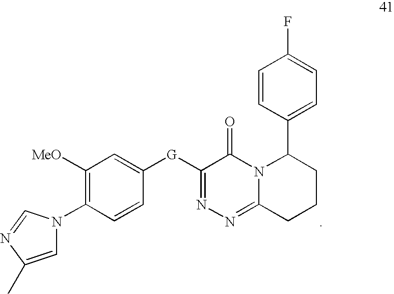

Another embodiment of this invention is directed to compounds of formula (I) having the formula:

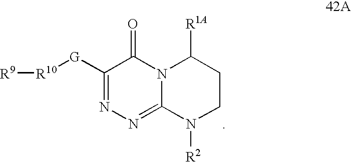

In one embodiment of this invention R^{1A} in compound 42A is aryl or substituted aryl. In another embodiment R^{1A} in compound 42A is phenyl or substituted phenyl. In another embodiment R^{1A} in compound 42A is phenyl substituted with 1 to 3 independently selected R²¹ groups. In another embodiment of this invention R^{1A} in compound 42A is phenyl substituted with 1 to 3 (e.g., 1 to 3, or 1 to 2, or 1) independently selected halos. In another embodiment of this invention R^{1A} in compound 42A is phenyl substituted with 1 to 3 (e.g., 1 to 3, or 1 to 2, or 1) F atoms. In another embodiment of this invention R^{1A} in compound 42A is phenyl substituted with 1 F atom

Another embodiment of this invention is directed to compounds of formula (I) having the formula:

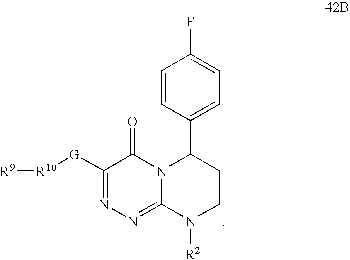

Another embodiment of this invention is directed to compounds of formula (I) having the formula:

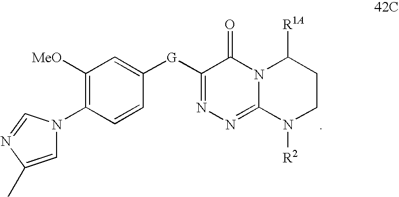

In one embodiment of this invention R^{1A} in compound 42C is aryl or substituted aryl. In another embodiment R^{1A} in compound 42C is phenyl or substituted phenyl. In another embodiment R^{1A} in compound 42C is phenyl substituted with 1 to 3 independently selected R²¹ groups. In another embodiment of this invention R^{1A} in compound 42C is phenyl substituted with 1 to 3 (e.g., 1 to 3, or 1 to 2, or 1) independently selected halos. In another embodiment of this invention R^{1A} in compound 42C is phenyl substituted with 1 to 3 (e.g., 1 to 3, or 1 to 2, or 1) F atoms. In another embodiment of this invention R^{1A} in compound 42C is phenyl substituted with 1 F atom.

Another embodiment of this invention is directed to compounds of formula (I) having the formula:

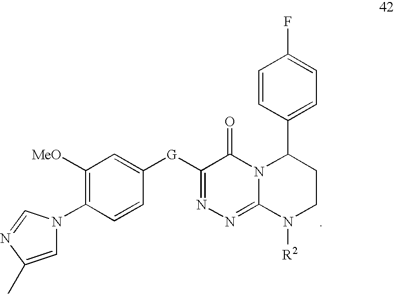

Another embodiment of this invention is directed to compounds of formula (I) having the formula:

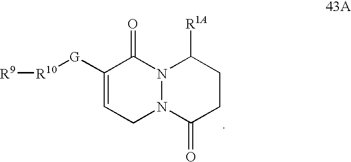

In one embodiment of this invention R^{1A} in compound 43A is aryl or substituted aryl. In another embodiment R^{1A} in compound 43A is phenyl or substituted phenyl. In another embodiment R^{1A} in compound 43A is phenyl substituted with 1 to 3 independently selected R²¹ groups. In another embodiment of this invention R^{1A} in compound 43A is phenyl substituted with 1 to 3 (e.g., 1 to 3, or 1 to 2, or 1) independently selected halos. In another embodiment of this invention R^{1A} in compound 43A is phenyl substituted with 1 to 3 (e.g., 1 to 3, or 1 to 2, or 1) F atoms. In another embodiment of this invention R^{1A} in compound 43A is phenyl substituted with 1 F atom.

Another embodiment of this invention is directed to compounds of formula (I) having the formula:

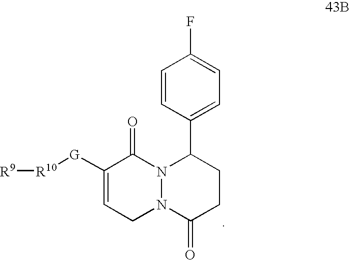

Another embodiment of this invention is directed to compounds of formula (I) having the formula:

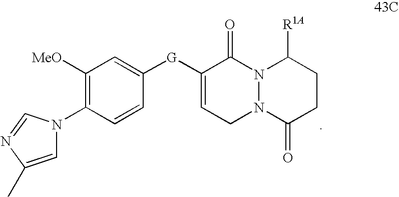

In one embodiment of this invention R^{1A} in compound 43C is aryl or substituted aryl. In another embodiment R^{1A} in compound 43C is phenyl or substituted phenyl. In another embodiment R^{1A} in compound 43C is phenyl substituted with 1 to 3 independently selected R²¹ groups. In another embodiment of this invention R^{1A} in compound 43C is phenyl substituted with 1 to 3 (e.g., 1 to 3, or 1 to 2, or 1) independently selected halos. In another embodiment of this invention R^{1A} in compound 43C is phenyl substituted with 1 to 3 (e.g., 1 to 3, or 1 to 2, or 1) F atoms. In another embodiment of this invention R^{1A} in compound 43C is phenyl substituted with 1 F atom.

Another embodiment of this invention is directed to compounds of formula (I) having the formula:

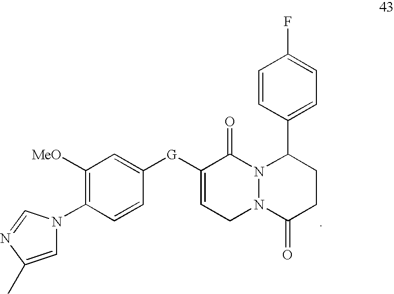

Another embodiment of this invention is directed to compounds of formula (I) having the formula:

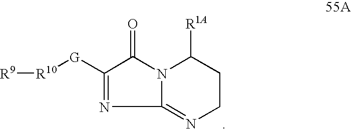

In one embodiment of this invention R^{1A} in compound 55A is aryl or substituted aryl. In another embodiment R^{1A} in compound 55A is phenyl or substituted phenyl. In another embodiment R^{1A} in compound 55A is phenyl substituted with 1 to 3 independently selected R²¹ groups. In another embodiment of this invention R^{1A} in compound 55A is phenyl substituted with 1 to 3 (e.g., 1 to 3, or 1 to 2, or 1) independently selected halos. In another embodiment of this invention R^{1A} in compound 55A is phenyl substituted with 1 to 3 (e.g., 1 to 3, or 1 to 2, or 1) F atoms. In another embodiment of this invention R^{1A} in compound 55A is phenyl substituted with 1 F atom.

Another embodiment of this invention is directed to compounds of formula (I) having the formula:

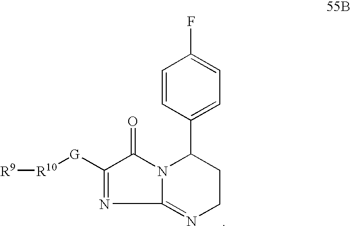

Another embodiment of this invention is directed to compounds of formula (I) having the formula:

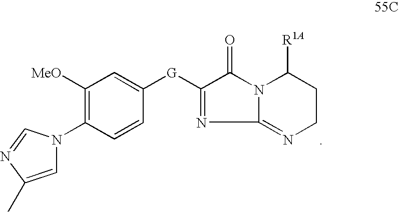

In one embodiment of this invention R^{1A} in compound 55C is aryl or substituted aryl. In another embodiment R^{1A} in compound 55C is phenyl or substituted phenyl. In another embodiment R^{1A} in compound 55C is phenyl substituted with 1 to 3 independently selected R²¹ groups. In another embodiment of this invention R^{1A} in compound 55C is phenyl substituted with 1 to 3 (e.g., 1 to 3, or 1 to 2, or 1) independently selected halos. In another embodiment of this invention R^{1A} in compound 55C is phenyl substituted with 1 to 3 (e.g., 1 to 3, or 1 to 2, or 1) F atoms. In another embodiment of this invention R^{1A} in compound 55C is phenyl substituted with 1 F atom.

Another embodiment of this invention is directed to compounds of formula (I) having the formula:

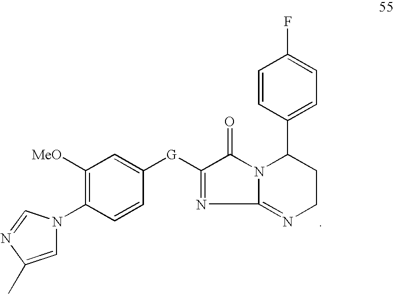

Examples of the R²¹ moiety in the embodiments of this invention include, but are not limited to: (a) —OR¹⁵, (b) —OR¹⁵ wherein R¹⁵ is alkyl, (c) —OR¹⁵ wherein R¹⁵ is alkyl and said alkyl is methyl or ethyl, (d) —OR¹⁵ wherein R¹⁵ is cycloalkylalkyl, (e) —OR¹⁵ wherein R¹⁵ is -alkyl-(R¹⁸)ₙ, (f) —OR¹⁵ wherein R¹⁵ is -alkyl-(R¹⁸), and wherein said R¹⁸ is —OR²⁰, (g) —OR¹⁵ wherein R¹⁵ is -alkyl-(R¹⁸)ₙ and wherein said R¹⁸ is —OR²⁰ and said R²⁰ is alkyl. Examples of the R²¹ moiety include but are not limited to: —OCH₃, —OCH₂CH₃, —O(CH₂)₂OCH₃, and —CH₂-cyclopropyl.

Examples of R²¹ also include —C(O)OR¹⁵ wherein, for example, R¹⁵ is alkyl, such as, for example, methyl).

Examples of R²¹ also include —C(O)NR¹⁵R¹⁶, wherein, for example, one of R¹⁵ or R¹⁶ is H, and the other is selected from the group consisting of: (R¹⁸)ₙ-arylalkyl-, (R¹⁸)ₙ-alkyl-, and cycloalkyl. In one example of this —C(O)NR¹⁵R¹⁶ moiety the R¹⁸ is —OR²⁰, n is 1, R²⁰ is alkyl, said cycloalkyl is cyclobutyl, and said arylalkyl- is benzyl.

Examples of R²¹ also include halo (e.g., Br, Cl or F).

Examples of R²¹ also include arylalkyl, such as, for example, benzyl.

In another embodiment of this invention the compound of formula (I) is a compound selected from the group consisting of: (IA) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, (IC), (ID), (IE), (IF), (IG), and I(H) wherein (a) R^{1A} is a phenyl, or R^{1A} is a phenyl substituted with one, two or three F.

In another embodiment of this invention the compound of formula (I) is a compound selected from the group consisting of: 2A, 3A, 4A, 5A, 6A, 7A, 8A, 9A, 12A, 13A, 14A, 15A, 16A, 17A, 18A, 20A, 21A, 40A, 41A, 42A, 43A, and 55A wherein (a) R^{1A} is a phenyl, or R^{1A} is a phenyl substituted with one, two or three F.

In another embodiment of this invention the compound of formula (I) is a compound selected from the group consisting of: 2C, 3C, 4C, 5C, 6C, 7C, 8C, 9C, 12C, 13C, 14C, 15C, 16C, 17C, 18C, 20C, 21C, 40C, 41C, 42C, 43C, and 55C wherein (a) R^{1A} is a phenyl, or R^{1A} is a phenyl substituted with one, two or three F.

In another embodiment of this invention the compound of formula (I) is a compound selected from the group consisting of: (IA) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, (IC), (ID), (IE), (IF), (IG), and I(H) wherein (a) R^{1A} is a phenyl, or R^{1A} is a phenyl substituted with one, two or three F and (b) R¹⁰ is phenyl substituted with one —OR¹⁵ group, wherein R¹⁵ is methyl, and (c) R⁹ is selected from the group consisting of imidazolyl and imidazolyl substituted with one methyl group.

In another embodiment of this invention the compound of formula (I) is a compound selected from the group consisting of: (IA) wherein the optional bond between G¹ and G² is absent. (IA) wherein the optional bond between G¹ and G² is present, (IB) wherein the optional bond between G¹ and G² is absent, (IB) wherein the optional bond between G¹ and G² is present, (10), (ID), (IE), (IF), (IG), and I(H) wherein (a) R^{1A} is a phenyl substituted with one, two or three F and (b) R¹⁰ is phenyl substituted with one —OR¹⁵ group, wherein R¹⁵ is methyl, and (c) R⁹ is imidazolyl substituted with one methyl group.

In another embodiment of this invention the compound of formula (I) is a compound selected from the group consisting of: 2A, 3A, 4A, 5A, GA, 7A, 8A, 9A, 12A, 13A, 14A, 15A, 16A, 17A, 18A, 20A, 21A, 40A, 41A, 42A, 43A, and 55A wherein (a) R^{1A} is a phenyl, or R^{1A} is a phenyl substituted with one, two or three F and (b) R¹⁰ is phenyl substituted with one —OR¹⁵ group, wherein R¹⁵ is methyl, and (c) R⁹ is selected from the group consisting of imidazolyl and imidazolyl substituted with one methyl group.

In another embodiment of this invention the compound of formula (I) is a compound selected from the group consisting of: 2A, 3A, 4A, 5A, 6A, 7A, 8A, 9A, 12A, 13A, 14A, 15A, 16A, 17A, 18A, 20A, 21A, 40A, 41A, 42A, 43A, and 55A wherein (a) R^{1A} is a phenyl substituted with one, two or three F and (b) R¹⁰ is phenyl substituted with one —OR¹⁵ group, wherein R¹⁵ is methyl, and (c) R⁹ is imidazolyl substituted with one methyl group.

In another embodiment of this invention the compound of formula (I) is a compound selected from the group consisting of: (IA) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, (IC), (ID), (IE), (IF), (IG), I(H), 2A, 3A, 4A, 5A, 6A, 7A, 8A, 9A, 12A, 13A, 14A, 15A, 16A, 17A, 18A, 20A, 21A, 40A, 41A, 42A, 43A, and 55A wherein R^{1A} is selected from the group consisting of:

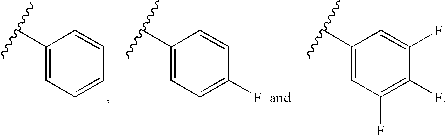

In another embodiment of this invention the compound of formula (I) is a compound selected from the group consisting of: (IA) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, (IC), (ID), (IE), (IF), (IG), I(H), 2A, 3A, 4A, 5A, 6A, 7A, 8A, 9A, 12A, 13A, 14A, 15A, 16A, 17A, 18A, 20A, 21A, 40A, 41A, 42A, 43A, and 55A wherein R^{1A} is selected from the group consisting of:

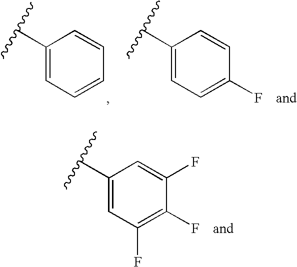

wherein the R⁹-R¹⁰- moiety is:

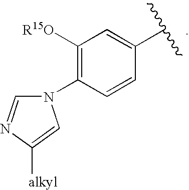

In another embodiment of this invention the compound of formula (I) is a compound selected from the group consisting of: (IA) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, (IC), (ID), (IE), (IF), (IG), I(H), 2A, 3A, 4A, 5A, 6A, 7A, 8A, 9A, 12A, 13A, 14A, 15A, 16A, 17A, 18A, 20A, 21A, 40A, 41A, 42A, 43A, and 55A wherein R^{1A} is selected from the group consisting of:

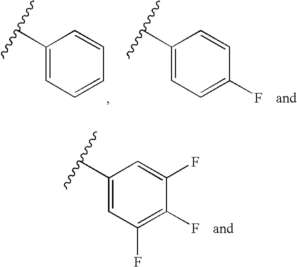

wherein the R⁹-R¹⁰- moiety is:

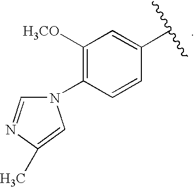

In another embodiment of this invention the compound of formula (I) is a compound selected from the group consisting of: (IA) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, (IC), (ID), (IE), (IF), (IG), I(H), 2A, 3A, 4A, 5A, 6A, 7A, 8A, 9A, 12A, 13A, 14A, 15A, 16A, 17A, 18A, 20A, 21A, 40A, 41A, 42A, 43A, and 55A wherein R^{1A} is selected from the group consisting of:

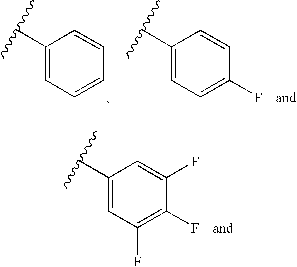

wherein the R⁹-R¹⁰- moiety is:

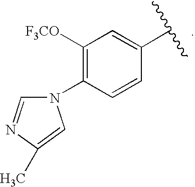

In another embodiment of this invention the compound of formula (I) is a compound selected from the group consisting of: (IA) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, (IC), (ID), (IE), (IF), (IG), I(H), 2A, 3A, 4A, 5A, 6A, 7A, 8A, 9A, 12A, 13A, 14A, 15A, 16A, 17A, 18A, 20A, 21A, 40A, 41A, 42A, 43A, and 55A wherein R^{1A} is selected from the group consisting of:

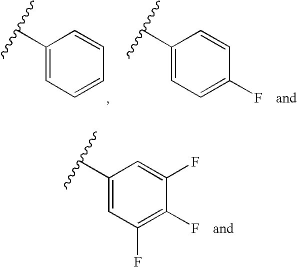

wherein the R⁹-R¹⁰- moiety is:

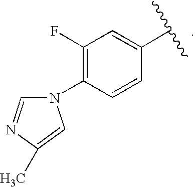

In another embodiment of this invention the compound of formula (I) is a compound selected from the group consisting of: (IA) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, (IC), (ID), (IE), (IF), (IG), I(H), 2A, 3A, 4A, 5A, 6A, 7A, 8A, 9A, 12A, 13A, 14A, 15A, 16A, 17A, 18A, 20A, 21A, 40A, 41A, 42A, 43A, and 55A wherein R^{1A} is selected from the group consisting of:

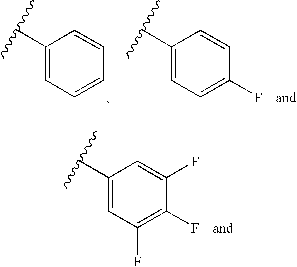

wherein the R⁹-R¹⁰- moiety is:

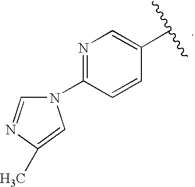

Another embodiment of this invention is directed to compounds of formula (I) selected from the group consisting of: compounds of formulas (IA) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, IC to IH, 2 to 9, 12 to 18, 20, 21, 40 to 43, 55, 2A to 9A, 12A to 18A, 20A, 21A, 40A to 43A, 55A, 2B to 9B, 12B to 18B, 20B, 21B, 40B to 43B, 55B, 2C to 9C, 12C to 18C, 20C, 21C, 40C to 43C, 55C, 6.2, 9.1, 10.1, 10.2, 10.3, 14.1, 16.1, 16.2, 18.1, 19.1, 20.2, 21.2, 23.2, 25.1, 26.1, 27.1, 28.1, 30.1, 36.1, 37.1, 38.1, 39.1, 41.1, 43.1, 45.1, 46.1, 47.1, 48.1, 49.1, 50.1, 51.1, 52.1, 59.1, 60.1, 61.1, 64.1, 65.1, 68.1, 70.1, E1, E2, and E3.

Another embodiment of this invention is directed to compounds of formula (I) selected from the group consisting of: compounds of formulas (IA) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, and IC to IH.

Another embodiment of this invention is directed to compounds of formula (I) selected from the group consisting of: compounds of formulas 2 to 9, 12 to 18, 20, 21, 40 to 43, and 55.

Another embodiment of this invention is directed to compounds of formula (I) selected from the group consisting of: compounds of formulas 2A to 9A, 12A to 18A, 20A, 21A, 40A to 43A, and 55A.

Another embodiment of this invention is directed to compounds of formula (I) selected from the group consisting of: compounds of formulas 2B to 9B, 12B to 18B, 20B, 21B, 40B to 43B, and 55B.

Another embodiment of this invention is directed to compounds of formula (I) selected from the group consisting of: compounds of formulas 2C to 9C, 12C to 18C, 20C, 21C, 40C to 43C, and 55C.

Another embodiment of this invention is directed to compounds of formula (I) selected from the group consisting of: compounds of formulas 6.2, 9.1, 10.1, 10.2, 10.3, 14.1, 16.1, 16.2, 18.1, 19.1, 20.2, 21.2, 23.2, 25.1, 26.1, 27.1, 28.1, 30.1, 36.1, 37.1, 38.1, 39.1, 41.1, 43.1, 45.1, 46.1, 47.1, 48.1, 49.1, 50.1, 51.1, 52.1, 59.1, 60.1, 61.1, 64.1, 65.1, 68.1, and 70.1.

Another embodiment of this invention is directed to compounds of formula (I) selected from the group consisting of: compounds of formulas E1, E2, and E3.

Another embodiment of this invention is directed to compound E1.

Another embodiment of this invention is directed to compound E2.

Another embodiment of this invention is directed to compound E3.

In the embodiments below Groups A, B, C, D, E, F, G and H are as defined as follows:
(1) Group A: compounds (IA) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, IC to IH, 2 to 9, 12 to 18, 20, 21, 40 to 43, 55, 2A to 9A, 12A to 18A, 20A, 21A, 40A to 43A, 55A, 2B to 9B, 12B to 18B, 20B, 21B, 40B to 43B, 55B, 2C to 9C, 12C to 18C, 20C, 21C, 40C to 43C, 55C, 6.2, 9.1, 10.1, 10.2, 10.3, 14.1, 16.1, 16.2, 18.1, 19.1, 20.2, 21.2, 23.2, 25.1, 26.1, 27.1, 28.1, 30.1, 36.1, 37.1, 38.1, 39.1, 41.1, 43.1, 45.1, 46.1, 47.1, 48.1, 49.1, 50.1, 51.1, 52.1, 59.1, 60.1, 61.1, 64.1, 65.1, 68.1, 70.1, E1, E2, and E3;
(2) Group B: compounds (1A) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, and IC to IH;
(3) Group C: compounds 2 to 9, 12 to 18, 20, 21, 40 to 43, and 55;
(4) Group D: compounds 2A to 9A, 12A to 18A, 20A, 21A, 40A to 43A, and 55A;
(5) Group E: compounds 2B to 9B, 12B to 18B, 20B, 21B, 40B to 43B, and 55B;
(6) Group F: compounds 20 to 9C, 12C to 18C, 20C, 21C, 40C to 43C, and 55C;
(7) Group G: compounds 6.2, 9.1, 10.1, 10.2, 10.3, 14.1, 16.1, 16.2, 18.1, 19.1, 20.2, 21.2, 23.2, 25.1, 26.1, 27.1, 28.1, 30.1, 36.1, 37.1, 38.1, 39.1, 41.1, 43.1, 45.1, 46.1, 47.1, 48.1, 49.1, 50.1, 51.1, 52.1, 59.1, 60.1, 61.1, 64.1, 65.1, 68.1, and 70.1; and
(8) Group H: compounds E1, E2, and E3.

Another embodiment of this invention is directed to a compound of formula (I).

Another embodiment of this invention is directed to a pharmaceutically acceptable salt of a compound of formula (I). And in one example the salt is a salt of a compound selected from the group consisting of Group A. And in another example the salt is a salt of a compound selected from the group consisting of Group 8. And in another example the salt is a salt of a compound selected from the group consisting of Group C. And in another example the salt is a salt of a compound selected from the group consisting of Group D. And in another example the salt is a salt of a compound selected from the group consisting of Group E. And in another example the salt is a salt of a compound selected from the group consisting of Group F. And in another example the salt is a salt of a compound selected from the group consisting of Group G. And in another example the salt is a salt of a compound selected from the group consisting of Group H.

Another embodiment of this invention is directed to a pharmaceutically acceptable ester of a compound of formula (I). And in one example the ester is an ester of a compound selected from the group consisting of Group A. And in another example the ester is an ester of a compound selected from the group consisting of Group B. And in another example the ester is an ester of a compound selected from the group consisting of Group C. And in another example the ester is an ester of a compound selected from the group consisting of Group D. And in another example the ester is an ester of a compound selected from the group consisting of Group E. And in another example the ester is an ester of a compound selected from the group consisting of Group F. And in another example the ester is an ester of a compound selected from the group consisting of Group G. And in another example the ester is an ester of a compound selected from the group consisting of Group H.

Another embodiment of this invention is directed to a solvate of a compound of formula (I). And in one example the solvate is a solvate of a compound selected from the group consisting of Group A, And in another example the solvate is a solvate of a compound selected from the group consisting of Group B. And in another example the solvate is a solvate of a compound selected from the group consisting of Group C. And in another example the solvate is a solvate of a compound selected from the group consisting of Group D. And in another example the solvate is a solvate of a compound selected from the group consisting of Group E. And in another example the solvate is a solvate of a compound selected from the group consisting of Group F. And in another example the solvate is a solvate of a compound selected from the group consisting of Group G. And in another example the solvate is a solvate of a compound selected from the group consisting of Group H.

Another embodiment of this invention is directed to a compound of formula (I) in isolated form. And in one example the compound of formula (I) is selected from the group consisting of Group A. And in one example the compound of formula (I) is selected from the group consisting of Group D. And in one example the compound of formula (I) is selected from the group consisting of Group E. And in one example the compound of formula (I) is selected from the group consisting of Group F. And in one example the compound of formula (I) is selected from the group consisting of Group G. And in one example the compound of formula (I) is selected from the group consisting of Group H.

Another embodiment of this invention is directed to a compound of formula (I) in pure form. And in one example the compound of formula (I) is selected from the group consisting of Group A. And in one example the compound of formula (I) is selected from the group consisting of Group D. And in one example the compound of formula (I) is selected from the group consisting of Group E. And in one example the compound of formula (I) is selected from the group consisting of Group F. And in one example the compound of formula (I) is selected from the group consisting of Group G. And in one example the compound of formula (I) is selected from the group consisting of Group H.

Another embodiment of this invention is directed to a compound of formula (I) in pure and isolated form, And in one example the compound of formula (I) is selected from the group consisting of Group A. And in one example the compound of formula (I) is selected from the group consisting of Group D. And in one example the compound of formula (I) is selected from the group consisting of Group E. And in one example the compound of formula (I) is selected from the group consisting of Group F. And in one example the compound of formula (I) is selected from the group consisting of Group G. And in one example the compound of formula (I) is selected from the group consisting of Group H.

Another embodiment of this invention is directed to a pharmaceutical composition comprising an effective amount of one or more (e.g., one) compounds of Formula (I), or a pharmaceutically acceptable salt, solvate, or ester thereof, and one or more (e.g., one) pharmaceutically acceptable carriers.

Another embodiment is directed to a pharmaceutical composition comprising an effective amount of one or more (e.g., one) compounds of formula (I) and a pharmaceutically acceptable carrier.

Another embodiment is directed to a pharmaceutical composition comprising an effective amount of a pharmaceutically acceptable salt of one or more (e.g., one) compounds of formula (I) and a pharmaceutically acceptable carrier.

Another embodiment is directed to a pharmaceutical composition comprising an effective amount of a pharmaceutically acceptable ester of one or more (e.g., one) compounds of formula (I) and a pharmaceutically acceptable carrier.

Another embodiment is directed to a pharmaceutical composition comprising an effective amount of a solvate of one or more (e.g., one) compounds of formula (I) and a pharmaceutically acceptable carrier.

Another embodiment of this invention is directed to a pharmaceutical composition comprising an effective amount of one or more (e.g., one) compounds of formula (I), and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising a therapeutically effective amount of one or more (e.g. one) compounds of Formula (I), or a pharmaceutically acceptable salt, solvate, or ester thereof, and one or more (e.g., one) pharmaceutically acceptable carriers, and an effective amount of one or more compounds selected from the group consisting of cholinesterase inhibitors, Aβ antibody inhibitors, gamma secretase inhibitors and beta secretase inhibitors.

Another embodiment of this invention is directed to a pharmaceutical composition comprising an effective amount of one or more (e.g., one) compounds of formula (I), and effective amount of one or more BACE inhibitors, and a pharmaceutically acceptable carrier.

Another embodiment of this invention is directed to a pharmaceutical composition comprising an effective amount of one or more (e.g., one) compounds of formula (I), and effective amount of one or more cholinesterase inhibitors (e.g., acetyl- and/or butyrylcholinesterase inhibitors), and a pharmaceutically acceptable carrier.

Another embodiment of this invention is directed to a pharmaceutical composition comprising an effective amount of one or more (e.g., one) compounds of formula (I).

Another embodiment of this invention is directed to a pharmaceutical composition comprising an effective amount of one or more (e.g., one) compounds of formula (I), and effective amount of one or more muscarinic antagonists (e.g., m₁ agonist or m₂ antagonists), and a pharmaceutically acceptable carrier.

Another embodiment of this invention is directed to a pharmaceutical composition comprising an effective amount of one or more (e.g., one) compounds of formula (I), and effective amount of Exelon (rivastigmine), and a pharmaceutically acceptable carrier.

Another embodiment of this invention is directed to a pharmaceutical composition comprising an effective amount of one or more (e.g., one) compounds of formula (I), and effective amount of Cognex (tacrine), and a pharmaceutically acceptable carrier.

Another embodiment of this invention is directed to a pharmaceutical composition comprising an effective amount of one or more (e.g., one) compounds of formula (I), and effective amount of a Tau kinase inhibitor, and a pharmaceutically acceptable carrier.

Another embodiment of this invention is directed to a pharmaceutical composition comprising an effective amount of one or more (e.g., one) compounds of formula (I), and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds of formula (I), and effective amount of one anti-Abeta vaccine (active immunization), and a pharmaceutically acceptable carrier.

Another embodiment of this invention is directed to a pharmaceutical composition comprising an effective amount of one or more (e.g., one) compounds of formula (I), and effective amount of one or more APP ligands, and a pharmaceutically acceptable carrier.

Another embodiment of this invention is directed to a pharmaceutical composition comprising an effective amount of one or more (e.g., one) compounds of formula (I), and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds of formula (I), and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds of formula (I), and effective amount of one or more fibrates (for example, clofibrate, Clofibride, Etofibrate, Aluminium Clofibrate), and a pharmaceutically acceptable carrier

Another embodiment of this invention is directed to a pharmaceutical composition comprising an effective amount of one or more (e.g., one) compounds of formula (I), and effective amount of one or more LXR agonists, and a pharmaceutically acceptable carrier.

Another embodiment of this invention is directed to a pharmaceutical composition comprising an effective amount of one or more (e.g., one) compounds of formula (I), and effective amount of one or more LRP mimics, and a pharmaceutically acceptable carrier.

Another embodiment of this invention is directed to a pharmaceutical composition comprising an effective amount of one or more (e.g., one) compounds of formula (I), and effective amount of one or more 5-HT6 receptor antagonists, and a pharmaceutically acceptable carrier.

Another embodiment of this invention is directed to a pharmaceutical composition comprising an effective amount of one or more (e.g., one) compounds of formula (I), and effective amount of one or more nicotinic receptor agonists, and a pharmaceutically acceptable carrier.

Another embodiment of this invention is directed to a pharmaceutical composition comprising an effective amount of one or more (e.g., one) compounds of formula (I), and effective amount of one or more H3 receptor antagonists, and a pharmaceutically acceptable carrier.

Another embodiment of this invention is directed to a pharmaceutical composition comprising an effective amount of one or more (e.g., one) compounds of formula (I), and effective amount of one or more histone deacetylase inhibitors, and a pharmaceutically acceptable carrier.

Another embodiment of this invention is directed to a pharmaceutical composition comprising an effective amount of one or more (e.g., one) compounds of formula (I), and effective amount of one or more hsp90 inhibitors, and a pharmaceutically acceptable carrier.

Another embodiment of this invention is directed to a pharmaceutical composition comprising an effective amount of one or more (e.g., one) compounds of formula (I), and effective amount of one or more m1 muscarinic receptor agonists, and a pharmaceutically acceptable carrier.

Another embodiment of this invention is directed to combinations, i.e., a pharmaceutical composition, comprising a pharmaceutically acceptable carrier, an effective (i.e., therapeutically effective) amount of one or more compounds of formula (I), in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors.

Another embodiment of this invention is directed to a pharmaceutical composition comprising an effective amount of one or more (e.g., one) compounds of formula (I), and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds of formula (I), and effective amount of one or more one mGluR2/3 antagonists, and a pharmaceutically acceptable carrier.

Another embodiment of this invention is directed to a pharmaceutical composition comprising an effective amount of one or more (e.g., one) compounds of formula (I), and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds of formula (I), and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds of formula (I), and effective amount of one or more PAI-1 inhibitors, and a pharmaceutically acceptable carrier.

Another embodiment of this invention is directed to a pharmaceutical composition comprising an effective amount of one or more (e.g., one) compounds of formula (I), and effective amount of one or more agents that can induce Abeta efflux such as gelsolin, and a pharmaceutically acceptable carrier.

Other embodiments of this invention are directed to any one of the above embodiments directed to pharmaceutical compositions wherein the compound of formula (I) is selected from the group consisting of Group A.

Other embodiments of this invention are directed to any one of the above embodiments directed to pharmaceutical compositions wherein the compound of formula (I) is selected from the group consisting of Group B.

Other embodiments of this invention are directed to any one of the above embodiments directed to pharmaceutical compositions wherein the compound of formula (I) is selected from the group consisting of Group C.

Other embodiments of this invention are directed to any one of the above embodiments directed to pharmaceutical compositions wherein the compound of formula (I) is selected from the group consisting of Group D.

Other embodiments of this invention are directed to any one of the above embodiments directed to pharmaceutical compositions wherein the compound of formula (I) is selected from the group consisting of Group E.

Other embodiments of this invention are directed to any one of the above embodiments directed to pharmaceutical compositions wherein the compound of formula (I) is selected from the group consisting of Group F.

Other embodiments of this invention are directed to any one of the above embodiments directed to pharmaceutical compositions wherein the compound of formula (I) is selected from the group consisting of Group G.

Other embodiments of this invention are directed to any one of the above embodiments directed to pharmaceutical compositions wherein the compound of formula (I) is selected from the group consisting of Group H.

The compounds of formula (I) can be useful as gamma secretase modulators and can be useful in the treatment and prevention of diseases such as, for example, central nervous system disorders (such as Alzheimers disease and Downs Syndrome), mild cognitive impairment, glaucoma, cerebral amyloid angiopathy, stroke, dementia, microgliosis, brain inflammation, and olfactory function loss. Another embodiment of this invention is directed to a method of treating a central nervous system disorder comprising administering a therapeutically effective amount of at least one compound of formula (I) to a patient in need of such treatment.

Another embodiment of this invention is directed to a method of treating a central nervous system disorder comprising administering a therapeutically effective amount of a pharmaceutical composition comprising a therapeutically effective amount of at least one compound of formula (I), or a pharmaceutically acceptable salt, solvate, or ester thereof, and at least one pharmaceutically acceptable carrier.

Another embodiment of this invention is directed to a method of treating a central nervous system disorder comprising administering a therapeutically effective amount of a pharmaceutical composition comprising a therapeutically effective amount of at least one compound of formula (I), or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

Thus, another embodiment of this invention is directed to a method for modulating (including inhibiting, antagonizing and the like) gamma-secretase comprising administering an effective (i.e., therapeutically effective) amount of one or more (e.g., one) compounds of formula (I) to a patient in need of such treatment.

Another embodiment of this invention is directed to a method for modulating (including inhibiting, antagonizing and the like) gamma-secretase, comprising administering an effective (i.e., therapeutically effective) amount of a compound of formula (I) to a patient in need of treatment.

Another embodiment of this invention is directed to a method of treating one or more neurodegenerative diseases, comprising administering an effective (i.e., therapeutically effective) amount of one or more (e.g., one) compounds of formula (I) to a patient in need of treatment.

Another embodiment of this invention is directed to a method of treating one or more neurodegenerative diseases, comprising administering an effective (i.e., therapeutically effective) amount of a compound of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of one or more (e.g., one) compounds of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of a compound of formula (I) to a patient in need of treatment.

Another embodiment of this invention is directed to a method of treating Alzheimer's disease, comprising administering an effective (i.e., therapeutically effective) amount of one or more (e.g., one) compounds of formula (I) to a patient in need of treatment.

Another embodiment of this invention is directed to a method of treating Alzheimer's disease, comprising administering an effective (i.e., therapeutically effective) amount of a compound of formula (I) to a patient in need of treatment.

Another embodiment of this invention is directed to a method of treating Alzheimer's disease, comprising administering an effective (i.e., therapeutically effective) amount of a compound of formula (I) to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of one or more (e.g., one) compounds of formula (I) to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of a compound of formula (I) to a patient in need of treatment.

Another embodiment of this invention is directed to a method of treating mild cognitive impairment, comprising administering an effective amount of one or more (e.g., one) compounds of formula (I) to a patient in need of treatment.

Another embodiment of this invention is directed to a method of treating glaucoma, comprising administering an effective amount of one or more (e.g., one) compounds of formula (I) to a patient in need of treatment.

Another embodiment of this invention is directed to a method of treating cerebral amyloid angiopathy, comprising administering an effective amount of one or more (e.g., one) compounds of formula (I) to a patient in need of treatment.

Another embodiment of this invention is directed to a method of treating stroke, comprising administering an effective amount of one or more (e.g., one) compounds of formula (I) to a patient in need of treatment.

Another embodiment of this invention is directed to a method of treating dementia, comprising administering an effective amount of one or more (e.g., one) compounds of formula (I) to a patient in need of treatment.

Another embodiment of this invention is directed to a method of treating microgliosis, comprising administering an effective amount of one or more (e.g., one) compounds of formula (I) to a patient in need of treatment.

Another embodiment of this invention is directed to a method of treating brain inflammation, comprising administering an effective amount of one or more (e.g., one) compounds of formula (I) to a patient in need of treatment.

Another embodiment of this invention is directed to a method of treating olfactory function loss, comprising administering an effective amount of one or more (e.g., one) compounds of formula (I) to a patient in need of treatment.

Another embodiment of this invention is directed to a method of treating Downs syndrome, comprising administering an effective amount of one or more (e.g., one) compounds of formula (I) to a patient in need of treatment.

Another embodiment of this invention is directed to a method of treating Downs syndrome, comprising administering an effective amount of a compound of formula (I) to a patient in need of treatment.

Other embodiments of this invention are directed to any one of the above embodiments directed to methods of treating wherein the compound of formula (I) is selected from the group consisting of Group A.

Other embodiments of this invention are directed to any one of the above embodiments directed to methods of treating wherein the compound of formula (I) is selected from the group consisting of Group B.

Other embodiments of this invention are directed to any one of the above embodiments directed to methods of treating wherein the compound of formula (I) is selected from the group consisting of Group C.

Other embodiments of this invention are directed to any one of the above embodiments directed to methods of treating wherein the compound of formula (I) is selected from the group consisting of Group D.

Other embodiments of this invention are directed to any one of the above embodiments directed to methods of treating wherein the compound of formula (I) is selected from the group consisting of Group E.

Other embodiments of this invention are directed to any one of the above embodiments directed to methods of treating wherein the compound of formula (I) is selected from the group consisting of Group F.

Other embodiments of this invention are directed to any one of the above embodiments directed to methods of treating wherein the compound of formula (t) is selected from the group consisting of Group G.

Other embodiments of this invention are directed to any one of the above embodiments directed to methods of treating wherein the compound of formula (I) is selected from the group consisting of Group H.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I) and the administration of one or more (e.g., one) other pharmaceutical active ingredients (e.g., drugs). The compounds of formula (I) and the other drugs can be administered separately (i.e., each is in its own separate dosage form), or the compounds of formula (I) can be combined with the other drugs in the same dosage form.

Thus, other embodiments of this invention are directed to any one of the methods of treatment, or methods of inhibiting, described herein, wherein an effective amount of the compound of formula (I) is used in combination with an effective amount of one or more other pharmaceutically active ingredients (e.g., drugs). The other pharmaceutically active ingredients (i.e., drugs) are selected from the group consisting of: BACE inhibitors (beta secretase inhibitors), muscarinic antagonists (e.g., m₁ agonists or m₂ antagonists), cholinesterase inhibitors (e.g., acetyl- and/or butyrylcholinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Other embodiments of this invention are directed to any one of the methods of treatment, or methods of inhibiting, described herein, wherein the compound of formula (I) is used in combination with an effective amount of one or more other pharmaceutically active ingredients selected from the group consisting of: BACE inhibitors (beta secretase inhibitors), muscarinic antagonists (e.g., m₁ agonist or m₂ antagonists), cholinesterase inhibitors (e.g., acetyl- and/or butyrylcholinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors (e.g., ezetimibe).

Another embodiment of this invention is directed to a method of treating Alzheimer's disease, comprising administering an effective (i.e., therapeutically effective) amount of one or more (e.g., one) compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of a compound of formula (I), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of one or more (e.g., one) compounds of formula (I), in combination with an effective (i.e., therapeutically effective) amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective (i.e., therapeutically effective) amount of one or more (e.g., one) compounds of formula (I), in combination with an effective (i.e., therapeutically effective) amount of one or more BACE inhibitors.

Another embodiment of this invention is directed to a method of treating Alzheimer's disease, comprising administering an effective amount of one or more compounds of formula (I), in combination with an effective amount of Exelon (rivastigmine).

Another embodiment of this invention is directed to a method of treating Alzheimer's disease, comprising administering an effective amount of one or more compounds of formula (I), in combination with an effective amount of Cognex (twine).

Another embodiment of this invention is directed to a method of treating Alzheimer's disease, comprising administering an effective amount of one or more compounds of formula (I), in combination with an effective amount of a Tau kinase inhibitor.

Another embodiment of this invention is directed to a method of treating Alzheimer's disease, comprising administering an effective amount of one or more compounds of formula (I), in combination with an effective amount of one or more Tau kinase inhibitor (e.g., GSK3beta inhibitor, cdk5 inhibitor, ERK inhibitor).

This invention also provides a method of treating Alzheimer's disease, comprising administering an effective amount of one or more compounds of formula (I), in combination with an effective amount of one anti-Abeta vaccination (active immunization).

Another embodiment of this invention is directed to a method of treating Alzheimer's disease, comprising administering an effective amount of one or more compounds of formula (I), in combination with an effective amount of one or more APP ligands.

Another embodiment of this invention is directed to a method of treating Alzheimer's disease, comprising administering an effective amount of one or more compounds of formula (I), in combination with an effective amount of one or more agents that upregulate insulin degrading enzyme and/or neprilysin.

Another embodiment of this invention is directed to a method of treating Alzheimer's disease, comprising administering an effective amount of one or more compounds of formula (I), in combination with an effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe).

This invention also provides a method of treating Alzheimer's disease, comprising administering an effective amount of one or more compounds of formula (I), in combination with an effective amount of one or more fibrates (for example, clofibrate, Clofibride, Etofibrate, Aluminium Clofibrate).

Another embodiment of this invention is directed to a method of treating Alzheimer's disease, comprising administering an effective amount of one or more compounds of formula (I), in combination with an effective amount of one or more LXR agonists.

Another embodiment of this invention is directed to a method of treating Alzheimer's disease, comprising administering an effective amount of one or more compounds of formula (I), in combination with an effective amount of one or more LRP mimics.

Another embodiment of this invention is directed to a method of treating Alzheimer's disease, comprising administering an effective amount of one or more compounds of formula (I), in combination with an effective amount of one or more 5-HT6 receptor antagonists.

Another embodiment of this invention is directed to a method of treating Alzheimer's disease, comprising administering an effective amount of one or more compounds of formula (I), in combination with an effective amount of one or more nicotinic receptor agonists.

Another embodiment of this invention is directed to a method of treating Alzheimer's disease, comprising administering an effective amount of one or more compounds of formula (I), in combination with an effective amount of one or more H3 receptor antagonists.

This invention also provides a method of treating Alzheimer's disease, comprising administering an effective amount of one or more compounds of formula (I), in combination with an effective amount of one or more histone deacetylase inhibitors.

Another embodiment of this invention is directed to a method of treating Alzheimer's disease, comprising administering an effective amount of one or more compounds of formula (I), in combination with an effective amount of one or more hsp90 inhibitors.

Another embodiment of this invention is directed to a method of treating Alzheimer's disease, comprising administering an effective amount of one or more compounds of formula (I), in combination with an effective amount of one or more m1 muscarinic receptor agonists.

Another embodiment of this invention is directed to a method of treating Alzheimer's disease, comprising administering an effective amount of one or more compounds of formula (I), in combination with an effective amount of one or more 5-HT6 receptor antagonists mGluR1 or mGluR5 positive allosteric modulators or agonists.

Another embodiment of this invention is directed to a method of treating Alzheimer's disease, comprising administering an effective amount of one or more compounds of formula (I), in combination with an effective amount of one or more mGluR2/3 antagonists.

Another embodiment of this invention is directed to a method of treating Alzheimer's disease, comprising administering an effective amount of one or more compounds of formula (I), in combination with an effective amount of one or more anti-inflammatory agents that can reduce neuroinflammation.

Another embodiment of this invention is directed to a method of treating Alzheimer's disease, comprising administering an effective amount of one or more compounds of formula (I), in combination with an effective amount of one or more Prostaglandin EP2 receptor antagonists.

Another embodiment of this invention is directed to a method of treating Alzheimer's disease, comprising administering an effective amount of one or more compounds of formula (I), in combination with an effective amount of one or more PAI-1 inhibitors.

Another embodiment of this invention is directed to a method of treating Alzheimer's disease, comprising administering an effective amount of one or more compounds of formula (I), in combination with an effective amount of one or more agents that can induce Abeta efflux such as gelsolin.

Another embodiment of this invention is directed to a method of treating Downs syndrome, comprising administering an effective (i.e., therapeutically effective) amount of one or more (e.g., one) compounds of formula (I) to a patient in need of treatment.

Another embodiment of this invention is directed to a method of treating Downs syndrome, comprising administering an effective (i.e., therapeutically effective) amount of a compound of formula (I) to a patient in need of treatment.

Another embodiment of this invention is directed to a method of treating Downs syndrome, comprising administering an effective (i.e., therapeutically effective) amount of one or more (e.g., one) compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Downs syndrome, comprising administering an effective (i.e., therapeutically effective) amount of a compound of formula (I), in combination with an effective (i.e., therapeutically effective) amount of one or more (e.g., one) cholinesterase inhibitors (such as, for example, (±)-2,3-dihydro-5,8-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to combinations (i.e., pharmaceutical compositions) comprising an effective (i.e., therapeutically effective) amount of one or more (e.g., one) compounds of formula (I), in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors. The pharmaceutical compositions also comprise a pharmaceutically acceptable carrier.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group A.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group B.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group C.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group D.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group E.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group F.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group G.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group H.

Another embodiment of this invention is directed to a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase, or (e) mild cognitive impairment, or (f) glaucoma, or (g) cerebral amyloid angiopathy, or (h) stroke, or (i) dementia, or (j) microgliosis, or (k) brain inflammation, or (l) olfactory function loss.

Another embodiment of this invention is directed to a kit comprising, in separate containers, in a single package, pharmaceutical compositions for use in combination, wherein one container comprises an effective amount of one or more (e.g., one) compounds of formula (I) in a pharmaceutically acceptable carrier, and another container (i.e., a second container) comprises an effective amount of another pharmaceutically active ingredient (as described above), the combined quantities of the compounds of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Another embodiment of this invention is directed to a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Other embodiments of this invention are directed to any one of the above embodiments directed to kits wherein the compound of formula (I) is selected from the group consisting of Group A.

Other embodiments of this invention are directed to any one of the above embodiments directed to kits wherein the compound of formula (I) is selected from the group consisting of Group B.

Other embodiments of this invention are directed to any one of the above embodiments directed to kits wherein the compound of formula (I) is selected from the group consisting of Group C.

Other embodiments of this invention are directed to any one of the above embodiments directed to kits wherein the compound of formula (I) is selected from the group consisting of Group D.

Other embodiments of this invention are directed to any one of the above embodiments directed to kits wherein the compound of formula (I) is selected from the group consisting of Group E.

Other embodiments of this invention are directed to any one of the above embodiments directed to kits wherein the compound of formula (I) is selected from the group consisting of Group F.

Other embodiments of this invention are directed to any one of the above embodiments directed to kits wherein the compound of formula (I) is selected from the group consisting of Group G.

Other embodiments of this invention are directed to any one of the above embodiments directed to kits wherein the compound of formula (I) is selected from the group consisting of Group H.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ agonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published Feb. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 2, 2006 (see also WO2006/014762 published Feb. 9, 2006), WO2006/014944 published Feb. 2, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May 3, 2007 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. application Ser. No. 60/874,362 filed Dec. 12, 2006, and U.S. application Ser. No. 60/874,419 filed Dec. 12, 2006, the disclosures of each being incorporated incorporated herein by reference thereto.

It is noted that the carbons of formula (I) and other formulas herein may be replaced with 1 to 3 silicon atoms so long as all valency requirements are satisfied.

As used above, and throughout this disclosure, the following terms, unless otherwise indicated, shall be understood to have the following meanings:

“Patient” includes both human and animals.

“Mammal” means humans and other mammalian animals.

“One or more” means that there is at least one and there can be more than one, and examples include 1, 2 or 3, or 1 and 2, or 1.

“At least one” means there is at least one and there can be more than one, and examples include 1, 2 or 3, or 1 and 2, or 1.

“BINAP” means 2,2′-bis(diphenylphosphino)-1,1′-binaphthyl.

“Bn” means benzyl.

“DCM” means dichloromethane.

“DIEA” means N,N,-diisopropylethylamine.

“EDC” means 1-(3-dimethylaminopropyl)-3-ethylcarbodiimide.

“Et” means ethyl.

“HOBT” means 1-hydroxybenzotriazole.

“i-pr” means isopropyl.

“Me” means methyl.

“NBS” means N-bromosuccinimide.

“NMP” means 1-methyl-2-pyrrolidinone.

“OTMS” means trimethylsilyloxy.

“PEG” means polyethylene glycol

“Pr” means propyl.

“t-Bu” means tert-butyl.

“TMSOTf” means trimethylsilyl trifluoromethanesulfonate,

“Fused benzocycloalkyl ring” means a phenyl ring fused to a cycloalkyl ring (as cycloalkyl is defined below), such as, for example,

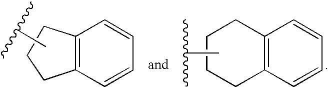

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., OH), —NH(alkyl), —NH(cycloalkyl), —N(alkyl)₂, —O—C(O)-alkyl, —O—C(O)-aryl, —O—C(O)— cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core, Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine. “Halo” refers to fluoro, chloro, bromo or iodo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, ═O, ═N—OY₁, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

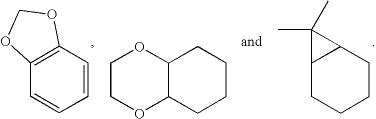

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” or “heterocycloalkyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes rings wherein ═O replaces two available hydrogens on the same carbon atom on a ring system (i.e., heterocyclyl includes rings having a carbonyl in the ring). An example of such moiety is pyrrolidone:

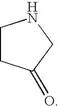

“Heterocyclylalkyl” (or “heterocycloalkylalkyl”) means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” (or “heterocycloalkenyl”) means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydroxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes rings wherein ═O replaces two available hydrogens on the same carbon atom on a ring system (i.e., heterocyclyl includes rings having a carbonyl in the ring). An example of such moiety is pyrrolidinone:

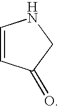

“Heterocyclenylalkyl” (or “heterocycloalkenylalkyl”) means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

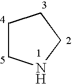

there is no —OH attached directly to carbons marked 2 and 5.

It should also be noted that tautomeric forms such as, for example, the

moieties:

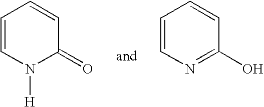

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl, Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group, Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound' or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g., from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((Cᵣ C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al., AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge at al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, butyl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of Formula (I), and salts, solvates, esters and prodrugs thereof, may exist in their tautomeric form (for example, as an amide, enol, keto or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula (I), and of the salts, solvates, esters and prodrugs of the compounds of Formula (I), are intended to be included in the present invention.

The compounds according to the invention can have pharmacological properties; in particular, the compounds of Formula (I) can be modulators of gamma secretase (including inhibitors, antagonists and the like).

More specifically, the compounds of Formula (I) can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Formula (I), or a pharmaceutically acceptable salt, solvate, ester or prodrug of said compound to the mammal.

A preferred dosage is about 0.001 to 500 mg/kg of body weight/day of the compound of Formula (I). An especially preferred dosage is about 0.01 to 25 mg/kg of body weight/day of a compound of Formula I, or a pharmaceutically acceptable salt or solvate of said compound.

The compounds of this invention may also be useful in combination (administered together or sequentially) with one or more additional agents listed above.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Formula (I), or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Formula I, or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula (I), or a pharmaceutically acceptable salt, solvate, ester or prodrug of said compound and a pharmaceutically acceptable carrier, vehicle or diluent.

Yet another aspect of this invention is a kit comprising an amount of at least one compound of Formula (I), or a pharmaceutically acceptable salt, solvate, ester or prodrug of said compound and an amount of at least one additional agent listed above, wherein the amounts of the two or more ingredients result in desired therapeutic effect.

The invention disclosed herein is exemplified by the following illustrative schemes and examples which should not be construed to limit the scope of the disclosure. Alternative mechanistic pathways and analogous structures will be apparent to those skilled in the art.

The compounds of the invention can be prepared by the schemes and examples below. Compounds of the invention wherein the G moiety is bound to G³ (i.e., position (2)) can be prepared by the same chemistry unless indicated otherwise.

In the reactions below R¹ represents:

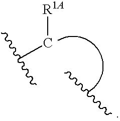

In the reactions below Ring B, formed by the dashed line between R¹ and G¹, is present (i.e., the dashed line between R¹ and G¹ shows the presence of Ring B).

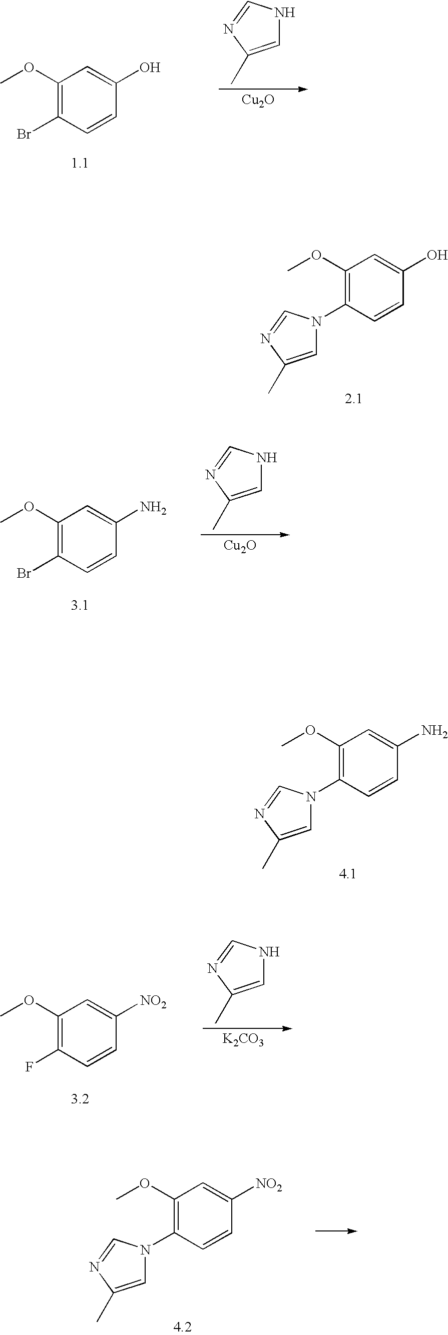

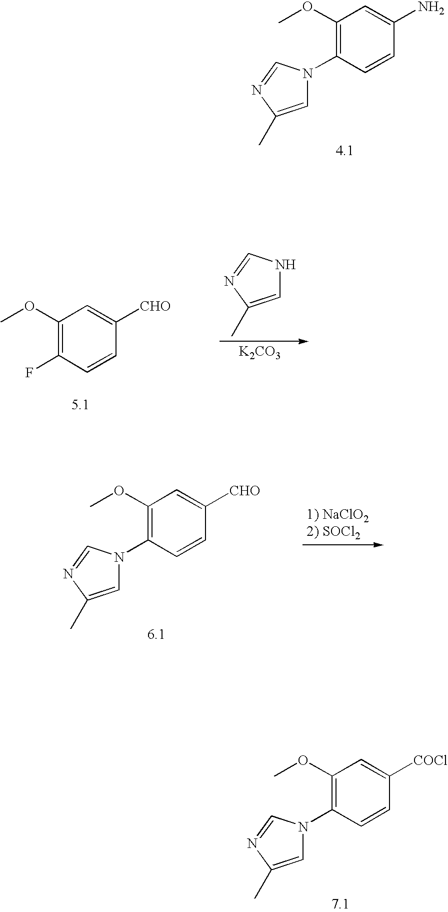

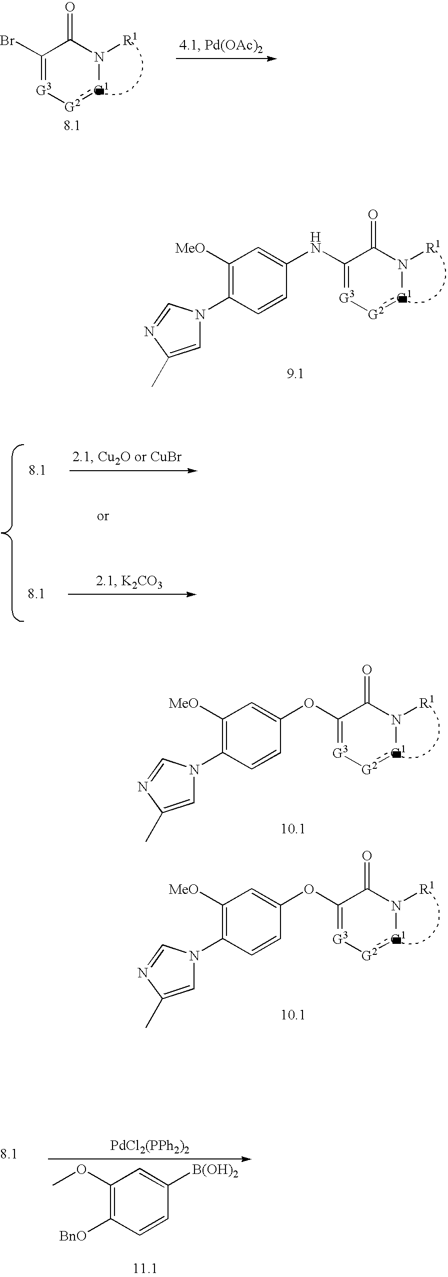

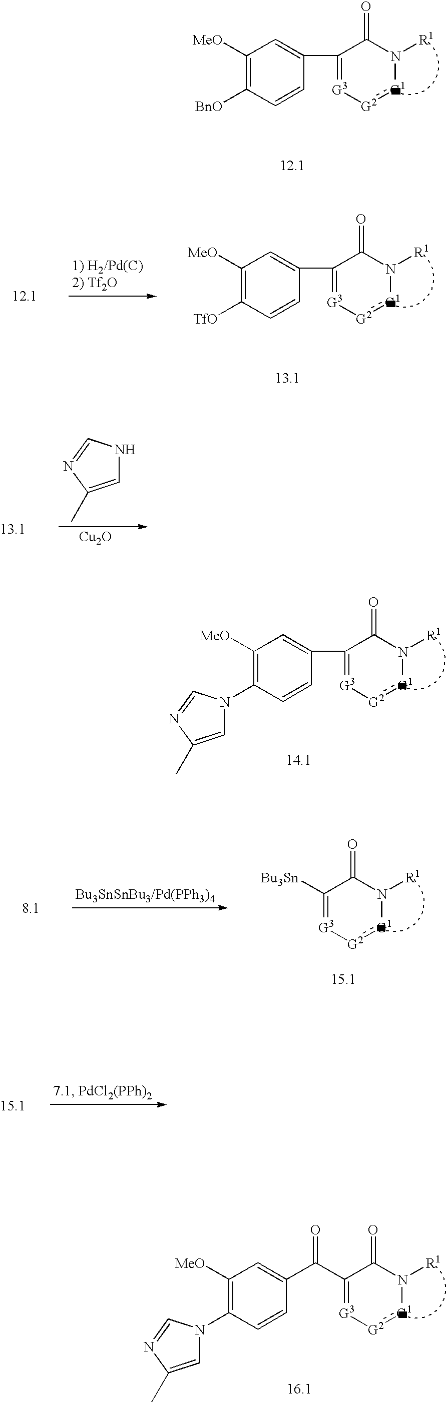

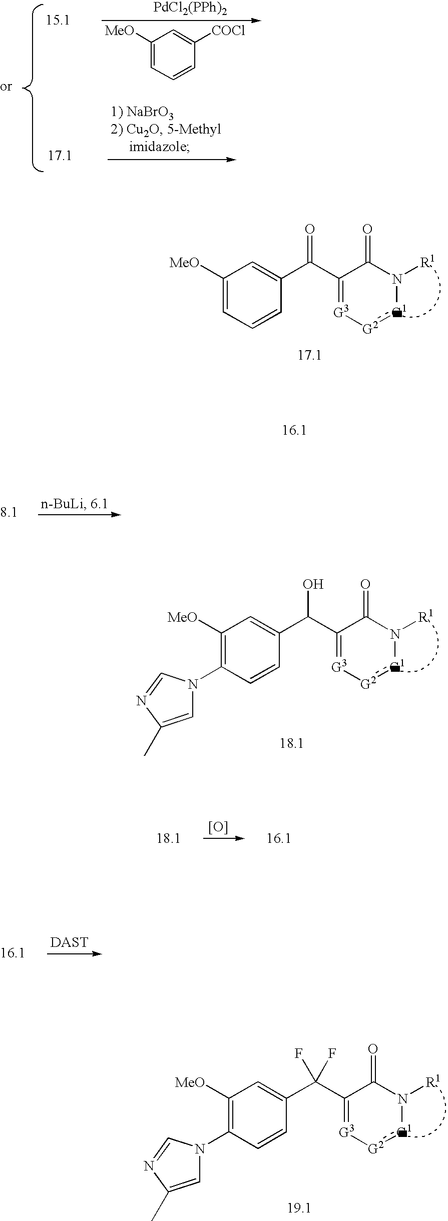

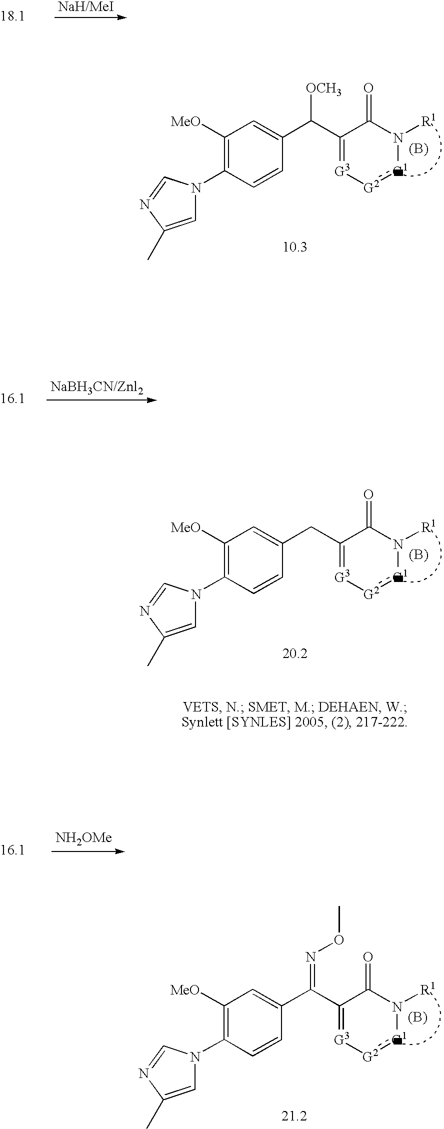

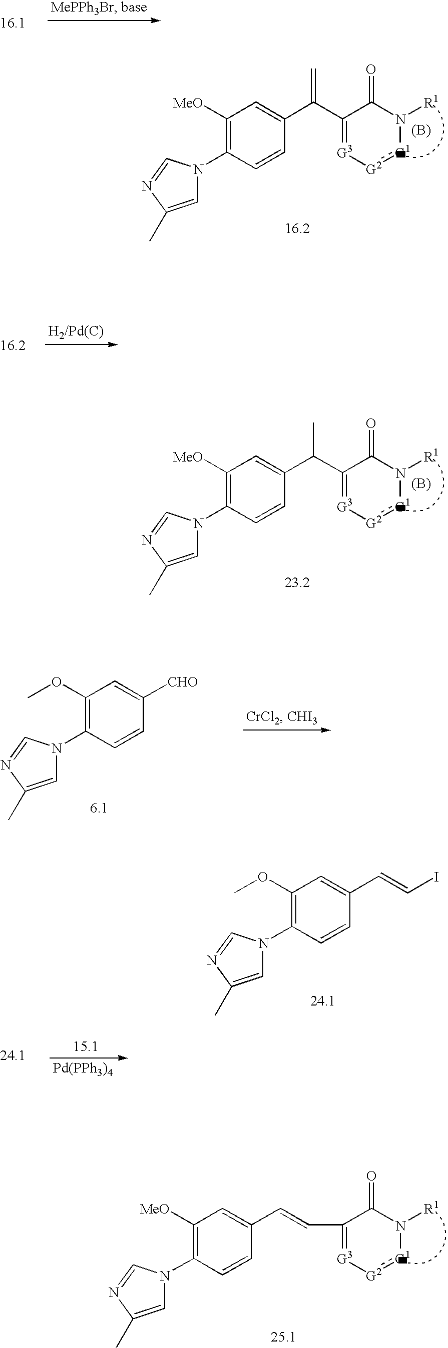

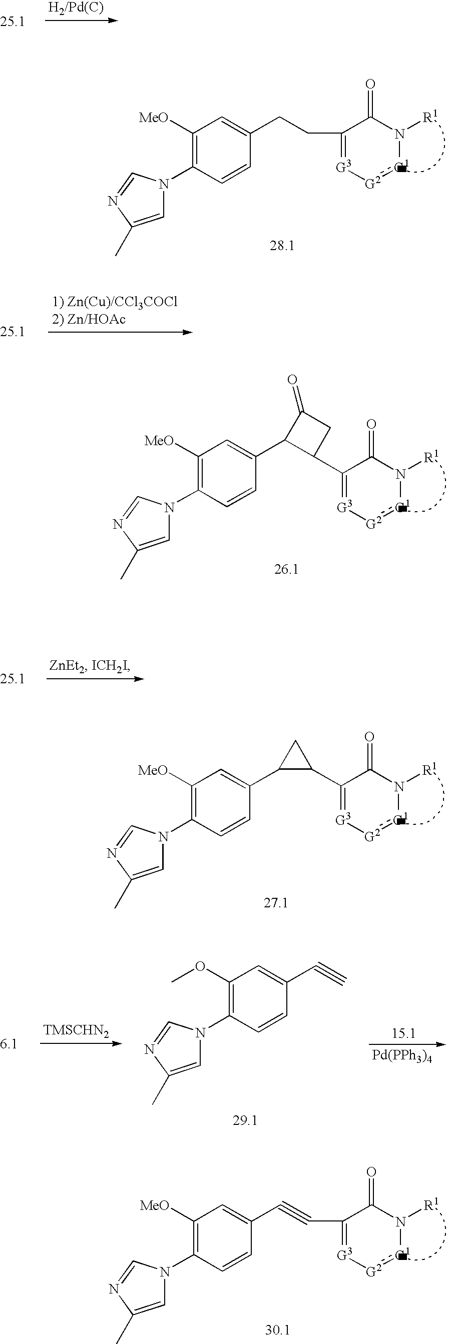

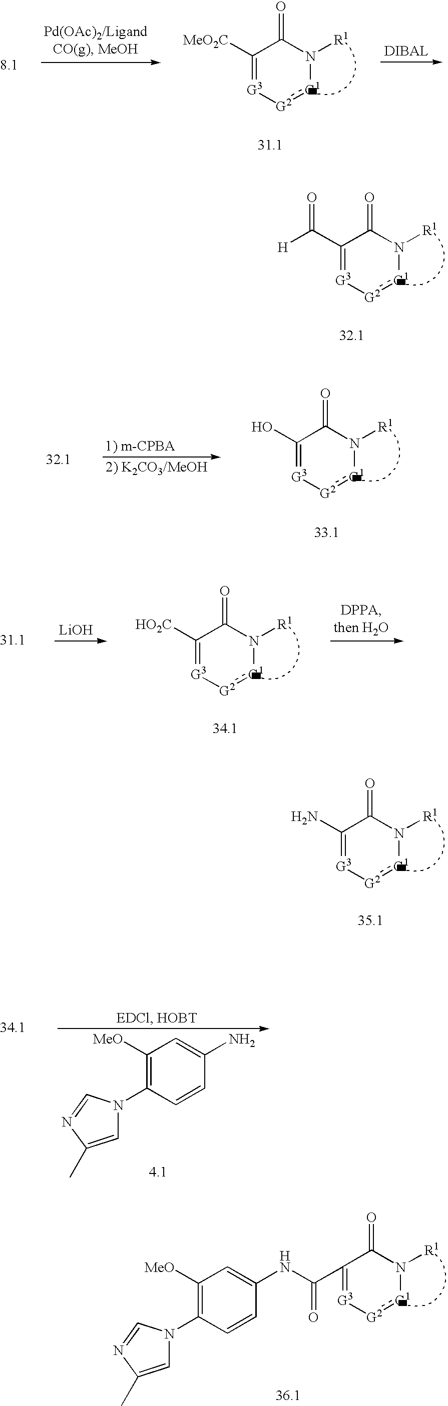

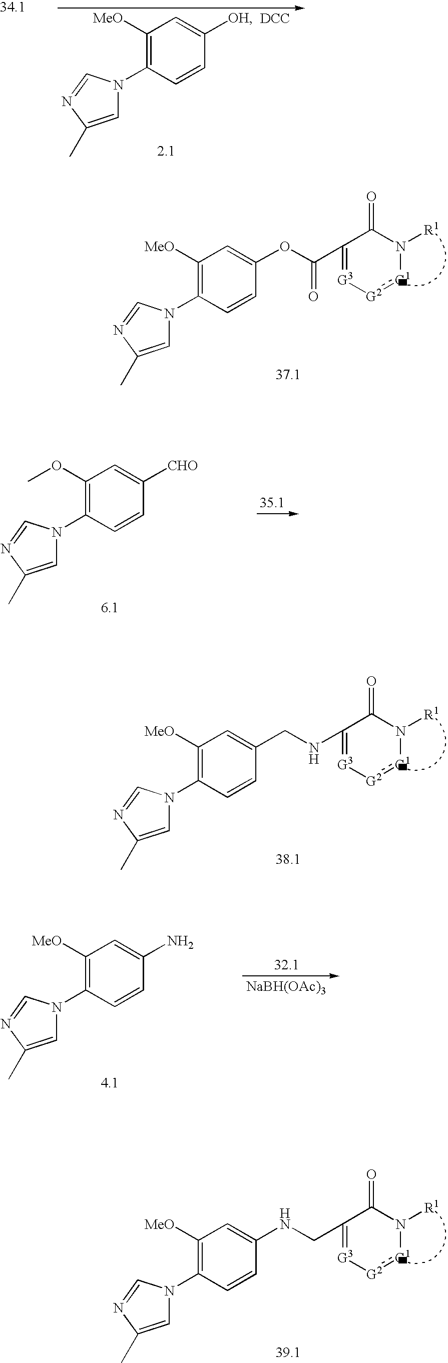

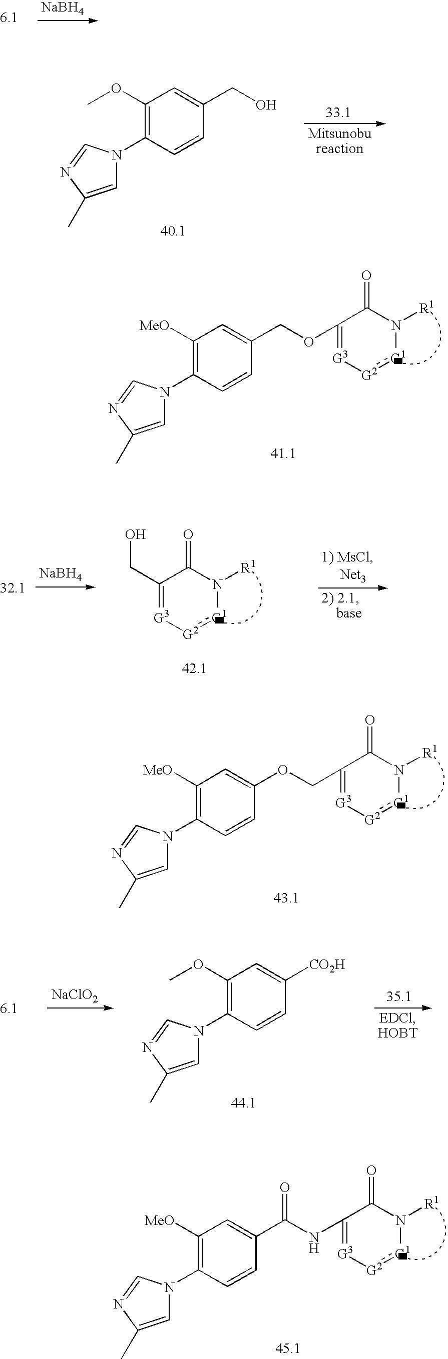

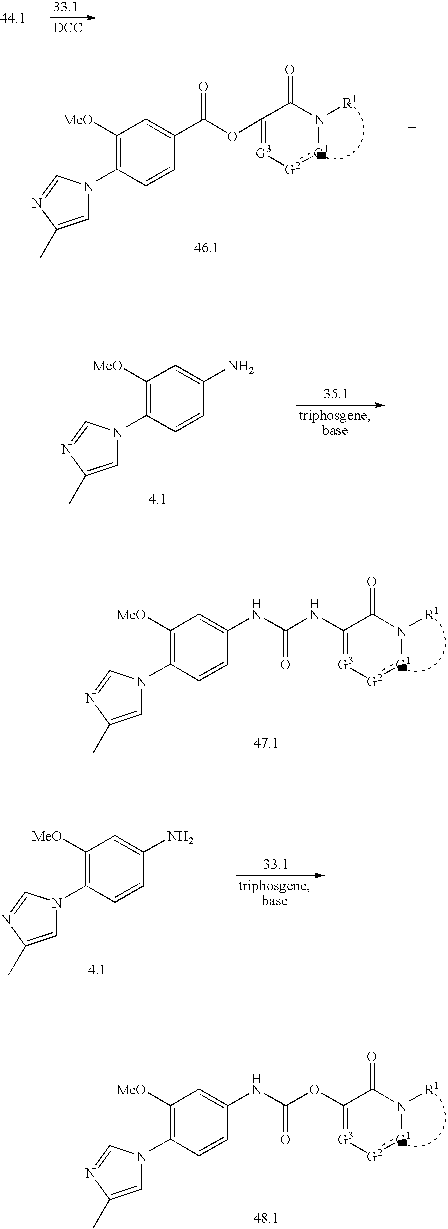

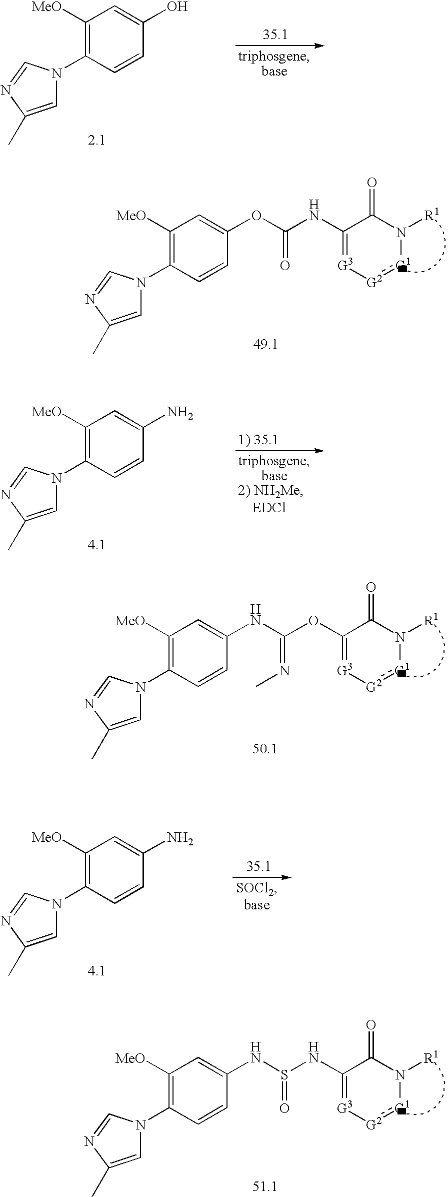

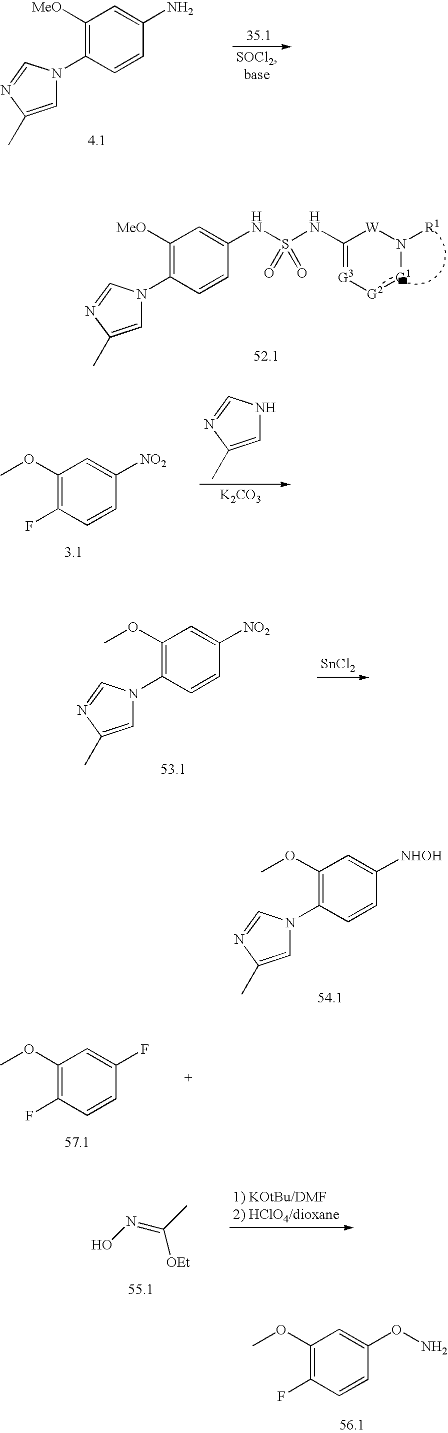

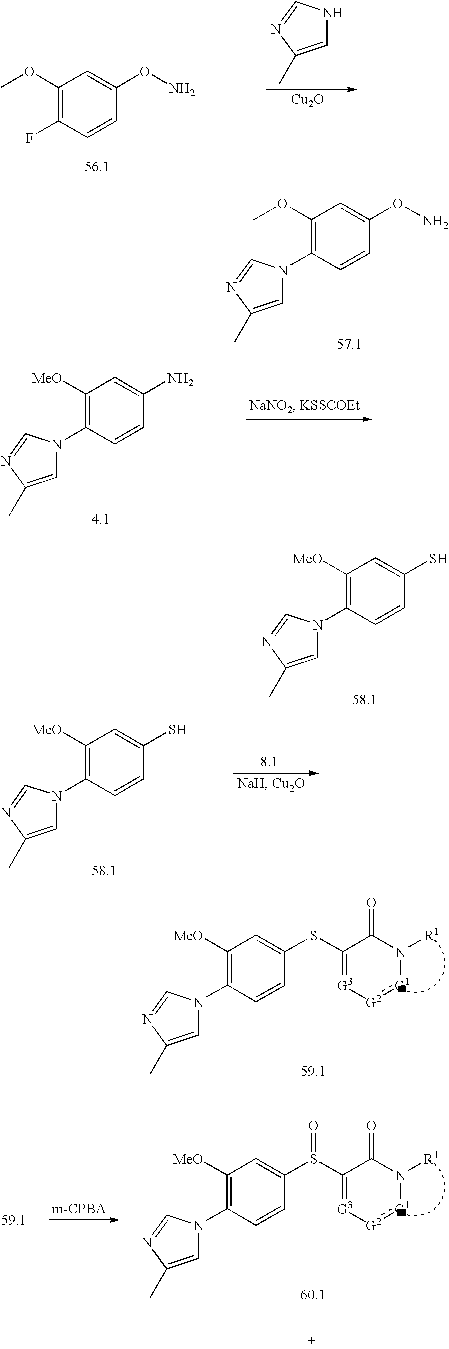

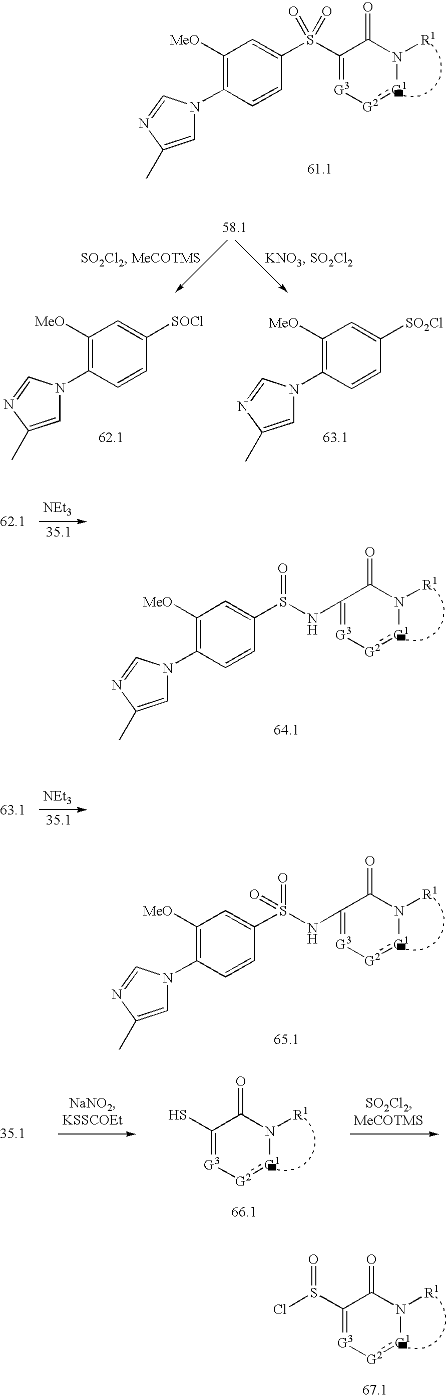

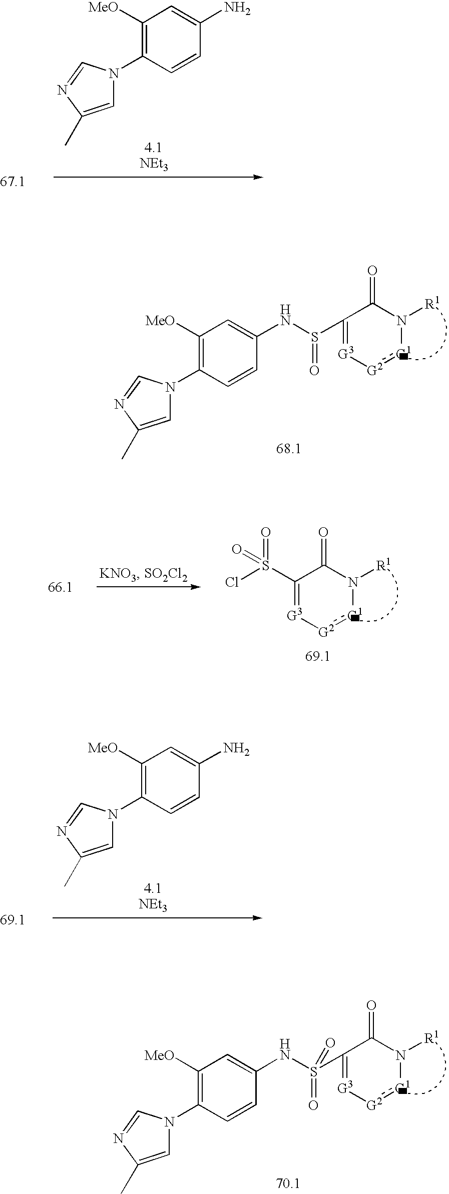

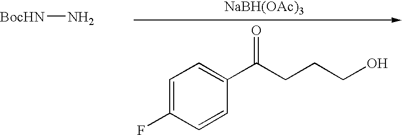

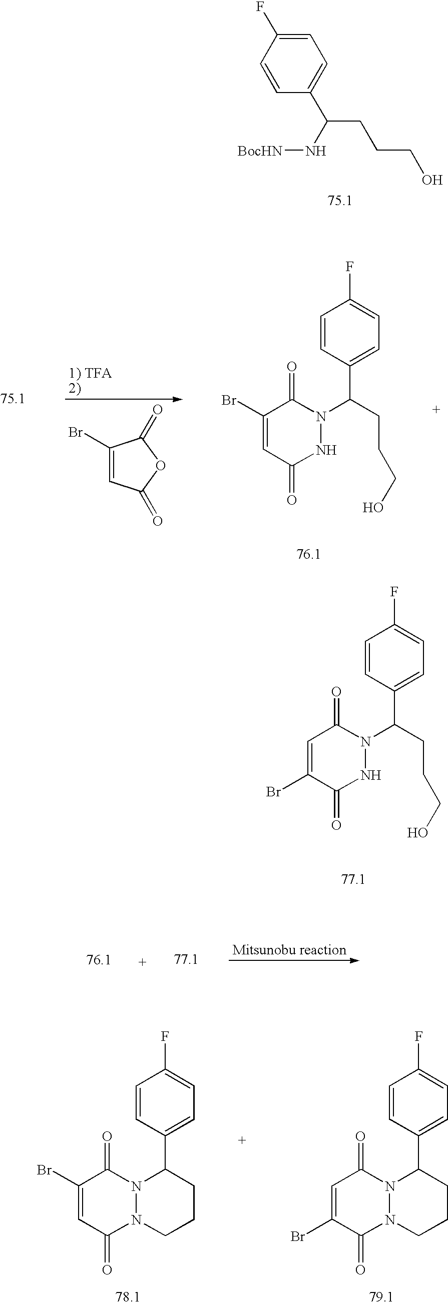

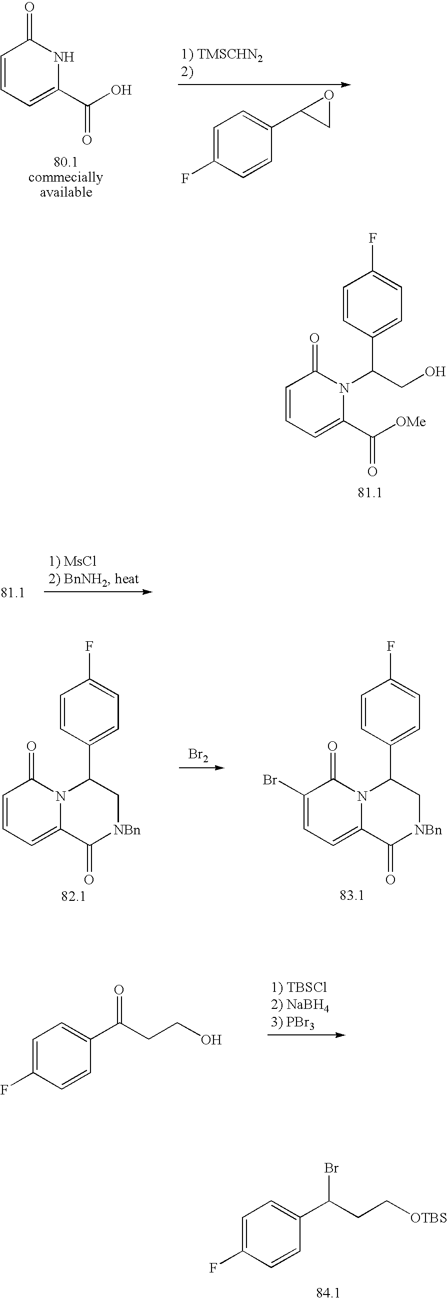

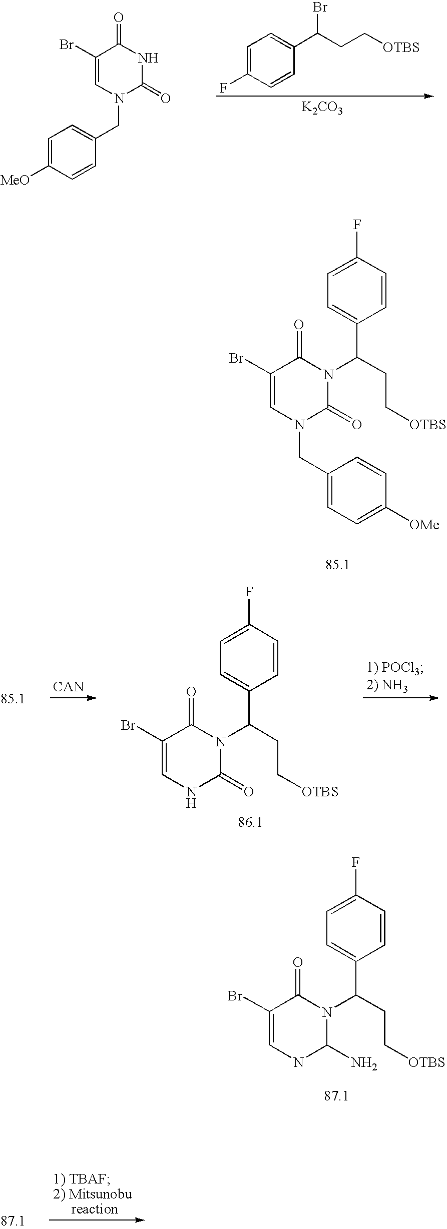

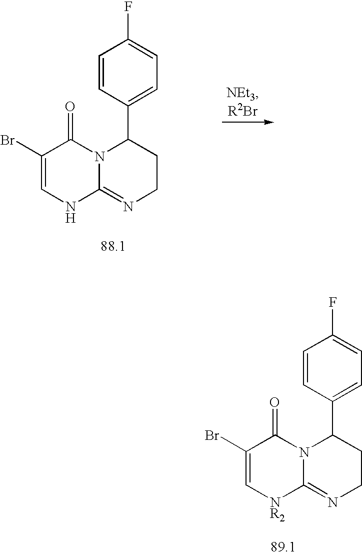

### Example 1

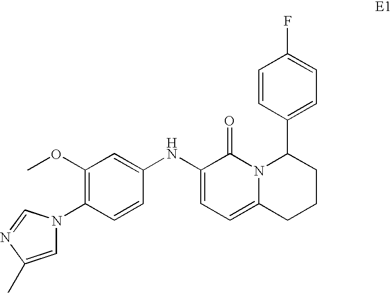

### Step A:

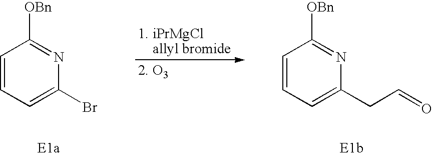

iPrMgCl.LiCl (39.3 mL, 1.3 M) was added to a solution of E1a (4.5 g) in THF (50 mL) at room temperature. The mixture was stirred for 8 hours before allylbromide (7.35 mL) was added dropwise. The reaction mixture was stirred at room temperature overnight. The mixture was diluted with EA (300 mL) and NH₄Cl solution (50 mL). The organic layer was washed with water, brine, dried over MgSO₄, and concentrated to give the crude product which was purified by column chromatography eluting with EtOAc/hexanes to yield allyl intermediate. This intermediate was taken up in CH₂Cl₂ and MeOH (v/v=75 mL/50 mL) and ozonized with an O₃ generator for 20 minutes before it was blowed with O₂ for 5 minutes until the blue color disappeared. The reaction was quenched with Me₂S (5 eq) and the mixture was stirred for 30 minutes. The mixture was diluted with EA (200 mL) and water (50 mL). The organic layer was washed with water, brine, dried over MgSO₄, and concentrated to give the crude product E1b which was used directly for next step without further purification.

### Step B:

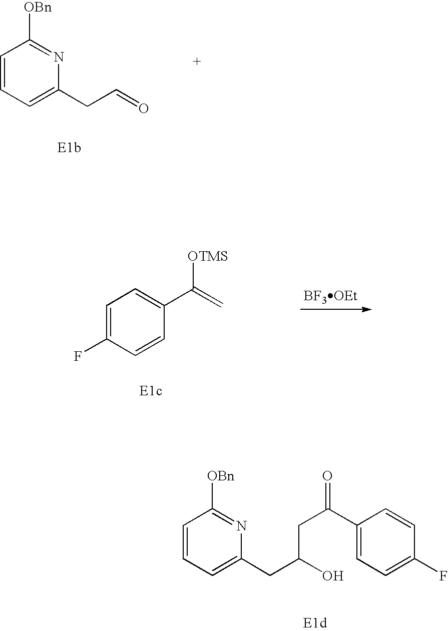

BF₃.OEt₂ (10 eq, 10.8 mL) was added dropwise to a mixture of compound E1b (8.5 mmol) and Etc (2.5 eq, 4.4 g, prepared from corresponding ketone and TMSOTf.) in CH₂Cl₂ (100 mL) containing 4A MS at −78° C. The mixture was kept at this low temperature overnight before it was diluted with EtOAc (200 mL) and careful addition of NaHCO₃ solution (50 mL). The organic layer was washed with water, brine, dried over MgSO₄, and concentrated to give the crude product which was purified by column chromatography eluting with EtOAc/hexanes to yield compound E1c (1.2 g).

### Step C:

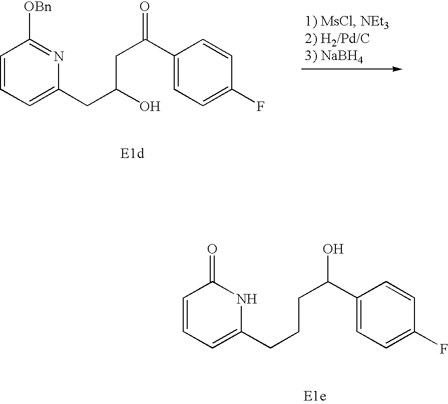

MsCl (0.31 mL, 2.5 eq) was added to a solution of compound E1d (0.59 g) and NEt₃ (0.9 mL, 4 eq) in CH₂Cl₂ (6.0 mL) at 0° C. The mixture was stirred for 1 hour before it was diluted with CH₂Cl₂ (100 mL) and water (40 mL). The organic layer was washed with brine, dried over MgSO₄, and concentrated to give the crude product which was taken up in CH₂Cl₂ and stirred with silica gel for 1 hour. The silica gel was filtered and the filtrate was concentrated to give the alkene intermediate. The alkene intermediate was hydrogenated with a hydrogen balloon with Pd/C (10 wt %) for 3 hours. The mixture was filtered through a celite pad. Solvent was removed and the crude residue was taken up in MeOH, and treated with NaBH₄ (1 eq). The mixture was stirred for 1 hour before it was diluted with EtOAc (100 mL) and NH₄Cl solution (40 mL). The organic layer was washed with water, brine, dried over MgSO₄, and concentrated to give the crude product which was purified by column chromatography eluting with EtOAc/hexanes to yield compound E1e (0.131 g).

### Step D:

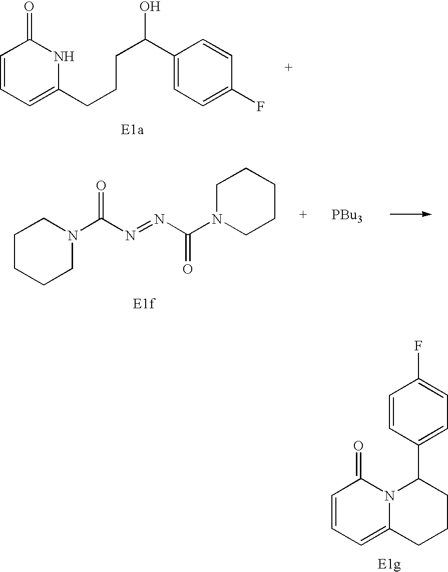

PBu₃ (0.21 mL, 2.0 eq) was added to a solution of compound E1e (0.112 g), E1f (0.21 g, 2.0 eq) in THF (4 mL) at room temperature. The resulting mixture was then heated at 80° C. for 2 hours. The mixture was diluted with EtOAc (100 mL) and NaHCO₃ solution (20 mL). The organic layer was washed with brine, dried over MgSO₄, and concentrated to give the crude product which was purified by column chromatography eluting with EtOAc/hexanes to yield compound E1g (0.1 g).

### Step E:

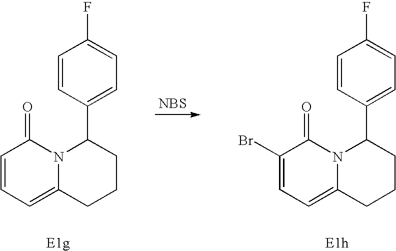

NBS (36.6 mg, 1.0 eq) was added to a solution of compound E1g (50 mg) in DMF (1.2 mL) at room temperature. The mixture was stirred overnight before it was diluted with EtOAc (50 mL) and careful addition of Na₂S₂O₃ solution (10 mL). The organic layer was washed with water, brine, dried over MgSO₄, and concentrated to give the crude product which was purified by column chromatography eluting with EtOAc/hexanes to yield compound E1h (20 mg).

### Step F:

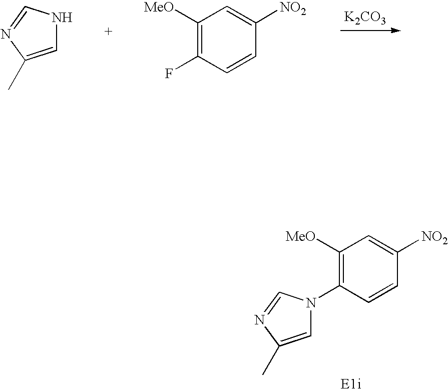

Two equivalent of 4-methylimidazole, 1 equivalent of 3-methoxy-4-fluoro-nitrobenzene and 5 eq. of K₂CO₃ were stirred in CH₃CN at room temperature over night. The reaction mixture was filtered and concentrated under reduced pressure. The crude product was recrystallized with EtOAc to give desired product E1i.

### Step G:

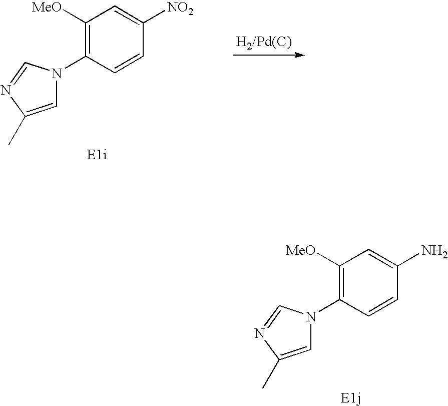

Compound E1i was hydrogenated with hydrogen balloon in the presence of Pd(C) as the catalyst (10 wt %) in MeOH over night. The mixture was filtered and concentrated under reduced pressure to give product E1j.

### Step H:

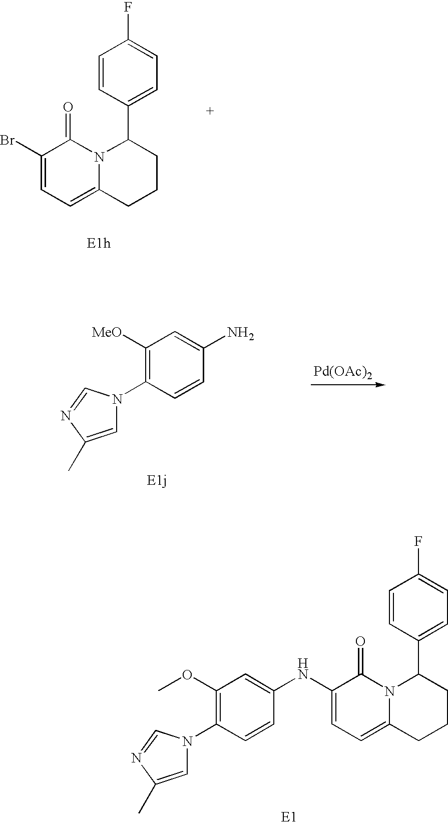

A mixture of compound E1 h (0.118 mmol), E1j (0.118 mmol), Pd(OAc)₂ (1.06 mg, 0.00472 mmol), BINAP (2.94 mg, 0.00472 mmol) and K₂CO₃ (81.4 mg, 0.59 mmol) in toluene will be vacuum/nitrogen exchange degassed for 3 times before it will be heated at 120° C. for 48 hours. The reaction mixture will be cooled and will be diluted with EtOAc (50 mL) and NH₄CJ solution (10 mL). The organic layer will be washed with water, brine, and will be dried over MgSO₄, and will be concentrated to give the crude product. The crude residue will be purified by Gilson reverse phase HPLC to yield Compound E1.

### Example 2

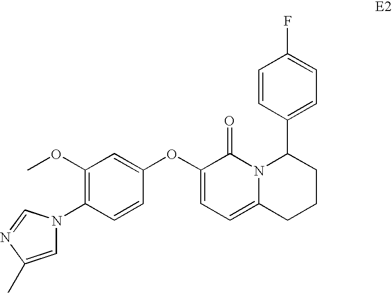

### Step A:

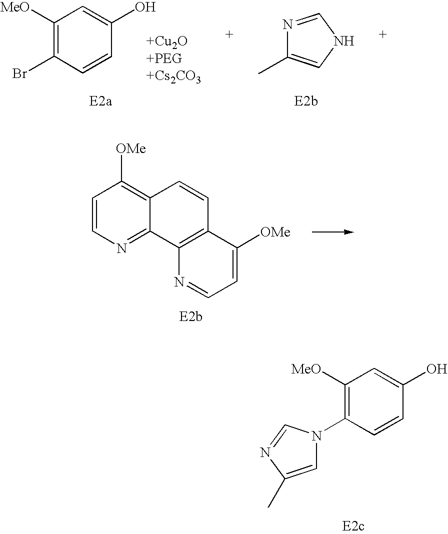

A mixture of compound E2a (2.03 g, 10 mmol), Cu₂O (0.288 g, 2 mmol), PEG (4.0), Cs₂CO₃ (9.77 g, 30 mmol), 4-methylimidazole (0.98 g, 12 mmol) and E2b (0.72 g, 3 mmol) in NMP (15 mL) was vacuum-nitrogen exchange degassed and stirred in a sealed tube at 120° C. for 48 hours. The mixture was cooled to room temperature and diluted with CH₂Cl₂ followed with addition of silica gel. The mixture was stirred for 20 minutes and filtered. The organic layer was washed with water (3×), brine, dried over MgSO₄, and concentrated to give the crude product. The crude residue was purified by column chromatography eluting with CH₂Cl₂/MeOH to yield compound E2 (0.2 g).

### Step B:

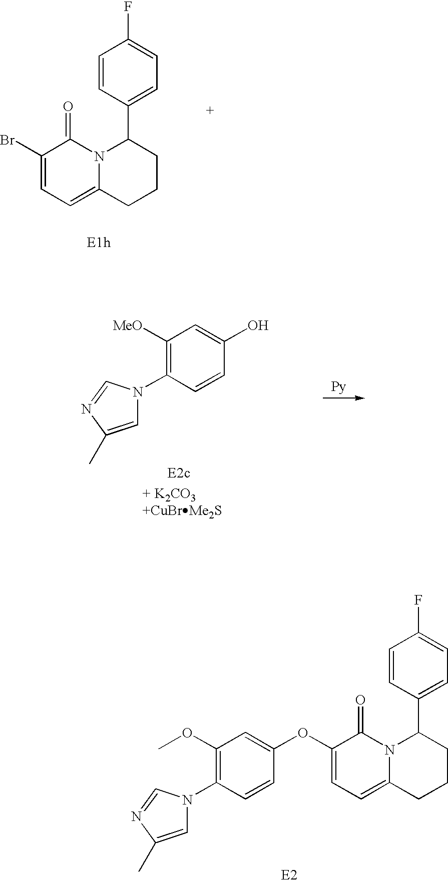

A mixture of compound E1h (0.141 mmol), E2 (28.8 mg, 0.141 mmol), K₂CO₃ (0.117 g, 0.846 mmol) and CuBr.Me₂S (58 mg, 0.282 mmol) in pyridine (1.0 mL) will be heated at 140° C. overnight. The mixture will be diluted with EtOAc (50 mL) and NH₄Cl solution (10 mL, saturated). The organic layer will be washed with water, brine, dried over MgSO₄, and concentrated to give the crude product. The crude residue will be purified by Gilson reverse phase HPLC to yield compound E2.

### Example 3

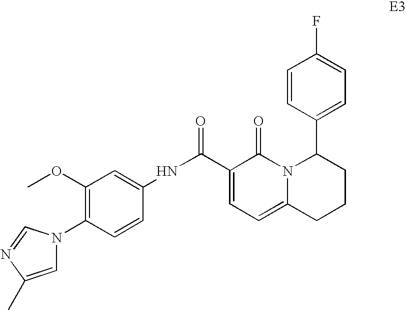

### Step A:

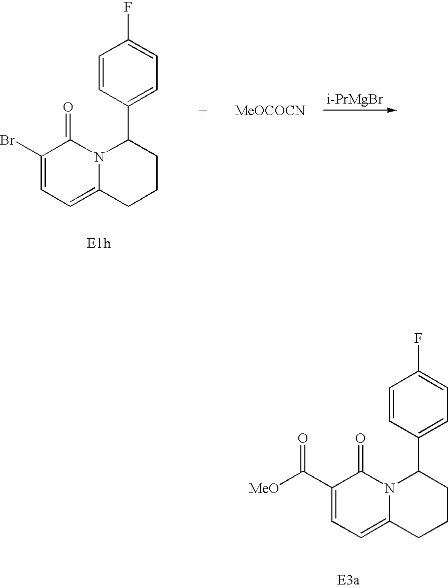

To a solution of compound E1 h (1 eq) in THF will be added ⁱPrMgCl.LiCl (1M in THF, 1.3 eq) and will be stirred for 20 min. This will be followed with addition of methyl cyanoformate (1.0 eq). The resulting mixture will be stirred at RT for 2 hours, then it will be diluted with saturated aqueous NH₄Cl, will be extracted with EtOAc, will be dried (Na₂SO₄), will be concentrated and will be purified by silica gel flash chromatography (Hex/EtOAc) to afford compound E3a.

### Step B

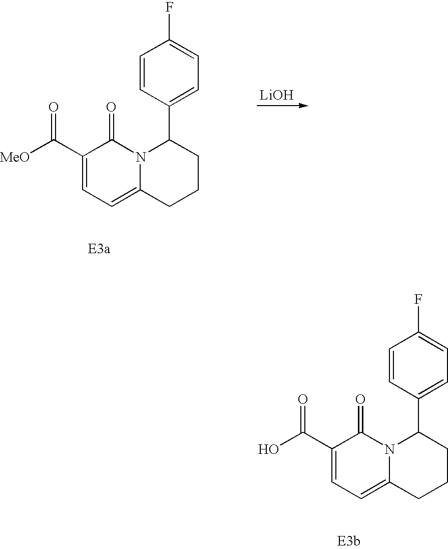

E3a will be hydrolysed with LiOH in water/MeOH/THF to give E3b.

### Step C:

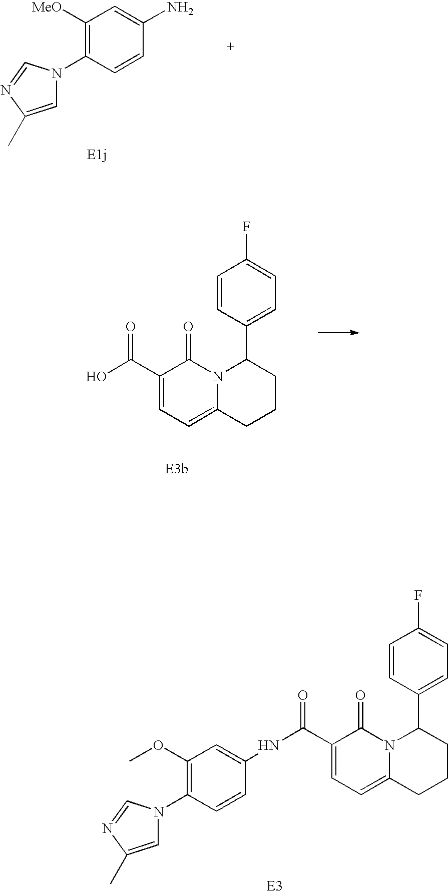

Compound E1j (0.596 mmol) and E3b (0.596 mmol) will be mixed in DCM (4 ml) at RT, which will then be followed by the addition of HOBT (96 mg, 0.715 mmol), EDC (136 mg, 0.715 mmol) and DIEA (300 μL, 1.2 mmol). The resultant mixture will be kept stirring at RT for 16 h. The mixture will be diluted with CH₂Cl₂ (10 ml), will be washed with NaHCO₃ (Sat) (6 ml), and brine (6 ml), respectively, will be dried over anhydrous MgSO₄, and will be concentrated. The residue will be purified via silica gel column (DCM/MeOH (2N NH₃)=30:1), which will be followed by PTLC (DCM/MeOH (2N NH₃)=20:1) to give E3.

### Assay:

Secretase Reaction and Aβ Analysis in Whole Cells: HEK293 cells overexpressing APP with Swedish and London mutations is treated with the specified compounds for 5 hour at 37° C. in 100 ml of DMEM medium containing 10% fetal bovine serum. At the end of the incubation, total Aβ, Aβ40 and Aβ42 is measured using electrochemiluminescence (ECL) based sandwich immunoassays. Total Aβ is determined using a pair of antibodies TAG-WO2 and biotin-4G8, Aβ40 is identified with antibody pairs TAG-G2-10 and biotin-4G8, while Aβ42 is identified with TAG-G2-11 and biotin-4G8. The ECL signal is measured using Sector Imager 2400 (Meso Scale Discovery).

MS Analysis of Aβ Profile: Aβ profile in conditioned media is determined using surface enhanced laser desorption/ionization (SELDI) mass spectrometry. Conditioned media is incubated with antibody WO2 coated PS20 ProteinChip array. Mass spectra of Aβ captured on the array is read on SELDI ProteinChip Reader (Bio-Rad) according to manufacturer's instructions.

CSF Aβ Analysis: Aβ in rat CSF is determined using MSD technology as described above. Aβ40 is measured using antibody pair Tag-G2-10 and biotin-4G8, while Aβ42 is measured using Tag-anti Aβ42 (Meso Scale Discovery) and biotin-4G8. The ECL signal is measured using Sector Imager 2400 (Meso Scale Discovery).

Matrix-assisted laser desorption/ionization mass spectrometric (MALDI MS) analysis of Aβ is performed on a Voyager-DE STR mass spectrometer (ABI, Framingham, Mass.). The instrument is equipped with a pulsed nitrogen laser (337 nm). Mass spectra is acquired in the linear mode with an acceleration voltage of 20 kV. Each spectrum presented in this work represents an average of 256 laser shots. To prepare the sample-matrix solution, 1 of immunoprecipitated Aβ sample is mixed with 3 μL of saturated α-cyano-4-hydroxycinnamic acid solution in 0.1% TFA/acetonitrile. The sample-matrix solution is then applied to the sample plate and dried at ambient temperature prior to mass spectrometric analysis. All the spectra are externally calibrated with a mixture of bovine insulin and ACTH (18-39 clip).

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1-91. (canceled)

92. A compound of the formula (I): 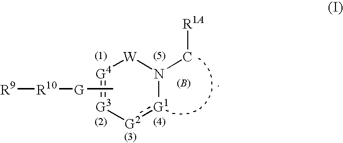
or a pharmaceutically acceptable salt, ester, or solvate thereof, wherein:
R^{1A}, G¹, G², G³, G⁴, (B), G, R⁹, R¹⁰, and W are independently selected;
letters (A) and (B) in formula (I) are reference letters to identify the rings present in formula (I);
the numbers (1), (2), (3), (4), and (5) are reference numbers to identify positions of the Ring (A); G⁴ is at position (1), G³ is at position (2), G² is at position (3), G¹ is at position (4) and the N is at position (5);
the moiety -G-R¹⁰-R⁹ is bound through G to G⁴ or G³, and when G is bound to G⁴ then G⁴ is a —C—, and when G is bound to G³ then G³ is a —C—;
the dotted line between G¹ and G² represents an optional bond;
Ring (B) is the ring formed from the N at position (5) and G¹, and G¹ is carbon or N, and when G¹ is N the optional bond between G¹ and G² is absent;
said Ring (B) is a 4 to 8 membered heterocycloalkyl, heteroaryl, or heterocycloalkenyl ring;
said heterocycloalkyl, heterocycloalkenyl, or a heteroaryl ring Ring (B), in addition to the nitrogen common to Ring (A) and Ring (B), optionally comprises, at least one other heteroatom selected from the group consisting of: —NR²—, —O—, —S—, —S(O)—, and —S(O)₂—;
said Ring (B) is optionally substituted with 1 to 6 independently selected R²¹ substituents;
d is 0 or 1;
m is 0 to 6;
n is 1 to 5;
p is 0 to 5;
q is 0, 1 or 2, and each q is independently selected;
r is 1 to 5;
t is 1 or 2
W is selected from the group consisting of: —C(O)—, —S(O)₂—, —S(O)—, and —C(═NR²)— (and in one example W is —C(O)—);
G is selected from the group consisting of: a direct bond, —C(O)—, —(C═NR²)—, —(C═C(R⁶)₂)—, —CHR³— (e.g., —CHOH), C(R⁴)₂, —CF₂—, —N(R²)—, —O—, —S—, —S(O)ₜ, —CR⁴(OH)—, —CR⁴(OR⁴)—, —C═C—, alkynyl, —(CH₂)ᵣN(R²)—, —(CHR⁴)ᵣN(R²)—, —(C(R⁴)₂)ᵣN(R²)—, —N(R²)(CH₂)ᵣ—, —N(R²)(CHR⁴)ᵣ—, —N(R²)(C(R⁴)₂)ᵣ—, —(CH₂)ᵣ—O—, —(CHR⁴)ᵣ—O—, —(C(R⁴)₂)ᵣ—O—, —O—(CH₂)ᵣ—, —O—(CHR⁴)ᵣ—, —O—(C(R⁴)₂)ᵣ—, —(CH₂)ᵣ—O—C(O)—, —(CHR⁴)ᵣ—O—C(O)—, —(C((R⁴)₂)ᵣ—O—C(O)—, —C(O)—O—(CH₂)ᵣ—, —C(O)—O—(CHR⁴)ᵣ—, —C(O)—O—(C(R⁴)₂)ᵣ—, —C(O)NR⁵—, —O—C(O)—, —C(O)—O—, —O—C(O)—NR⁵—, —NR⁵C(O)—, —(CH₂)ᵣNR⁵—C(O)—, —(CHR⁴)ᵣNR⁵—C(O)—, —(C(R⁴)₂)ᵣNR⁵—C(O)—, —C(O)NR⁵(CH₂)ᵣ—, —C(O)NR⁵ (CHR⁴)ᵣ—, —C(O)NR⁵ (C(R⁴)₂)ᵣ—, —NR⁵S(O)ₜ—, —(CH₂)ᵣNR⁵S(O)ₜ—, —(CHR⁴)ᵣNR⁵S(O)ₜ—, —(C(R⁴)₂)ᵣNR⁵S(O)ₜ—, —S(O)ₜNR⁵—, —S(O)ₜNR⁵(CH₂)ᵣ—, —S(O)ₜNR⁵(CHR⁴)ᵣ—, —S(O)ₜNR⁵(C(R⁴)₂)ᵣ—, —NR⁵—C(O)—O—, —NR⁵—C(O)—NR⁵—, —NR⁵—S(O)ₜ—NR⁵—, —NR⁵—C(═NR²)—NR⁵—, —NR⁵—C(═NR²)—O—, —O—C(═NR²)—NR⁵—, —C(R⁴)═N—O—, —O—N═C(R⁴)—, —O—C(R⁴)═N—, —N═C(R⁴)—O—, —(CH₂)₂₋₃—, —(C(R⁴)₂)₂₋₃—, and —(CHR⁴)₂₋₃—, cycloalkyl, heterocycloalkyl (comprising 1 to 4 heteroatoms independently selected from the group consisting of: —O—, —NR²—, —S—, —S(O)—, and —S(O)₂);
G¹ is selected from the group consisting of:
(1) —C(R²¹)_{q}— wherein q is 0 when the optional bond is present,
(2) —C(R²¹)_{q}— wherein q is 1 when the optional bond is absent,
(3) —CH— when the optional bond is absent, and
(4) —N(R²)_{d}— wherein d is 0, and the optional bond is absent;
G² is selected from the group consisting of: a direct bond, —C(R²¹)_{q}, —N(R²)_{d}—, —C(O)—, S(O), S(O)₂, —C(N(R²)₂)—, and —C(═NR²)—; and with the provisos that:
(1) when the optional bond between G¹ and G² is not present then G² is not —C(N(R²)₂)—, and
(2) when the optional bond between G¹ and G² is present, then:
(a) q for the —C(R²¹)_{q} group is 0 or 1 (and when q is 0 then there is a H on the carbon), and
(b) d for the —N(R²)_{d}— group is 0; and
(c) G² is not a direct bond, —C(O)—, —C(═NR²)—)-, —S(O)₂, or S(O)—;
G³ is selected from the group consisting of: (a) —C(R²¹)_{q} wherein q is 0,
(b) —CH—, (c) —C(R²¹)_{q} wherein q is 1, and (d) —N(R²)_{d} wherein d is 0; and with the proviso that: when moiety G is bound to G³, then G³ is carbon;
G⁴ is selected from the group consisting of: (a) —C(R²¹)_{q} wherein q is 0,
(b) —CH—, (c) —C(R²¹)_{c}, wherein q is 1, and (d) —N(R²)_{d} wherein d is 0; and with the proviso that: when moiety G is bound to G⁴, then G⁴ is carbon; and
provided that 0 to 2 of the G¹, G², G³, and G⁴ moieties are —N(R²)_{d}— and each R² is independently selected and each d is independently selected, and provided that Ring (A) does not have three consecutive ring nitrogen atoms;
R^{1A} is selected from the group consisting of: alkyl-, alkenyl-, alkynyl-, aryl-, arylalkyl-, alkylaryl-, cycloalkyl, cycloalkenyl, cycloalkylalkyl-, fused benzocycloalkyl-, fused benzoheterocycloalkyl-, fused heteroarylcycloalkyl-, fused heteroarylheterocycloalkyl-, fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, fused heterocycloalkylheteroarylalkyl-, heteroaryl-, heteroarylalkyl-, heterocyclyl-, heterocyclenyl-, and heterocyclyalkyl-; wherein each of said alkyl-, alkenyl-, alkynyl-, aryl-, arylalkyl-, alkylaryl-, cycloalkyl, cycloalkenyl, cycloalkylalkyl-, fused benzocycloalkyl, fused benzoheterocycloalkyl, fused heteroarylcycloalkyl, fused heteroarylheterocycloalkyl, fused cycloalkylaryl, fused heterocycloalkylaryl, fused cycloalkylheteroaryl, fused heterocycloalkylheteroaryl, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, fused heterocycloalkylheteroarylalkyl-, heteroaryl, heteroarylalkyl-, heterocyclyl, heterocyclenyl and heterocyclyalkyl- R^{1A} groups is optionally substituted with 1-5 independently selected R²¹ groups;
Each R² is independently selected from the group consisting of: H, —OH, —O-alkyl, —O-(halo substituted alky), —NH(R⁴), —N(R⁴)₂, —NH₂, —S(O)R⁴, S(O)(OR⁴), —S(O)₂R⁴, —S(O)₂(OR⁴), —S(O)NHR⁴, —S(O)N(R⁴)₂, —S(O)NH₂, —S(O)₂NHR⁴, —S(O)₂N(R⁴)₂, —S(O)₂NH₂, —CN, —C(O)₂R⁴, —C(O)NHR⁴, —C(O)N(R⁴)₂, —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;
R³ is selected from the group consisting of: H, —OH, halo, —O-alkyl, —O-(halo substituted alky), —NH(R⁴), —N(R⁴)₂, —NH₂, —S(R⁴), —S(O)R⁴, —S(O)(OR⁴), —S(O)₂R⁴, —S(O)₂(OR⁴), —S(O)NHR⁴, —S(O)N(R⁴)₂, —S(O)NH₂, —S(O)₂NHR⁴, —S(O)₂N(R⁴)₂, —S(O)₂NH₂, —CN, —C(O)₂R⁴, —C(O)NHR⁴, —C(O)N(R⁴)₂, —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;
each R⁴ is independently selected from the group consisting of: unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;
each R⁵ is independently selected from the group consisting of: H, unsubstituted alkyl, substituted alkyl, unsubstituted alkenyl, substituted alkenyl, unsubstituted alkynyl, substituted alkynyl, unsubstituted cycloalkyl, substituted cycloalkyl, unsubstituted aryl, substituted aryl, unsubstituted heteroaryl and substituted heteroaryl; wherein said substituted groups are substituted with one or more substituents independently selected from: R²;
Each R⁶ is independently selected from the group consisting of: H, halo, —CF₃, —O-alkyl, —O-(halo substituted alky), —S(O)R⁴, —S(O)(OR⁴), —S(O)NHR⁴, —S(O)N(R⁴)₂ (wherein each R⁴ is independently selected), —S(O)NH₂, —S(O)₂NHR⁴, —S(O)₂N(R⁴)₂ (wherein each R⁴ is independently selected), —S(O)₂NH₂, —C(═NOR²⁴)R²⁵, and —S(O)₂R²⁴; —CN, —C(O)₂R⁴, —C(O)NHR⁴, —C(O)N(R⁴)₂ (wherein each R⁴ is independently selected), —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;
R⁹ is selected from the group consisting of: arylalkoxy-, heteroarylalkoxy-, arylalkylamino-, heteroarylalkylamino-, aryl, arylalkyl-, heteroaryl, heteroarylalkyl-, heterocyclyl, heterocyclenyl, and heterocyclyalkyl-, wherein each of said R⁹ arylalkoxy-, heteroarylalkoxy-, arylalkylamino-, heteroarylalkylamino-, aryl, arylalkyl-, heteroaryl, heteroarylalkyl-, heterocyclyl, heterocyclenyl, and heterocyclyalkyl- is optionally substituted with 1-5 independently selected R²¹ groups;
R¹⁰ is selected from the group consisting of: aryl-, heteroaryl-, cycloalkyl-, cycloalkenyl, cycloalkylalkyl-, heterocyclyl-, heterocyclenyl-, heterocyclylalkyl-, heterocyclylalkenyl-, fused benzocycloalkyl-, fused benzoheterocycloalkyl-, fused heteroarylcycloalkyl-, fused heteroarylheterocycloalkyl-, fused cycloalkylaryl, fused heterocycloalkylaryl-, fused cycloalkylheteroaryl-, fused heterocycloalkylheteroaryl-, 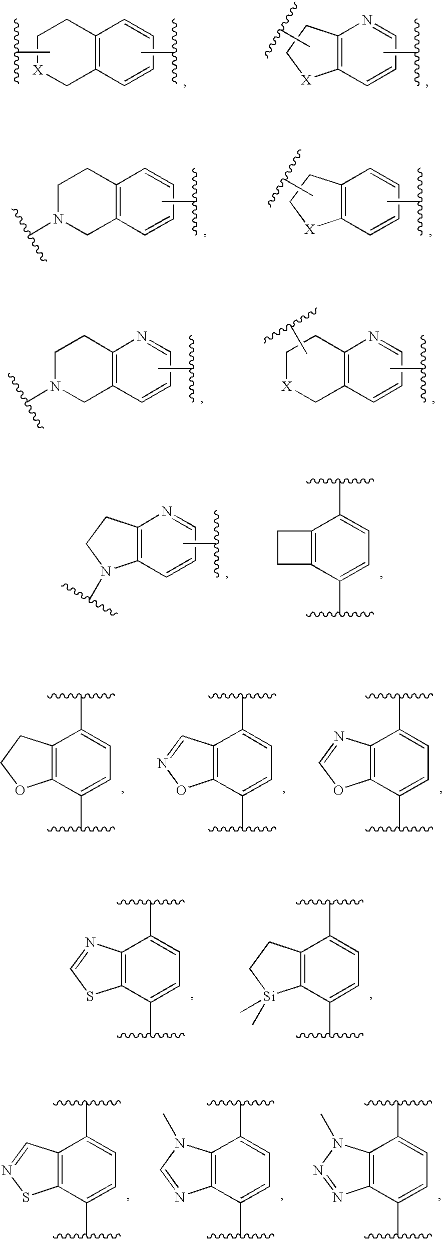 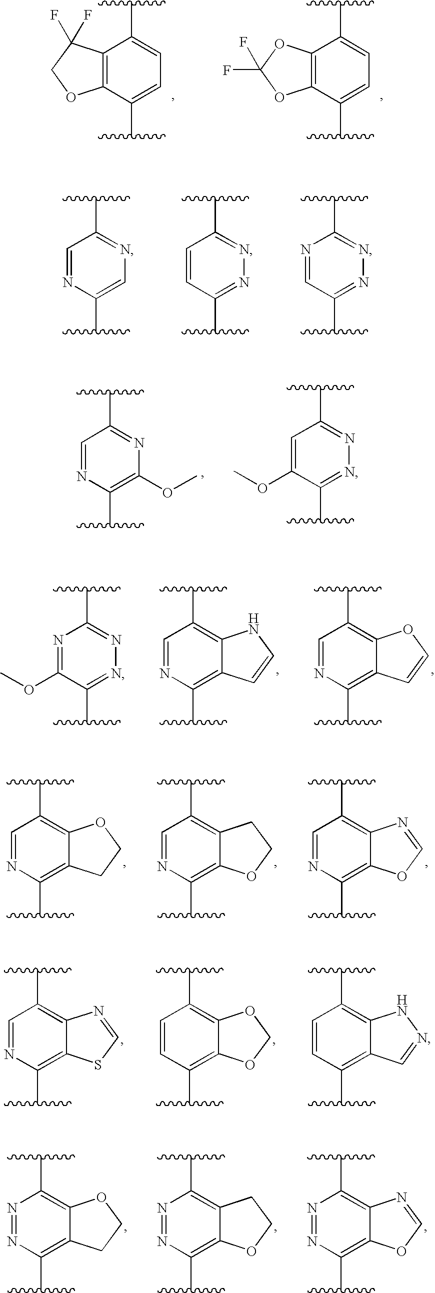 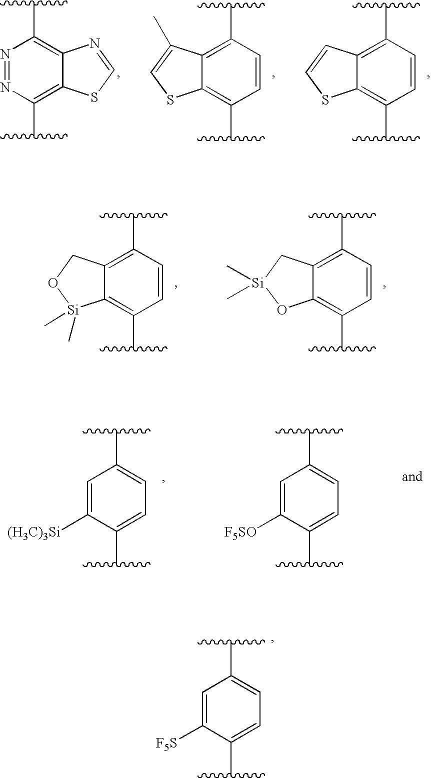
wherein X is selected from the group consisting of: O, —N(R¹⁴)— and —S—; and wherein each of said R¹⁰ moieties is optionally substituted with 1-5 independently selected R²¹ groups; or
R⁹ and R¹⁰ are linked together to form a fused tricyclic ring system wherein R⁹ and R¹⁰ are as defined above and the ring linking R⁹ and R¹⁰ is an alkyl ring, or a heteroalkyl ring, or an aryl ring, or a heteroaryl ring, or an alkenyl ring, or a heteroalkenyl ring (for example, the tricyclic ring system is formed by linking the atoms adjacent to the atoms by which R³ and R⁴ are bound together);
R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclylalkenyl-, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶, —C(═NOR¹⁵)R¹⁶, and —P(O)(OR¹⁵)(OR¹⁶);
R^{15A}, and R^{16A} are independently selected from the group consisting of alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁶)ₙ-heteroaryl and (R¹⁸)ₙ-heteroarylalkyl;
R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl;
each R¹⁸ is independently selected from the group consisting of alkyl, alkenyl, alkynyl, aryl, arylalkyl, arylalkenyl, arylalkynyl, —NO₂, halo, heteroaryl, HO-alkoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or
two R¹⁸ moieties on adjacent carbons can be linked together to form a 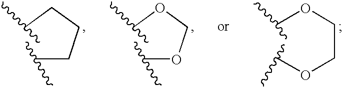
R¹⁹ is selected from the group consisting of: alkyl, cycloalkyl, aryl, arylalkyl and heteroarylalkyl;
R²⁰ is selected from the group consisting of: alkyl, cycloalkyl, aryl, halo substituted aryl, arylalkyl, heteroaryl and heteroarylalkyl;
each R²¹ is independently selected from the group consisting of: alkyl, alkenyl, alkynyl, cycloalkyl, cycloalkylalkyl, cycloalkenyl, heterocycloalkyl, ═O, ═N—R², heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —P(O)(CH₃)₂, —SO(═NR¹⁵)R¹⁶—, —SF₅, —OSF₅, —Si(R^{15A})₃ wherein each R^{15A} is independently selected —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁶)C(O)N(R¹⁶)(R¹⁷), —CH₂—R¹⁵; —CH₂N(R¹⁵)(R¹⁶), —N(R¹⁵)S(O)R^{16A}, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R^{15A}, ═NOR¹⁵, —N₃, —NO₂, —S(O)₂R^{15A}, —O—N═C(R⁴)₂ (wherein each R⁴ is independently selected), and —O—N═C(R⁴)₂ wherein R⁴ is taken together with the carbon atom to which they are bound to form a 5 to 10 membered ring, said ring optionally containing 1 to 3 heteroatoms selected from the group consisting of —O—, —S—, —S(O)—, —S(O)₂—, and —NR²—; wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups;
each R²² group is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R^{16A}, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R^{15A} and —S(O)₂R^{15A}; and
provided that:
when W is —(C═O)—, and G is bound to G⁴, and when G¹, G², G³, and G⁴ are the same or different —C(R²¹)_{q}— moiety, and G is —CHR³—, then R³ is not H, halo, unsubstituted aryl, substituted aryl, unsubstituted heteroaryl, substituted heteroaryl, unsubstituted arylalkyl, substituted arylalkyl, unsubstituted heteroarylalkyl, substituted heteroarylalkyl, unsubstituted alkyl, substituted alkyl, or —O-alkyl; and
when R²¹ is bound to a carbon that has three other filled valences then R²¹ is not ═O, ═NR², or ═NOR¹⁵; and
when G is bound to G⁴, and G¹ is a C(R²¹)_{q} group, and the carbon of said G¹ group is bound to a ═N— in Ring B, and the optional bond between G¹ and G² is not present, and G² is N(R²)_{d}, and G³ is —C(R²¹)_{q}—, then G is not CHR³; and
when G is bound to G⁴, and G¹ is a C(R²¹)_{q} group, and the carbon of said G¹ group is bound to an —NR²— in Ring B, and the optional bond between G¹ and G² is present, and G² is N(R²)_{d}, and G³ is —C(R²¹)_{q}—, then G is not CHR³; and
when G is bound to G⁴, and G¹ is a C(R²¹)_{q} group, and the carbon of said G¹ group is bound to a ═N— in Ring B, and G² is a direct bond, and G³ is N, then G is not CHR³.

93. The compound of claim 92, wherein R⁹ is selected from the group consisting of: 1gg to 13gg.

94. The compound of claim 92, wherein the R⁹-R¹⁰- moiety is selected from the group consisting of: 1bb to 40bb.

95. The compound of claim 92, wherein R^{1A} is selected from the group consisting of:

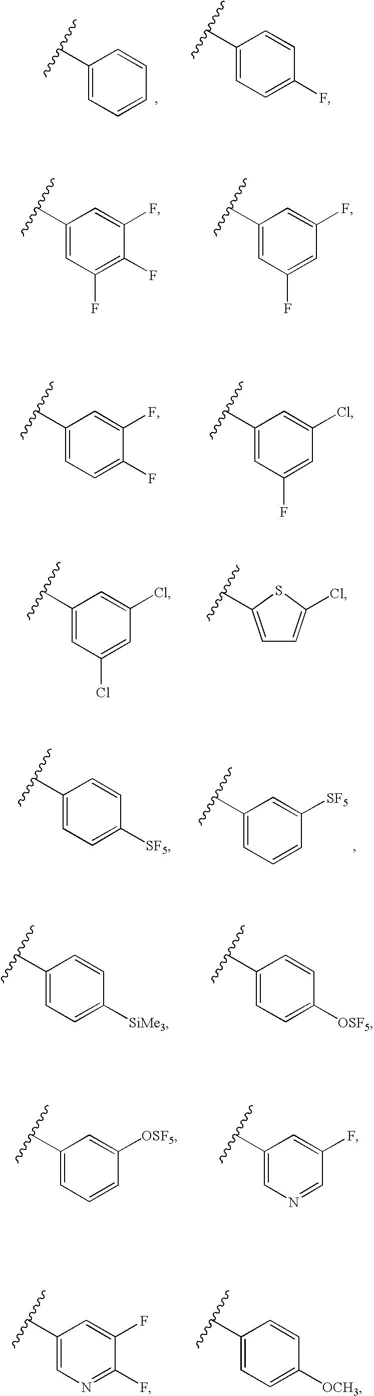

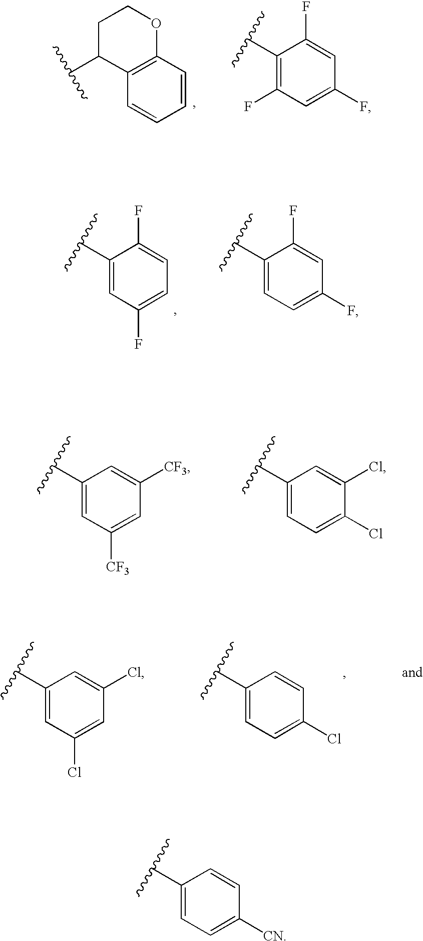

96. The compound of claim 92, wherein the R⁹-R¹⁰- moiety is:

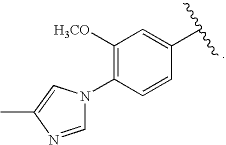

97. The compound of claim 92, wherein said R¹⁰ is selected from the group consisting of aryl and aryl substituted with one or more R²¹ groups, and said R⁹ group is selected from the group consisting of heteroaryl and heteroaryl substituted with one or more R²¹ groups, wherein each R²¹ is independently selected.

98. The compound of claim 92, wherein said R¹⁰ is phenyl substituted with one R²¹ group, and said R⁹ is imidazolyl substituted with one R²¹ group, wherein each R²¹ is independently selected.

99. The compound of claim 92, wherein:
(a) the R⁹-R¹⁰- moiety is: 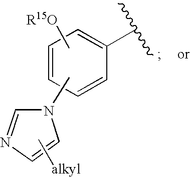
(b) the R⁹-R¹⁰- moiety is: 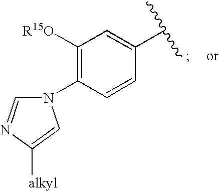
(c) the R⁹-R¹⁰- moiety is: 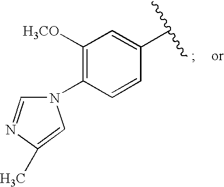
(d) the R⁹-R¹⁰- moiety is: 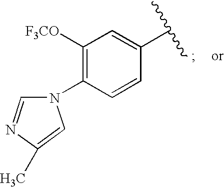
(e) the R⁹-R¹⁰- moiety is: 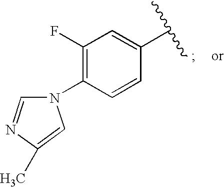
(f) the R⁹-R¹⁰- moiety is:

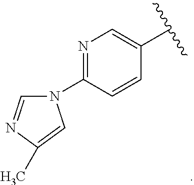

100. The compound of claim 92, wherein
R^{1A} is an unsubstituted aryl or an aryl substituted with one or more independently selected R²¹ groups; or
R^{1A} is phenyl, or
R^{1A} is phenyl, and said phenyl is substituted with 1 to 3 independently selected R²¹ groups, or
R^{1A} is phenyl, and said phenyl is substituted with 1 to 3 R²¹ groups, and each R²¹ group is the same or different halo, or
R^{1A} is phenyl, and said phenyl is substituted with three R²¹ halo groups, and each R²¹ group is the same or different halo, or
R^{1A} is phenyl, and said phenyl is substituted with two R²¹ halo groups, and each R²¹ group is the same or different halo, or
R^{1A} is phenyl, and said phenyl is substituted with one R²¹ halo group,
R^{1A} is phenyl, and said phenyl is substituted with one F, or
R^{1A} is phenyl, and said phenyl is substituted with two F atoms, or
R^{1A} is phenyl, and said phenyl is substituted with three F atoms.

101. The compound of claim 92, wherein said R^{1A} is selected from the group consisting of:

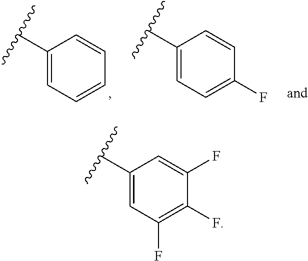

102. The compound of claim 92, wherein said R¹⁰ is selected from the group consisting of heteroaryl and heteroaryl substituted with one or more R²¹ groups, and said R⁹ group is selected from the group consisting of heteroaryl and heteroaryl substituted with one or more R²¹ groups, and wherein each R²¹ is independently selected.

103. The compound of claim 92, wherein:
(1) (a) R^{1A} is an aryl group, or R^{1A} is an aryl group substituted with 1 to 3 independently selected R²¹ groups, and (b) R¹⁰ is selected from the group consisting of aryl and aryl substituted with one or more independently selected R²¹ groups, and (c) R⁹ is selected from the group consisting of heteroaryl and heteroaryl substituted with one or more independently selected R²¹ groups, or
(2) (a) R^{1A} is phenyl, or R^{1A} is phenyl substituted with 1 to 3 independently selected R²¹ groups, and (b) R¹⁰ is selected from the group consisting of aryl and aryl substituted with one or more independently selected R²¹ groups, and (c) R⁹ is selected from the group consisting of heteroaryl and heteroaryl substituted with one or more independently selected R²¹ groups, or
(3) (a) R^{1A} is phenyl, or R^{1A} is phenyl substituted with 1 to 3 independently selected R²¹ groups, and (b) R¹⁰ is selected from the group consisting of phenyl and phenyl substituted with one or more independently selected R²¹ groups, and (c) R⁹ is selected from the group consisting of imidazolyl and imidazolyl substituted with one or more independently selected R²¹ groups, or
(4) (a) R^{1A} is phenyl, or R^{1A} is phenyl substituted with 1 to 3 independently selected R²¹ halo groups, and (b) R¹⁰ is selected from the group consisting of phenyl and phenyl substituted with one or more independently selected —OR¹⁵ groups, and (c) R⁹ is selected from the group consisting of imidazolyl and imidazolyl substituted with one or more independently selected alkyl groups groups, or
(5) (a) R^{1A} is phenyl, or R^{1A} is phenyl substituted with 1 to 2 independently selected R²¹ halo groups, and (b) R¹⁰ is selected from the group consisting of phenyl and phenyl substituted with one or two independently selected —OR¹⁵ groups, wherein R¹⁵ is alkyl, and (c) R⁹ is selected from the group consisting of imidazolyl and imidazolyl substituted with one or two independently selected alkyl groups groups, or
(6) (a) R^{1A} is phenyl, or R^{1A} is phenyl substituted with 1 R²¹ halo group, and (b) R¹⁰ is selected from the group consisting of phenyl and phenyl substituted with one or two independently selected —OR¹⁵ groups, wherein R¹⁵ is alkyl, and (c) R⁹ is selected from the group consisting of imidazolyl and imidazolyl substituted with one or two independently selected alkyl groups groups, or
(7) (a) R^{1A} is phenyl, or R^{1A} is phenyl, substituted with 1 to 3 F, and (b) R¹⁰ is selected from the group consisting of phenyl and phenyl substituted with one or two independently selected —OR¹⁵ groups, wherein R¹⁵ is methyl, and (c) R⁹ is selected from the group consisting of imidazolyl and imidazolyl substituted with one or two independently selected methyl groups groups, or
(8) (a) R^{1A} is phenyl, or R^{1A} is phenyl, substituted with 1 to 2 F, and (b) R¹⁰ is selected from the group consisting of phenyl and phenyl substituted with one or two independently selected —OR¹⁵ groups, wherein R¹⁵ is methyl, and (c) R⁹ is selected from the group consisting of imidazolyl and imidazolyl substituted with one or two independently selected methyl groups groups.
(9) (a) R^{1A} is phenyl, or R^{1A} is phenyl, substituted with 1 F, and (b) R¹⁰ is selected from the group consisting of phenyl and phenyl substituted with one or two independently selected —OR¹⁵ groups, wherein R¹⁵ is methyl, and (c) R⁹ is selected from the group consisting of imidazolyl and imidazolyl substituted with one or two independently selected methyl groups groups, or
(10) R^{1A} is selected from the group consisting of: 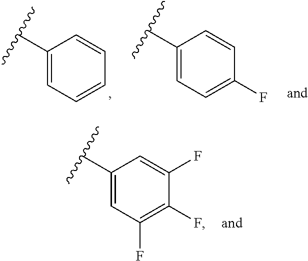
wherein the R⁹-R¹⁰- moiety is: 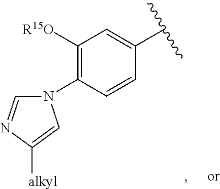
(11) R^{1A} is selected from the group consisting of: 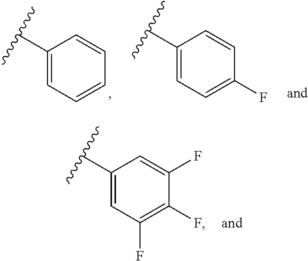
wherein the R⁹-R¹⁰- moiety is: 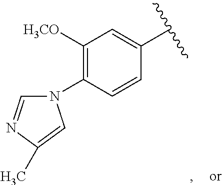
(12) R^{1A} is selected from the group consisting of: 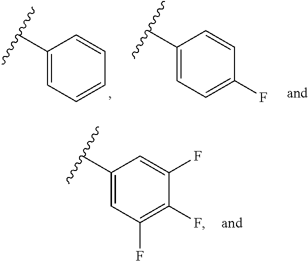
wherein the R⁹-R¹⁰- moiety is: 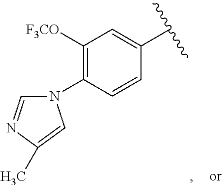
(13) R^{1A} is selected from the group consisting of: 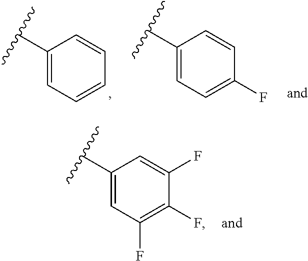
wherein the R⁹-R¹⁰- moiety is: 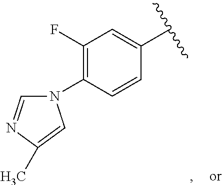
(14) R^{1A} is selected from the group consisting of: 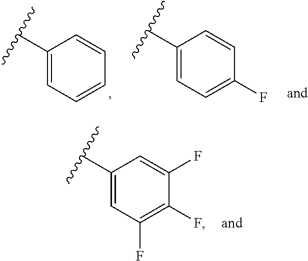
wherein the R⁹-R¹⁰- moiety is:

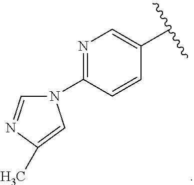

104. The compound of claim 92, wherein:
the R⁹-R¹⁰- moiety is: 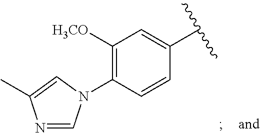
G is selected from the group consisting of: —C(O)—, —CH₂—, —C(CH₃)—, —(CHOH)—, —C(OCH₃)—, —C(═NOCH₃)—, —(C═NR²)—, —(C═C(R⁶)₂)—, —CHR³—, —NH—, —O—, —S—, —S(O)—, —S(O)₂— and a direct bond, and W is —C(O)—.

105. The compound of claim 92 selected from the group consisting of compounds of the formula: (IA) wherein the optional bond between G¹ and G² is absent, (IA) wherein the optional bond between G¹ and G² is present, (IB) wherein the optional bond between G¹ and G² is absent, (IB) wherein the optional bond between G¹ and G² is present, IC to IH, 2 to 9, 12 to 18, 20, 21, 40 to 43, 55, 2A to 9A, 12A to 18A, 20A, 21A, 40A to 43A, 55A, 2B to 9B, 12B to 18B, 20B, 21B, 40B to 43B, 55B, 2C to 9C, 12C to 18C, 20C, 21C, 40C to 43C, 55C, 6.2, 9.1, 10.1, 10.2, 10.3, 14.1, 16.1, 16.2, 18.1, 19.1, 20.2, 21.2, 23.2, 25.1, 26.1, 27.1, 28.1, 30.1, 36.1, 37.1, 38.1, 39.1, 41.1, 43.1, 45.1, 46.1, 47.1, 48.1, 49.1, 50.1, 51.1, 52.1, 59.1, 60.1, 61.1, 64.1, 65.1, 68.1, 70.1, E1, E2, and E3.

106. The compound of claim 92 selected from the group consisting of: E1, E2, and E3.

107. A pharmaceutical composition:
(1) comprising a therapeutically effective amount of at least one compound of claim 92, or a pharmaceutically acceptable salt, solvate, or ester thereof, and at least one pharmaceutically acceptable carrier, or
(2) comprising a therapeutically effective amount of at least one compound of claim 92, or a pharmaceutically acceptable salt, solvate, or ester thereof, and at least one pharmaceutically acceptable carrier, and an effective amount of one or more BACE inhibitors,
(3) comprising a therapeutically effective amount of at least one compound of claim 92, or a pharmaceutically acceptable salt, solvate, or ester thereof, and at least one pharmaceutically acceptable carrier, and effective amount of one or more cholinesterase inhibitors, or
(4) comprising a therapeutically effective amount of at least one compound of claim 92, or a pharmaceutically acceptable salt, solvate, or ester thereof, and at least one pharmaceutically acceptable carrier, and effective amount of one or more BACE inhibitors, muscarinic antagonists, cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors, or
(5) comprising a therapeutically effective amount of at least one compound of claim 92, or a pharmaceutically acceptable salt, solvate, or ester thereof, and at least one pharmaceutically acceptable carrier, and an effective amount of donepezil hydrochloride.

108. A method for treating Alzheimer's Disease, the method comprising administering to a patient in need thereof a therapeutically effective amount of a compound according to claim 92 or a pharmaceutically acceptable salt thereof.

109. A method for inhibiting the deposition of amyloid protein in, on or around neurological tissue, the method comprising administering to a patient in need thereof an effective amount of a compound according to claim 92.
